SEQUENCE LISTING (1 sequence)

SEQ ID NO: 1 - protein, 12 aa, Artificial Sequence
  Synthetic Construct
VLLPKKSFAEPG

(19) United States
(12) Patent Application Publication
(10) Pub. No.: US 2017/0305882 A1
(43) Pub. Date: Oct. 26, 2017

(54) BENZYL SUBSTITUTED INDAZOLES AS BUB1 KINASE INHIBITORS

(71) Applicant: Bayer Pharma Aktiengesellschaft, Berlin (DE)

(72) Inventors: Anne MENGEL, Berlin (DE); Hans-Georg LERCHEN, Leverkusen (DE); Thomas MÜLLER, Frankfurt (DE); Lars BÄRFACKER, Düsseldorf (DE); Marion HITCHCOCK, Brookline, MA (US); Arwed CLEVE, Berlin (DE); Hans BRIEM, Berlin (DE); Gerhard SIEMEISTER, Berlin (DE); Wilhelm BONE, Berlin (DE); Amaury Ernesto FERNÁNDEZ-MONTÁLVAN, Berlin (DE); Jens SCHRÖDER, Berlin (DE); Ursula MÖNNING, Woltersdorf (DE); Simon HOLTON, Berlin (DE)

(73) Assignee: Bayer Pharma Aktiengesellschaft, Berlin (DE)

(21) Appl. No.: 15/512,473

(22) PCT Filed: Sep. 17, 2015
(86) PCT No.: PCT/EP2015/071332

§ 371 Date: Mar. 17, 2017

(30) Foreign Application Priority Data
Sep. 19, 2014 (EP) 14185603.9

(51) Int. Cl. C07D 401/14; C07D 403/14; A61K 45/06; C07D 405/14; A61K 31/506

(52) U.S. Cl. CPC C07D 401/14; C07D 405/14; A61K 31/506; A61K 45/06; C07D 403/14

(57) ABSTRACT

Compounds of formula (I) and their use as pharmaceuticals.

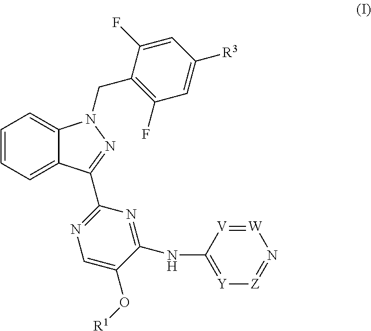

### FIELD OF APPLICATION OF THE INVENTION

The invention relates to substituted Benzyl Substituted Indazole compounds, a process for their production and the use thereof.

### BACKGROUND OF THE INVENTION

One of the most fundamental characteristics of cancer cells is their ability to sustain chronic proliferation whereas in normal tissues the entry into and progression through the cell division cycle is tightly controlled to ensure a homeostasis of cell number and maintenance of normal tissue function. Loss of proliferation control was emphasized as one of the six hallmarks of cancer [Hanahan D and Weinberg R A, Cell 100, 57, 2000: Hanahan D and Weinberg R A, Cell 144, 646, 2011].

The eukaryotic cell division cycle (or cell cycle) ensures the duplication of the genome and its distribution to the daughter cells by passing through a coordinated and regulated sequence of events. The cell cycle is divided into four successive phases:

1. The G1 phase represents the time before the DNA replication, in which the cell grows and is sensitive to external stimuli.
2. In the S phase the cell replicates its DNA, and
3. in the G2 phase preparations are made for entry into mitosis.
4. In mitosis (M phase), the duplicated chromosomes get separated supported by a spindle device built from microtubules, and cell division into two daughter cells is completed.

To ensure the extraordinary high fidelity required for an accurate distribution of the chromosomes to the daughter cells, the passage through the cell cycle is strictly regulated and controlled. The enzymes that are necessary for the progression through the cycle must be activated at the correct time and are also turned off again as soon as the corresponding phase is passed. Corresponding control points (“checkpoints”) stop or delay the progression through the cell cycle if DNA damage is detected, or the DNA replication or the creation of the spindle device is not yet completed. The mitotic checkpoint (also known as spindle checkpoint or spindle assembly checkpoint) controls the accurate attachment of mircrotubules of the spindle device to the kinetochors (the attachment site for microtubules) of the duplicated chromosomes. The mitotic checkpoint is active as long as unattached kinetochores are present and generates a wait-signal to give the dividing cell the time to ensure that each kinetochore is attached to a spindle pole, and to correct attachment errors. Thus the mitotic checkpoint prevents a mitotic cell from completing cell division with unattached or erroneously attached chromosomes [Suijkerbuijk S J and Kops G J, Biochem. Biophys. Acta 1786, 24, 2008; Musacchio A and Salmon E D, Nat. Rev. Mol. Cell. Biol. 8, 379, 2007]. Once all kinetochores are attached with the mitotic spindle poles in a correct bipolar (amphitelic) fashion, the checkpoint is satisfied and the cell enters anaphase and proceeds through mitosis.

The mitotic checkpoint is established by a complex network of a number of essential proteins, including members of the MAD (mitotic arrest deficient, MAD 1-3) and Bub (Budding uninhibited by benzimidazole, Bub 1-3) families, Mps1 kinase, cdc20, as well as other components [reviewed in Bolanos-Garcia V M and Blundell T L, Trends Biochem. Sci. 36, 141, 2010], many of these being over-expressed in proliferating cells (e.g. cancer cells) and tissues [Yuan B et al., Clin. Cancer Res. 12, 405, 2006]. The major function of an unsatisfied mitotic checkpoint is to keep the anaphase-promoting complex/cyclosome (APC/C) in an inactive state. As soon as the checkpoint gets satisfied the APC/C ubiquitin-ligase targets cyclin B and securin for proteolytic degradation leading to separation of the paired chromosomes and exit from mitosis.

Inactive mutations of the Ser/Thr kinase Bub1 prevented the delay in progression through mitosis upon treatment of cells of the yeast S. cerevisiae with microtubule-estabilizing drugs, which led to the identification of Bub1 as a mitotic checkpoint protein [Roberts B T et al., Mol. Cell Biol., 14, 8282, 1994]. A number of recent publications provide evidence that Bub1 plays multiple roles during mitosis which, have been reviewed by Elowe [Elowe S, Mol. Cell. Biol. 31, 3085, 2011]. In particular. Bub1 is one of the first mitotic checkpoint proteins that binds to the kinetochores of duplicated chromosomes and probably acts as a scaffolding protein to constitute the mitotic checkpoint complex. Furthermore, via phosphorylation of histone H2A. Bub1 localizes the protein shugoshin to the centromeric region of the chromosomes to prevent premature segregation of the paired chromosomes [Kawashima et al. Science 327, 172, 2010]. In addition, together with a Thr-3 phosphorylated Histone H3 the shugoshin protein functions as a binding site for the chromosomal passenger complex which includes the proteins survivin, borealin, INCENP and Aurora B. The chromosomal passenger complex is seen as a tension sensor within the mitotic checkpoint mechanism, which dissolves erroneously formed microtubule-kinetochor attachments such as syntelic (both sister kinetochors are attached to one spindle pole) or merotelic (one kinetochor is attached to two spindle poles) attachments [Watanabe Y, Cold Spring Harb. Symp. Quant. Biol. 75, 419, 2010]. Recent data suggest that the phosphorylation of histone H2A at Thr 121 by Bub1 kinase is sufficient to localize AuroraB kinase to fulfill the attachment error correction checkpoint [Ricke et al. J. Cell Biol. 199, 931-949, 2012].

Incomplete mitotic checkpoint function has been linked with aneuploidy and tumourigenesis [Weaver B A and Cleveland D W, Cancer Res. 67, 10103, 2007; King R W, Biochim Biophys Acta 1786, 4, 2008]. In contrast, complete inhibition of the mitotic checkpoint has been recognised to result in severe chromosome missegregation and induction of apoptosis in tumour cells [Kops G J et al., Nature Rev. Cancer 5, 773, 2005; Schmidt M and Medema R H, Cell Cycle 5, 159, 2006; Schmidt M and Bastians H, Drug Res. Updates 10, 162, 2007]. Thus, mitotic checkpoint abrogation through pharmacological Inhibition of components of the mitotic checkpoint, such as Bub1 kinase, represents a new approach for the treatment of proliferative disorders, including solid tumours such as carcinomas, sarcomas, leukaemias and lymphoid malignancies or other disorders, associated with uncontrolled cellular proliferation.

The present invention relates to chemical compounds that Inhibit Bub1 kinase.

Established anti-mitotic drugs such as vinca alkaloids, taxanes or epothilones activate the mitotic checkpoint, inducing a mitotic arrest either by stabilising or destabilising microtubule dynamics. This arrest prevents separation of the duplicated chromosomes to form the two daughter cells. Prolonged arrest in mitosis forces a cell either into mitotic exit without cytokinesis (mitotic slippage or adaption) or into mitotic catastrophe leading to cell death [Rieder C L and Maiato H, Dev. Cell 7, 637, 2004]. In contrast, inhibitors of Bub1 prevent the establishment and/or functionality of the mitotic checkpoint and/or microtubule-kinetochor attachment error correction mechanisms, which finally results in severe chromosomal missegregation, induction of apoptosis and cell death.

These findings suggest that Bub1 inhibitors should be of therapeutic value for the treatment of proliferative disorders associated with enhanced uncontrolled proliferative cellular processes such as, for example, cancer, inflammation, arthritis, viral diseases, cardiovascular diseases, or fungal diseases in a warm-blooded animal such as man.

WO 2013/050438, WO 2013/092512, WO 2013/167698 disclose substituted benzylindazoles, substituted benzylpyrazoles and substituted benzylcycloakylpyrazoles, respectively, which are Bub1 kinase inhibitors.

Furthermore, WO 2014/147203, WO 2014/147204, WO2014202590, WO2014202588, WO2014202584, WO2014202583, and WO2015/063003, disclose substituted indazoles, substituted pyrazoles, and substituted cycloakylpyrazoles, which are Bub1 kinase inhibitors.

Due to the fact that especially cancer disease as being expressed by uncontrolled proliferative cellular processes in tissues of different organs of the human- or animal body still is not considered to be a controlled disease in that sufficient drug therapies already exist, there is a strong need to provide further new therapeutically useful drugs, preferably inhibiting new targets and providing new therapeutic options (e.g. drugs with improved pharmacological properties, such as improved target Bub1 inhibition potency).

### DESCRIPTION OF THE INVENTION

Therefore, Inhibitors of Bub1 represent valuable compounds that should complement therapeutic options either as single agents or in combination with other drugs.

In accordance with a first aspect, the invention relates to compounds of formula (I),

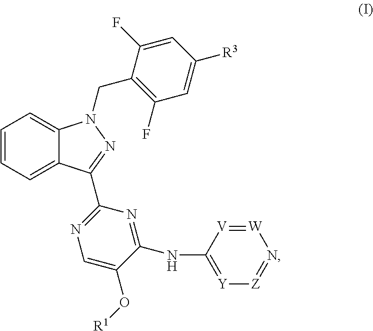

in which
V, W, Y and Z independently of each other represent CH or CR², wherein one of V, W, Y and Z represents CR², or,
V represents N, and W, Y and Z independently of each other represent CH or CR², or,
V and Y represent N, and W and Z independently of each other represent CH or CR²,
R¹ represents a group selected from:
   C₂-C₆-hydroxyalkyl, and R⁴, wherein said C₂-C₆-hydroxyalkyl group is optionally substituted with one, two or three halogen atoms selected from:
      fluorine, and chlorine,
R² represents, independently of each other, halogen or a group selected from:
   C₁-C₃-alkyl, C₃-C₄-cycloakyl, C₁-C₃-haloalkyl, C₁-C₃-alkoxy, C₁-C₃-haloalkoxy, —N(H)C(═O)—(C₁-C₃-alkyl),
   —N(H)C(═O)H, —N(H)C(═O)—(C₁-C₃-hydroxyalkyl), —N(H)C(═O)—(C₁-C₃-alkyl)-(C₁-C₃-alkoxy), —N(H)C(═O)-phenyl, —N(H)C(═O)—(C₃-C₄-cycloalkyl), —N(H)C(═O)—(C₁-C₅-alkyl)-(C₃-C₄-cycloakyl), and —N(H)C(═O)N(H)R⁸,
      said —N(H)C(═O)-phenyl being optionally substituted at the phenyl ring, one, two or three times, identically or differently, with a substituent selected from:
      halogen, hydroxy, cyano, C₁-C₄-alkyl, C₁-C₄-haloalkyl, C₁-C₄-alkoxy, C₁-C₄-haloalkoxy, C₃-C₄-cycloalkyl, and C₃-C₄-cycloalkyloxy,
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy,
R³ represents a group selected from:
   C₁-C₆-alkyl, C₁-C₆-hydroxyakyl, C₁-C₆-haloalkyl, C₃-C₆-cycloalkyl, (C₁-C₃-alkoxy)-(C₁-C₃-alkyl)-, (C₃-C₈-cycloalkyl)-(C₁-C₃-alkyl)-, C₁-C₆-alkoxy, (C₂-C₆-hydroxyakyl)-O—, C₁-C₆-haloalkoxy, C₃-C₆-cycloalkyloxy, (C₁-C₃-alkoxy)-(C₂-C₃-alkoxy)-, and (C₃-C₆-cycloalkyl)-(C₁-C₃-alkoxy)-,
      wherein said C₂-C₆-hydroxyalkyl group is optionally substituted with one, two or three halogen atoms selected from:
      fluorine, and chlorine,
R⁴ represents —(C₂-C₆-alkyl)-OC(═O)—C(H)(R⁵)—N(H)C(═O)—C(H)(R⁷)—NH₂,
   in which C₂-C₆-alkyl is optionally substituted with one, two or three halogen atoms selected from:
   fluorine, and chlorine,
R⁵ and R⁷ independently of each other represent hydrogen (glycine) or a group selected from:
   —CH₃ (alanine), —C(H)(CH₃)₂ (valine), —(CH₂)₂CH₃ (norvaline), —CH₂C(H)(CH₃)₂ (leucine), —C(H)(CH₃)CH₂CH₃ (isoleucine), —(CH₂)₃CH₃ (norleucine), —C(CH₃)₃ (2-tert-butylglycine), benzyl (phenylalanine), 4-hydroxybenzyl (tyrosine), —(CH₂)₃NH₂ (omithine), —(CH₂)₄NH₂ (lysine), —(CH₂)₂C(H)(OH)CH₂NH₂ (hydroxylysine), —CH₂OH (serine), —(CH₂)₂OH (homoserine), —C(H)(OH)CH₃ (threonine), —(CH₂)₂N(H)C(═NH)NH₂ (arginine), —(CH₂)₃N(H)C(═O)NH₂ (citrulline), —CH₂C(═O)NH₂ (asparagine), —CH₂C(═O)OH (aspartic acid), —(CH₂)₂C(═O)OH (glutamic acid), —(CH₂)₂C(═O)NH₂ (glutamine), —CH₂SH (cysteine), —(CH₂)₂SH (homocysteine), —(CH₂)₂SCH₃ (methionine), —CH₂SCH₃ (S-methylcysteine), (1H-imidazol-4-yl)methyl-(histidine), (1H-indol-3-yl)methyl-(thryptophan), —CH₂NH₂ (2,3-diaminopropanoic acid), and —(CH₂)₂NH₂ (2,4-diaminobutanoic acid),
R⁸ represents hydrogen or a group selected from:
   C₁-C₃-alkyl, C₁-C₃-haloalkyl, C₂-C₃-hydroxyalkyl, C₃-C₄-cycloakyl, (C₃-C₄-cycloalkyl)-(C₁-C₃-alkyl)-, and (C₁-C₃-alkoxy)-(C₂-C₃-alkyl)-,
      or an N-oxide, a salt, a tautomer or a stereoisomer of said compound, or a salt of said N-oxide, tautomer or stereoisomer.

Another aspect of the invention are compounds of formula (I) as defined herein,

wherein
V, W, Y and Z independently of each other represent CH or CR², wherein one of V, W, Y and Z represents CR²,
   or,
V represents N, and W, Y and Z independently of each other represent CH or CR²,
R¹ represents a group selected from:
   C₂-C₆-hydroxyalkyl, and R⁴,
      wherein said C₂-C₆-hydroxyalkyl group is optionally substituted with one, two or three halogen atoms selected from:
      fluorine, and chlorine,
R² represents, independently of each other, halogen or a group selected from:
   C₁-C₃-alkyl C₃-C₄-cycloakyl, C₁-C₃-haloalkyl, C₁-C₃-alkoxy, C₁-C₃-haloalkoxy, —N(H)C(═O)—(C₁-C₃-alkyl), —N(H)C(═O)H,
   —N(H)C(═O)—(C₁-C₃-hydroxyalkyl), —N(H)C(═O)—(C₁-C₃-alkyl)-(C₁-C₃-alkoxy), —N(H)C(═O)-phenyl, —N(H)C(═O)—(C₃-C₄-cycloalkyl), —N(H)C(═O)—(C₁-C₃-alkyl)-(C₃-C₄-cycloakyl), and —N(H)C(═O)N(H)R⁸,
      said —N(H)C(═O)-phenyl being optionally substituted at the phenyl ring, one, two or three times, identically or differently, with a substituent selected from:
      halogen, hydroxy, cyano, C₁-C₄-alkyl, C₁-C₄-haloalkyl, C₁-C₄-alkoxy, C₁-C₄-haloalkoxy, C₃-C₄-cycloalkyl, and C₃-C₄-cycloalkyloxy,
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy,
R³ represents a group selected from:
   C₁-C₆-hydroxyalkyl, C₁-C₆-alkoxy, (C₂-C₅-hydroxyalkyl)-O—, C₁-C₆-haloalkoxy, and (C₃-C₆-cycloalkyl)-(C₁-C₃-alkoxy)-,
      wherein said C₂-C₅-hydroxyalkyl group is optionally substituted with one, two or three halogen atoms selected from:
      fluorine, and chlorine,
R⁴ represents —(C₂-C₆-alkyl)-OC(═O)—C(H)(R⁵)—N(H)C(═O)—C(H)(R⁷)—NH₂,
   in which C₂-C₆-alkyl is optionally substituted with one, two or three halogen atoms selected from:
   fluorine, and chlorine,
R⁵ and R⁷ independently of each other represent a group selected from:
   —CH₃ (alanine), —C(H)(CH₃)₂ (valine), —(CH₂)₂CH₃ (norvaline), —(CH₂)₃NH₂ (ornithine), —(CH₂)₄NH₂ (lysine), and —(CH₂)₃N(H)C(═NH)NH₂ (arginine),
R⁸ represents hydrogen or a group selected from:
   C₁-C₃-alkyl, C₁-C₃-haloalkyl, C₂-C₃-hydroxyalkyl, C₃-C₄-cycloakyl, (C₃-C₄-cycloalkyl)-(C₁-C₃-alkyl)-, and (C₁-C₃-alkoxy)-(C₂-C₃-alkyl)-,
      or an N-oxide, a salt, a tautomer or a stereoisomer of said compound, or a salt of said N-oxide, tautomer or stereoisomer.

Another aspect of the invention are compounds of formula (I) as defined herein,

wherein
V, W, Y and Z Independently of each other represent CH or CR², wherein one of V, W, Y and Z represents CR²,
   or,
V represents N, and W, Y and Z independently of each other represent CH or CR²,
R¹ represents a group selected from:
   C₂-C₆-hydroxyalkyl, and R⁴,
R² represents, independently of each other, halogen or a group selected from:
   C₁-C₃-alkyl, C₃-C₄-cycloakyl, C₁-C₃-haloalkyl, and
   —N(H)C(═O)—(C₁-C₃-alkyl),
R³ represents a group selected from:
   C₁-C₆-alkoxy, and (C₃-C₆-cycloalkyl)-(C₁-C₃-alkoxy)-.
R⁴ represents —(C₂-C₆-alkyl)-OC(═O)—C(H)(R⁵)—N(H)C(═O)—C(H)(R⁷)—NH₂,
R⁵ and R⁷ independently of each other represent a group selected from:
   —CH₃ (alanine), —(CH₂)₄NH₂ (lysine), and —(CH₂)₃N(H)C(═NH)NH₂ (arginine).
      or an N-oxide, a salt, a tautomer or a stereoisomer of said compound, or a salt of said N-oxide, tautomer or stereoisomer.

Another aspect of the invention are compounds of formula (I) as defined herein,

wherein
V, W, Y and Z independently of each other represent CH or CR², wherein one of V, W, Y and Z represents CR²,
   or,
V represents N, and W, Y and Z independently of each other represent CH or CR²,
R¹ represents a group selected from:
   —(CH₂)₃OH, and R⁴,
R² represents, independently of each other, fluorine, chlorine or a group selected from:
   methyl, cyclopropyl, difluoromethyl, and —N(H)C(═O)—CH₃,
R³ represents a group selected from:
   ethoxy, and cyclopropylmethoxy.
R⁴ represents —(CH₂)₃—OC(═O)—C(H)(R⁵)—N(H)C(═O)—C(H)(R⁷)—NH₂,
R⁵ represents —CH₃ (alanine),
R⁷ represents a group selected from:
   —(CH₂)₄NH₂ (lysine), and —(CH₂)₃N(H)C(═NH)NH₂ (arginine),
      or an N-oxide, a salt, a tautomer or a stereoisomer of said compound, or a salt of said N-oxide, tautomer or stereoisomer.

Another aspect of the invention are compounds of formula (I) as defined herein,

wherein
V, W, Y and Z independently of each other represent CH or CR², wherein one of V, W, Y and Z represents CR²,
   or,
V represents N, and W, Y and Z independently of each other represent CH or CR²,
R¹ represents a group selected from:
   —(CH₂)₃OH, and R⁴,
R² represents, independently of each other, fluorine, chlorine or a group selected from:
   methyl, cyclopropyl, difluoromethyl, 2,2,2-trifluoroethoxy, —N(H)C(═O)—CH₃, and —N(H)C(═O)-(cyclopropyl),
R³ represents a group selected from:
   ethoxy, 2,2-difluoroethoxy, 2,2,2-trifluoroethoxy, 2-methoxyethoxy, and cyclopropylmethoxy,
R⁴ represents —(CH₂)₃—OC(═O)—C(H)(R⁵)—N(H)C(═O)—C(H)(R⁷)—NH₂,
R⁵ represents a group selected from:
   —CH₃ (alanine), and —C(H)(CH₃)₂ (valine),
R⁷ represents a group selected from:
   —(CH₂)₄NH₂ (lysine), and —(CH₂)₃N(H)C(═NH)NH₂ (arginine),
      or an N-oxide, a salt, a tautomer or a stereoisomer of said compound, or a salt of said N-oxide, tautomer or stereoisomer.

In a further aspect of the invention compounds of formula (I) as described above are selected from the group consisting of:
3-[(4-{bis[2-(difluoromethyl)pyridin-4-yl]amino}-2-[1-(4-ethoxy-2,6-difluorobenzyl)-1H-indazol-3-yl]pyrimidin-5-yl)oxy]propan-1-ol,
3-({2-[1-(4-ethoxy-2,6-difluorobenzyl)-1H-indazol-3-yl]-4-[(3-methylpyridin-4-yl)amino]pyrimidin-5-yl}oxy)propan-1-ol,
3-({4-[(3-cyclopropylpyridin-4-yl)amino]-2-[1-(4-ethoxy-2,6-difluorobenzyl)-1H-indazol-3-yl]pyrimidin-5-yl}oxy)propan-1-ol,
3-({2-[1-(4-ethoxy-2,6-difluorobenzyl)-1H-indazol-3-yl]-4-[(2-methylpyridin-4-yl)amino]pyrimidin-5-yl}oxy)propan-1-ol,
N-[4-({2-[1-(4-ethoxy-2,6-difluorobenzyl)-1H-indazol-3-yl]-5-(3-hydroxy-propoxy)pyrimidin-4-yl}amino)pyridin-2-yl]acetamide,
3-({4-[(3-chloropyridin-4-yl)amino]-2-[1-(4-ethoxy-2,6-difluorobenzyl)-1H-indazol-3-yl]pyrimidin-5-yl}oxy)propan-1-ol,
3-({2-[1-(4-ethoxy-2,6-difluorobenzyl)-1H-indazol-3-yl]-4-[(2-methylpyrimidin-4-yl)amino]pyrimidin-5-yl}oxy)propan-1-ol,
3-({2-[1-(4-ethoxy-2,6-difluorobenzyl)-1H-indazol-3-yl]-4-(pyrimidin-4-ylamino)-pyrimidin-5-yl}oxy)propan-1-ol,
3-({2-[1-(4-ethoxy-2,6-difluorobenzyl)-1H-indazol-3-yl]-4-[(2-fluoropyridin-4-yl)amino]pyrimidin-5-yl}oxy)propan-1-ol,
3-({4-[(2,5-dimethylpyridin-4-yl)amino]-2-[1-(4-ethoxy-2,6-difluorobenzyl)-1H-indazol-3-yl]pyrimidin-5-yl}oxy)propan-1-ol,
N-[4-({2-[1-(4-ethoxy-2,6-difluorobenzyl)-1H-indazol-3-yl]-5-(3-hydroxy-propoxy)pyrimidin-4-yl}amino)-5-methylpyridin-2-yl]acetamide,
3-[(4-[(3-chloropyridin-4-yl)amino]-2-{1-[4-(cyclopropylmethoxy)-2,6-difluorobenzyl]-1H-indazol-3-yl}pyrimidin-5-yl)oxy]propan-1-ol,
3-[(4-{[2-(difluoromethyl)pyridin-4-yl]amino}-2-[1-(4-ethoxy-2,6-difluorobenzyl)-1H-indazol-3-yl]pyrimidin-5-yl)oxy]propyl L-lysyl-L-alaninate, salt with trifluoroaceticacid,
3-[(4-{[2-(difluoromethyl)pyridin-4-yl]amino}-2-[1-(4-ethoxy-2,6-difluorobenzyl)-1H-indazol-3-yl]pyrimidin-5-yl)oxy]propyl L-lysyl-L-alaninate,
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-lysyl-L-alaninate, salt with trifluoroaceticacid,
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-lysyl-L-alaninate,
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl D-lysyl-L-alaninate, salt with trifluoroaceticacid,
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl D-lysyl-L-alaninate,
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-arginyl-L-alaninate, salt with trifluoroaceticacid,
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-arginyl-L-alaninate,
3-({4-[(3-chloropyridin-4-yl)amino]-2-[1-(4-ethoxy-2,6-difluorobenzyl)-1H-indazol-3-yl]pyrimidin-5-yl}oxy)propyl L-lysyl-L-alaninate, salt with trifluoroaceticacid,
3-({4-[(3-chloropyridin-4-yl)amino]-2-[1-(4-ethoxy-2,6-difluorobenzyl)-1H-indazol-3-yl]pyrimidin-5-yl}oxy)propyl L-lysyl-L-alaninate,
3-({4-[(2>6-Dimethylpyrimidin-4-yl)amino]-2-[1-(4-ethoxy-2,6-difluorbenzyl)-1H-indazol-3-yl]pyrimidin-5-yl}oxy)propan-1-ol,
N-[4-({2-[1-(4-Ethoxy-2,6-difluorobenzyl)-1H-indazol-3-yl]-5-(3-hydroxypropoxy)-pyrimidin-4-yl}amino)pyridin-2-yl]cyclopropancarboxamide,
3-({2-[1-(4-Ethoxy-2,6-difluorobenzyl)-1H-indazol-3-yl]-4-[(5-fluor-2-methylpyridin-4-yl)-amino]pyrimidin-5-yl}oxy)propan-1-ol,
N-[4-({2-[1-(4-ethoxy-2,6-difluorobenzyl)-1H-indazol-3-yl]-5-(3-hydroxypropoxy)-pyrimidin-4-yl}amino)-3-methylpyridin-2-yl]acetamide,
3-[(2-{1-[2,6-difluoro-4-(2,2,2-trifluoroethoxy)benzyl]-1H-indazol-3-yl}-4-[(2-methylpyrimidin-4-yl)amino]pyrimidin-5-yl)oxy]propan-1-ol,
3-[(4-[(3-chloropyridin-4-yl)amino]-2-{1-[2,6-difluoro-4-(2,2,2-trifluoroethoxy)benzyl]-1H-indazol-3-yl}pyrimidin-5-yl)oxy]propan-1-ol,
3-[(2-{1-[4-(2,2-difluoroethoxy)-2,6-difluorobenzyl]-1H-indazol-3-yl}-4-[(2-methylpyrimidin-4-yl)amino]pyrimidin-5-yl)oxy]propan-1-ol,
3-[(2-[1-(4-ethoxy-2,6-difluorobenzyl)-1H-indazol-3-yl]-4-{[3-(2,2,2-trifluoroethoxy)-pyridin-4-yl]amino}pyrimidin-5-yl)oxy]propan-1-ol,
3-[(2-{1-[4-(cyclopropylmethoxy)-2,6-difluorobenzyl]-1H-indazol-3-yl}-4-[(2,5-dimethylpyridin-4-yl)amino]pyrimidin-5-yl)oxy]propan-1-ol,
3-[(2-{1-[4-(cyclopropylmethoxy)-2,6-difluorobenzyl]-1H-indazol-3-yl}-4-[(2-methylpyrimidin-4-yl)amino]pyrimidin-5-yl)oxy]propan-1-ol,
N-(4-{[2-{1-[2,6-difluoro-4-(2,2,2-trifluoroethoxy)benzyl]-1H-indazol-3-yl}-5-(3-hydroxy-propoxy)pyrimidin-4-yl]amino}pyridin-2-yl)acetamide,
3-[(4-[(3-chloropyridin-4-yl)amino]-2-{1-[4-(2,2-difluoroethoxy)-2,6-difluorobenzyl]-1H-indazol-3-yl}pyrimidin-5-yl)oxy]propan-1-ol,
3-{[2-{1-[2,6-difluoro-4-(2-methoxyethoxy)benzyl]-1H-indazol-3-yl}-4-(pyrimidin-4-yl-amino)pyrimidin-5-yl]oxy}propan-1-ol,
3-({4-[(2,5-dimethylpyridin-4-yl)amino]-2-[1-(4-ethoxy-2,6-difluorobenzyl)-1H-indazol-3-yl]pyrimidin-5-yl}oxy)propyl D-lysyl-L-alaninate, salt with trifluoroaceticacid,
3-({4-[(2,5-dimethylpyridin-4-yl)amino]-2-[1-(4-ethoxy-2,6-difluorobenzyl)-1H-indazol-3-yl]pyrimidin-5-yl}oxy) propyl D-lysyl-L-alaninate,
3-({4-[(2,5-dimethylpyridin-4-yl)amino]-2-[1-(4-ethoxy-2,6-difluorobenzyl)-1H-indazol-3-yl]pyrimidin-5-yl}oxy)propyl L-lysyl-L-alaninate, salt with trifluoroaceticacid,
3-({4-[(2,5-dimethylpyridin-4-yl)amino]-2-[1-(4-ethoxy-2,6-difluorobenzyl)-1H-indazol-3-yl]pyrimidin-5-yl}oxy)propyl L-lysyl-L-alaninate,
3-({4-[(3-chloropyridin-4-yl)amino]-2-[1-(4-ethoxy-2,6-difluorobenzyl)-1H-indazol-3-yl]-pyrimidin-5-yl}oxy)propyl L-arginyl-L-alaninate,
3-({4-[(3-chloropyridin-4-yl)amino]-2-[1-(4-ethoxy-2,6-difluorobenzyl)-1H indazol-3 yl]pyrimidin-5-yl}oxy)propyl L-lysyl-L-valinate, and
3-({4-[(3-chloropyridin-4-yl)amino]-2-[1-(4-ethoxy-2,6-difluorobenzyl)-1H-indazol-3-yl]-pyrimidin-5-yl}oxy)propyl L-arginyl-L-valinate,
   or an N-oxide, a salt, a tautomer or a stereoisomer of said compound, or a salt of said N-oxide, tautomer or stereoisomer.

In a further aspect of the invention compounds of formula (I) as described above are selected from the group consisting of:
3-[(4-{bis[2-(difluoromethyl)pyridin-4-yl]amino}-2-[1-(4-ethoxy-2,6-difluorobenzyl)-1H-indazol-3-yl]pyrimidin-5-yl)oxy]propan-1-ol,
3-({2-[1-(4-ethoxy-2,6-difluorobenzyl)-1H-indazol-3-yl]-4-[(3-methylpyridin-4-yl)amino]pyrimidin-5-yl}oxy)propan-1-ol,
3-({4-[(3-cyclopropylpyridin-4-yl)amino]-2-[1-(4-ethoxy-2,6-difluorobenzyl)-1H-indazol-3-yl]pyrimidin-5-yl}oxy)propan-1-ol,
3-({2-[1-(4-ethoxy-2,6-difluorobenzyl)-1H-indazol-3-yl]-4-[(2-methylpyridin-4-yl)amino]pyrimidin-5-yl}oxy)propan-1-ol,
N-[4-({2-[1-(4-ethoxy-2,6-difluorobenzyl)-1H-indazol-3-yl]-5-(3-hydroxy-propoxy)pyrimidin-4-yl}amino)pyridin-2-yl]acetamide,
3-({4-[(3-chloropyridin-4-yl)amino]-2-[1-(4-ethoxy-2,6-difluorobenzyl)-1H-indazol-3-yl]pyrimidin-5-yl}oxy)propan-1-ol,
3-({2-[1-(4-ethoxy-2,6-difluorobenzyl)-1H-indazol-3-yl]-4-[(2-methylpyrimidin-4-yl)amino]pyrimidin-5-yl}oxy)propan-1-ol,
3-({2-[1-(4-ethoxy-2,6-difluorobenzyl)-1H-indazol-3-yl]-4-(pyrimidin-4-ylamino)-pyrimidin-5-yl}oxy)propan-1-ol,
3-({2-[1-(4-ethoxy-2,6-difluorobenzyl)-1H-indazol-3-yl]-4-[(2-fluoropyridin-4-yl)amino]pyrimidin-5-yl}oxy)propan-1-ol,
3-({4-[(2,5-dimethylpyridin-4-yl)amino]-2-[1-(4-ethoxy-2,6-difluorobenzyl)-1H-indazol-3-yl]pyrimidin-5-yl}oxy)propan-1-ol,
N-[4-({2-[1-(4-ethoxy-2,6-difluorobenzyl)-1H-indazol-3-yl]-5-(3-hydroxy-propoxy)pyrimidin-4-yl}amino)-5-methylpyridin-2-yl]acetamide,
3-[(4-[(3-chloropyridin-4-yl)amino]-2-{1-[4-(cyclopropylmethoxy)-2,6-difluorobenzyl]-1H-indazol-3-yl}pyrimidin-5-yl)oxy]propan-1-ol,
3-[(4-{[2-(difluoromethyl)pyridin-4-yl]amino}-2-[1-(4-ethoxy-2,6-difluorobenzyl)-1H-indazol-3-yl]pyrimidin-5-yl)oxy]propyl L-lysyl-L-alaninate, salt with trifluoroaceticacid,
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-lysyl-L-alaninate, salt with trifluoroaceticacid,
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl D-lysyl-L-alaninate, salt with trifluoroaceticacid,
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-arginyl-L-alaninate, salt with trifluoroaceticacid, 3-({4-[(3-chloropyridin-4-yl)amino]-2-[1-(4-ethoxy-2,6-difluorobenzyl)-1H-indazol-3-yl]pyrimidin-5-yl}oxy)propyl L-lysyl-L-alaninate, salt with trifluoroaceticacid,
3-({4-[(2,6-Dimethylpyrimidin-4-yl)amino]-2-[1-(4-ethoxy-2,6-difluorbenzyl)-1H-indazol-3-yl]pyrimidin-5-yl}oxy)propan-1-ol.
N-[4-({2-[1-(4-Ethoxy-2,6-difluorobenzyl)-1H-indazol-3-yl]-5-(3-hydroxypropoxy)-pyrimidin-4-yl}amino)pyridin-2-yl]cyclopropancarboxamide,
3-({2-[1-(4-Ethoxy-2,6-difluorobenzyl)-1H-indazol-3-yl]-4-[(5-fluor-2-methylpyridin-4-yl)-amino]pyrimidin-5-yl}oxy)propan-1-ol,
N-[4-({2-[1-(4-ethoxy-2,6-difluorobenzyl)-1H-indazol-3-yl]-5-(3-hydroxypropoxy)-pyrimidin-4-yl}amino)-3-methylpyridin-2-yl]acetamide,
3-[(2-{1-[2,6-difluoro-4-(2,2,2-trifluoroethoxy)benzyl]-1H-indazol-3-yl}-4-[(2-methylpyrimidin-4-yl)amino]pyrimidin-5-yl)oxy]propan-1-ol,
3-[(4-[(3-chloropyridin-4-yl)amino]-2-{1-[2,6-difluoro-4-(2,2,2-trifluoroethoxy)benzyl]-1H-indazol-3-yl}pyrimidin-5-yl)oxy]propan-1-ol,
3-[(2-{1-[4-(2,2-difluoroethoxy)-2,6-difluorobenzyl]-1H-indazol-3-yl}-4-[(2-methylpyrimidin-4-yl)amino]pyrimidin-5-yl)oxy]propan-1-ol,
3-[(2-[1-(4-ethoxy-2,6-difluorobenzyl)-1H-indazol-3-yl]-4-{[3-(2,2,2-trifluoroethoxy)-pyrimidin-4-yl]amino}pyrimidin-5-yl)oxy]propan-1-ol,
3-[(2-{1-[4-(cyclopropylmethoxy)-2,6-difluorobenzyl]-1H-indazol-3-yl}-4-[(2,5-dimethylpyridin-4-yl)amino]pyrimidin-5-yl)oxy]propan-1-ol,
3-[(2-{1-[4-(cyclopropylmethoxy)-2,6-difluorobenzyl]-1H-indazol-3-yl}-4-[(2-methylpyrimidin-4-yl)amino]pyrimidin-5-yl)oxy]propan-1-ol,
N-(4-{[2-{1-[2,6-difluoro-4-(2,2,2-trifluoroethoxy)benzyl]-1H-indazol-3-yl}-5-(3-hydroxy-propoxy)pyrimidin-4-yl]amino}pyridin-2-yl)acetamide,
3-[(4-[(3-chloropyridin-4-yl)amino]-2-{1-[4-(2,2-difluoroethoxy)-2,6-difluorobenzyl]-1H-indazol-3-yl}pyrimidin-5-yl)oxy]propan-1-ol,
3-{[2-{1-[2,6-difluoro-4-(2-methoxyethoxy)benzyl]-1H-indazol-3-yl}-4-(pyrimidin-4-yl-amino)pyrimidin-5-yl]oxy}propan-1-ol,
3-({4-[(2,5-dimethylpyridin-4-yl)amino]-2-[1-(4-ethoxy-2,6-difluorobenzyl)-1H-indazol-3-yl]pyrimidin-5-yl}oxy)propyl D-lysyl-L-alaninate, salt with trifluoroaceticacid,
3-({4-[(2,5-dimethylpyridin-4-yl)amino]-2-[1-(4-ethoxy-2,6-difluorobenzyl)-1H-indazol-3-yl]pyrimidin-5-yl}oxy)propyl L-lysyl-L-alaninate, salt with trifluoroaceticacid,
3-({4-[(3-chloropyridin-4-yl)amino]-2-[1-(4-ethoxy-2,6-difluorobenzyl)-1H-indazol-3-yl]-pyrimidin-5-yl}oxy)propyl L-arginyl-L-alaninate,
3-({4-[(3-chloropyridin-4-yl)amino]-2-[1-(4-ethoxy-2,6-difluorobenzyl)-1H-indazol-3-yl]-pyrimidin-5-yl}oxy)propyl L-lysyl-L-valinate, and
3-({4-[(3-chloropyridin-4-yl)amino]-2-[1-(4-ethoxy-2,6-difluorobenzyl)-1H-indazol-3-yl]-pyrimidin-5-yl}oxy)propyl L-arginyl-L-valinate,
or an N-oxide, a salt, a tautomer or a stereoisomer of said compound, or a salt of said N-oxide, tautomer or stereoisomer.

A further aspect of the invention are compounds of formula (I), wherein:
V, W, Y and Z independently of each other represent CH or CR², wherein one of V, W, Y and Z represents CR²,
   or,
V represents N, and W, Y and Z independently of each other represent CH or CR²,
R¹ represents a group selected from:
   C₂-C₆-hydroxyalkyl, and R⁴,
      wherein said C₂-C₃-hydroxyalkyl group is optionally substituted with one, two or three halogen atoms selected from:
      fluorine, and chlorine,
R² represents, independently of each other, halogen or a group selected from:
   C₁-C₃-alkyl, C₃-C₄-cycloakyl, C₁-C₃-haloalkyl, C₁-C₃-alkoxy, C₁-C₃-haloalkoxy, —N(H)C(═O)—(C₁-C₃-alkyl), —N(H)C(═O)H,
   —N(H)C(═O)—(C₁-C₃-hydroxyalkyl), —N(H)C(═O)—(C₁-C₃-alkyl)-(C₁-C₃-alkoxy), —N(H)C(═O)-phenyl, —N(H)C(═O)—(C₃-C₄-cycloalkyl), —N(H)C(═O)—(C₁-C₃-alkyl)-(C₃-C₄-cycloakyl), and —N(H)C(═O)N(H)R⁸,
      said —N(H)C(═O)-phenyl being optionally substituted at the phenyl ring, one, two or three times, identically or differently, with a substituent selected from:
      halogen, hydroxy, cyano. C₁-C₄-alkyl, C₁-C₄-haloalkyl, C₁-C₄-alkoxy, C₁-C₄-haloalkoxy, C₃-C₄-cycloalkyl, and C₃-C₄-cycloalkyloxy,
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy,
R³ represents a group selected from:
   C₁-C₆-hydroxyalkyl, C₃-C₄-alkoxy, (C₂-C₆-hydroxyalkyl)-O—, C₁-C₆-haloalkoxy, (C₁-C₃-alkoxy)-(C₂-C₃-alkoxy)-, and (C₃-C₆-cycloalkyl)-(C₁-C₃-alkoxy)-,
      wherein said C₂-C₆-hydroxyalkyl group is optionally substituted with one, two or three halogen atoms selected from:
      fluorine, and chlorine,
R⁴ represents —(C₂-C₆-alkyl)-OC(═O)—C(H)(R⁵)—N(H)C(═O)—C(H)(R⁷)—NH₂,
   in which C₂-C₆-alkyl is optionally substituted with one, two or three halogen atoms selected from:
   fluorine, and chlorine,
R⁵ and R⁷ independently of each other represent a group selected from:
   —CH₃ (alanine), —C(H)(CH₃)₂ (valine), —(CH₂)₂CH₃ (norvaline), —(CH₂)₃NH₂ (ornithine), —(CH₂)₄NH₂ (lysine), and —(CH₂)₃N(H)C(═NH)NH₂ (arginine),
R⁸ represents hydrogen or a group selected from:
   C₁-C₃-alkyl, C₁-C₃-haloalkyl, C₂-C₃-hydroxyalkyl, C₃-C₄-cycloakyl, (C₃-C₄-cycloalkyl)-(C₁-C₃-alkyl)-, and (C₁-C₃-alkoxy)-(C₂-C₃-alkyl)-,
      or an N-oxide, a salt, a tautomer or a stereoisomer of said compound, or a salt of said N-oxide, tautomer or stereoisomer.

A further aspect of the invention are compounds of formula (I), wherein:
V, W, Y and Z independently of each other represent CH or CR², wherein one of V, W, as Y and Z represents CR²,
   or,
V represents N, and W, Y and Z independently of each other represent CH or CR²,
R¹ represents a group selected from:
   C₂-C₆-hydroxyalkyl, and R⁴,
R² represents, independently of each other, halogen or a group selected from:
   C₁-C₃-alkyl, C₃-C₄-cycloakyl, C₁-C₃-haloalkyl, C₁-C₃-haloalkoxy —N(H)C(═O)—(C₁-C₃-alkyl), and —N(H)C(═O)—(C₃-C₄-cycloalkyl)
R³ represents a group selected from:
   C₁-C₆-alkoxy, C₁-C₆-haloalkoxy, (C₁-alkoxy)-(C₂-C₃-alkoxy)-, and (C₃-C₆-cycloalkyl)-(C₁-C₃-alkoxy)-,
R⁴ represents —(C₂-C₆-alkyl)-OC(═O)—C(H)(R⁵)—N(H)C(═O)—C(H)(R⁷)—NH₂,
R⁵ and R⁷ independently of each other represent a group selected from:
   —CH₃ (alanine), —C(H)(CH₃)₂ (valine), —(CH₂)₄NH₂ (lysine), and —(CH₂)₃N(H)C(═NH)NH₂ (arginine),
      or an N-oxide, a salt, a tautomer or a stereoisomer of said compound, or a salt of said N-oxide, tautomer or stereoisomer.

A further aspect of the invention are compounds of formula (I), wherein:
V represents N, Y represents CH and W, and Z independently of each other represent CH or CR²,
R¹ represents a group selected from:
   —(CH₂)₃OH,
R² represents, independently of each other, methyl,
R³ represents a group selected from:
   ethoxy, 2,2-difluoroethoxy, 2,2,2-trifluoroethoxy, 2-methoxyethoxy, and cyclopropylmethoxy,
      or an N-oxide, a salt, a tautomer or a stereoisomer of said compound, or a salt of said N-oxide, tautomer or stereoisomer.

A further aspect of the invention are compounds of formula (I), wherein:

V represents N, Y represents CH and W, and Z independently of each other represent CH or CR²,
R¹ represents a group selected from:
   —(CH₂)₃OH,
R² represents, independently of each other, methyl,
R³ represents a group selected from:
   ethoxy, 2,2-difluoroethoxy, 2,2,2-trifluoroethoxy, and cyclopropylmethoxy,
      or an N-oxide, a salt, a tautomer or a stereoisomer of said compound, or a salt of said N-oxide, tautomer or stereoisomer.

A further aspect of the invention are compounds of formula (I), wherein
V, W, Y and Z Independently of each other represent CH or CR², wherein one of V, W, Y and Z represents CR²,
   or,
V represents N, and W, Y and Z independently of each other represent CH or CR²,
   or,
V and Y represent N, and W and Z independently of each other represent CH or CR².

Yet another aspect of the invention are compounds of formula (I) in which,
V, W, Y and Z independently of each other represent CH or CR², wherein one of V, W, Y and Z represents CR².

Yet another aspect of the invention are compounds of formula (I) in which,
V represents N, and W, Y and Z independently of each other represent CH or CR².

Yet another aspect of the invention are compounds of formula (I) in which,
V and Y represent N, and W and Z independently of each other represent CH or CR².

Yet another aspect of the invention are compounds of formula (I) supra in which,
V, W and Y each represent CH, and Z represents CR².

Yet another aspect of the invention are compounds of formula (I) in which,
V, W, Z represent CH and Y represents CR².

Yet another aspect of the invention are compounds of formula (I) in which.
Z, W, Y represent CH and V represents CR².

Yet another aspect of the Invention are compounds of formula (I) supra in which,
V and W each represent CH, and Y and Z independently of each other represent CR².

Yet another aspect of the invention are compounds of formula (I) supra in which,
V represents N, and W represents CH or CR², and Y and Z each represent CH.

Yet another aspect of the invention are compounds of formula (I) in which,
V represents N, W represents CR², Y and Z each represent CH.

Yet another aspect of the invention are compounds of formula (I) in which,
V represents N, W and Z independently of each other represent CR², Y represents CH.

Yet another aspect of the invention are compounds of formula (I) in which,
V represents N, W and Y independently of each other represent CR², Z represents CH.

A further aspect of the invention are compounds of formula (I), wherein
R¹ represents a group selected from:
   C₂-C₆-hydroxyalkyl, and R⁴,
      wherein said C₂-C₆-hydroxyalkyl group is optionally substituted with one, two or three halogen atoms selected from:
      fluorine, and chlorine,

Yet another aspect of the invention are compounds of formula (I) in which.
R¹ represents a group selected from:
   C₂-C₆-hydroxyalkyl,
      wherein said C₂-C₆-hydroxyalkyl group is optionally substituted with one, two or three halogen atoms selected from:
      fluorine, and chlorine.

Yet another aspect of the invention are compounds of formula (I) in which,
R¹ represents a group selected from:
   C₂-C₆-hydroxyalkyl.

Yet another aspect of the invention are compounds of formula (I) in which,

Yet another aspect of the Invention are compounds of formula (I) in which.
R¹ represents —(CH₂)₃OH.

Yet another aspect of the invention are compounds of formula (I) in which,
R¹ represents a group selected from R⁴.

A further aspect of the invention are compounds of formula (I), wherein
R² represents, independently of each other, halogen or a group selected from:
   C₁-C₃-alkyl, C₃-C₄-cycloakyl, C₁-C₃-haloalkyl, C₁-C₃-alkoxy, C₁-C₃-haloalkoxy, —N(H)C(═O)—(C₁-C₃-alkyl),
   —N(H)C(═O)H, —N(H)C(═O)—(C₁-C₃-hydroxyalkyl), —N(H)C(═O)—(C₁-C₃-alkyl)-(C₁-C₃-alkoxy), —N(H)C(═O)-phenyl, —N(H)C(═O)—(C₃-C₄-cycloalkyl), —N(H)C(═O)—(C₁-C₃-alkyl)-(C₃-C₄-cycloakyl), and —N(H)C(═O)N(H)R⁸,
      said —N(H)C(═O)-phenyl being optionally substituted at the phenyl ring, one, two or three times, identically or differently, with a substituent selected from:
      halogen, hydroxy, cyano, C₁-C₄-alkyl, C₁-C₄-haloalkyl, C₁-C₄-alkoxy, C₁-C₄-haloalkoxy, C₃-C₄-cycloalkyl, and C₃-C₄-cycloalkyloxy,
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy.

Yet another aspect of the invention are compounds of formula (I) in which,
R² represents, independently of each other, halogen or a group selected from:
   C₁-C₃-alkyl, C₃-C₄-cycloakyl, C₁-C₃-haloalkyl,
   —N(H)C(═O)—(C₁-C₃-alkyl).

Yet another aspect of the Invention are compounds of formula (I) in which.
R² represents, independently of each other, fluorine, chlorine or a group selected from:
methyl, cyclopropyl, difluoromethyl, and —N(H)C(═O)—CH₃.

Yet another aspect of the invention are compounds of formula (I) in which,
R² represents, independently of each other, fluorine, chlorine or a group selected from:
   methyl, cyclopropyl, difluoromethyl, 2,2,2-trifluoroethoxy, —N(H)C(═O)—CH₃, and —N(H)C(═O)-(cyclopropyl).

Yet another aspect of the invention are compounds of formula (I) in which.
R² represents a group selected from:
   2,2,2-trifluoroethoxy, and —N(H)C(═O)-(cyclopropyl).

A further aspect of the invention are compounds of formula (I), wherein
R³ represents a group selected from:
   C₁-C₆-alkyl, C₁-C₆-hydroxyakyl, C₁-C₆-haloalkyl, C₃-C₆-cycloalkyl, (C₁-C₃-alkoxy)-(C₁-C₃-alkyl)-, (C₃-C₆-cycloalkyl)-(C₁-C₃-alkyl)-, C₁-C₆-alkoxy, (C₂-C₆-hydroxyakyl)-O—, C₁-C₆-haloalkoxy, C₃-C₆-cycloalkyloxy, (C₁-C₃-alkoxy)-(C₂-C₃-alkoxy)-, and (C₃-C₆-cycloalkyl)-(C₁-C₃-alkoxy)-,
      wherein said C₂-C₆-hydroxyalkyl group is optionally substituted with one, two or three halogen atoms selected from:
      fluorine, and chlorine.

Yet another aspect of the invention are compounds of formula (I) in which,
R³ represents a group selected from:
   C₁-C₆-hydroxyalkyl, C₁-C₆-alkoxy, (C₂-C₆-hydroxyalkyl)-O—, C₁-C₆-haloalkoxy, and (C₃-C₆-cycloalkyl)-(C₁-C₃-alkoxy)-,
      wherein said C₂-C₅-hydroxyalkyl group is optionally substituted with one, two or three halogen atoms selected from:
      fluorine, and chlorine,

Yet another aspect of the invention are compounds of formula (I) in which,
R³ represents a group selected from: C₁-C₆-alkoxy, and (C₃-C₆-cycloalkyl)-(C₁-C₃-alkoxy)-.

Yet another aspect of the invention are compounds of formula (I) in which,
R³ represents a group selected from:
   ethoxy, and cyclopropylmethoxy.

Yet another aspect of the invention are compounds of formula (I) in which,
R³ represents a group selected from:
   ethoxy, 2,2-difluoroethoxy, 2,2,2-trifluoroethoxy, 2-methoxyethoxy, and cyclopropylmethoxy.

Yet another aspect of the invention are compounds of formula (I) in which.
R³ represents a group selected from:
   2,2-difluoroethoxy, 2,2,2-trifluoroethoxy, and 2-methoxyethoxy.

A further aspect of the invention are compounds of formula (I), wherein
R⁴ represents —(C₂-C₆-alkyl)-OC(═O)—C(H)(R⁵)—N(H)C(═O)—C(H)(R⁷)—NH₂,
   in which C₂-C₆-alkyl is optionally substituted with one, two or three halogen atoms selected from:
   fluorine, and chlorine.

Yet another aspect of the invention are compounds of formula (I) in which,
R⁴ represents —(C₂-C₆-alkyl)-OC(═O)—C(H)(R⁵)—N(H)C(═O)—C(H)(R⁷)—NH₂.

Yet another aspect of the invention are compounds of formula (I) in which,
R⁴ represents —(CH₂)₃—OC(═O)—C(H)(R⁵)—N(H)C(═O)—C(H)(R⁷)—NH₂.

A further aspect of the invention are compounds of formula (I), wherein
R⁵ and R⁷ independently of each other represent hydrogen (glycine) or a group selected from:
   —CH₃ (alanine), —C(H)(CH₃)₂ (valine), —(CH₂)₂CH₃ (norvaline), —CH₂C(H)(CH₃)₂ (leucine), —C(H)(CH₃)CH₂CH₃ (isoleucine), —(CH₂)₃CH₃ (norleucine), —C(CH₃)₃ (2-tert-butylglycine), benzyl (phenylalanine), 4-hydroxybenzyl (tyrosine), —(CH₂)₃NH₂ (omithine), —(CH₂)₄NH₂ (lysine), —(CH₂)₂C(H)(OH)CH₂NH₂ (hydroxylysine), —CH₂OH (serine), —(CH₂)₂OH (homoserine), —C(H)(OH)CH₃ (threonine), —(CH₂)₃N(H)C(═NH)NH₂ (arginine), —(CH₂)₃N(H)C(═O)NH₂ (citrulline), —CH₂C(═O)NH₂ (asparagine), —CH₂C(═O)OH (aspartic acid), —(CH₂)₂C(═O)OH (glutamic acid), —(CH₂)₂C(═O)NH₂ (glutamine), —CH₂SH (cysteine), —(CH₂)₂SH (homocysteine), —(CH₂)₂SCH₃ (methionine), —CH₂SCH₃ (S-methylcysteine), (1H-imidazol-4-yl)methyl-(histidine), (1H-indol-3-yl)methyl-(thryptophan), —CH₂NH₂ (2,3-diaminopropanoic acid), and —(CH₂)₂NH₂ (2,4-diaminobutanoic acid).

Yet another aspect of the invention are compounds of formula (I) in which,
R⁵ and R⁷ independently of each other represent a group selected from:
   —CH₃ (alanine), —C(H)(CH₃)₂ (valine), —(CH₂)₂CH₃ (norvaline), —(CH₂)₃NH₂ (ornithine), —(CH₂)₄NH₂ (lysine), and —(CH₂)₃N(H)C(═NH)NH₂ (arginine).

Yet another aspect of the invention are compounds of formula (I) in which,
R⁵ and R⁷ independently of each other represent a group selected from:
—CH₃ (alanine), —(CH₂)₄NH₂ (lysine), and —(CH₂)₃N(H)C(═NH)NH₂ (arginine).

Yet another aspect of the invention are compounds of formula (I) in which,
R⁵ represents —CH₃ (alanine), and,

Yet another aspect of the invention are compounds of formula (I) in which,
R⁵ represents a group selected from:
   —CH₃ (alanine), and —C(H)(CH₃)₂ (valine).

Yet another aspect of the invention are compounds of formula (I) in which,
R⁵ represents —C(H)(CH₃)₂ (valine).
R⁷ represents a group selected from:
   —(CH₂)₄NH₂ (lysine), and —(CH₂)₃N(H)C(═NH)NH₂ (arginine).

Yet another aspect of the invention are compounds of formula (I) in which.
R⁵ represents —CH₃ (alanine).

Yet another aspect of the invention are compounds of formula (I) in which,
R⁷ represents a group selected from:
   —(CH₂)₄NH₂ (lysine), and —(CH₂)₃N(H)C(═NH)NH₂ (arginine).

A further aspect of the invention are compounds of formula (I), wherein
R⁸ represents hydrogen or a group selected from:
   C₁-C₃-alkyl, C₁-C₃-haloalkyl, C₂-C₃-hydroxyalkyl, C₃-C₄-cycloakyl, (C₃-C₄-cycloalkyl)-(C₁-C₃-alkyl)-, and (C₁-C₃-alkoxy)-(C₂-C₃-alkyl)-.

One aspect of the invention are compounds of formula (I) as described in the examples, as characterized by their names in the title, as claimed in claims 5 or 6, and their structures as well as the subcombinations of all residues specifically disclosed in the compounds of the examples.

Another aspect of the present invention are the intermediates as used for their synthesis.

Particularly, the present invention relates to an intermediate compound of formula (1-7):

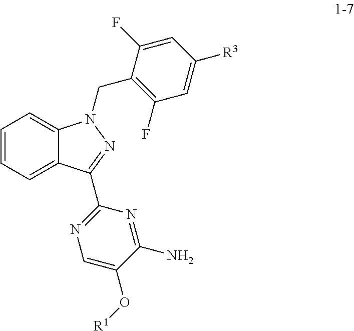

in which R¹, R³ are as defined as defined herein for the compound of formula (I).

Another aspect of the present invention relates to the use of a compound of formula (1-7), for the preparation of a compound of general formula (I)

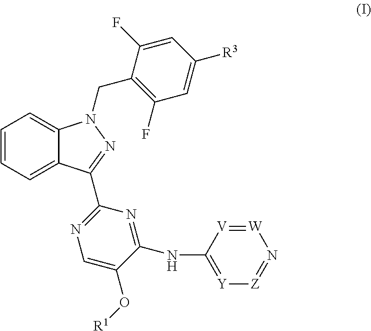

in which R¹, R³, V, W, Y, and Z are as defined herein for the compound of formula (I).

A further aspect of the invention are compounds of formula (I), which are present as their salts.

Yet another aspect of the Invention are compounds of formula (I) in which the salt is a pharmaceutically acceptable salt.

It is to be understood that the present invention relates to any sub-combination within any embodiment or aspect of the present invention of compounds of general formula (I), supra.

More particularly still, the present invention covers compounds of general formula (I) which are disclosed in the Example section of this text, infra.

In accordance with another aspect, the present invention covers methods of preparing compounds of the present invention, said methods comprising the steps as described in the Experimental Section herein.

In particular, the present invention relates to a method of preparing a compound of general formula (I), said method comprising the step of allowing an intermediate compound of general formula (1-7):

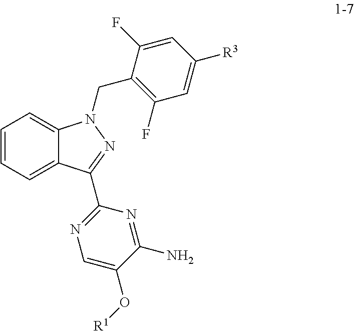

in which R¹, R³, are as defined herein for the compound of formula (I),
to react with a compound of general formula (1-8).

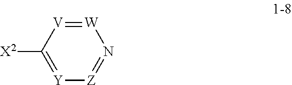

in which V, W, Y, and Z are as defined herein for the compound of formula (I), and X² represents F, Cl, Br, I, boronic acid or a boronic acid ester, such as, for example, 4,4,5,5-tetramethyl-2-phenyl-1,3,2-dioxaborolane (boronic acid pinacole ester).
thereby giving a compound of general formula (I):

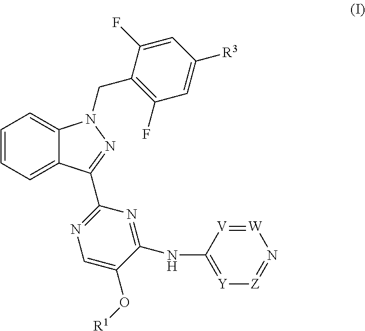

in which R¹, R³, V, W, Y, and Z are as defined herein for the compound of formula (I).

Another embodiment of the invention are compounds according to the claims as disclosed in the Claims section wherein the definitions are limited according to the preferred or more preferred definitions as disclosed below or specifically disclosed residues of the exemplified compounds and subcombinations thereof.

### BRIEF DESCRIPTION OF THE FIGURES

FIG. 1: binding affinities over time of example 2-1-1 and example 4-1-1 (prodrug), together with the prodrug stability and pIC₅₀ to be expected if only the drug present.

FIG. 2: binding affinities over time of example 2-1-5 and example 4-1-3 (prodrug), together with the prodrug stability and pIC₅₀ to be expected if only the drug present.

FIG. 3: binding affinities over time of example 2-1-5 and example 4-1-4 (prodrug), together with the prodrug stability and pIC₅₀ to be expected if only the drug present.

FIG. 4: binding affinities over time of example 2-1-6 and example 4-1-5 (prodrug), together with the prodrug stability and pIC₅₀ to be expected if only the drug present.

### DEFINITIONS

Constituents which are optionally substituted as stated herein, may be substituted, unless otherwise noted, one or more times, independently from one another at any possible position. When any variable occurs more than one time in any constituent, each definition is independent. For example, whenever R¹, R², R³, R⁴, R⁵, R⁷, R⁸ V, W, Y and/or Z occur more than one time for any compound of formula (I) each definition of R¹, R², R³, R⁴, R⁵, R⁷, R⁸, V, W, Y and Z is independent.

Should a constituent be composed of more than one part, e.g. C₁-C₄-alkoxy-C₁-C₄-alkyl-, the position of a possible substituent can be at any of these parts at any suitable position. A hyphen at the beginning or at the end of the constituent marks the point of attachment to the rest of the molecule. Should a ring be substituted the substitutent could be at any suitable position of the ring, also on a ring nitrogen atom if suitable.

The term “comprising” when used in the specification includes “consisting of”.

If it is referred to “as mentioned above” or “mentioned above” within the description it is referred to any of the disclosures made within the specification in any of the preceding pages.

“suitable” within the sense of the invention means chemically possible to be made by methods within the knowledge of a skilled person.

The terms as mentioned in the present text have preferably the following meanings:

The term “halogen atom”, “halo-” or “Hal-” Is to be understood as meaning a fluorine, chlorine, bromine or iodine atom.

The term “C₁-C₆-alkyl” is to be understood as meaning a linear or branched, saturated, monovalent hydrocarbon group having 1, 2, 3, 4, 5, or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group.

The term “C₁-C₆-haloalkyl” is to be understood as meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said C₁-C₆-haloalkyl group is, for example, —CF₃, —CHF₂, —CH₂F, —CF₂CF₃, —CH₂CH₂F, —CH₂CHF₂, —CH₂CF₃, —CH₂CH₂CF₃, or —CH(CH₂F)₂.

The term “C₁-C₆-alkoxy” is to be understood as meaning a linear or branched, saturated, monovalent, hydrocarbon group of formula —O-alkyl, in which the term “alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “C₁-C₆-haloalkoxy” is to be understood as meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said C₁-C₆-haloalkoxy group is, for example, —OCF₃, —OCHF₂, —OCH₂F, —OCF₂CF₃, —OCH₂CHF₂, or —OCH₂CF₃.

The term “C₁-C₃-hydroxyalkyl” is to be understood as preferably meaning a linear or branched, saturated, monovalent hydrocarbon group in which the term “C₁-C₆-alkyl” is defined supra, and in which one or more hydrogen atoms is replaced by a hydroxy group, e.g. a hydroxymethyl, 1-hydroxyethyl, 2-hydroxyethyl, 1,2-dihydroxyethyl, 3-hydroxypropyl, 2-hydroxypropyl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₃-hydroxyalkyl” is to be understood as preferably meaning a linear or branched, saturated, monovalent hydrocarbon group in which the term “C₁-C₃-alkyl” is defined supra, and in which one or more hydrogen atoms is replaced by a hydroxy group, e.g. a hydroxymethyl, 1-hydroxyethyl, 2-hydroxyethyl, 1,2-dihydroxyethyl, 3-hydroxypropyl, 2-hydroxypropyl, 2,3-dihydroxypropyl, 1,3-dihydroxypropan-2-yl.

The term “C₂-C₆-hydroxyalkyl” is to be understood as meaning a linear or branched, saturated, monovalent hydrocarbon group having 2, 3, 4, 5, or 6 carbon atoms, in which one or more hydrogen atoms is replaced by a hydroxy group, e.g. a 2-hydroxyethyl, 3-hydroxypropyl, 2-hydroxypropyl, 2,3-dihydroxypropyl, 3-hydroxy-2-methyl-propyl, 2-hydroxy-2-methyl-propyl. Preferred is “C₂-C₄-hydroxyalkyl”, more preferred is “C₂-hydroxyalkyl”, i.e. a 2-hydroxyethyl group.

The term “C₃-C₆-cycloalkyl” is to be understood as meaning a saturated, monovalent, monocyclic hydrocarbon ring which contains 3, 4, 5 or 6 carbon atoms (“C₃-C₆-cycloalkyl”). Said C₃-C₆-cycloalkyl group is for example, a monocydic hydrocarbon ring, e.g. a cyclopropyl, cyclobutyl, cyclopentyl or cyclohexyl ring.

The term “C₃-C₆-cycloalkyloxy” is to be understood as meaning a saturated, monovalent, monocyclic hydrocarbon group of formula —O-cycloalkyl, in which the term “cycloalkyl” is defined supra, e.g. a. a cyclopropyloxy, cyclobutyloxy, cyclopentyloxy or cyclohexyloxy group.

The term “C₁-C₆”, as used throughout this text, e.g. in the context of the definition of “C₁-C₆-alkyl”, “C₁-C₆-haloalkyl”, “C₁-C₆-hydroxyalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅. C₃-C₄, C₁-C₂, C₁-C₃, C₁-C₄, C₁-C₅ particularly C₁-C₂, C₁-C₃, C₁-C₄, C₁-C₅, C₁-C₆: more particularly C₁-C₄; in the case of “C₁-C₆-haloalkyl” or “C₁-C₆-haloalkoxy” even more particularly C₁-C₂.

The term “C₂-C₆”, as used throughout this text, e.g. in the context of the definition of “C₂-C₆-hydroxyalkyl”, is to be understood as meaning a hydroxyalkyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₂-C₅, C₃-C₄, particularly C₂-C₃, C₂-C₄, C₂-C₅, C₂-C₆.

Further, as used herein, the term “C₃-C₆”, as used throughout this text, e.g. in the context of the definition of “C₃-C₆-cycloakyl”, is to be understood as meaning a cycloalkyl group having a finite number of carbon atoms of 3 to 6, i.e. 3, 4, 5 or 6 carbon atoms. It is to be understood further that said term “C₃-C₅” is to be interpreted as any sub-range comprised therein, e.g. C₃-C₆, C₄-C₅. C₃-C₅, C₃-C₄, C₄-C₆, C₅-C₆; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

Ring system substituent means a substituent attached to an aromatic or nonaromatic ring system which, for example, replaces an available hydrogen on the ring system.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The term “V, W, Y and Z independently of each other represent CH or CR², wherein one of V, W, Y and Z represents CR²”, is to be understood as meaning that at least one of V, W, Y and Z represents CR², and the remaining, independently from each other, represent CH or CR², as it is known to a skilled person. For example, according to certain embodiments of the Invention, V, W, Y and Z independently of each other represent CH or CR², wherein one of V, W, Y and Z represents CR² and the remaining represent CH; according to other embodiments of the invention. V, W, Y and Z 2 independently of each other represent CH or CR², wherein two of V, W, Y and Z, independently of each other, represent CR² and the remaining represent CH; still according to other embodiments of the invention, V, W, Y and Z independently of each other represent CH or CR², wherein three of V, W, Y and Z, independently of each other, represent CR² and the remaining represents CH, for example.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, Isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence is preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of this invention optionally contain one or more asymmetric centre, depending upon the location and nature of the various substituents desired. Asymmetric carbon atoms is present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds of the present invention optionally contain sulphur atoms which are asymmetric, such as an asymmetric sulfoxide, of structure:

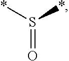

for example,
in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, the compounds of the present invention may exist as tautomers.

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et a. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, Itaconic, sulfamic, trifluoromethanesulfonic, dodecylsulfuric, ethanesulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; diakyl sulfates like dimethyl, diethyl, and dibutyl sulfate; and diamyl sulfates, long chain halides such as decyl, lauryl, myristyl and strearyl chlorides, bromides and iodides, arakyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃COOH”, “x Na⁺”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

As used herein, the term “in vivo hydrolysable ester” is understood as meaning an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloakyl and optionally substituted phenylalkyl, in particular benzyl esters. C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-2 methoxycarbonyloxyethyl, and may be formed at any carboxy group in the compounds of this invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyioxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorph, or as a mixture of more than one polymorph, in any ratio.

In the context of the properties of the compounds of the present Invention the term “pharmacokinetic profile” means one single parameter or a combination thereof including permeability, bioavailability, exposure, and pharmacodynamic parameters such as duration, or magnitude of pharmacological effect, as measured in a suitable experiment. Compounds with improved pharmacokinetic profiles can, for example, be used in lower doses to achieve the same effect, may achieve a longer duration of action, or a may achieve a combination of both effects.

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered. Any such combination of a compound of formula (I) of the present invention with an anti-cancer agent as defined below is an embodiment of the invention.

The term “(chemotherapeutic) anti-cancer agents”, includes but is not limited to 131I-chTNT, abarelix, abiraterone, aclarubicin, ado-trastuzumab emtansine, afatinb, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, copanlisib, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim. GM-CSF, goserelin, granisetron, granulocyte colony stimulating factor, histamine dihydrochloride, histrelin, hydroxycarbamide, 1-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, lobitridol, iobenguane (123I), iomeprol, ipilimumab, irinotecan, Itraconazole, ixabepibne, lanreotide, lapatinib, lasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradlol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, stanozoblol, streptozocin, sunitinib, talaporfin, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

It has now been found, and this constitutes the basis of the present invention, that said compounds of the present invention have surprising and advantageous properties.

In particular, said compounds of the present invention have surprisingly been found to effectively inhibit Bub1 kinase and may therefore be used for the treatment or prophylaxis of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular Inflammatory responses is mediated by Bub1 kinase, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The intermediates used for the synthesis of the compounds of claims 1 to 6 as described below, as well as their use for the synthesis of the compounds of claims 1 to 6, are one further aspect of the present invention. Preferred intermediates are the Intermediate Examples as disclosed below.

General Procedures

The compounds according to the invention can be prepared according to the following schemes 1 through 16.

The schemes and procedures described below illustrate synthetic routes to the compounds of general formula (I) of the Invention and are not Intended to be limiting. It is obvious to the person skilled in the art that the order of transformations as exemplified in the Schemes can be modified in various ways. The order of transformations exemplified in the Schemes is therefore not intended to be limiting. In addition, interconversion of any of the substituents, R¹, R³, R⁵, R⁷, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T.W. Greene and P.G.M. Wuts in Protective Groups in Organic Synthesis, 3^{rd} edition, Wiley 1999). Specific examples are described in the subsequent paragraphs.

One route for the preparation of compounds of general formula (Ia) is described in Scheme 1.

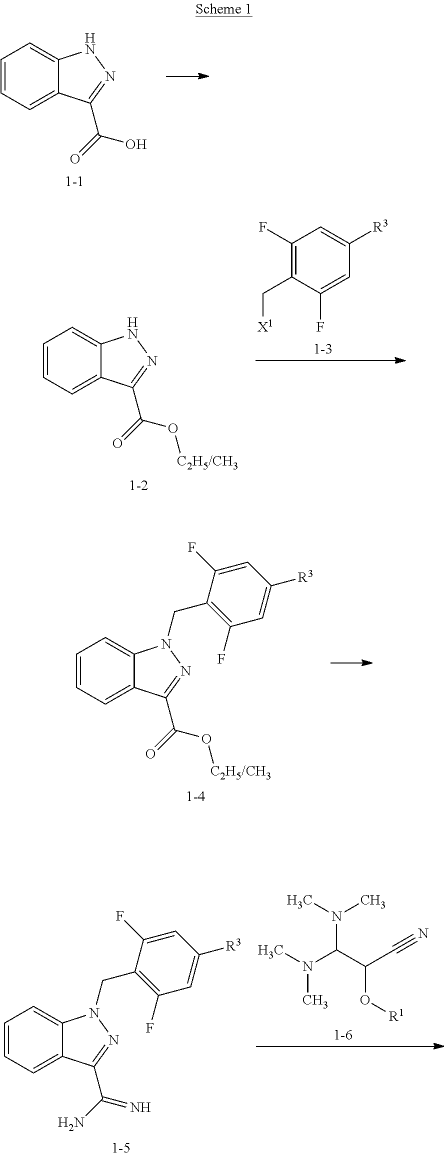

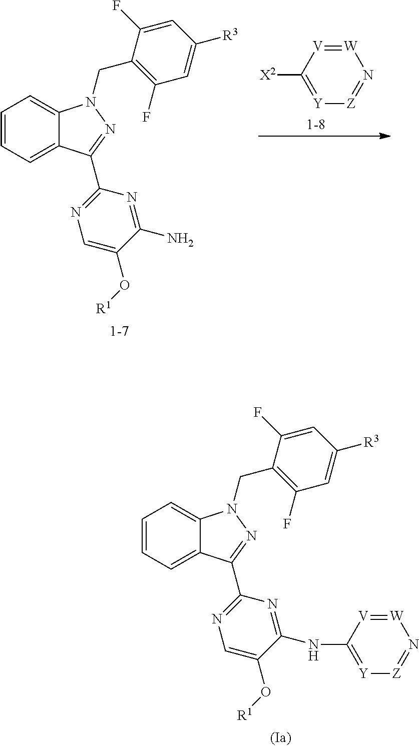

Scheme 1: Route for the preparation of compounds of general formula (Ia), wherein R¹, R³, V, W, Y and Z have the meaning as given for general formula (I), supra.

X¹ represents a leaving group such as for example a Cl, Br or I, or an aryl sulfonate such as for example p-toluene sulfonate, or a alkyl sulfonate such as for example methane sulfonate or trifluoromethane sulfonate (triflate group), and X² represents F, Cl, Br, I, boronic acid or a boronic acid ester, such as for example 4,4,5,5-tetramethyl-2-phenyl-1,3,2-dioxaborolane (boronic acid pinacole ester).

In addition, interconversion of any of the substituents R¹, R³, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T.W. Greene and P.G.M. Wuts in Protective Groups in Organic Synthesis, 3^{rd} edition. Wiley 1999). Specific examples are described in the subsequent paragraphs.

Compounds 1-3, 1-6, and 1-8 are either commercially available or can be prepared according to procedures available from the public domain, as understandable to the person skilled in the art. Specific examples are described in the subsequent paragraphs.

A suitably substituted 1H-indazole-3-carboxylic acid of the general formula (1-1) can be reacted with methanol or ethanol in the presence of catalytic amounts of a Broensted acid, such as, for example, hydrochloric acid or sulphuric acid, at temperatures ranging from 0° C. to boiling point of the respective alcohol, preferably the reaction is carried out at 85° C., to furnish alkyl 1H-indazole-3-carboxylat e intermediates of general formula (1-2).

Alkyl 1H-indazole-3-carboxylate Intermediates of the general formula (1-2) can be converted to intermediates of general formula (1-4) by reaction with a suitable alkylating agent, such as, for example a substituted benzyl halide (1-3), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-4) are treated with the reagent methylchloroaluminiumamide prepared in situ by addition of ammonium chloride to commercially available trimethylaluminium, in a suitable solvent system, such as, for example, toluene, at a temperature between 0° C. and the boiling point of the respective solvent, preferably the reaction is carried out at 80° C. and are quenched with a suitable solvent system, such as, for example, methanol, to form the desired intermediate of general formula (1-5).

Intermediates of general formula (1-5) can be converted to intermediates of general formula (1-7) by reaction with a suitably substituted 3,3-bis-(dimethylamino)propanenitrile of the general formula (1-6), in the presence of a suitable base, such as, for example piperidine, in a suitable solvent system, such as, for example, 3-methylbutan-1-ol, in a temperature range from room temperature to the boiling point of the respective solvent, preferably the reaction is carried out at 100° C.

Intermediates of general formula (1-7) can be reacted with a suitable halo substituted sixmembered heterocycle of the general formula (1-8), such as, for example 4-bromo-2-methyl-pyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-dylbis(diphenylphosphane), in a suitable solvent system, such as, for example, DMF, In a temperature range from room temperature to the boiling point of the respective solvent, preferably the reaction is carried out at at 1000° C. to furbish compounds of general formula (Ia). Alternatively the following s palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(μ-acetato)bis[o-(di-o-tolylphosphino)benzyl]dipalladium(II) [cataCXium@ C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium(II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl) palladium(II), dichloro(di-μ-chloro)bis[1,3-bis(2,6-di-iso-propylphenyl) imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl [2′,4′,6′-tri(propan-2-yl)biphenyl-2-yl]-lambda5-phosphanyl}palladium, (2′-aminobi-phenyl-2-yl)(methariesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl) phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methane-sulfonato-kappaO)palladium-[2′,6′-bis(propan-2-yloxy)biphenyl-2-yl](dicyclohexyl) phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′16′-tri(propan-2-yl)biphenyl-2-yl]phosphane, (2′-aminobiphenyl-2-yl)(methanesulfonato-kappaO)palladium-dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphos-phonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phosphine, di-tert-butyl(2′,4′,6-triisopropyl-3,4,5,6-tetramethylbiphenyl-2-yl) phosphine, adamantan-1-yl(adamantan-2-yl)(246′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethylbiphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phenyl](2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine; sodium 2′-(dicyclohexylphosphino)-2,6-diisopropylbiphenyl-4-sulfonate; sodium 2′-(dicyclohexylphosphino)-2,6-dimethoxybiphenyl-3-sulfonate, 1,1′-binaphthalen-2-yl(di-tert-butyl)phosphine, 1,3-bis(2,4,6-trimethylphenyl)-1,3-dihydro-2H-imidazol-2-ylidene, 1,3-bis[2,6-di(propan-2-yl)phenyl]-1,3-dihydro-2H-imidazol-2-ylidene.

Alternatively intermediates of general formula (1-7) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (1a).

Alternatively intermediates of general formula (1-7) can be reacted with a suitable halo substituted sixmembered heterocycle of the general formula (1-8), such as for example 4-fluoro-2-methyl-pyridine, in the presence of a suitable base, such as, for example sodium hydride, in a suitable solvent system, such as, for example DMF, in a temperature range from room temperature to the boiling point of the respective solvent, preferably the reaction is carried out at 90° C. to furnish compounds of general formula (Ia).

One route for the preparation of compounds of general formula (1a) is described in Scheme 2.

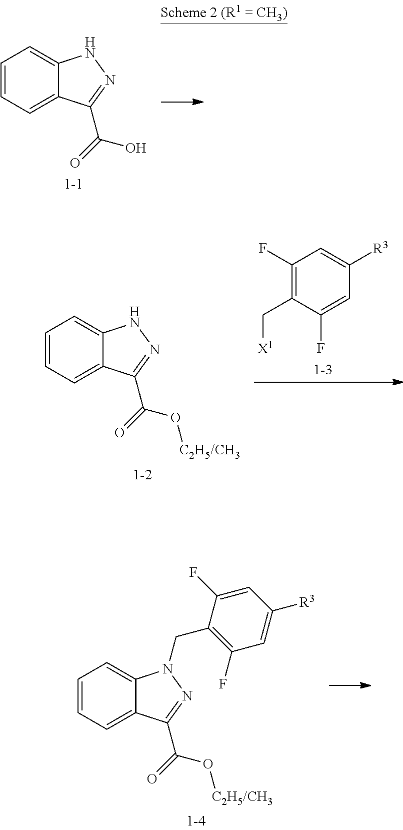

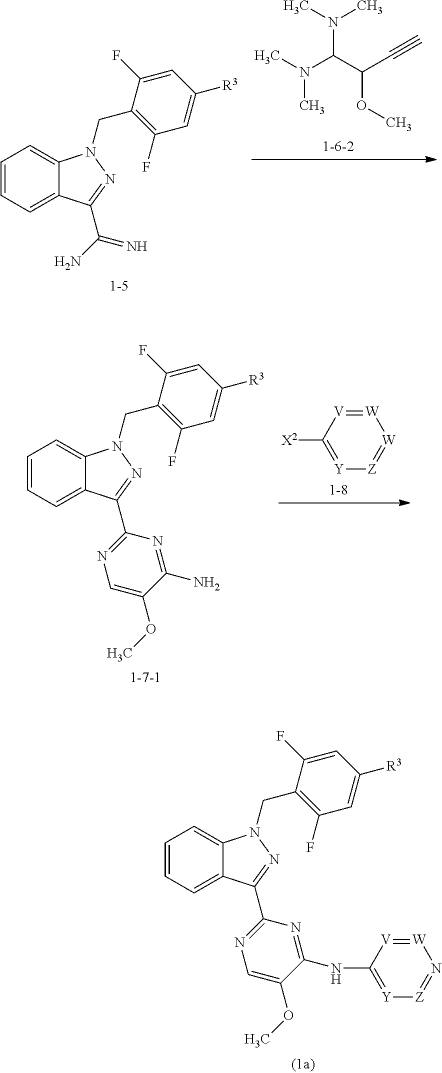

Scheme 2: Route for the preparation of compounds of general formula (1a), wherein R³, V, W, Y and Z have the meaning as given for general formula (I), supra X¹ represents a leaving group such as for example a Cl, Br or I, or an aryl sulfonate such as for example p-toluene sulfonate, or a alkyl sulfonate such as for example methane sulfonate or trifluoromethane sulfonate (triflate group), and X² represents F, Cl, Br, I, boronic acid or a boronic acid ester, such as for example 4,4,5,5-tetramethyl-2-phenyl-1,3,2-dioxaborolane (boronic acid pinacole ester).

In addition, interconversion of any of the substituents R³, V, W, Y or Z can be achieved before and/or alter the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T.W. Greene and P.G.M. Wuts in Protective Groups in Organic Synthesis, 3^{rd} edition, Wiley 1999). Specific examples are described in the subsequent paragraphs.

Compounds 1-3, 1-6 and 1-8 are either commercially available or can be prepared according to procedures available from the public domain, as understandable to the person skilled in the art. Specific examples are described in the subsequent paragraphs.

A suitably substituted 1H-indazole-3-carboxylic acid of the general formula (1-1) can be reacted with methanol or ethanol in the presence of catalytic amounts of a Broensted acid, such as, for example, hydrochloric acid or sulphuric acid, at temperatures ranging from 0° C. to boiling point of the respective alcohol, preferably the reaction is carried out at 85° C., to furnish alkyl 1H-indazole-3-carboxylat e intermediates of general formula (1-2).

Alkyl 1H-Indazole-3-carboxylate Intermediates of the general formula (1-2) can be converted to intermediates of general formula (1-4) by reaction with a suitable alkylating agent, such as, for example a substituted benzyl halide (1-3), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-4) are treated with the reagent methylchloroaluminiumamide prepared in situ by addition of ammonium chloride to commercially available trimethylaluminium, in a suitable solvent system, such as, for example, toluene, at a temperature between 0° C. and the boiling point of the respective solvent, preferably the reaction is carried out at 80° C. and are quenched with a suitable solvent system, such as, for example, methanol, to form the desired intermediate of general formula (1-5).

Intermediates of general formula (1-5) can be converted to intermediates of general formula (1-7-1) by reaction with a suitably substituted 3,3-bis-(dimethylamino)propanenitrile of the general formula (1-6), such as, for example 3,3-bis(dimethylamino)-2-methoxypropanenitrile, in the presence of a suitable base, such as, for example piperidine, in a suitable solvent system, such as, for example, 3-methylbutan-1-ol, in a temperature range from room temperature to the boiling point of the respective solvent, preferably the reaction is carried out at 100° C.

Intermediates of general formula (1-7-1) can be reacted with a suitable halo substituted sixmembered heterocycle of the general formula (1-8), such as, for example 4-bromo-2-methyl-pyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at at 100° C. to furnish compounds of general formula (1a). Alternatively the following palladium catalysts can be used:
allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(μ-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium(II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl) palladium(II), dichloro(di-j-chloro)bis[1,3-bis(2,6-di-iso-propylphenyl) imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,8′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl [2′,4′,6′-tri(propan-2-yl)biphenyl-2-yl]-lambda5-phosphanyl}palladium, (2′-aminobi-phenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl) phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,8′-tri-iso-propyl-1,1′-biphenyl)[2-(2-aminoethyl)phenyl]palladium(II), (2′-aminobiphenyl-2-yl)(methane-sulfonato-kappaO)palladium-[2′,6′-bis(propan-2-yloxy)biphenyl-2-yl](dicyclohexyl) phosphane, (2′-aminobiphenyl-2-yl)(methanesulfonato-kappaO)palladium-dicyclohexyl[2′,4′,8′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methariesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium-dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphos-phonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phosphine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethylbiphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl](2′,4′,6′-triisopropyl-3,6-dimethoxybiphenyl-2-yl)phosphine) biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine, 1,3-bis(2,4,6-trimethylphenyl)-1,3-dihydro-2H-imidazol-2-ylidene, 1,3-bis[2,6-di(propan-2-yl)phenyl]-1,3-dihydro-2H-imidazol-2-ylidene.

Alternatively intermediates of general formula (1-7-1) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (1a).

Alternatively intermediates of general formula (1-7-1) can be reacted with a suitable halo substituted sixmembered heterocycle of the general formula (1-8), such as for example 4-fluoro-2-methyl-pyridine, in the presence of a suitable base, such as, for example sodium hydride, in a suitable solvent system, such as, for example DMF, in a temperature range from room temperature to the boiling point of the respective solvent, preferably the reaction is carried out at 90° C. to furnish compounds of general formula (1a).

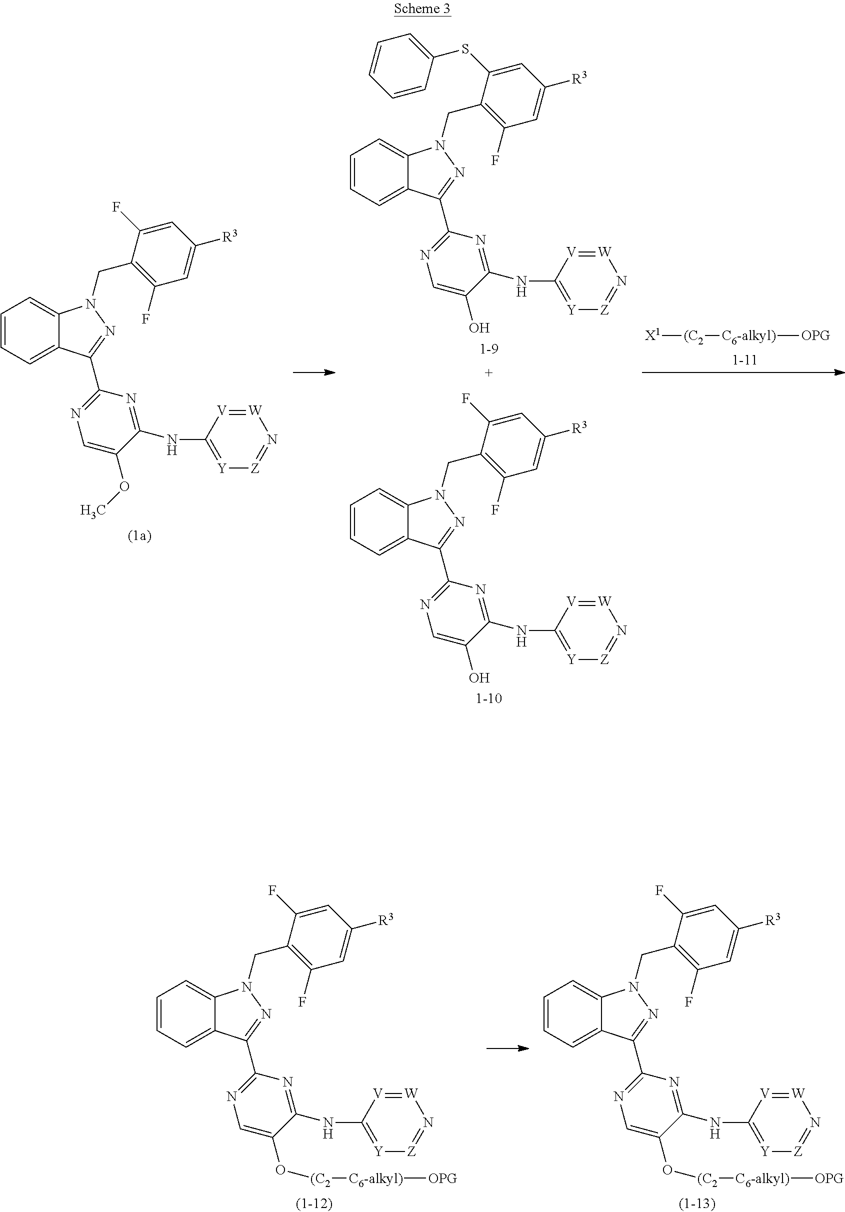

Scheme 3: Process for the preparation of compounds of general formula (1-13) via de-methylation of compounds of general formula (1a) to furnish compounds of general formula 1-10 and subsequent etherification and deprotection to furnish compounds of general formula (I-13), wherein R³. V, W, Y and Z have the meaning as given for general formula (I), supra, X¹ represents a leaving group such as for example a Cl, Br or I, or an aryl sulfonate such as for example p-toluene sulfonate, or a alkyl sulfonate such as for example methane sulfonate or trifluoromethane sulfonate (triflate group), and PG represents an alcohol protecting group as for example tert-butyldimethylsilyl, tert-butyldiphenylsilyl, triethylsilyl, triisopropylsilyl or tetrahydropyranyl. In addition, interconversion of any of the substituents R³, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T.W. Greene and P.G.M. Wuts in Protective Groups in Organic Synthesis, 3^{rd} edition, Wiley 1999). Specific examples are described in the subsequent paragraphs.

Compounds of general formula 1-11 are commercially available, wherein X represents leaving group such as for example a Cl, Br or I, or X stands for an aryl sulfonate such as for example p-toluene sulfonate, or for an alkyl sulfonate such as for example methane sulfonate or trifluoromethane sulfonate (triflate group).

Compounds of general formula (1a) are converted to compounds of general formula (1-10) by treatment with a suitable demethylating agent, such as for example benzenethiol, in a suitable solvent, such as, for example, 1-methylpyrrolidin-2-one, in the presence of a suitable base, such as, for example potassium carbonate, in a temperature range from room temperature to the boiling point of the respective solvent, preferably the reaction Is carried out at 190° C. The side product (1-9) can be Isolated.

Compounds of general formula (1-10) are then reacted with a compound of general formula (1-11) as mentioned above, in a suitable solvent, such as, for example, DMF, in the presence of a suitable base, such as, for example, potassium carbonate in a temperature range from room temperature to the boiling point of the respective solvent, preferably the reaction is carried out at room temperature, to furnish compounds of general formula (1-12).

Compounds of general formula (1-12) are then reacted a suitable Broensted acid, such as, for example, hydrogen chloride, in a suitable solvent, such as, for example, dioxane, in a temperature range from room temperature to the boiling point of the respective solvent, preferably the reaction is carried out at room temperature, to furnish compounds of general formula (1-13).

Compounds of general formula (1-13) can be converted into compounds of general formula (1-19) according to the procedure depicted in Scheme 4.

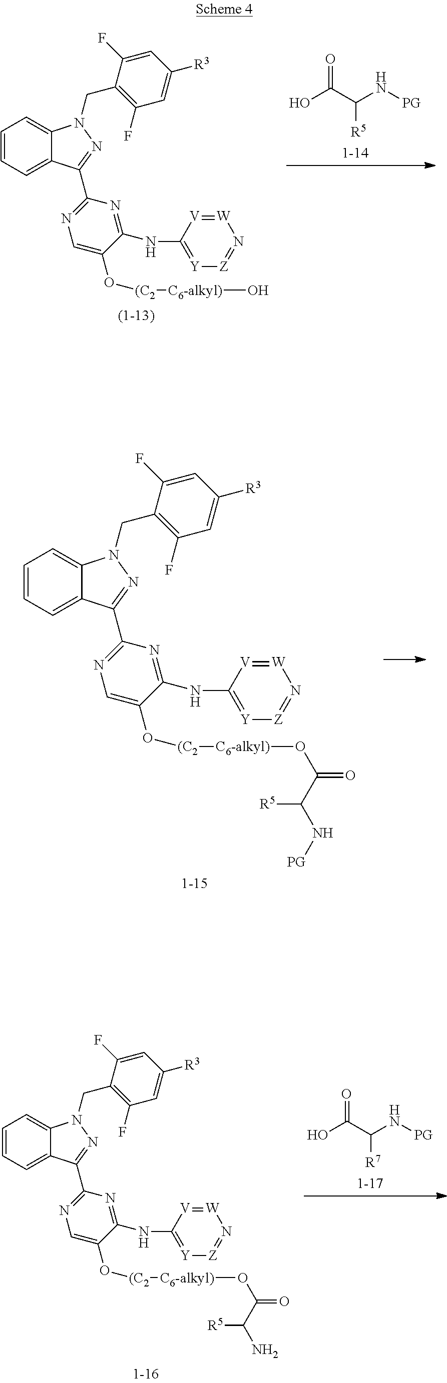

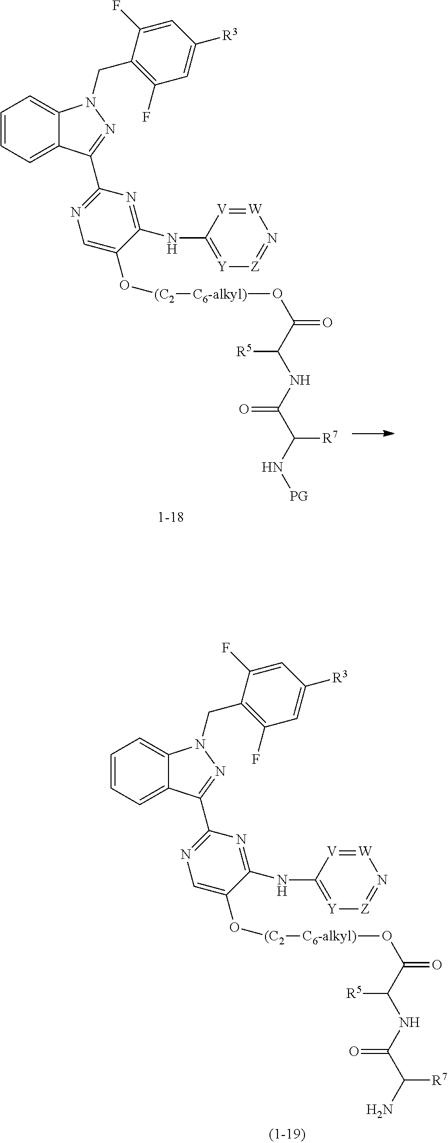

Scheme 4: Route for the preparation of compounds of general formula (1-19), wherein R³, R⁵, R⁷, V, W, Y and Z have the meaning as given for general formula (I), supra, and PG represents an amino protecting group, as for example fluorenylmethyloxycarbonyl, benzyloxycarbonyl, allyloxycarbonyl or tert-butyloxycarbonyl. In addition, interconversion of any of the substituents R³, R⁵, R⁷, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further Interconversion of substituents. Appropriate protecting groups and their introduction and cleavage are well-known to the person skilled in the art (see for example T.W. Greene and P.G.M. Wuts in Protective Groups in Organic Synthesis, 3^{rd} edition, Wiley 1999). Specific examples are described in the subsequent paragraphs.

Compounds of general formula (1-13) are reacted with a compound of general formula (1-14) as mentioned above with a peptide coupling agent, for example N-[(dimethylamino)(3H-[1,2,3]triazolo[4,5-b]pyridin-3-yloxy)methylidene]-N-methylmethanaminium hexafluorophosphate, in a suitable solvent, such as, for example, dichloromethane, in the presence of a suitable base, such as, for example, diisopropylethylamine in a temperature range from −10° C. to the boiling point of the respective solvent, preferably the reaction is carried out at room temperature, to furnish compounds of general formula (1-15).

Appropriate peptide synthesis methods and their applications are well-known to the person skilled in the art (see for example N. Leo Benoitin in Chemistry of Peptide Synthesis, CRC Press 2005; John Jones in Amino Acids and Peptide Synthesis, Oxford University Press, 2002 and Norbert Sewald and Hans-Dieter Jakubke in Peptides: Chemistry and Biology, Wiley-VCH, 2009).

Intermediates of general formula (1-15) can be converted to intermediates of general formula (1-16) by reaction with Broensted acid, such as, for example trifluoroacetic acid, in a suitable solvent system, such as, for example, dichloromethane, in a temperature range from room temperature to the boiling point of the respective solvent, preferably the reaction is carried out at room temperature.

Compounds of general formula (1-16) are then reacted with a compound of general formula (1-17) as mentioned above with a peptide coupling agent, for example N-[(dimethylamino)(3H-[1,2,3]triazolo[4,5-b]pyridin-3-yloxy)methylidene]-N-methylmethanaminium hexafluorophosphate, in a suitable solvent, such as, for example, dichloromethane, in the presence of a suitable base, such as, for example, diisopropylethylamine in a temperature range from −10° C. to the boiling point of the respective solvent, preferably the reaction is carried out at room temperature, to furnish compounds of general formula (1-18).

Appropriate peptide synthesis methods and their applications are well-known to the person skilled in the art (see for example N. Leo Benoitin in Chemistry of Peptide Synthesis, CRC Press 2005; John Jones in Amino Acids and Peptide Synthesis, Oxford University Press, 2002 and Norbert Sewald and Hans-Dieter Jakubke in Peptides: Chemistry and Biology, Wiley-VCH, 2009).

Intermediates of general formula (1-18) can be converted to the compounds of general formula (1-19) by reaction with Broensted acid, such as, for example trifluoroacetic acid, in a suitable solvent system, such as, for example, dichloromethane, in a temperature range from room temperature to the boiling point of the respective solvent, preferably the reaction is carried out at room temperature.

Compounds of general formula (1-20) can be converted into compounds of general formula (1-6) according to the procedure depicted in Scheme 5.

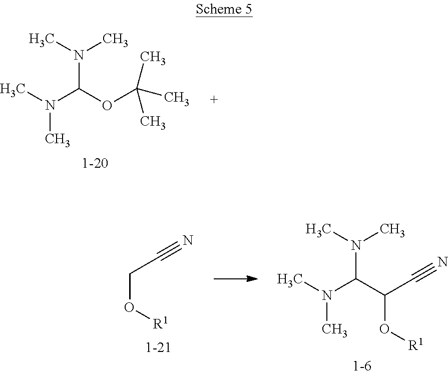

Scheme 5: Process for the transformation of compounds of general formula (1-20) into compounds of general formula (1-6), wherein R¹ has the meaning as given for general formula (I), supra.

Compounds of general formula (1-20) can be converted into compounds of general formula (1-6) by reaction with a suitable substituted cyanoalkyl, such as, for example methoxyacetonitrile, in a temperature range from room temperature to the boiling point of the respective solvent, preferably the reaction is carried out at 800.

Compounds of general formula (1-22) can be converted into compounds of general formula (1-3) according to the procedure depicted in Scheme 6.

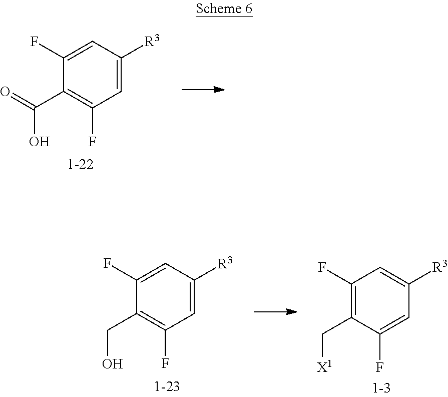

Scheme 6: Process for the transformation of compounds of general formula (1-22) into compounds of general formula (1-3), wherein R³ has the meaning as given for general formula (I). X¹ represents F, Cl, Br, I or a sulfonate, e.g. trifluormethylsulfonate or p-toluolsulfonate.

Compounds of general formula (1-23) can be converted into compounds of general formula (1-3) by reaction with a suitable reducing agent, such as, for example borane, in a suitable solvent system, such as, for example, tetrahydrofuran, in a temperature range from −78° C. to boiling point of the respective solvent, preferably the reaction is carried out at room temperature.

Compounds of general formula (1-23) can be converted into compounds of general formula (1-3) by reaction with a suitable halogenation or sulfonylation agent, such as for example hydrogen bromide, in a suitable solvent, such as, for example, acetic acid, in a temperature range from 0° C. to the boiling point of the respective solvent, preferably the reaction is carried out at room temperature.

Compounds of general formula (1-28) can be converted into compounds of general formula (1-30) according to the procedure depicted in Scheme 7.

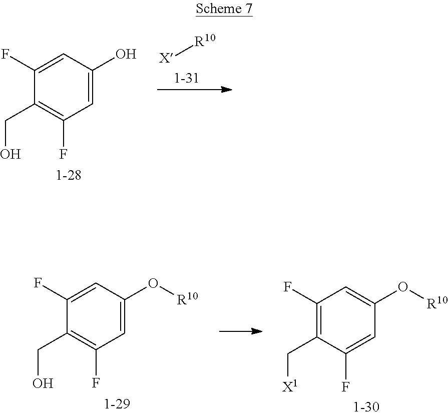

Scheme 7: Route for the transformation of compounds of general formula (1-28) into compounds of general formula (1-30), wherein R¹⁰ represents a C₁-C₆-alkyl, C₁-C₆-haloalkyl, C₃-C₆-cycloalkyl, (C₁-C₆-alkoxy)-(C₂-C₃-alkyl)- or (C₂-C₆-cycloalkyl)-(C₁-C₆-alkyl)- group, or a protected C₂-C₆-hydroxyalkyl group. X′ and X¹ represent F, Cl, Br, I or a sulfonate, e.g. trifluormethylsulfonate or p-toluolsulfonate.

Compounds of general formula (1-28) are reacted with a compound of general formula (1-31), in a suitable solvent, such as, for example, DMF, in the presence of a suitable base, such as, for example, sodium hydride in a temperature range from room temperature to the boiling point of the respective solvent, preferably the reaction is carried out at room temperature, to furnish compounds of general formula (1-29).

Compounds of general formula (1-29) can be converted into compounds of general formula (1-30) by reaction with a suitable halogenation or sulfonylation agent, such as for example hydrogen bromide, in a suitable solvent, such as, for example, acetic acid, in a temperature range from 0° C. to the boiling point of the respective solvent, preferably the reaction is carried out at room temperature.

Compounds of general formula (1-30) can be used for the synthesis of the respective compounds of general formula (I) employing the synthetic methods according to the procedures depicted in Scheme 1.

Compounds of general formula (1-32) can be converted into compounds of general formula (1-38) according to the procedure depicted in Scheme 8.

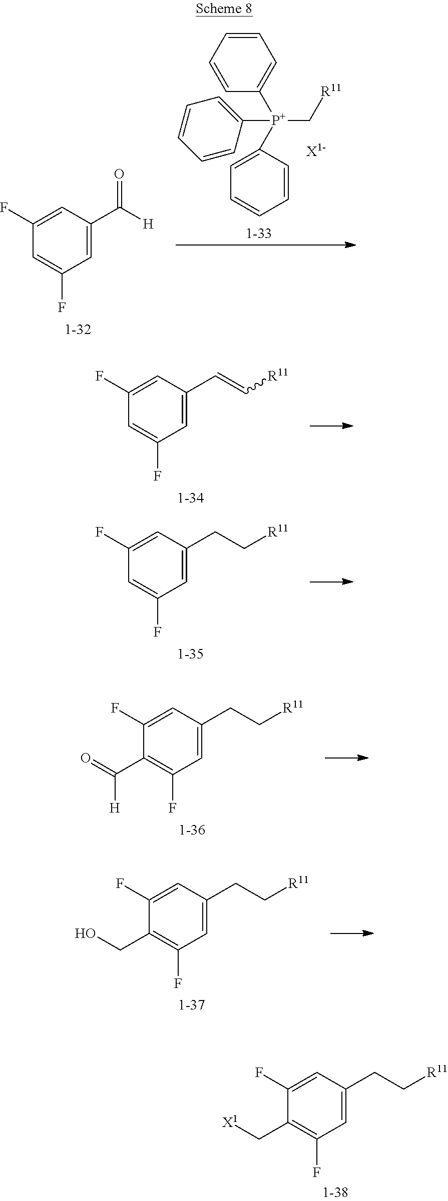

Scheme 8: Route for the transformation of compounds of general formula (1-32) Into compounds of general formula (1-38), wherein R¹¹ represents a C₁-C₄-alkyl- or a (C₃-C₆-cycloalkyl)-CH₂-group. X¹ represents F, Cl, Br, I or a sulfonate, e.g. trifluormethylsulfonate or p-toluolsulfonate.

Compounds of general formula (1-33) are reacted with a compound of general formula (1-32), in a suitable solvent, such as, for example, THF, in the presence of a suitable base, such as, for example, n-butyllithium in a temperature range from −78° C. to the boiling point of the respective solvent, preferably the reaction is carried out at room temperature, to furnish compounds of general formula (1-34).

Compounds of general formula (1-34) can be converted into compounds of general formula (1-35) by reaction with hydrogen in the presence of a suitable catalyst, such as for example palladium on charcoal, in a suitable solvent, such as, for example, methanol, in a temperature range from 0° C. to the b oiling point of the respective solvent, preferably the reaction is carried out at room temperature.

Compounds of general formula (1-35) are then reacted with DMF, in a suitable solvent, such as, for example, THF, in the presence of a suitable base, such as, for example, n-butyllithium in a temperature range from −78° C. to the boiling point of the respective solvent, preferably the reaction is carried out at −78° C., to furnish compounds of general formula (1-36).

Compounds of general formula (1-36) can be converted into compounds of general formula (1-37) by reaction with a suitable reducing agent, such as, for example sodium borohydrate, in a suitable solvent system, such as, for example, tetrahydrofuran, in a temperature range from −78° C. to boiling point of the respective solvent, preferably the reaction is carried out at room temperature.

Compounds of general formula (1-37) can be converted into compounds of general formula (1-38) by reaction with a suitable halogenation or sulfonylation agent, such as for example hydrogen bromide, in a suitable solvent, such as, for example, acetic acid, in a temperature range from 0° C. to the boiling point of the respective solvent, preferably the reaction is carried out at room temperature.

Compounds of general formula (1-38) can be used for the synthesis of the respective compounds of general formula (I) employing the synthetic methods according to the procedures depicted in Scheme 1.

Compounds of general formula (1-39) can be converted into compounds of general formula (1-38-1) according to the procedure depicted in Scheme 9.

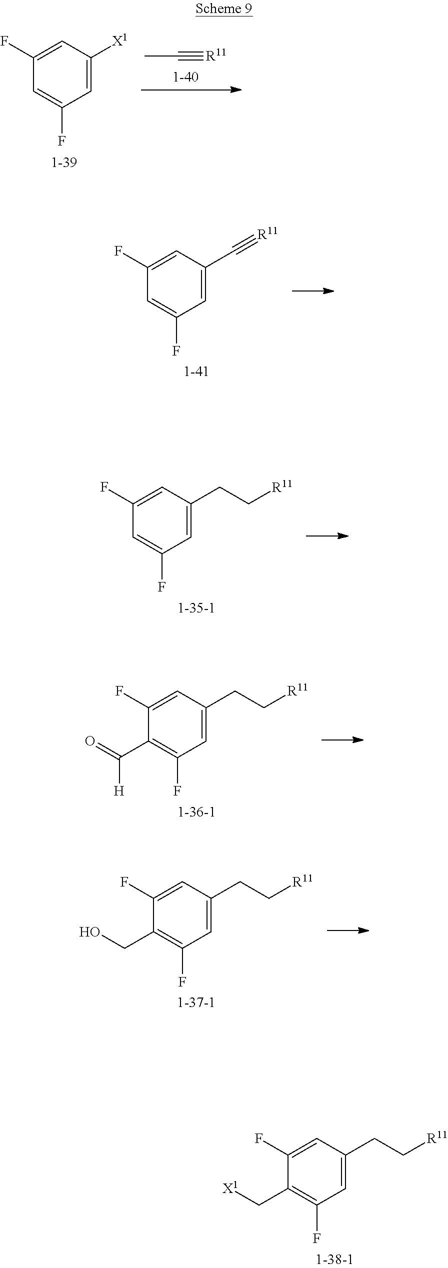

Scheme 9: Route for the transformation of compounds of general formula (1-39) into compounds of general formula (1-38-1), wherein R¹¹ represents a C₁-C₄-alkyl- or a C₁-C₄-hydroxyalkyl- group. X¹ represents F, Cl, Br, I or a sulfonate, e.g. trifluormethylsulfonate or p-toluolsulfonate.

Compounds of general formula (1-39) are reacted with a compound of general formula (1-40), in a suitable solvent, such as, for example, DMF, in the presence of a suitable catalyst system, such as, for example, bis(triphenylphosphine)palladium dichloride and copper(I)iodide, in the presence of suitable base, such as, for example, diisopropylamine, in a temperature range from −78° C. to the boiling point of the respective solvent, preferably the reaction is carried out at room temperature, to furnish compounds of general formula (1-41).

Compounds of general formula (1-41) can be converted into compounds of general formula (1-35-1) by reaction with hydrogen in the presence of a suitable catalyst, such as for example palladium on charcoal, in a suitable solvent, such as, for example, methanol, in a temperature range from 0° C. to the boiling point of the respective solvent, preferably the reaction is carried out at room temperature.

Compounds of general formula (1-35-1) are then reacted with DMF, in a suitable solvent, such as, for example, THF, in the presence of a suitable base, such as, for example, n-butyllithium in a temperature range from −78° C. to the boiling point of the respective solvent, preferably the reaction is carried out at −78° C., to furnish compounds of general formula (1-36-1).

Compounds of general formula (1-36-1) can be converted into compounds of general formula (1-37-1) by reaction with a suitable reducing agent, such as, for example sodium borohydrate, in a suitable solvent system, such as, for example, tetrahydrofuran, in a temperature range from −78° C. to boiling point of the respective solvent, preferably the reaction is carried out at room temperature.

Compounds of general formula (1-37-1) can be converted into compounds of general formula (1-38-1) by reaction with a suitable halogenation or sulfonylation agent, such as for example hydrogen bromide, in a suitable solvent, such as, for example, acetic acid, in a temperature range from 0° C. to the boiling point of the respective solvent, preferably the reaction is carried out at room temperature.

Compounds of general formula (1-38-1) can be used for the synthesis of the respective compounds of general formula (I) employing the synthetic methods according to the procedures depicted in Scheme 1.

Compounds of general formula (1-5) can be converted into compounds of general formula 1a) according to the procedure depicted in Scheme 10.

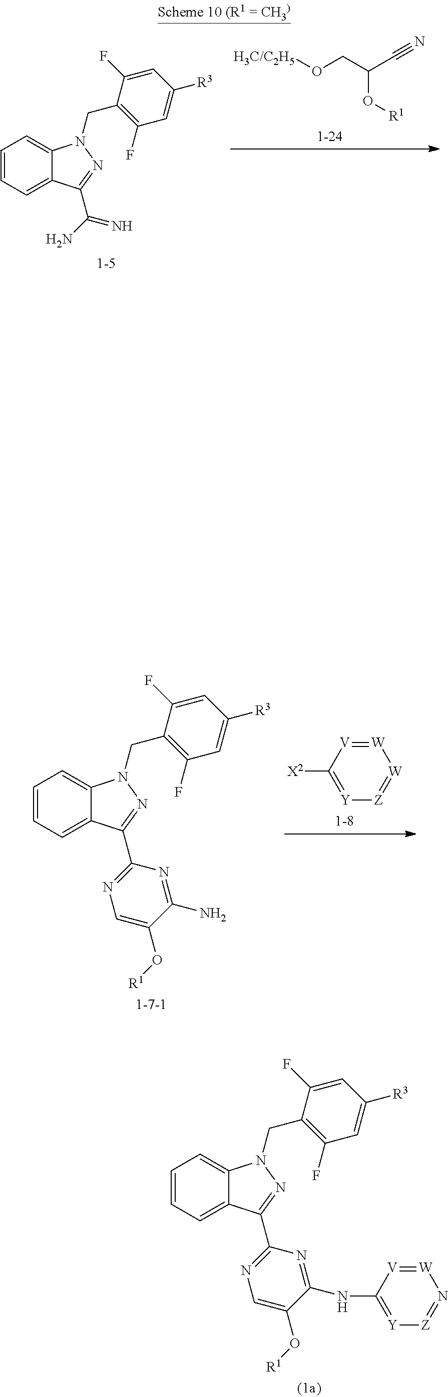

Scheme 10: Alternative route for the preparation of compounds of general formula (1a), wherein R³, V, W, Y and Z have the meaning as given for general formula (I), supra, R¹ represents methyl, and X² represents F, Cl, Br, I, boronic acid or a boronic acid ester, such as for example 4,4,5,5-tetramethyl-2-phenyl-1,3,2-dioxaborolane (boronic acid pinacole ester).

In addition, interconversion of any of the substituents R¹, R³, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which Introduce a functionality which allows for further Interconversion of substituents. Appropriate protecting groups and their introduction and cleavage are well-known to the person skilled in the art (see for example T.W. Greene and P.G.M. Wuts in Protective Groups in Organic Synthesis, 3^{rd} edition, Wiley 1999). Specific examples are described in the subsequent para-graphs.

Compound C is either commercially available or can be prepared according to procedures available from the public domain, as understandable to the person skilled in the art as referred to below.

Intermediates of general formula (1-5) can be converted to intermediates of general formula (1-7-1) by reaction with a suitably substituted 3-methoxyacrylonitrile of the general formula (1-24), in the presence of a suitable base, such as, for example sodium methanolate, in a suitable solvent system, such as, for example, methanol, in a temperature range from room temperature to the boiling point of the respective solvent, preferably the reaction is carried out at 65° C.

Intermediates of general formula (1-7-1) can be reacted with a suitable halo substituted sixmembered heterocycle of the general formula (1-8), such as, for example 4-bromo-2-methyl-pyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (1a). Alternatively the following palladium catalysts can be used:
allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(μ-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium(II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium(II), dichloro(di-μ-chloro)bis[1,3-bis(2,6-di-iso-propylphenyl)imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphos-phonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phosphine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethylbiphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl](2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-7) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (1a).

Alternatively intermediates of general formula (1-7-1) can be reacted with a suitable 4-halopyridine of the general formula (1-8), such as for example 4-fluoro-2-methyl-pyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of genera I formula (1a).

Compounds of general formula (1-5) can be converted into compounds of general formula (Ia) according to the procedure depicted in Scheme 11.

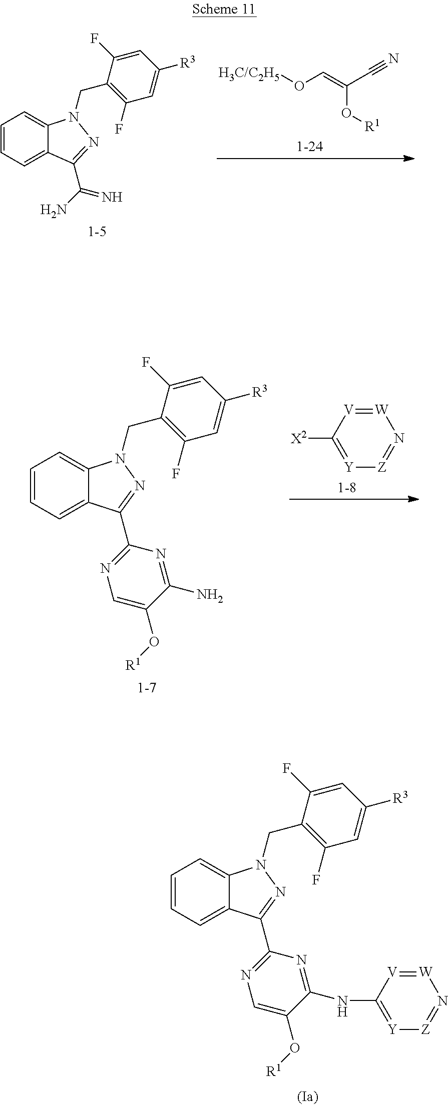

Scheme 11: Alternative route for the preparation of compounds of general formula (Ia), wherein R¹, R³, V, W, Y and Z have the meaning as given for general formula (I), supra. X² represents F, Cl, Br, I, boronic acid or a boronic acid ester, such as for example 4,4,5,5-tetramethyl-2-phenyl-1,3,2-dioxaborolane (boronic acid pinacole ester).

In addition, interconversion of any of the substituents R¹, R³, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T.W. Greene and P.G.M. Wuts in Protective Groups in Organic Synthesis, 3^{rd} edition, Wiley 1999). Specific examples are described in the subsequent para-graphs.

Compound C is either commercially available or can be prepared according to procedures available from the public domain, as understandable to the person skilled in the art as referred to below.

Intermediates of general formula (1-5) can be converted to intermediates of general formula (1-7) by reaction with a suitably substituted 3-methoxyacrylonitrile of the general formula (1-24), in the presence of a suitable base, such as, for example sodium methanolate, in a suitable solvent system, such as, for example, methanol, in a temperature range from room temperature to the boiling point of the respective solvent, preferably the reaction is carried out at 65° C.

Intermediates of general formula (1-7) can be reacted with a suitable halo substituted sixmembered heterocycle of the general formula (1-8), such as, for example 4-bromo-2-methyl-pyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-dylbis(diphenylphosphane), in a suitable solvent system, such as, for example, DMF, in a temperature range from room temperature to the boiling point of the respective solvent, preferably the reaction is carried out at 100° C. to furnish compounds of general formula (Ia). Alternatively the following palladium catalysts can be used:
allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(μ-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl}palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium(II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium(II), dichloro(di-μ-chloro)bis[1,3-bis(2,6-di-iso-propylphenyl)imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methariesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphos-phonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phosphine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethylbiphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl](2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine) sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-7) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (Ia).

Alternatively intermediates of general formula (1-7) can be reacted with a suitable 4-halopyridine of the general formula (1-8), such as for example 4-fluoro-2-methyl-pyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of genera I formula (Ia).

Compounds of general formula (1-1) can be converted into compounds of general formula (1-2) according to the procedure depicted in Scheme 12.

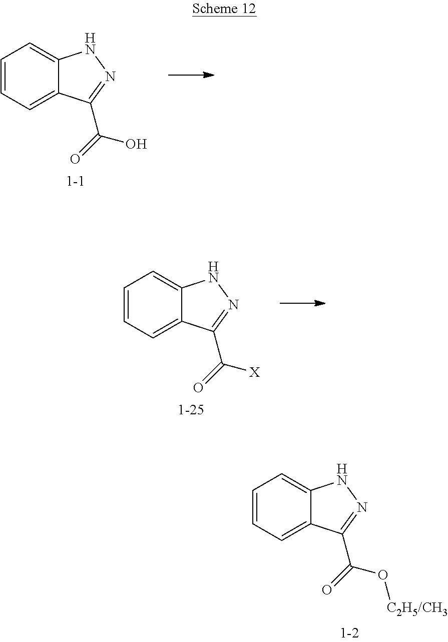

Scheme 12: Route for the preparation of compounds of formula (1-2), supra. X represents Cl and Br.

Alternatively substituted 1H-indazole-3-carboxylic acid of general formula (1-1) can be converted to the corresponding substituted 1H-indazole-3-carbonyl halide of the general formula (1-25) by treatment with thionyl halides, for example thionyl chloride in a suitable solvent system, such as, for example, toluene, at a temperature between 0° C. and boiling point of the respective solvent, preferably the reaction is carried out at 120° C. The substituted 1H-indazole-3-carbonyl halide c an be reacted with methanol or ethanol in the presence of a base, such as, for example, triethylamine, in an suitable solvent system, such as, for example, dichloromethane, at a temperature between −20° C. and boiling point of the respective solvent, preferably the reaction is carried out at 0° C. to yield the desired alkyl 1H-indazole-3-carboxy late intermediates of general formula (1-2).

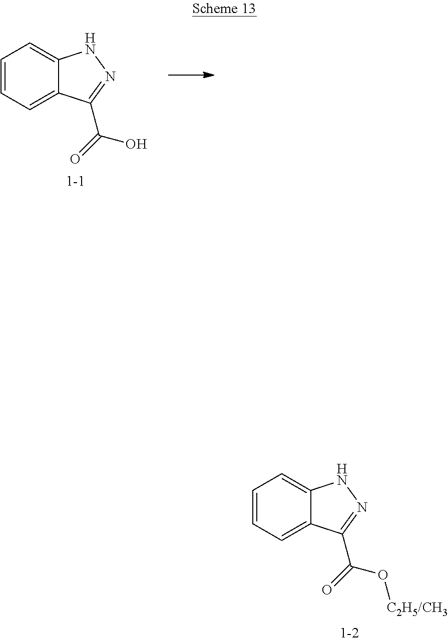

Scheme 13: Route for the preparation of compounds of formula (1-2), supra.

Alternatively compounds of general formula (1-1) are then reacted with methanol or ethanol as mentioned above with a peptide coupling agent, for example N-[(dimethylamino)(3H-[1,2,3]triazolo[4,5-b]pyridin-3-yloxy)methylidene]-N-methylmethanaminium hexafluorophosphate, in a suitable solvent, such as, for example, dichloromethane, in the presence of a suitable base, such as, for example, diisopropylethylamine in a temperature range from −10° C. to the boiling point of the respective solvent, preferably the reaction is carried out at room temperature, to furnish compounds of general formula (1-2).

Appropriate peptide synthesis methods and their applications are well-known to the person skilled in the art (see for example N. Leo Benoitin in Chemistry of Peptide Synthesis, CRC Press 2005; John Jones in Amino Acids and Peptide Synthesis, Oxford University Press, 2002 and Norbert Sewald and Hans-Dieter Jakubke in Peptides: Chemistry and Biology, Wiley-VCH, 2009).

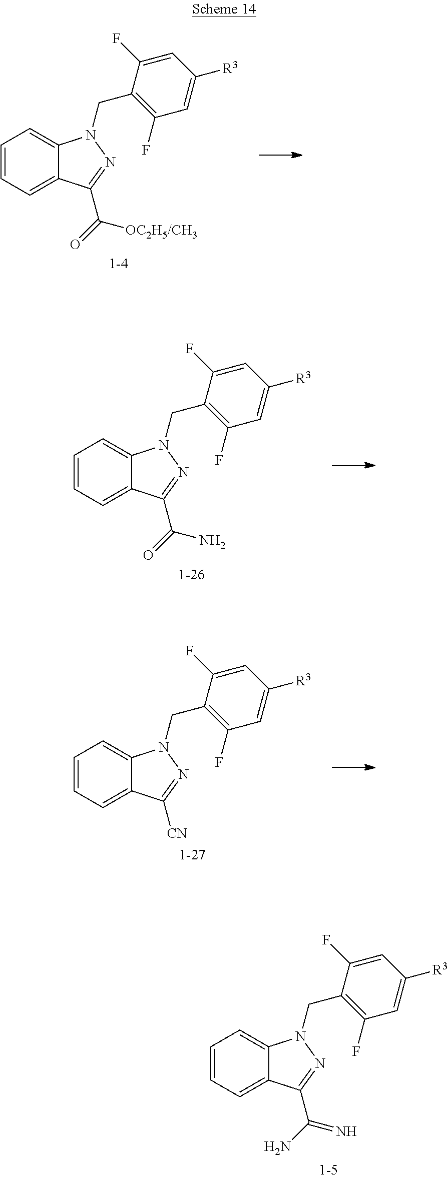

Scheme 14: Route for the preparation of compounds of general formula (1-5), wherein R³ has the meaning as given for general formula (I), supra. In addition, interconversion of R³ can be achieved before and/or after the exemplified transformations. These modifications can be such as the Introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T.W. Greene and P.G.M. Wuts in Protective Groups in Organic Synthesis, 3^{rd} edition, Wiley 1999). Specific examples are described in the subsequent paragraphs.

Compound is either commercially available or can be prepared according to procedures available from the public domain, as understandable to the person skilled in the art. Specific examples are described in the subsequent paragraphs.

Intermediates of general formula (1-4) can be converted to Intermediates of general formula (1-26) by reaction with ammonia, in a suitable solvent system, such as, for example, methanol, at a temperature between 0° C. an d boiling point of the respective solvent, preferably the reaction is carried out at 50° C., at a pressure between 1 and 10 bar, preferably the reaction is carried in a sealed vessel.

Intermediates of general formula (1-26) are treated with triflic anhydride, in a suitable solvent system, such as, for example, tetrahydrofuran, in the presence of a suitable base, such as, for example, pyridine, at a temperature between 0° C. and the boiling point of the respective solvent, preferably the reaction is carried out at room temperature, to form the desired intermediate of general formula (1-27).

Intermediates of general formula (1-27) can be converted to intermediates of general formula (1-5) by reaction with a suitable alcoholate, such as, for example sodium methanolate, in a suitable solvent system, such as, for example, the corresponding alcohol, e.g. methanol, at a temperature between room temperature and the boiling point of the respective solvent, preferably the reaction is carried out at room temperature, and subsequent treatment with a suitable source of ammonium, such as for example, ammonium chloride in the presence of a suitable acid, such as for example acetic acid in a temperature range from room temperature to the boiling point of the respective solvent, preferably the reaction is carried out at 50° C.

An alternative route for the preparation of compounds of general formula (Ia) is described in Scheme 15.

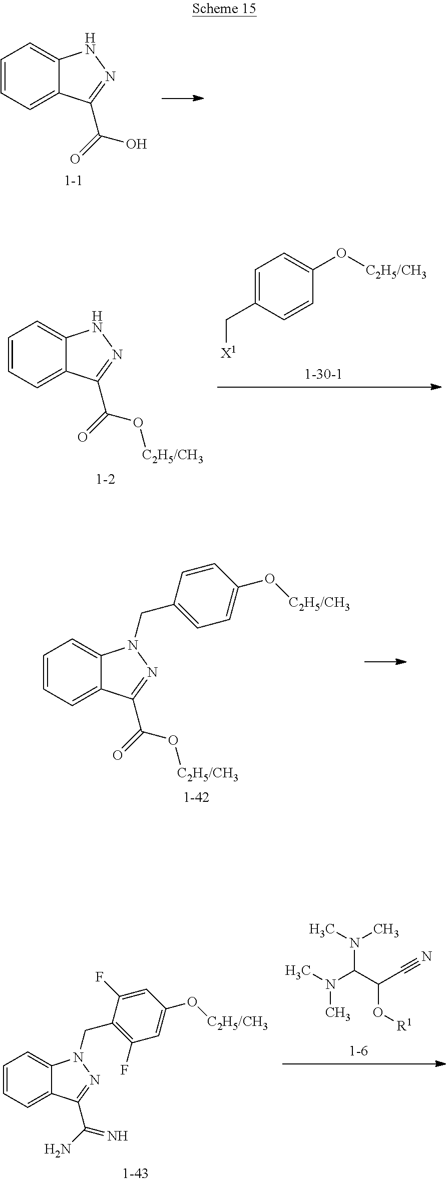

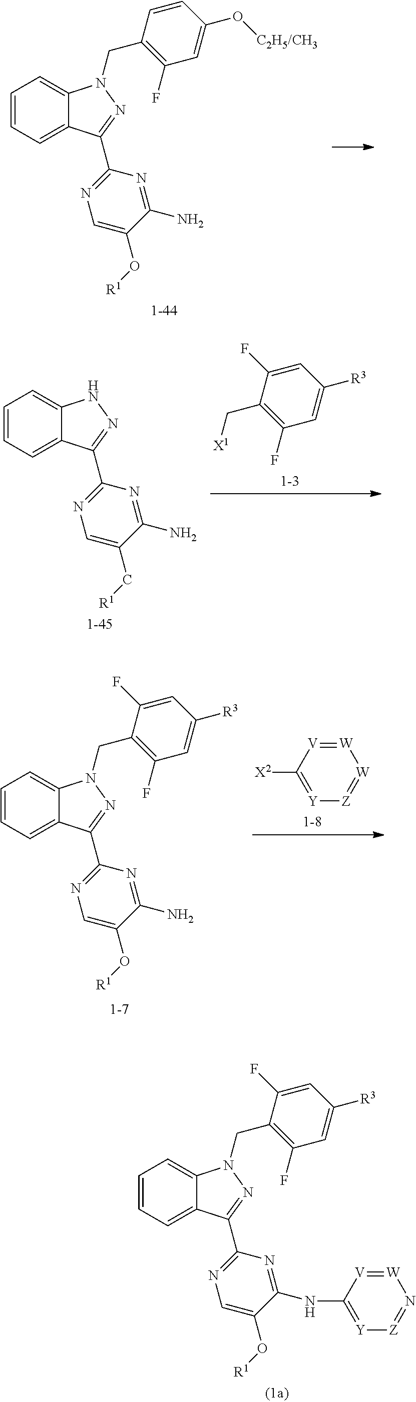

Scheme 15: Route for the preparation of compounds of general formula (Ia), wherein R¹, R³, V, W, Y and Z have the meaning as given for general formula (I), supra, X¹ represents F, Cl, Br, I or a sulfonate, e.g. trifluormethylsulfonate or p-toluolsulfonate, and X² represents F, Cl, Br, I, boronic acid or a boronic acid ester, such as for example 4,4,5,5-tetramethyl-2-phenyl-1,3,2-dioxaborolane (boronic acid pinacole ester). In addition, interconversion of any of the substituents R¹, R³, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T.W. Greene and P.G.M. Wuts in Protective Groups in Organic Synthesis, 3^{rd} edition, Wiley 1999). Specific examples are described in the subsequent paragraphs.

Compounds 1-3, 1-6 and 1-8 are either commercially available or can be prepared according to procedures available from the public domain, as understandable to the person skilled in the art. Specific examples are described in the subsequent paragraphs.

A suitably substituted 1H-indazole-3-carboxylic acid of the general formula (1-1) can be reacted with methanol or ethanol in the presence of catalytic amounts of a Broensted acid, such as, for example, hydrochloric acid or sulphuric acid, at temperatures ranging from 0° C. to boiling point of the respective alcohol, preferably the reaction is carried out at 85° C., to furnish alkyl 1H-indazole-3-carboxylat e intermediates of general formula (1-2).

Alkyl 1H-Indazole-3-carboxylate Intermediates of the general formula (1-2) can be converted to intermediates of general formula (1-42) by reaction with a suitable alkylating agent, such as, for example a substituted benzyl halide (1-30-1), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-42) are treated with the reagent methylchloroaluminiumamide prepared in situ by addition of ammonium chloride to commercially available trimethylaluminium, in a suitable solvent system, such as, for example, toluene, at a temperature between 0° C. and the boiling point of the respective solvent, preferably the reaction is carried out at 80° C. and are quenched with a suitable solvent system, such as, for example, methanol, to form the desired intermediate of general formula(1-43).

Intermediates of general formula (1-43) can be converted to intermediates of general formula (1-44) by reaction with a suitably substituted 3,3-bis-(dimethylamino)propanenitrile of the general formula (1-6), such as, for example 3,3-bis(dimethylamino)-2-methoxypropanenitrile, in the presence of a suitable base, such as, for example piperidine, in a suitable solvent system, such as, for example, 3-methylbutan-1-ol, in a temperature range from room temperature to the boiling point of the respective solvent, preferably the reaction is carried out at 100° C.

Intermediates of general formula (1-44) can be converted to intermediates of general formula (1-45) by reaction with a suitable Broensted acid, such as, for example methanesulfonic acid and trifluoroacetic acid, in a suitable solvent system, such as, for example, dichloromethane, in a temperature range from room temperature to the boiling point of the respective solvent, preferably the reaction is carried out at room temperature.

Intermediates of the general formula (1-45) can be converted to intermediates of general formula (1-7) by reaction with a suitable alkylating agent, such as, for example a substituted benzyl halide (1-3). in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-7) can be reacted with a suitable six membered heterocycle of the general formula (1-8), such as, for example 4-bromo-2-methyl-pyridine, in the presence of a suitable base, such as, for example sodium 2-methylpropan-2-olate, and a suitable palladium catalyst, such as for example (1E,4E)-1,5-diphenylpenta-1,4-dien-3-one-palladium. in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at at 100° C. to furnish compounds of general formula (Ia). Alternatively the following palladium catalysts can be used:
allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(μ-acetato)bis[o-(di-o-tolylphosphino)benzyl]dipalladium(II) [cataCXium® C], allylchlora[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium(II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl) palladium(II), dichloro(di-μ-chloro)bis[1,3-bis(2,6-di-iso-propylphenyl) imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl [2′,4′,6′-tri(propan-2-yl)biphenyl-2-yl]-lambda5-phosphanyl}palladium, (2′-aminobi-phenyl-2-yl)(methariesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl) phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methane-sulfonato-kappaO)palladium-[2′,6′-bis(propan-2-yloxy)biphenyl-2-yl](dicyclohexyl) phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,8′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphos-phonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phosphine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl (28′-diisopropoxybiphenyl-2-yl)phosphine, 2′-(dicyclohexylphosphino)-N,N-dimethylbiphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl](2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine, 1,3-bis(2,4,6-trimethylphenyl)-1,3-dihydro-2H-imidazol-2-ylidene, 1,3-bis[2,6-di(propan-2-yl)phenyl]-1,3-dihydro-2H-imidazol-2-ylidene.

Alternatively intermediates of general formula (1-7) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (Ia).

Alternatively intermediates of general formula (1-7) can be reacted with a suitable six membered heterocycle of the general formula (1-8), such as for example 4-fluoro-2-methyl-pyridine, in the presence of a suitable base, such as, for example sodium hydride, in a suitable solvent system, such as, for example DMF, in a temperature range from room temperature to the boiling point of the respective solvent, preferably the reaction is carried out at 90° C. to furnish corn pounds of general formula (Ia).

Compounds of general formula (1-46) can be converted Into compounds of general formula (1-48) according to the procedure depicted in Scheme 16.

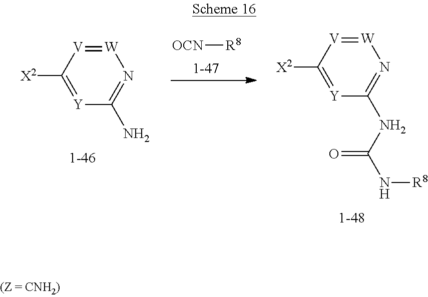

Scheme 16: Route for the preparation of compounds of general formula (1-48), wherein V, W, Y and R⁸ have the meaning as given for general formula (I), supra. X² represents F, Cl, Br and I.

Intermediates of general formula (1-46) can be converted to intermediates of general formula (1-48) by reaction with isocyanate derivative (1-47), in a suitable solvent system, such as, for example, THF, in a temperature range from room temperature to the boiling point of the respective solvent, preferably the reaction is carried out at 70′C.

Compounds of general formula (1-48) can be used for the synthesis of the respective compounds of general formula (I) employing the synthetic methods according to the procedures described above.

It is known to the person skilled in the art that, if there are a number of reactive centers on a starting or intermediate compound, it may be necessary to block one or more reactive centers temporarily by protective groups in order to allow a reaction to proceed specifically at the desired reaction center. A detailed description for the use of a large number of proven protective groups is found, for example, in T. W. Greene, Protective Groups in Organic Synthesis, John Wiley & Sons, 1999, 3rd Ed., or in P. Kocienski, Protecting Groups, Thieme Medical Publishers, 2000.

The compounds according to the invention are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC of compounds of the present invention which possess a sufficiently basic or acidic functionality, may result in the formation of a salt, such as, in the case of a compound of the present invention which is sufficiently basic, a trifluoroacetate or formate salt for example, or, in the case of a compound of the present invention which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of compounds of the present invention may not fully remove traces of cosolvents, especially such as formic acid or trifluoroacetic acid, to give solvates or inclusion complexes. The person skilled in the art will recognise which solvates or inclusion complexes are acceptable to be used in subsequent biological assays. It is to be understood that the specific form (e.g. salt, free base, solvate, inclusion complex) of a compound of the present invention as isolated as described herein is not necessarily the only form in which said compound can be applied to a biological assay in order to quantify the specific biological activity.

Salts of the compounds of formula (I) according to the invention can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar quantitative ratio or one differing therefrom. The salts are obtained by filtering, reprecipitating, precipitating with a non-solvent for the salt or by evaporating the solvent. Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art. Especially preferred are hydrochlorides and the process used in the example section.

Pure diastereomers and pure enantiomers of the compounds and salts according to the invention can be obtained e.g. by asymmetric synthesis, by using chiral starting compounds in synthesis and by splitting up enantiomeric and diasteriomeric mixtures obtained in synthesis.

Enantiomeric and diastereomeric mixtures can be split up into the pure enantiomers and pure diastereomers by methods known to a person skilled in the art. Preferably, diastereomeric mixtures are separated by crystallization, in particular fractional crystallization, or chromatography. Enantiomeric mixtures can be separated e.g. by forming diastereomers with a chiral auxiliary agent, resolving the diastereomers obtained and removing the chiral auxiliary agent. As chiral auxiliary agents, for example, chiral acids can be used to separate enantiomeric bases such as e.g. mandelic acid and chiral bases can be used to separate enantiomeric acids via formation of diastereomeric salts. Furthermore, diastereomeric derivatives such as diastereomeric esters can be formed from enantiomeric mixtures of alcohols or enantiomeric mixtures of acids, respectively, using chiral acids or chiral alcohols, respectively, as chiral auxiliary agents. Additionally, diastereomeric complexes or diastereomeric clathrates may be used for separating enantiomeric mixtures. Alternatively, enantiomeric mixtures can be split up using chiral separating columns in chromatography. Another suitable method for the isolation of enantiomers is the enzymatic separation.

One preferred aspect of the invention is the process for the preparation of the compounds of claims 1 to 7 according to the examples.

Optionally, compounds of the formula (I) can be converted into their salts, or, optionally, salts of the compounds of the formula (I) can be converted into the free compounds. Corresponding processes are customary for the skilled person.

Optionally, compounds of the formula (I) can be converted into their N-oxides. The N-oxide may also be introduced by way of an intermediate. N-oxides may be prepared by treating an appropriate precursor with an oxidizing agent, such as meta-chloroperbenzoic acid, in an appropriate solvent, such as dichioromethane, at suitable temperatures, such as from 0° C. to 40° C., whereby room temperature is generally preferred. Further corresponding processes for forming N-oxides are customary for the skilled person.

One preferred aspect of the invention is the process for the preparation of the compounds of claims 1 to 7 according to the examples, as well as the intermediates used for their preparation.

Optionally, compounds of the formula (I) can be converted into their salts, or, optionally, salts of the compounds of the formula (I) can be converted into the free compounds. Corresponding processes are customary for the skilled person.

Commercial Utility

As mentioned supra, the compounds of the present invention have surprisingly been found to effectively inhibit Bub1 finally resulting in cell death e.g. apoptosis and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by Bub1, such as, for example, benign and malignant neoplasia, more specifically haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof,

especially haematological tumours, solid tumours, and/or metastases of breast, bladder, bone, brain, central and peripheral nervous system, cervix, colon, endocrine glands (e.g. thyroid and adrenal cortex), endocrine tumours, endometrium, esophagus, gastrointestinal tumours, germ cells, kidney, liver, lung, larynx and hypopharynx, mesothelioma, ovary, pancreas, prostate, rectum, renal, small intestine, soft tissue, stomach, skin, testis, ureter, vagina and vulva as well as malignant neoplasias including primary tumors in said organs and corresponding secondary tumors in distant organs (“tumor metastases”). Haematological tumors can e.g be exemplified by aggressive and Indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

A further aspect of the invention is the use of the compounds according to formula (I) for the treatment of cer-vical-, breast-, non-small cell lung-, prostate-, colon- and melanoma tumors and/or metastases thereof, especially preferred for the treatment thereof as well as a method of treatment of cervical-, breast-, non-small cell lung-, prostate-, colon- and melanoma tumors and/or metastases thereof comprising administering an effective amount of a compound of formula (I).

One aspect of the invention is the use of the compounds according to formula (I) for the treatment of cervix tumors as well as a method of treatment of cervix tumors comprising administering an effective amount of a compound of formula (I).

In accordance with an aspect of the present invention therefore the invention relates to a compound of general formula I, or an N-oxide, a salt, a tautomer or a stereoisomer of said compound, or a salt of said N-oxide, tautomer or stereoisomer particularly a pharmaceutically acceptable salt thereof, or a mixture of same, as described and defined herein, for use in the treatment or prophylaxis of a disease, especially for use in the treatment of a disease.

Another particular aspect of the present invention is therefore the use of a compound of general formula I, described supra, or a stereoisomer, a tautomer, an N-oxide, a hydrate, a solvate, or a salt thereof, particularly a pharmaceutically acceptable salt thereof, or a mixture of same, for the prophylaxis or treatment of hyperproliferative disorders or disorders responsive to induction of cell death i.e apoptosis.

The term “inappropriate” within the context of the present invention, in particular in the context of “inappropriate cellular Immune responses, or Inappropriate cellular inflammatory responses”, as used herein, is to be understood as preferably meaning a response which is less than, or greater than normal, and which is associated with, responsible for, or results in, the pathology of said diseases.

Preferably, the use is in the treatment or prophylaxis of diseases, especially the treatment, wherein the diseases are haematological tumours, solid tumours and/or metastases thereof.

Another aspect is the use of a compound of formula (I) is for the treatment of cervical-, breast-, non-small cell lung-, prostate-, colon- and melanoma tumors and/or metastases thereof, especially preferred for the treatment thereof. A preferred aspect Is the use of a compound of formula (I) for the prophylaxis and/or treatment of cervical tumors especially preferred for the treatment thereof.

Another aspect of the present invention is the use of a compound of formula (I) or a stereoisomer, a tautomer, an N-oxide, a hydrate, a solvate, or a salt thereof, particularly a pharmaceutically acceptable salt thereof, or a mixture of same, as described herein, in the manufacture of a medicament for the treatment or prophylaxis of a disease, wherein such disease is a hyperproliferative disorder or a disorder responsive to induction of cell death e.g. apoptosis. In an embodiment the disease is a haematological tumour, a solid tumour and/or metastases thereof. In another embodiment the disease is cervical-, breast-, non-small cell lung-, prostate-, colon- and melanoma tumor and/or metastases thereof, in a preferred aspect the disease is cervical tumor.

Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce cell death e.g. apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias. Examples of breast cancer include, but are not limited to invasive ductal carcinoma, invasive lobular carcinoma, ductal carcinoma in situ, and lobular carcinoma in situ. Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma. Examples of liver cancers include, but are not limited to hepatocellular carcinoma (liver cell carcinomas with or without librolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally. e.g., the management or care of a subject for the purpose of combating, alleviating, reducing, relieving, improving the condition of, etc., of a disease or disorder, such as a carcinoma.

Methods of Treating Kinase Disorders

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant tyrosine kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting a kinase activity, especially of mitogen extracellular kinase, comprising administering an effective amount of a compound of the present invention, including salts, polymorphs, metabolites, hydrates, solvates, prodrugs (e.g.: esters) thereof, and diastereoisomeric forms thereof. Kinase activity can be inhibited in cells (e.g., in vitro), or in the cells of a mammalian subject, especially a human patient in need of treatment.

Methods of Treating Angiogenic Disorders

The present invention also provides methods of treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 6381, age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death e.g. apoptosis of such cell types.

Preferably, the diseases of said method are haematological tumours, solid tumour and/or metastases thereof.

The compounds of the present invention can be used in particular in therapy and prevention i.e. prophylaxis, especially in therapy of tumour growth and metastases, especially in solid tumours of all indications and stages with or without pre-treatment of the tumour growth.

### Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease.

Therefore, the present invention includes pharmaceutical compositions that are comprised of a pharmaceutically acceptable carrier or auxiliary and a pharmaceutically effective amount of a compound, or salt thereof, of the present invention.

Another aspect of the invention is a pharmaceutical composition comprising a pharmaceutically effective amount of a compound of formula (I) and a pharmaceutically acceptable auxiliary for the treatment of a disease mentioned supra, especially for the treatment of haematological tumours, solid tumours and/or metastases thereof.

A pharmaceutically acceptable carrier or auxiliary is preferably a carrier that is non-toxic and innocuous to a patient at concentrations consistent with effective activity of the active ingredient so that any side effects ascribable to the carrier do not vitiate the beneficial effects of the active ingredient. Carriers and auxiliaries are all kinds of additives assisting to the composition to be suitable for administration.

A pharmaceutically effective amount of compound is preferably that amount which produces a result or exerts the intended influence on the particular condition being treated.

The compounds of the present invention can be administered with pharmaceutically-acceptable carriers or auxiliaries well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing auxiliaries, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents Include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quartemary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight.

The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, Isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for administration, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema. S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);
alkalinizing agents (examples Include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal;
aerosol propellants (examples include but are not limited to carbon dioxide, CCl₂F₂, F₂ClC—CClF₂ and CClF₃)
air displacement agents—examples include but are not limited to nitrogen and argon;
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate);
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);
chelating agents (examples include but are not limited to edetate disodium and edetic acid);
colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples Include but are not limited to gelatin and cellulose acetate phthalate),
flavourants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas),
plasticizers (examples include but are not limited to diethyl phthalate and glycerol):
solvents (examples include but are not limited to ethanol, corn oil, cottonseed oil, glycerol, Isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax); suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, crosslinked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to camuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be Illustrated as follows:

Sterile i.v. solution: A 5 mg/mL solution of the desired compound of this invention can be made using sterile, injectable water, and the pH is adjusted if necessary. The solution is diluted for administration to 1-2 mg/mL with sterile 5% dextrose and is administered as an i.v. Infusion over about 60 minutes.
Lyophilised powder for i.v. administration: A sterile preparation can be prepared with (i) 100-1000 mg of the desired compound of this invention as a lyophilised powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.
Intramuscular suspension: The following solution or suspension can be prepared, for intramuscular injection:
50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol
Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.
Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.
Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.
Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Combination Therapies

The compounds of this invention can be administered as the sole pharmaceutical agent or in combination with one or more other pharmaceutical agents where the combination causes no unacceptable adverse effects. Those combined pharmaceutical agents can be other agents having antiproliferative effects such as for example for the treatment of haematological tumours, solid tumours and/or metastases thereof and/or agents for the treatment of undesired side effects. The present invention relates also to such combinations.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, especially (chemotherapeutic) anti-cancer agents as defined supra. The combination can be a non-fixed combination or a fixed-dose combination as the case may be.

Methods of testing for a particular pharmacological or pharmaceutical property are well known to persons skilled in the art.

The example testing experiments described herein serve to illustrate the present invention and the Invention is not limited to the examples given.

As will be appreciated by persons skilled in the art, the invention is not limited to the particular embodiments described herein, but covers all modifications of said embodiments that are within the spirit and scope of the invention as defined by the appended claims.

The following examples illustrate the invention in greater detail, without restricting it. Further compounds according to the invention, of which the preparation is not explicitly described, can be prepared in an analogous way.

The compounds, which are mentioned in the examples and the salts thereof represent preferred embodiments of the invention as well as a claim covering all subcombinations of the residues of the compound of formula (I) as disclosed by the specific examples.

The term “according to” within the experimental section is used in the sense that the procedure referred to is to be used “analogously to”.

Experimental Part

The following table lists the abbreviations used in this paragraph and in the Intermediate Examples and Examples section as far as they are not explained within the text body.

| Abbreviation | Meaning |
|---|---|
| br | broad |
| Cl | chemical ionisation |
| d | doublet |
| dd | doublet of doublet |
| DAD | diode array detector |
| DCM | dichloromethane |
| DMF | N,N-dimethylformamide |
| ELSD | Evaporative Light Scattering Detector |
| eq. | equivalent |
| ESI | electrospray (ES) ionisation |
| HPLC | high perlormance liquid chromatography |
| LC-MS | liquid chromatography mass spectrometry |
| m | multiplet |
| MS | mass spectrometry |
| NMR | nuclear magnetic resonance spectroscopy: chemical |
| | shifts (δ) are given in ppm. The chemical shifts were |
| | corrected by setting the DMSO signal to 2.50 ppm |
| | using unless otherwise stated. |
| PDA | Photo Diode Array |
| PoraPak ™; | a HPLC column obtainable from Waters |
| q | quartet |
| quin | quintet |
| r.t. or rt | room temperature |
| RT | retention time (as measured either with |
| | HPLC or UPLC) in minutes |
| s | singlet |
| SM | starting material |
| SQD | Single-Ouadrupol-Detector |
| t | triplet |
| td | triplet of doublet |
| tt | triplet of triplet |
| THF | tetrahydrofuran |
| UPLC | ultra performance liquid chromatography |

Other abbreviations have their meanings customary per se to the skilled person.

The various aspects of the invention described in this application are Illustrated by the following examples which are not meant to limit the invention in any way.

Specific Experimental Descriptions

NMR peak forms in the following specific experimental descriptions are stated as they appear in the spectra, possible higher order effects have not been considered. Reactions employing microwave Irradiation may be run with a Biotage Initator® microwave oven optionally equipped with a robotic unit. The reported reaction times employing microwave heating are intended to be understood as fixed reaction times after reaching the indicated reaction temperature. The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. from Separtis such as Isolute® Flash silica gel or Isolute® Flash NH₂ silica gel in combination with a Isolera® autopurifler (Biotage) and eluents such as gradients of e.g. hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia. In some cases, purification methods as described above can provide those compounds of the present invention which possess a sufficiently basic or so acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc) of a compound of the present invention as isolated as described herein is not necessarily the only form in which said compound can be applied to a biological assay in order to quantify the specific biological activity.

The percentage yields reported in the following examples are based on the starting component that was used in the lowest molar amount. Air and moisture sensitive liquids and solutions were transferred via syringe or cannula, and introduced into reaction vessels through rubber septa. Commercial grade reagents and solvents were used without further purification. The term “concentrated in vacuo” refers to use of a Buchi rotary evaporator at a minimum pressure of approximately 15 mm of Hg. All temperatures are reported uncorrected in degrees Celsius (° C.).

In order that this invention may be better understood, the following examples are set forth. These examples are for the purpose of illustration only, and are not to be construed as limiting the scope of the invention in any manner. All publications mentioned herein are incorporated by reference in their entirety.

Analytical LC-MS Conditions

LC-MS-data given in the subsequent specific experimental descriptions refer (unless otherwise noted) to the following conditions:

| | |
|---|---|
| System: | Waters Acquity UPLC-MS: Binary Solvent Manager, |
| | Sample Manager/Organizer, Column Manager, PDA, |
| | ELSD, SQD 3001 or ZQ4000 |
| Column: | Acquity UPLC BEH C18 1.7 50 × 2.1 mm |
| Solvent: | A1 = water + 0.1% vol. formic acid (99%) |
| | A2 = water + 0.2% vol. ammonia (32%) |
| | B1 = acetonitrile |
| Gradient: | 0-1.6 min 1-99% B, 1.6-2.0 min 99% B |
| Flow: | 0.8 mL/min |
| Temperature: | 60° C. |
| Injection: | 2.0 μl |
| Detection: | DAD scan range 210-400 nm -> Peak table |
| | ELSD |
| Methods: | MS ESI+, ESI-Switch -> various scan ranges |
| | (Report Header) |
| | Method 1: A1 + B1 = C:\MassLynx\Mass_100_1000.flp |
| | Method 2: A1 + B1 = C:\MassLynx\Mass_160_1000.flp |
| | Method 3: A1 + B1 = C:\MassLynx\Mass_160_2000.flp |
| | Method 4: A1 + B1 = C:\MassLynx\ |
| | Mass_160_1000_BasicReport.flp |
| | Method 5: A2 + B1 = C:\MassLynx\ |
| | NH₃_Mass_100_1000.flp |
| | Method 6: A2 + B1 = C:\MassLynx\NH₃_Mass_160- |
| | _1000_BasicReport.flp |

Preparative HPLC Conditions

“Purification by preparative HPLC” in the subsequent specific experimental descriptions refers to (unless otherwise noted) the following conditions:

Analytics (Pre- and Post-Analytics: Method B):

| | |
|---|---|
| System: | Waters Acicuity UPLC-MS: Binary Solvent |
| | Manager, Sample Manager/Organizer, |
| | Column Manager, FDA, ELSD, SQD 3001 |
| Column: | Aqcuity BEH C18 1.7 50 × 2.1 mm |
| Solvent: | A = water + 0.1% vol. formic acid (99%) |
| | B = acetonitrile |
| Gradient: | 0-1.6 min 1-99% B, 1.6-2.0 min 99% B |
| Flow: | 0.8 mL/min |
| Temperature: | 60° C. |
| Injection: | 2.0 μl |
| Detection: | DAD scan range 210-400 nm |
| | MS ESI+, ESI−, scan range 160-1000 m/z |
| | ELSD |
| Methods: | Purify_pre.flp |
| | Purify_post.flp |

Preparation:

| | |
|---|---|
| System: | Waters Autopurificationsystem: Pump 2545, |
| | Sample Manager 2767, CFO, |
| | DAD 2996, ELSD 2424, SQD 3001 |
| Column: | XBrigde C18 5 μm 100 × 30 mm |
| Solvent: | A = water + 0.1% vol. formic acid (99%) |
| | B = acetonitrile |
| Gradient: | 0-1 min 1% B, 1-8 min 1-99% B, 8-10 min 99% B |
| Flow: | 50 mL/min |
| Temperature: | RT |
| Solution: | max. 250 mg/2.5 mL dimethyl sufoxide or DMF |
| Injection: | 1 × 2.5 mL |
| Detection: | DAD scan range 210-400 nm |
| | MS ESI+, ESI−, scan range 160-1000 m/z |

Chiral HPLC Conditions

If not specified otherwise, chiral HPLC-data given in the subsequent specific experimental descriptions refer to the following conditions:

Analytics:

| | |
|---|---|
| System: | Dionex: Pump 680, ASI 100, Waters: UV-Detektor 2487 |
| Column: | Chiralpak IC 5 μm 150 × 4.6 mm |
| Solvent: | hexane/ethanol 80:20 + 0.1% diethylamine |
| Flow: | 1.0 mL/min |
| Temperature: | 25° C. |
| Solution: | 1.0 mg/mL ethanol/methanol 1:1 |
| Injection: | 5.0 μl |
| Detection: | UV 280 nm |

Preparation:

| | |
|---|---|
| System: | Agilent: Prep 1200, 2xPrep Pump, DLA, MWD, |
| | Prep FC, ESA: Corona |
| Column: | Chiralpak IC 5 μm 250 × 30 mm |
| Solvent: | hexane/ethanol 80:20 + 0.1% diethylamine |
| Flow: | 40 mL/min |
| Temperature: | RT |
| Solution: | 660 mg/5.6 mL ethanol |
| Injection: | 8 × 0.7 mL |
| Detection: | UV 280 nm |

Flash Column Chromatography Conditions

“Purification by (flash) column chromatography” as stated in the subsequent specific experimental descriptions refers to the use of a Biotage Isolera purification system. For technical specifications see “Biotage product catalogue” on www.biotage.com.

Determination of Optical Rotation Conditions

Optical rotations were measured in dimethyl sulfoxide at 589 nm wavelength, 20° C., concentration 1.0000 g/100 ml, integration time 10 s, film thickness 100 00 mm.

### EXAMPLES

### Synthetic Intermediates

### Intermediate 1-1-1

### Preparation of 5-(3-{[tert-butyl(dimethyl)silyl]oxy}propoxy)-N-[2-(difluoromethyl)pyridin-4-yl]-2-[1-(4-ethoxy-2,6-difluorobenzyl)-1H-indazol-3-yl]pyrimidin-4-amine

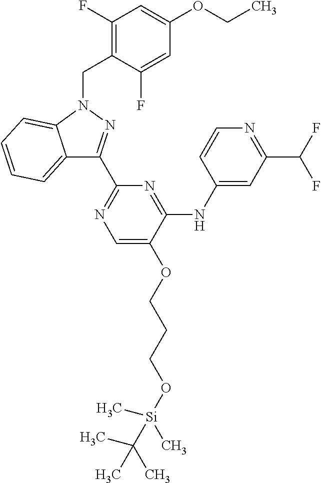 [SMILES: CCOc1cc(F)c(Cn2nc(-c3ncc(OCCCO[Si](C)(C)C(C)(C)C)c(Nc4ccnc(C(F)F)c4)n3)c3ccccc32)c(F)c1]

1.17 g of 4-{[2-(difluoromethyl)pyridin-4-yl]amino}-2-[1-(4-ethoxy-2,6-difluorobenzyl)-1H-indazol-3-yl]pyrimidin-5-ol 1-2-1 (2.23 mmol, 1.0 eq.) was dissolved in 51 mL DMF. 1.54 g of potassium carbonate (11.2 mmol. 5.0 eq.) and 0.85 g of (3-Brompropoxy)-tert-butyldimethylsilan (3.35 mmol, 1.5 eq.) were added. The reaction mixture was stirred at 60° C. over night. The reaction mixture was diluted with ethyl acetate and water. The aqueous layer was extracted with ethyl acetate twice. The combined organic layers were dried using a water resistant filter and the filtrate was dried under reduced pressure by rotary evaporation. The crude product was purified by flash chromatopgraphy to provide the target compound in 98% purity: 1.64 g, 2.3 mmol, 100%.

¹H-NMR (400 MHz, DMSO-ds): δ [ppm]=0.00 (s, 6H), 0.83 (s, 9H), 1.27 (t, 3H), 1.99-2.10 (m, 2H), 3.84 (t, 2H), 4.02 (q. 2H), 4.31 (t, 2H), 5.68 (s, 2H), 6.69-7.04 (m, 3H), 7.20-7.28 (m, 1H). 7.43-7.53 (m, 1H), 7.81 (d, 1H), 8.27-8.31 (m, 1H), 8.39 (s, 1H), 8.44 (d, 1H), 8.48-8.58 (m, 2H), 9.40 (s, 1H).

The following intermediates were prepared according to the same procedure from the indicated starting material (SM=starting material):

| | | | |
|---|---|---|---|
| 1-1-2 SM = 1-2-2 | 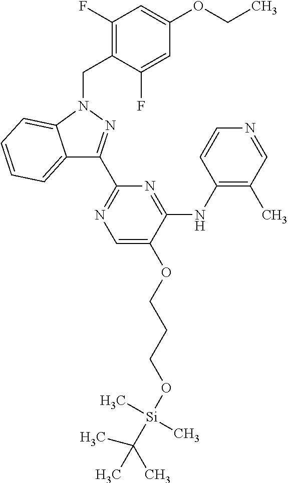 [SMILES: CCOc1cc(F)c(Cn2nc(-c3ncc(OCCCO[Si](C)(C)C(C)(C)C)c(Nc4ccncc4C)n3)c3ccccc32)c(F)c1] | 5-(3-{[tert- butyl(dimethyl)silyl] oxy}propoxy)-2- [1-(4-ethoxy-2,6- difluorobenzyl)- 1H-indazol-3-yl]- N-(3- methylpyridin-4- yl)pyrimidin-4- amine | LC-MS: retention time: 1.77 min MS ES+: 661.3 [M + H]⁺ Method 5 |
| 1-1-3 SM = 1-2-3 | 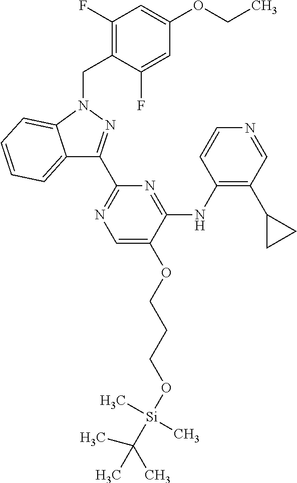 [SMILES: CCOc1cc(F)c(Cn2nc(-c3ncc(OCCCO[Si](C)(C)C(C)(C)C)c(Nc4ccncc4C4CC4)n3)c3ccccc32)c(F)c1] | 5-(3-{[tert- butyl(dimethyl)silyl] oxy}propoxy)-N- (3- cyolopropylpyridin- 4-yl)-2-[1-(4- ethoxy-2,6- difluorobenzyl)- 1H-indazol-3- yl]pyrimidin-4- amine | LC-MS: retention time: 1.86 min MS ES+: 687.5 [M + H]⁺ Method 5 |
| 1-1-4 SM = 1-2-4 | 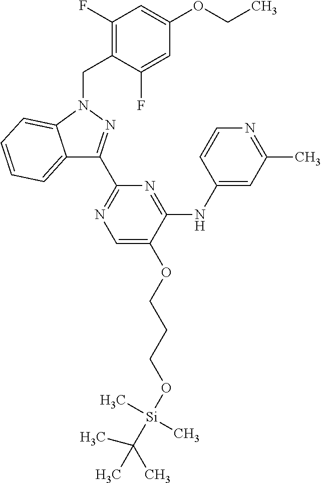 [SMILES: CCOc1cc(F)c(Cn2nc(-c3ncc(OCCCO[Si](C)(C)C(C)(C)C)c(Nc4ccnc(C)c4)n3)c3ccccc32)c(F)c1] | 5-(3-{[tert- butyl(dimethyl)silyl] oxy}propoxy)-2- [1-(4-ethoxy-2,6- difluorobenzyl)- 1H-indazol-3-yl]- N-(2- methylpyridin-4- yl)pyrimidin-4- amine | LC-MS: retention time: 1.76 min MS ES+: 661.3 [M + H]⁺ Method 5 |
| 1-1-5 SM = 1-2-5 | 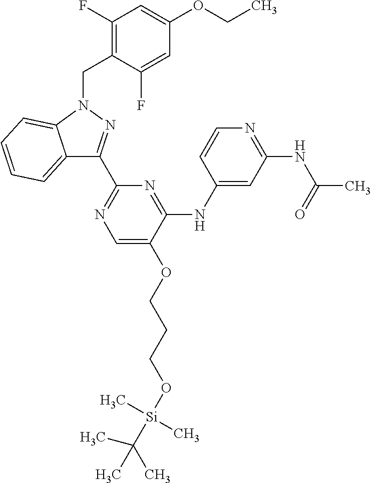 [SMILES: CCOc1cc(F)c(Cn2nc(-c3ncc(OCCCO[Si](C)(C)C(C)(C)C)c(Nc4ccnc(NC(C)=O)c4)n3)c3ccccc32)c(F)c1] | N-[4-({5-(3-{[tert- butyl(dimethyl)silyl] oxy}propoxy)-2- [1-(4-ethoxy-2,6- difluorobenzyl)- 1H-indazol-3- yl]pyrimidin-4- yl}amino)pyridin- 2-yl]acetamide | LC-MS: retention time: 1.69 min MS ES+: 704.3 [M + H]⁺ Method 5 |
| 1-1-6 SM = 1-2-6 | 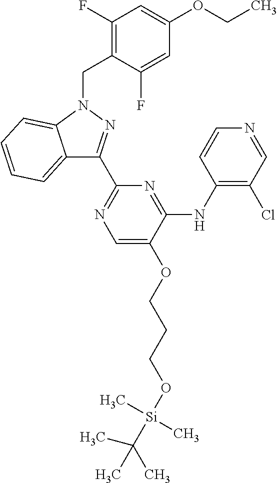 [SMILES: CCOc1cc(F)c(Cn2nc(-c3ncc(OCCCO[Si](C)(C)C(C)(C)C)c(Nc4ccncc4Cl)n3)c3ccccc32)c(F)c1] | 5-(3-{[tert- butyl(dimethyl)silyl] oxy}propoxy)-N- (3-chloropyridin- 4-yl)-2-[1-(4- ethoxy-2,6- difluorobenzyl)- 1H-indazol-3- yl]pyrimidin-4- amine | ¹H-NMR (400 MHz, DMSO-d₆): δ [ppm] = 0.01 (s, 6H), 0.83 (s, 9H), 1.28 (t, 3H), 1.98-2.08 (m, 2H), 3.84 (t, 2H), 4.03 (q, 2H), 4.35 (t, 2H), 5.68 (3, 2H), 6.76- 6.85 (m, 2H), 7.23- 7.30 (m, 1H), 7.45- 7.51 (m, 1H), 7.84 (d, 1H), 8.23 (s, 1H), 8.40 (d, 1H), 8.43- 8.49 (m, 2H), 8.64 (s, 1H), 9.01 (d, 1H). |
| 1-1-7 SM = 1-2-7 | 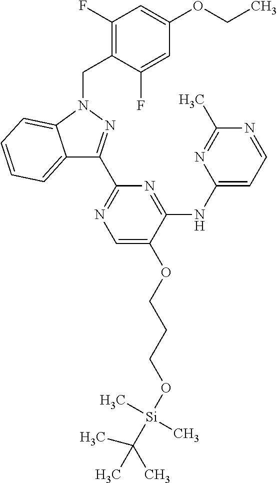 [SMILES: CCOc1cc(F)c(Cn2nc(-c3ncc(OCCCO[Si](C)(C)C(C)(C)C)c(Nc4ccnc(C)n4)n3)c3ccccc32)c(F)c1] | 5-(3-{[tert- butyl(dimethyl)silyl] oxy}propoxy)-2- [1-(4-ethoxy-2,6- difluorobenzyl)- 1H-indazol-3-yl]- N-(2- methylpyrimidin- 4-yl)pyrimidin-4- amine | LC-MS: retention time: 1.84 min MS ES+: 662.7 [M + H]⁺ Method 5 ¹H-NMR (400 MHz, DMSO-d₆): δ [ppm] = 0.02 (s, 6H), 0.84 (s, 9H), 1.28 (t, 3H), 2.02 (quint, 2H), 2.55 (s, 3H), 3.82 (t, 2H), 3.99-4.07 (m, 2H), 4.28 (t, 2H), 5.67 (s, 2H), 6.76-6.85 (m, 2H), 7.25-7.31 (m, 1H), 7.45-7.52 (m, 1H), 7.84 (d, 1H), 8.41-8.48 (m, 2H), 8.52 (d, 1H), 8.58 (d, 1H), 8.95 (s, 1H). |
| 1-1-8 SM = 1-2-8 | 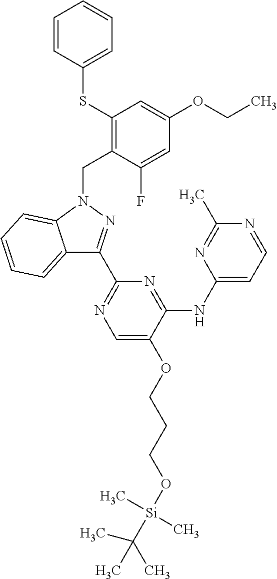 [SMILES: CCOc1cc(F)c(Cn2nc(-c3ncc(OCCCO[Si](C)(C)C(C)(C)C)c(Nc4ccnc(C)n4)n3)c3ccccc32)c(Sc2ccccc2)c1] | 5-(3-{[tert- butyl(dimethyl)silyl] oxy}propoxy)-2- {1-[4-ethoxy-2- fluoro-6- (phenylsulfanyl) benzyl]-1H- indazol-3-yl}-N- (2- methylpyrimidin- 4-yl)pyrimidin-4- amine | LC-MS: retention time: 1.95 min MS ES+: 752.5 [M + H]⁺ Method 5 |
| 1-1-9 SM = 1-3-8 | 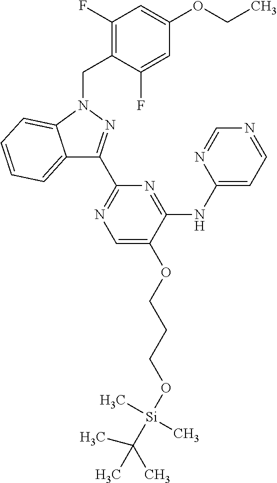 [SMILES: CCOc1cc(F)c(Cn2nc(-c3ncc(OCCCO[Si](C)(C)C(C)(C)C)c(Nc4ccncn4)n3)c3ccccc32)c(F)c1] | 5-(3-{[tert- butyl(dimethyl)silyl] oxy}propoxy)-2- [1-(4-ethoxy-2,6- difluorobenzyl)- 1H-indazol-3-yl]- N-(pyrimidin-4- yl)pyrimidin-4- amine | ¹H-NMR (400 MHz, DMSO-d₆): δ [ppm] = 0.03 (s, 6H), 0.85 (s, 9H), 1.30 (t, 3H), 1.98-2.11 (m, 2H), 3.84 (t, 2H), 3.99- 4.10 (m, 2H), 4.31 (t, 2H), 5.71 (s, 2H), 6.79-6.89 (m, 2H), 7.26-7.34 (m, 1H), 7.48-7.55 (m, 1H), 7.87 (d, 1H), 8.45- 8.52 (m, 2H), 8.63 (d, 1H), 8.75-8.82 (m, 1H), 8.89 (s, 1H), 9.18 (s, 1H). |
| 1-1-10 SM = 1-2-9 | 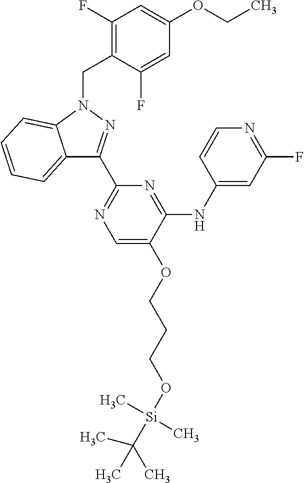 [SMILES: CCOc1cc(F)c(Cn2nc(-c3ncc(OCCCO[Si](C)(C)C(C)(C)C)c(Nc4ccnc(F)c4)n3)c3ccccc32)c(F)c1] | 5-(3-{[tert- butyl(dimethyl)silyl] oxy}propoxy)-2- [1-(4-ethoxy-2,6- difluorobenzyl)- 1H-indazol-3-yl]- N-(2- fluoropyridin-4- yl)pyrimidin-4- amine | LC-MS: retention time: 1.81 min MS ES+: 665.5 [M + H]⁺ Method 5 |
| 1-1-11 SM = 1-2-10 | 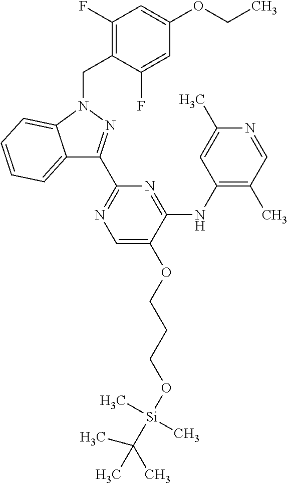 [SMILES: CCOc1cc(F)c(Cn2nc(-c3ncc(OCCCO[Si](C)(C)C(C)(C)C)c(Nc4cc(C)ncc4C)n3)c3ccccc32)c(F)c1] | 5-(3-{[tert- butyl(dimethyl)silyl] oxy}propoxy)-N- (2,5- dimethyipyridin-4- yl)-2-[1-(4-ethoxy- 2,6- difluorobenzyl)- 1H-indazol-3- yl]pyrimidin-4- amine | LC-MS: retention time: 1.81 min MS ES+: 675.5 [M + H]⁺ Method 5 |
| 1-1-12 SM = 1-2-11 | 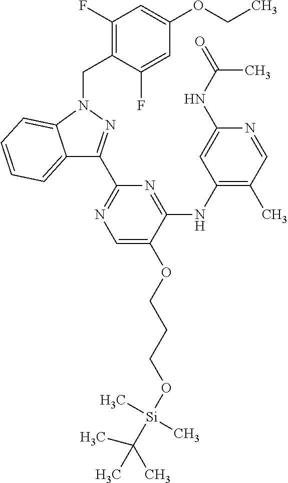 [SMILES: CCOc1cc(F)c(Cn2nc(-c3ncc(OCCCO[Si](C)(C)C(C)(C)C)c(Nc4cc(NC(C)=O)ncc4C)n3)c3ccccc32)c(F)c1] | N-[4-({5-(3-{[tert- butyl(dimethyl)silyl] oxy}propoxy)-2- [1-(4-ethoxy-2,6- difluorobenzyl)- 1H-indazol-3- yl]pyrimidin-4- yl}amino)-5- methylpyridin-2- yl]acetamide | ¹H-NMR (400 MHz, DMSO-d₆): δ [ppm] = 0.04 (s, 6H), 0.87 (s, 9H), 1.30 (t, 3H), 1.96-2.09 (m, 5H), 2.13 (s, 3H), 3.86 (t, 2H), 4.04 (q, 2H), 4.28 (t, 2H), 5.62 (s, 2H), 6.69-6.81 (m, 2H), 6.96-7.05 (m, 1H), 7.36-7.46 (m, 1H), 7.72 (d, 1H), 8.06 (d, 1H), 8.21 (d, 2H), 8.33 (s, 1H), 8.72 (s, 1H), 10.44 (s, 1H). |
| 1-1-13 SM = 1-2-12 | 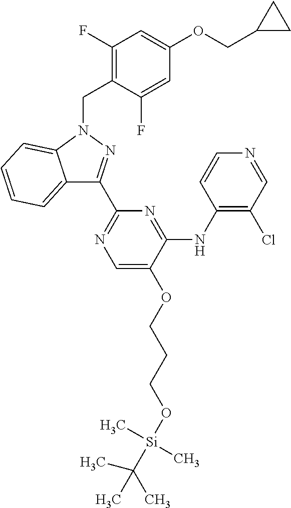 | 5-(3-{[tert- butyl(dimethyl)silyl] oxy}propoxy)-N- (3-chloropyridin- 4-yl)-2-{1-[4- (cyclopropylmethoxy)- 2,6- difluorobenzyl]- 1H-indazol-3- yl}pyrimidin-4- amine | LC-MS: retention time: 1.97 min MS ES+: 525.2 [M + H]⁺ Method 1 |
| 1-1-14 SM = 1-2-13 | 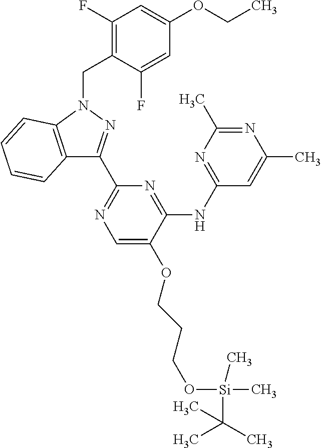 [SMILES: CCOc1cc(F)c(Cn2nc(-c3ncc(OCCCO[Si](C)(C)C(C)(C)C)c(Nc4cc(C)nc(C)n4)n3)c3ccccc32)c(F)c1] | 5-(3-{[tert- Butyl(dimethyl)silyl] oxy}propoxy)- N-(2,6- dimethylpyrimidin- 4-yl)-2-[1-(4- ethoxy-2,6- difluorobenzyl)- 1H-indazol-3- yl]pyrimidin-4- amine | ¹H-NMR (400 MHz, CHLOROFORM-d): δ [ppm] = 0.09 (s, 6H), 0.92 (s, 9H), 1.40 (t, 3H), 2.13 (quint, 2H), 2.60 (s, 3H), 2.65 (s, 3H), 3.88 (t, 2H), 3.98 (q, 2H), 4.34 (t, 2H), 5.74 (s, 2H), 6.43-6.51 (m, 2H), 7.25-7.32 (m, 1H), 7.41-7.48 (m, 1H), 7.62 (d, 1H), 7.98 (s, 1H), 8.30 (s, 1H), 8.58-8.64 (m, 2H). |
| 1-1-15 SM = 1-2-14 | 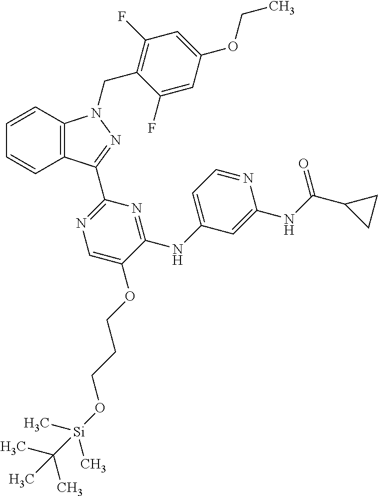 [SMILES: CCOc1cc(F)c(Cn2nc(-c3ncc(OCCCO[Si](C)(C)C(C)(C)C)c(Nc4ccnc(NC(=O)C5CC5)c4)n3)c3ccccc32)c(F)c1] | N-[4-({5-(3-{[tert- Butyl(dimethyl)silyl] oxy}propoxy)-2- [1-(4-ethoxy-2,6- difluorobenzyl)- 1H-indazol-3- yl]pyrimidin-4- yl}amino)pyridin- 2- yl]cyclopropan- carboxamide | ¹H-NMR (400 MHz, DMSO-d₆): δ [ppm] = 0.02 (s, 6H), 0.72- 0.82 (m, 4H), 0.85 (s, 9H), 1.30 (t, 3H), 1.99-2.10 (m, 3H), 3.85 (t, 2H), 4.05 (q, 2H), 4.27 (t, 2H), 5.68 (s, 2H), 6.75- 6.84 (m, 2H), 7.23 (t, 1H), 7.48 (t, 1H), 7.83 (d, 1H), 8.18 (d, 1H), 8.29 (s, 1H), 8.31-8.35 (m, 2H), 8.43 (d, 1H), 9.20 (s, 1H), 10.66 (s, 1H). |
| 1-1-16 SM = 1-2-15 | 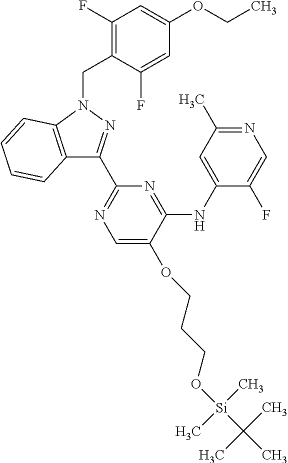 [SMILES: CCOc1cc(F)c(Cn2nc(-c3ncc(OCCCO[Si](C)(C)C(C)(C)C)c(Nc4cc(C)ncc4F)n3)c3ccccc32)c(F)c1] | 5-(3-{[tert- Butyl(dimethyl)silyl] oxy}propoxy)-2- [1-(4-ethoxy-2,6- difluorobenzyl)- 1H-indazol-3-yl]- N-(5-fluor-2- methylpyridin-4- yl)pyrimidin-4- amine | LC-MS: retention time: 1.88 min MS ES+: 679.4 [M + H]⁺ Method 5 |
| 1-1-17 SM = 1-2-16 | 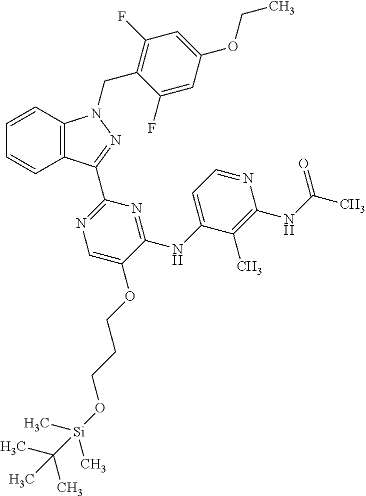 [SMILES: CCOc1cc(F)c(Cn2nc(-c3ncc(OCCCO[Si](C)(C)C(C)(C)C)c(Nc4ccnc(NC(C)=O)c4C)n3)c3ccccc32)c(F)c1] | N-[4-({5-(3-{[tert- butyl(dimethyl)silyl] oxy}propoxy)-2- [1-(4-ethoxy-2,6- difluorobenzyl)- 1H-indazol-3- yl]pyrimidin-4- yl}amino)-3- methylpyridin-2- yl]acetamide | ¹H-NMR (400 MHz, DMSO-d₆): δ [ppm] = 0.03 (s, 6H), 0.85 (s, 9H), 1.29 (t, 3H), 1.97-2.06 (m, 2H), 2.07 (s, 3H), 2.11 (s, 3H), 3.86 (t, 2H), 4.04 (q, 2H), 4.31 (t, 2H), 5.66 (s, 2H), 6.73-6.83 (m, 2H), 7.19 (t, 1H), 7.41- 7.50 (m, 1H), 7.81 (d, 1H), 8.21-8.29 (m, 4H), 8.32 (s, 1H), 10.05 (s, 1H). |
| 1-1-19 SM = 1-2-18 | 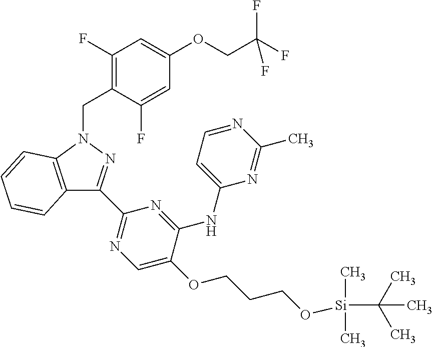 [SMILES: Cc1nccc(Nc2nc(-c3nn(Cc4c(F)cc(OCC(F)(F)F)cc4F)c4ccccc34)ncc2OCCCO[Si](C)(C)C(C)(C)C)n1] | 5-(3-{[tert- butyl(dimethyl)silyl] oxy}propoxy)-2- {1-[2,6-difluoro-4- (2,2,2- trifluoroethoxy) benzyl]-1H-indazol- 3-yl}-N-{2- methylpyrimidin- 4-yl)pyrimidin-4- amine | ¹H-NMR (400 MHz, DMSO-d₆): δ [ppm] = 0.03 (s, 6H), 0.85 (s, 9H), 2.03 (tt, 2H), 2.56 (s, 3H), 3.84 (t, 2H), 4.29 (t, 2H), 4.83 (q, 2H), 5.72 (s, 2H), 7.04 (m, 2H), 7.29 (dd, 1H), 7.51 (dd, 1H), 7.87 (d, 1H), 8.44 (s, 1H), 8.47 (d, 1H), 8.55 (d, 1H), 8.61 (d, 1H), 9.02 (br. s., 1H). |
| 1-1-20 SM = 1-2-19 | 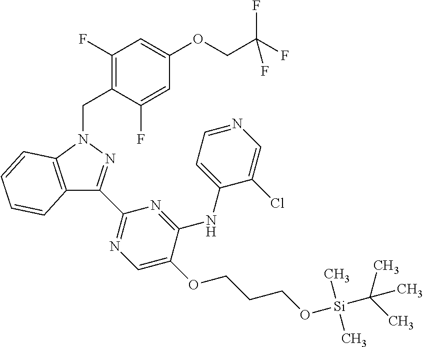 [SMILES: CC(C)(C)[Si](C)(C)OCCCOc1cnc(-c2nn(Cc3c(F)cc(OCC(F)(F)F)cc3F)c3ccccc23)nc1Nc1ccncc1Cl] | 5-(3-{[tert- butyl(dimethyl)silyl] oxy}propoxy)-N- (3-chloropyridin- 4-yl)-2-{1-[2,6- difluoro-4-(2,2,2- trifluoroethoxy) benzyl]-1H-indazol- 3-yl}pyrimidin-4- amine | ¹H-NMR (400 MHz, DMSO-d₆): δ [ppm] = 0.02 (s, 6H), 0.83 (s, 9H), 2.04 (tt, 2H), 3.86 (t, 2H), 4.36 (t, 2H), 4.83 (q, 2H), 5.72 (s, 2H), 7.04 (m, 2H), 7.28 (dd, 1H), 7.51 (dd, 1H), 7.88 (d, 1H), 8.26 (br. s., 1H), 8.41 (s, 1H), 8.48 (d, 1H), 8.49 (s, 1H), 8.66 (s, 1H), 9.03 (d, 1H). |
| 1-1-21 SM = 1-2-20 | 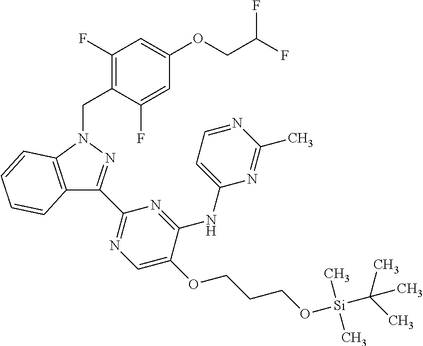 [SMILES: Cc1nccc(Nc2nc(-c3nn(Cc4c(F)cc(OCC(F)F)cc4F)c4ccccc34)ncc2OCCCO[Si](C)(C)C(C)(C)C)n1] | 5-(3-{[tert- butyl(dimethyl)silyl] oxy}propoxy)-2- {1-[4-(2,2- difluoroethoxy)- 2,6- difluorobenzyl]- 1H-indazol-3-yl}- N-(2- methylpyrimidin- 4-yl)pyrimidin-4- amine | ¹H-NMR (400 MHz, DMSO-d₆): δ [ppm] = 0.03 (s, 6H), 0.85 (s, 9H), 2.04 (tt, 2H), 2.56 (s, 3H), 3.84 (t, 2H), 4.29 (t, 2H), 4.37 (td, 2H), 5.71 (s, 2H), 6.37 (tt, 1H), 6.96 (m, 2H), 7.29 (dd, 1H), 7.51 (dd, 1H), 7.87 (d, 1H), 8.44 (s, 1H), 8.47 (d, 1H), 8.56 (d, 1H), 8.61 (d, 1H), 9.02 (br. s., 1H). |
| 1-1-22 SM = 1-2-21 Reaction time: 4 h at 70° C. | 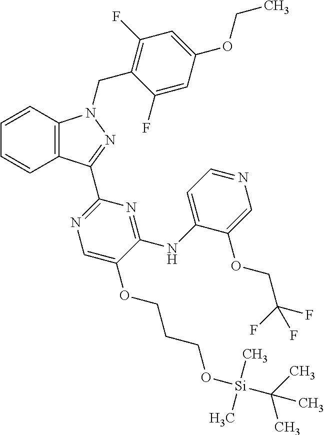 [SMILES: CCOc1cc(F)c(Cn2nc(-c3ncc(OCCCO[Si](C)(C)C(C)(C)C)c(Nc4ccncc4OCC(F)(F)F)n3)c3ccccc32)c(F)c1] | 5-(3-{[tert- butyl(dimethyl)silyl] oxy}propoxy)-2- [1-(4-ethoxy-2,6- difluorobenzyl)- 1H-indazol-3-yl]- N-[3-(2,2,2- trifluoroethoxy) pyridin-4- yl]pyrimidin-4- amine | ¹H-NMR (400 MHz, DMSO-d₆): δ [ppm] = −0.01 (s, 6H), 0.82 (s, 9H), 1.29 (t, 3H), 1.99 (tt, 2H), 3.82 (t, 2H), 4.04 (q, 2H), 4.32 (t, 2H), 5.07 (q, 2H), 5.69 (s, 2H), 6.82 (m, 2H), 7.29 (dd, 1H), 7.51 (dd, 1H), 7.87 (d, 1H), 8.17 (br. s., 1H), 8.28 (d, 1H), 8.43 (s, 1H), 8.47 (s, 1H), 8.47 (d, 1H), 8.98 (d, 1H). |
| 1-1-23 SM = 1-2-22 Reaction time: 19 h at 70° C. | 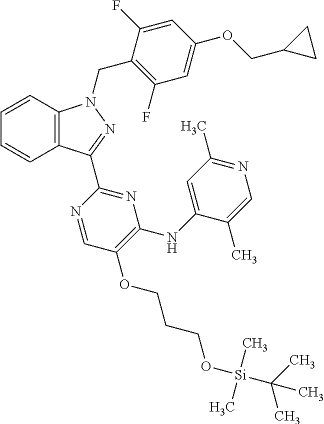 [SMILES: Cc1cc(Nc2nc(-c3nn(Cc4c(F)cc(OCC5CC5)cc4F)c4ccccc34)ncc2OCCCO[Si](C)(C)C(C)(C)C)c(C)cn1] | 5-(3-{[tert- butyl(dimethyl)silyl] oxy}propoxy)-2- {1-[4- (cyclopropylmethoxy)- 2,6- difluorobenzyl]- 1H-indazol-3-yl}- N-(2,5- dimethylpyridin-4- yl)pyrimidin-4- amine | ¹H-NMR (400 MHz, DMSO-d₆): δ [ppm] = 0.03 (s, 6H), 0.29 (m, 2H), 0.54 (m, 2H), 0.85 (s, 9H), 1.17 (m, 1H), 2.03 (tt, 2H), 2.24 (s, 3H), 2.47 (s, 3H), 3.82 (d, 2H), 3.85 (t, 2H), 4.31 (t, 2H), 5.67 (s, 2H), 6.75 (m, 2H), 7.19 (dd, 1H), 7.46 (dd, 1H), 7.81 (d, 1H), 8.03 (br. s., 1H), 8.24 (s, 1H), 8.27 (s, 1H), 8.32 (s, 1H), 8.34 (d, 1H). |
| 1-1-24 SM = 1-2-23 | 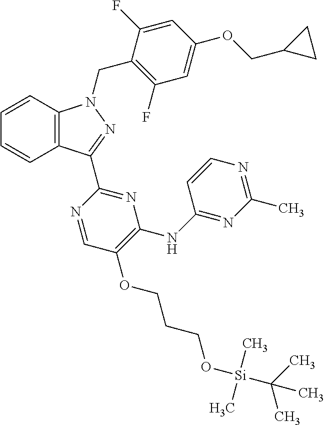 [SMILES: Cc1nccc(Nc2nc(-c3nn(Cc4c(F)cc(OCC5CC5)cc4F)c4ccccc34)ncc2OCCCO[Si](C)(C)C(C)(C)C)n1] | 5-(3-{[tert- butyl(dimethyl)silyl] oxy}propoxy)-2- {1-[4- (cyclopropylmethoxy)- 2,6- difluorobenzyl]- 1H-indazol-3-yl}- N-(2- methylpyrimidin- 4-yl)pyrimidin-4- amine | ¹H-NMR (400 MHz, DMSO-d₆): δ [ppm] = 0.02 (s, 6H), 0.29 (m, 2H), 0.54 (m, 2H), 0.85 (s, 9H), 1.18 (m, 1H), 2.03 (tt, 2H), 2.55 (s, 3H), 3.82 (d, 2H), 3.83 (t, 2H), 4.29 (t, 2H), 5.68 (s, 2H), 6.82 (m, 2H), 7.28 (dd, 1H), 7.50 (dd, 1H), 7.86 (d, 1H), 8.43 (s, 1H), 8.46 (d, 1H), 8.53 (d, 1H), 8.60 (d, 1H), 9.01 (br. s., 1H). |
| 1-1-25 SM = 1-2-24 Reaction time: 19 h at 70° C. | 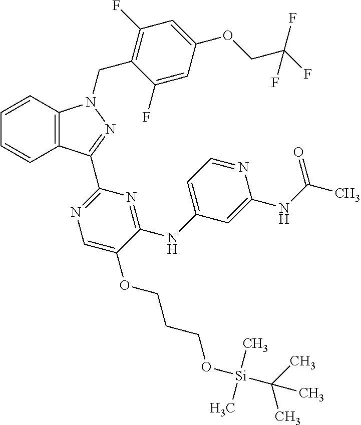 [SMILES: CC(=O)Nc1cc(Nc2nc(-c3nn(Cc4c(F)cc(OCC(F)(F)F)cc4F)c4ccccc34)ncc2OCCCO[Si](C)(C)C(C)(C)C)ccn1] | N-(4-{[5-(3-{[tert- butyl(dimethyl)silyl] oxy}propoxy)-2- {1-[2,6-difluoro-4- (2,2,2- trifluoroethoxy) benzyl]-1H-indazol- 3-yl}pyrimidin-4- yl]amino}pyridin- 2-yl)acetamide | ¹H-NMR (400 MHz, DMSO-d₆): δ [ppm] = 0.02 (s, 6H), 0.85 (s, 9H), 2.04 (tt, 2H), 2.08 (s, 3H), 3.85 (t, 2H), 4.28 (t, 2H), 4.83 (q, 2H), 5.71 (s, 2H), 7.00 (m, 2H), 7.22 (dd, 1H), 7.48 (dd, 1H), 7.83 (d, 1H), 8.18 (d, 1H), 8.28 (br s., 1H), 8.31 (dd, 1H), 8.32 (s, 1H), 8.43 (d, 1H), 9.26 (br. s., 1H), 10.34 (br. s., 1H). |
| 1-1-26 SM = 1-2-25 | 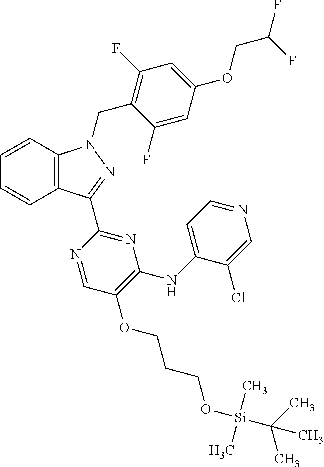 [SMILES: CC(C)(C)[Si](C)(C)OCCCOc1cnc(-c2nn(Cc3c(F)cc(OCC(F)F)cc3F)c3ccccc23)nc1Nc1ccncc1Cl] | 5-(3-{[tert- butyl(dimethyl)silyl] oxy}propoxy)-N- (3-chloropyridin- 4-yl)-2-{1-[4-(2,2- difluoroethoxy)- 2,6- difluorobenzyl]- 1H-indazol-3- yl}pyrimidin-4- amine | ¹H-NMR (400 MHz, DMSO-d₆): δ [ppm] = 0.02 (s, 6H), 0.83 (s, 9H), 2.03 (tt, 2H), 3.85 (t, 2H), 4.36 (t, 2H), 4.37 (td, 2H), 5.71 (s, 2H), 6.37 (tt, 1H), 6.96 (m, 2H), 7.27 (dd, 1H), 7.50 (dd, 1H), 7.87 (d, 1H), 8.25 (br. s., 1H), 8.41 (d, 1H), 8.48 (s, 1H), 8.48 (d, 1H), 8.66 (s, 1H), 9.03 (d, 1H). |
| 1-1-27 SM = 1-2-26 | 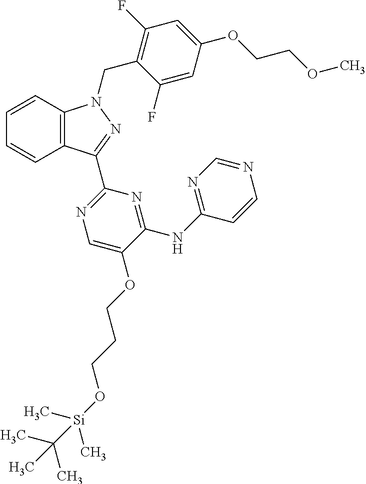 [SMILES: COCCOc1cc(F)c(Cn2nc(-c3ncc(OCCCO[Si](C)(C)C(C)(C)C)c(Nc4ccncn4)n3)c3ccccc32)c(F)c1] | 5-(3-{[tert- butyl(dimethyl)silyl] oxy}propoxy)-2- {1-[2,6-difluoro-4- (2- methoxyethoxy) benzyl]-1H- indazol-3-yl}-N- (pyrimidin-4- yl)pyrimidin-4- amine | ¹H-NMR (400 MHz, DMSO-d₆): δ [ppm] = ] = 0.03 (s, 6H), 0.85 (s, 9H), 1.98-2.08 (m, 2H), 3.26 (s, 3H), 3.57-3.66 (m, 2H), 3.84 (t, 2H), 4.07- 4.17 (m, 2H), 4.30 (t, 2H), 5.70 {s, 2H), 6.82-6.93 (m, 2H), 7.25-7.34 (m, 1H), 7.46-7.56 (m, 1H), 7.87 (d, 1H), 8.43- 8.51 (m, 2H), 8.65 (d, 1H), 8.74-8.82 (m, 1H), 8.86-8.92 (m, 1H), 9.21 (s, 1H). |

### Intermediate 1-2-1

### Preparation of 4-{[2-(difluoromethyl)pyridin-4-yl]amino}-2-[1-(4-ethoxy-2,6-difluorobenzyl)-1H-indazol-3-yl]pyrimidin-5-ol

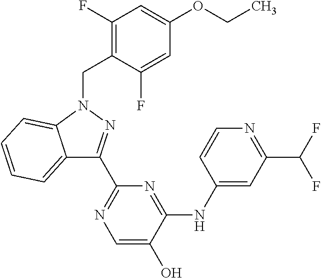 [SMILES: CCOc1cc(F)c(Cn2nc(-c3ncc(O)c(Nc4ccnc(C(F)F)c4)n3)c3ccccc32)c(F)c1]

1.61 g of N-[2-(difluoromethyl)pyridin-4-yl]-2-[1-(4-ethoxy-2,6-difluorobenzyl)-1H-indazol-3-yl]-5-methoxypyrimidin-4-amine 1-3-1 (90% purity, 2.69 mmol, 1.0 eq.) was dissolved in 77 mL 1-Methyl-2-pyrrolidon. 1.49 g of potassium carbonate (10.8 mmol, 4.0 eq.) and molecular sieves were added. Then 414 μL benzenethiol (4.04 mmol, 1.5 eq.) was added and the mixture was stirred at 150° C. for 1 hour.

The reaction mixture was dried at 70′C under reduce d pressure by rotary evaporation. The 83% pure crude product was used without further purification: 1.17 g, 1.85 mmol, 69%.

LC-MS:

retention time: 0.85 min

MS ES+: 707.4 [M+H]⁺

Method 5

The following intermediates were prepared according to the same procedure from the indicated starting material (SM=starting material):

| | | | |
|---|---|---|---|
| 1-2-2 SM = 1-3-2 | 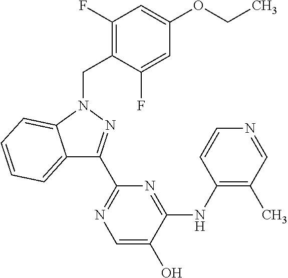 [SMILES: CCOc1cc(F)c(Cn2nc(-c3ncc(O)c(Nc4ccncc4C)n3)c3ccccc32)c(F)c1] | 2-[1-(4-ethoxy-2,6- difluorobenzyl)-1H-indazol- 3-yl]-4-[(3-methylpyridin-4- yl)amino]pyrimidin-5-ol | LC-MS: retention time: 0.84 min MS ES+: 489.3 [M + H]⁺ Method 5 |
| 1-2-3 SM = 1-3-3 | 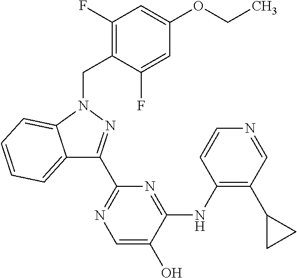 [SMILES: CCOc1cc(F)c(Cn2nc(-c3ncc(O)c(Nc4ccncc4C4CC4)n3)c3ccccc32)c(F)c1] | 4-[(3-cyclopropylpyridin-4- yl)amino]-2-[1-(4-ethoxy- 2,6-difluorobenzyl)-1H- indazol-3-yl]pyrimidin-5-ol | LC-MS: retention time: 0.87 min MS ES+: 515.2 [M + H]⁺ Method 5 |
| 1-2-4 SM = 1-3-4 | 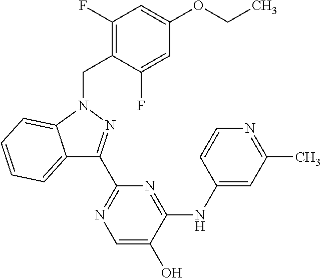 [SMILES: CCOc1cc(F)c(Cn2nc(-c3ncc(O)c(Nc4ccnc(C)c4)n3)c3ccccc32)c(F)c1] | 2-[1-(4-ethoxy-2,6- difluorobenzyl)-1H-indazol- 3-yl]-4-[(2-methylpyridin-4- yl)amino]pyrimidin-5-ol | LC-MS: retention time: 0.84 min MS ES+: 489.2 [M + H]⁺ Method 5 |
| 1-2-5 SM = 1-3-5 | 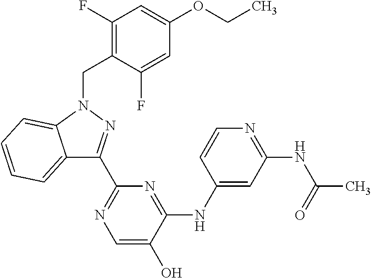 [SMILES: CCOc1cc(F)c(Cn2nc(-c3ncc(O)c(Nc4ccnc(NC(C)=O)c4)n3)c3ccccc32)c(F)c1] | N-{4-[(2-{1-[4-ethoxy-2- fluoro-6- (phenylsulfanyl)benzyl]- 1H-indazol-3-yl}-5- hydroxypyrimidin-4- yl)amino]pyridin-2- yl}acetamide | LC-MS: retention time: 0.78 min MS ES+: 532.2 [M + H]⁺ Method 5 |
| 1-2-6 SM = 1-3-6 | 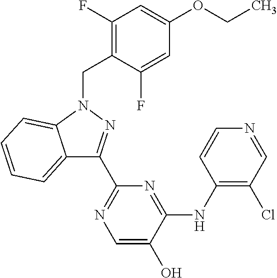 [SMILES: CCOc1cc(F)c(Cn2nc(-c3ncc(O)c(Nc4ccncc4Cl)n3)c3ccccc32)c(F)c1] | 4-[(3-chloropyridin-4- yl)amino]-2-[1-(4-ethoxy- 2,6-difluorobenzyl)-1H- indazol-3-yl]pyrimidin-5-ol | LC-MS: retention time: 0.83 min MS ES+: 509.2 [M + H]⁺ Method 5 |
| 1-2-7 SM = 1-3-7 | 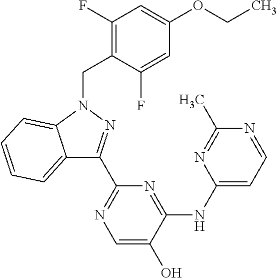 [SMILES: CCOc1cc(F)c(Cn2nc(-c3ncc(O)c(Nc4ccnc(C)n4)n3)c3ccccc32)c(F)c1] | 2-[1-(4-ethoxy-2,6- difluorobenzyl)-1H-indazol- 3-yl]-4-[(2- methylpyrimidin-4- yl)amino]pyrimidin-5-ol | LC-MS: retention time: 0.84 min MS ES+: 490.2 [M + H]⁺ Method 5 |
| 1-2-8 as side product of 1-2-7 SM = 1-3-7 | 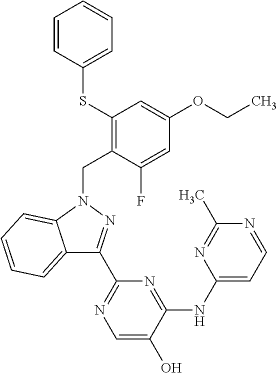 [SMILES: CCOc1cc(F)c(Cn2nc(-c3ncc(O)c(Nc4ccnc(C)n4)n3)c3ccccc32)c(Sc2ccccc2)c1] | 2-{1-[4-ethoxy-2-fluoro-6- (phenylsulfanyl)benzyl]- 1H-indazol-3-yl}-4-[(2- methylpyrimidin-4- yl)amino]pyrimidin-5-ol | LC-MS: retention time: 0.98 min MS ES+: 580.2 [M + H]⁺ Method 5 |
| 1-2-9 SM = 1-3-9 | 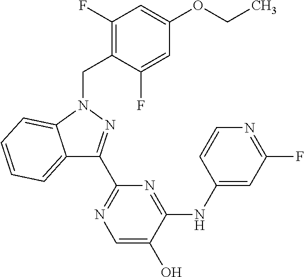 [SMILES: CCOc1cc(F)c(Cn2nc(-c3ncc(O)c(Nc4ccnc(F)c4)n3)c3ccccc32)c(F)c1] | 2-[1-(4-ethoxy-2,6- difluorobenzyl)-1H-indazol- 3-yl]-4-[(2-fluoropyridin-4- yl)amino]pyrimidin-5-ol | LC-MS: retention time: 0.86 min MS ES+: 493.2 [M + H]⁺ Method 5 |
| 1-2-10 SM = 1-3-10 | 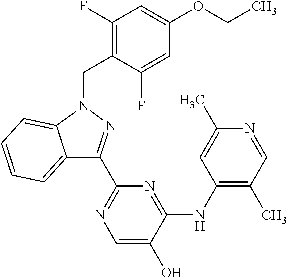 [SMILES: CCOc1cc(F)c(Cn2nc(-c3ncc(O)c(Nc4cc(C)ncc4C)n3)c3ccccc32)c(F)c1] | 4-[(2,5-dimethylpyridin-4- yl)amino]-2-{1-[4-ethoxy-2- fluoro-6- (phenylsulfanyl)benzyl]- 1H-indazol-3-yl}pyrimidin- 5-ol | LC-MS: retention time: 0.84 min MS ES+: 503.3 [M + H]⁺ Method 5 |
| 1-2-11 SM = 1-8-1 | 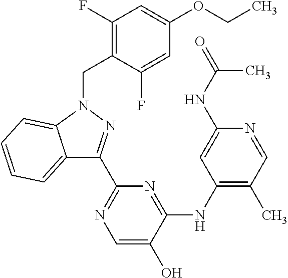 [SMILES: CCOc1cc(F)c(Cn2nc(-c3ncc(O)c(Nc4cc(NC(C)=O)ncc4C)n3)c3ccccc32)c(F)c1] | N-[4-({2-[1-(4-ethoxy-2,6- difluorobenzyl)-1H-indazol- 3-yl]-5-hydroxypyrimidin- 4-yl}amino)-5- methylpyridin-2- yl]acetamide | LC-MS: retention time: 0.78 min MS ES+: 546.3 [M + H]⁺ Method 5 |
| 1-2-12 SM = 1-3-11 | 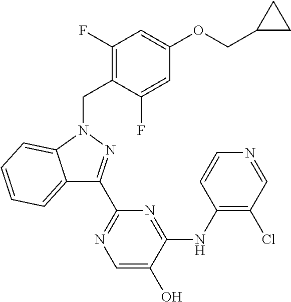 [SMILES: Oc1cnc(-c2nn(Cc3c(F)cc(OCC4CC4)cc3F)c3ccccc23)nc1Nc1ccncc1Cl] | 4-[(3-chloropyridin-4- yl)amino]-2-{1-[4- (cyclopropylmethoxy)-2,6- difluorobenzyl]-1H-indazol- 3-yl}pyrimidin-5-ol | LC-MS: retention time: 0.87 min MS ES+: 536.5 [M + H]⁺ Method 5 |
| 1-2-13 SM = 1-3-12 | 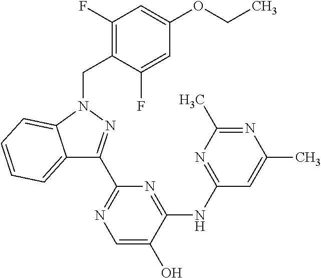 [SMILES: CCOc1cc(F)c(Cn2nc(-c3ncc(O)c(Nc4cc(C)nc(C)n4)n3)c3ccccc32)c(F)c1] | 4-[(2,6-Dimethylpyrimidin- 4-yl)amino]-2-[1-(4-ethoxy- 2,6-difluorobenzyl)-1H- indazol-3-yl]pyrimidin-5-ol | LC-MS: retention time: 0.85 min MS ES+: 504.3 [M + H]⁺ Method 5 |
| 1-2-14 SM = 1-3-13 | 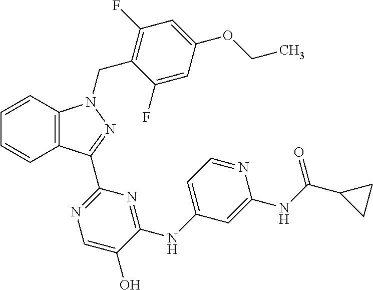 [SMILES: CCOc1cc(F)c(Cn2nc(-c3ncc(O)c(Nc4ccnc(NC(=O)C5CC5)c4)n3)c3ccccc32)c(F)c1] | N-[4-({2-[1-(4-Ethoxy-2,6- difluorobenzyl)-1H-indazol- 3-yl]-5-hydroxypyrimidin- 4-yl}amino)pyridin-2- yl]cyclopropancarboxamide | LC-MS: retention time: 0.80 min MS ES+: 558.3 [M + H]⁺ Method 5 |
| 1-2-15 SM = 1-3-14 | 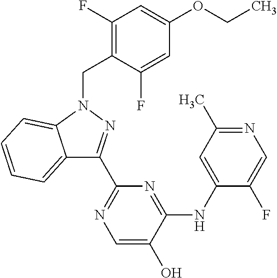 [SMILES: CCOc1cc(F)c(Cn2nc(-c3ncc(O)c(Nc4cc(C)ncc4F)n3)c3ccccc32)c(F)c1] | 2-[1-(4-Ethoxy-2,6- difluorobenzyl)-1H-indazol- 3-yl]-4-[(5-fluor-2- methylpyridin-4- yl)amino]pyrimidin-5-ol | LC-MS: retention time: 0.83 min MS ES+: 507.4 [M + H]⁺ Method 5 |
| 1-2-16 SM = 1-3-15 | 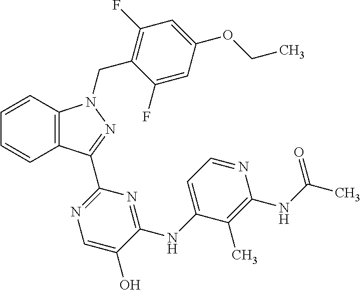 [SMILES: CCOc1cc(F)c(Cn2nc(-c3ncc(O)c(Nc4ccnc(NC(C)=O)c4C)n3)c3ccccc32)c(F)c1] | N-[4-({2-[1-(4-ethoxy-2,6- difluorobenzyl)-1H-indazol- 3-yl]-5-hydroxypyrimidin- 4-yl}amino)-3- methylpyridin-2- yl]acetamide | LC-MS: retention time: 0.78 min MS ES+: 546 [M + H]⁺ Method 5 |
| 1-2-18 SM = 1-3-17 | 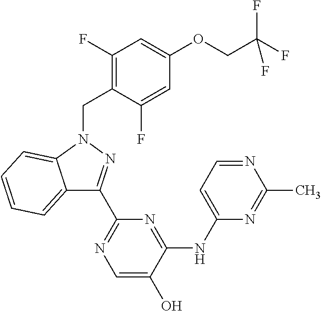 [SMILES: Cc1nccc(Nc2nc(-c3nn(Cc4c(F)cc(OCC(F)(F)F)cc4F)c4ccccc34)ncc2O)n1] | 2-{1-[2,6-difluoro-4-(2,2,2- trifluoroethoxy)benzyl]-1H- indazol-3-yl}-4-[(2- methylpyrimidin-4- yl)amino]pyrimidin-5-ol | LC-MS: retention time: 1.20 min MS ES+: 544 [M + H]⁺ Method 1 |
| 1-2-19 SM = 1-3-18 | 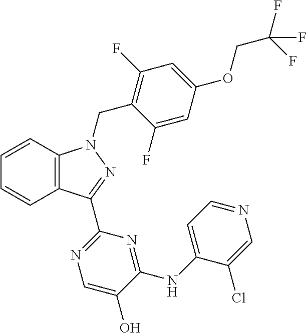 [SMILES: Oc1cnc(-c2nn(Cc3c(F)cc(OCC(F)(F)F)cc3F)c3ccccc23)nc1Nc1ccncc1Cl] | 4-[(3-chloropyridin-4- yl)amino]-2-{1-[2,6- difluoro-4-(2,2,2- trifluoroethoxy)benzyl]-1H- indazol-3-yl}pyrimidin-5-ol | LC-MS: retention time: 1.25 min MS ES+: 563 [M + H]⁺ Method 1 |
| 1-2-20 SM = 1-3-19 | 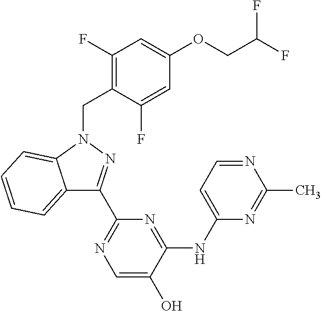 [SMILES: Cc1nccc(Nc2nc(-c3nn(Cc4c(F)cc(OCC(F)F)cc4F)c4ccccc34)ncc2O)n1] | 2-{1-[4-(2,2- difluoroethoxy)-2,6- difluorobenzyl]-1H-indazol- 3-yl}-4-[(2- methylpyrimidin-4- yl)amino]pyrimidin-5-ol | LC-MS: retention time: 1.09 min MS ES+: 526 [M + H]⁺ Method 1 |
| 1-2-21 SM = 1-18-1 | 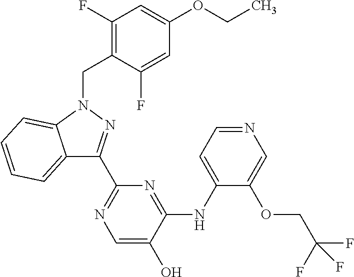 [SMILES: CCOc1cc(F)c(Cn2nc(-c3ncc(O)c(Nc4ccncc4OCC(F)(F)F)n3)c3ccccc32)c(F)c1] | 2-[1-(4-ethoxy-2,6- difluorobenzyl)-1H-indazol- 3-yl]-4-{[3-(2,2,2- trifluoroethoxy)pyridin-4- yl]amino}pyrimidin-5-ol | LC-MS: retention time: 0.88 min MS ES+: 573 [M + H]⁺ Method 5 |
| 1-2-22 SM = 1-18-2 | 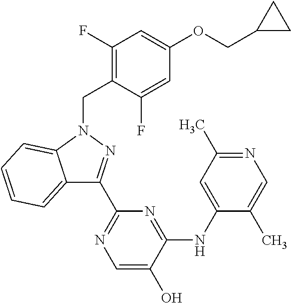 [SMILES: Cc1cc(Nc2nc(-c3nn(Cc4c(F)cc(OCC5CC5)cc4F)c4ccccc34)ncc2O)c(C)cn1] | 2-{1-[4- (cyclopropylmethoxy)-2,6- difluorobenzyl]-1H-indazol- 3-yl}-4-[(2,5- dimethylpyridin-4- yl)amino]pyrimidin-5-ol | LC-MS: retention time: 0.83 min MS ES+: 529 [M + H]⁺ Method 6 |
| 1-2-23 SM = 1-18-3 | 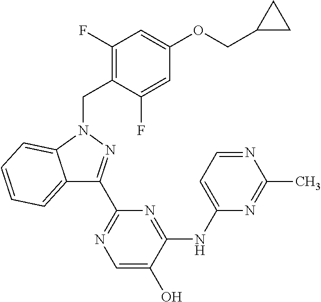 [SMILES: Cc1nccc(Nc2nc(-c3nn(Cc4c(F)cc(OCC5CC5)cc4F)c4ccccc34)ncc2O)n1] | 2-{1-[4- (cyclopropylmethoxy)-2,6- difluorobenzyl]-1H-indazol- 3-yl}-4-[(2- methylpyrimidin-4- yl)amino]pyrimidin-5-ol | LC-MS: retention time: 1.22 min MS ES+: 516 [M + H]⁺ Method 1 |
| 1-2-24 SM = 1-3-20 | 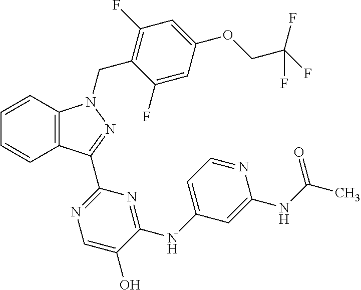 [SMILES: CC(=O)Nc1cc(Nc2nc(-c3nn(Cc4c(F)cc(OCC(F)(F)F)cc4F)c4ccccc34)ncc2O)ccn1] | N-{4-[(2-{1-[2,6-difluoro- 4-(2,2,2- trifluoroethoxy)benzyl]-1H- indazol-3-yl}-5- hydroxypyrimidin-4- yl)amino]pyridin-2- yl}acetamide | LC-MS: retention time: 1.07 min MS ES+: 586 [M + H]⁺ Method 1 |
| 1-2-25 SM = 1-3-21 | 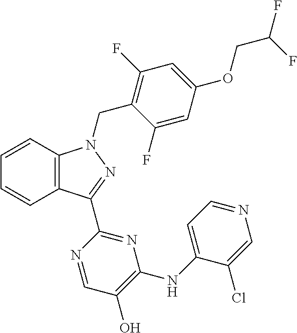 [SMILES: Oc1cnc(-c2nn(Cc3c(F)cc(OCC(F)F)cc3F)c3ccccc23)nc1Nc1ccncc1Cl] | 4-[(3-chloropyridin-4- yl)amino]-2-{1-[4-(2,2- difluoroethoxy)-2,6- difluorobenzyl]-1H-indazol- 3-yl}pyrimidin-5-ol | LC-MS: retention time: 1.18 min MS ES+: 545 [M + H]⁺ Method 1 |
| 1-2-26 SM = 1-3-22 Side product of coupling reaction | 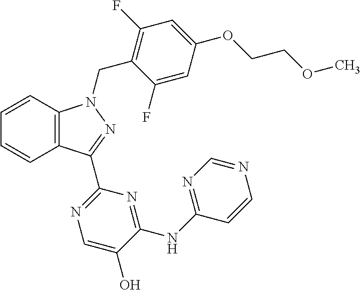 [SMILES: COCCOc1cc(F)c(Cn2nc(-c3ncc(O)c(Nc4ccncn4)n3)c3ccccc32)c(F)c1] | 2-{1-[2,6-difluoro-4-(2- methoxyethoxy)benzyl]-1H- indazol-3-yl}-4-(pyrimidin- 4-ylamino)pyrimidin-5-ol | LC-MS: retention time: 0.75 min MS ES+: 506 [M + H]⁺ Method 5 |

### Intermediate 1-3-1

### Preparation of N-[2-(difluoromethyl)pyridin-4-yl]-2-[1-(4-ethoxy-2,6-difluorobenzyl)-1H-indazol-3-yl]-5-methoxypyrimidin-4-amine

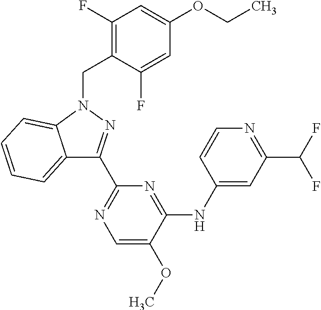 [SMILES: CCOc1cc(F)c(Cn2nc(-c3ncc(OC)c(Nc4ccnc(C(F)F)c4)n3)c3ccccc32)c(F)c1]

100 mg of 2-[1-(4-ethoxy-2,6-difluorobenzyl)-1H-indazol-3-yl]-5-methoxypyrimidin-4-amine 1-4-1 (0.24 mmol, 1.0 eq.) was dissolved in 0.9 mL DMF. 56 mg of 4-bromo-2-(difluoromethyl)pyridine (0.27 mmol, 1.1 eq.), 238 mg of cesium carbonate (0.73 mmol, 3.0 eq), 5.5 mg of palladium(II)acetate (0.024 mmol, 0.1 eq.) and 21 mg of 4,5-Bis(diphenylphosphino)-9,9-dimethylxanthene (0.036 mmol, 0.15 eq.) were added and the mixture was stirred at 105° C. over night. The reaction mixture was diluted with water and dichloromethane. The layers were separated and the aqueous layer was extracted with dichloromethane twice. The combined organic layers were dried using a water resistant filter and the filtrate was concentrated under reduced pressure. The crude product was purified by flash chromatography and HPLC to provide the analytically pure target compound: 39 mg, 0.07 mmol, 30%.

¹H-NMR (400 MHz, DMSO-ds): δ [ppm]=1.27 (t, 3H), 3.95-4.10 (m, 5H), 5.69 (s, 2H), 6.73-6.82 (m, 2H), 6.85 (t, 1H), 7.19-7.29 (m, 1H), 7.44-7.53 (m, 1H), 7.81 (d, 1H), 8.35 (d, 1H), 8.39 (s, 1H), 8.45 (d, 1H), 8.47-8.56 (m, 2H), 9.69 (s, 1H).

The following intermediates were prepared according to the same procedure from the indicated starting material (SM=starting material):

| | | | |
|---|---|---|---|
| 1-3-2 SM = 1-4-1 | 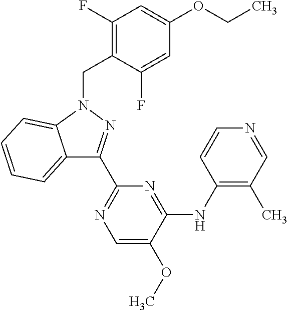 [SMILES: CCOc1cc(F)c(Cn2nc(-c3ncc(OC)c(Nc4ccncc4C)n3)c3ccccc32)c(F)c1] | 2-[1-(4-ethoxy-2,6- difluorobenzyl)-1H- indazol-3-yl]-5-methoxy- N-(3-methylpyridin-4- yl)pyrimidin-4-amine | ¹H-NMR (400 MHz, DMSO-d₆): δ [ppm] = 1.30 (t, 3H), 2.27 (s, 3H), 3.98-4.11 (m, 5H), 5.65 (s, 2H), 6.70-6.83 (m, 2H), 7.10-7.21 (m, 1H), 7.41-7.50 (m, 1H), 7.80 (d, 1H), 8.20-8.35 (m, 4H), 8.38 (d, 1H), 8.42 (s, 1H). |
| 1-3-3 SM = 1-4-1 | 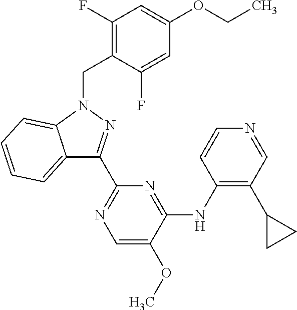 [SMILES: CCOc1cc(F)c(Cn2nc(-c3ncc(OC)c(Nc4ccncc4C4CC4)n3)c3ccccc32)c(F)c1] | N-(3-cyclopropylpyridin-4- yl)-2-[1-(4-ethoxy-2,6- difluorobenzyl)-1H- indazol-3-yl]-5- methoxypyrimidin-4-amine | ¹H-NMR (400 MHz, DMSO-d₆): δ [ppm] = 0.65-0.75 (m, 2H), 0.98-1.08 (m, 2H), 1.29 (t, 3H), 1.86-1.99 (m, 1H), 3.99-4.13 (m, 5H), 5.69 (s, 2H), 6.71-6.87 (m, 2H), 7.26 (t, 1H), 7.50 (t, 1H), 7.85 (d, 1H), 8.32-8.48 (m, 5H), 8.77 (d, 1H). |
| 1-3-4 SM = 1-4-1 | 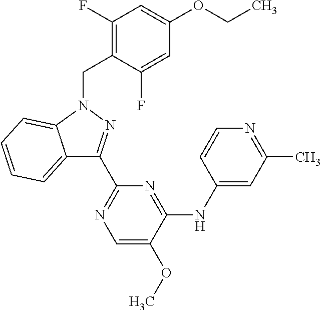 [SMILES: CCOc1cc(F)c(Cn2nc(-c3ncc(OC)c(Nc4ccnc(C)c4)n3)c3ccccc32)c(F)c1] | 2-[1-(4-ethoxy-2,6- difluorobenzyl)-1H- indazol-3-yl]-5-methoxy- N-(2-methylpyridin-4- yl)pyrimidin-4-amine | ¹H-NMR (300 MHz, DMSO-d₆): δ [ppm] = 1.27 (t, 3H), 2.43 (s, 3H), 3.95-4.09 (m, 5H), 5.68 (s, 2H), 6.63-6.84 (m, 2H), 7.25 (t, 1H), 7.43-7.53 (m, 1H), 7.78-7.92 (m, 2H), 8.13 (d, 1H), 8.27 (d, 1H), 8.33 (s, 1H), 8.48 (d, 1H), 9.29 (s, 1H). |
| 1-3-5 SM = 1-4-1 | 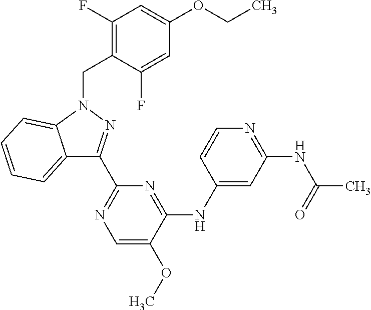 [SMILES: CCOc1cc(F)c(Cn2nc(-c3ncc(OC)c(Nc4ccnc(NC(C)=O)c4)n3)c3ccccc32)c(F)c1] | N-[4-({2-[1-(4-ethoxy-2,6- difluorobenzyl)-1H- indazol-3-yl]-5- methoxypyrimidin-4- yl}amino)pyridin-2- yl]acetamide | ¹H-NMR (400 MHz, DMSO-d₆): δ [ppm] = 1.29 (t, 3H), 2.09 (s, 3H), 3.96-4.10 (m, 5H), 5.68 (s, 2H), 6.72-6.85 (m, 2H), 7.23 (t, 1H), 7.48 (t, 1H), 7.82 (d, 1H), 8.16 (d, 1H), 8.28-8.35 (m, 2H), 8.37 (dd, 1H), 8.45 (d, 1H), 9.50 (s, 1H), 10.31 (s, 1H). |
| 1-3-6 SM = 1-4-1 | 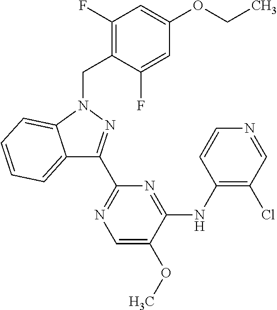 [SMILES: CCOc1cc(F)c(Cn2nc(-c3ncc(OC)c(Nc4ccncc4Cl)n3)c3ccccc32)c(F)c1] | N-(3-chloropyridin-4-yl)-2- [1-(4-ethoxy-2,6- difluorobenzyl)-1H- indazol-3-yl]-5- methoxypyrimidin-4-amine | ¹H-NMR (400 MHz, CHLOROFORM-d): δ [ppm] = 1.40 (t, 3H), 3.99 (q, 2H), 4.11 (s, 3H), 5.72 (s, 2H), 6.42-6.56 (m, 2H), 7.23-7.33 (m, 1H), 7.46 (t, 1H), 7.63 (d, 1H), 8.11 (s, 1H), 8.29 (s, 1H), 8.49-8.62 (m, 3H), 9.20 (d, 1H). |
| 1-3-7 SM = 1-4-1 | 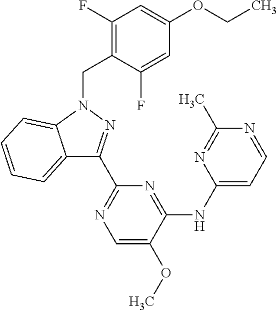 [SMILES: CCOc1cc(F)c(Cn2nc(-c3ncc(OC)c(Nc4ccnc(C)n4)n3)c3ccccc32)c(F)c1] | 2-[1-(4-ethoxy-2,6- difluorobenzyl)-1H- indazol-3-yl]-5-methoxy- N-(2-methylpyrimidin-4- yl)pyrimidin-4-amine | ¹H-NMR (400 MHz, DMSO-d₆): δ [ppm] = 1.28 (t, 3H), 2.54 (s, 3H), 3.96-4.08 (m, 5H), 5.67 (s, 2H), 6.76-6.85 (m, 2H), 7.27 (t, 1H), 7.45-7.53 (m, 1H), 7.84 (d, 1H), 8.43 (s, 1H), 8.47 (d, 1H), 8.50-8.57 (m, 2H), 8.89 (s, 1H), |
| 1-3-8 SM = 1-4-1 The Methyl- Ether was cleaved under this reaction conditions | 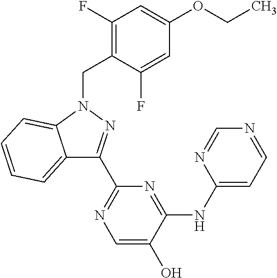 [SMILES: CCOc1cc(F)c(Cn2nc(-c3ncc(O)c(Nc4ccncn4)n3)c3ccccc32)c(F)c1] | 2-[1-(4-ethoxy-2,6- difluorobenzyl)-1H- indazol-3-yl]-4-(pyrimidin- 4-ylamino)pyrimidin-5-ol | LC-MS: retention time: 0.79 min MS ES+: 476.3 [M + H]⁺ Method 5 |
| 1-3-9 SM = 1-4-1 | 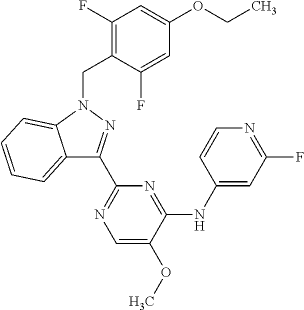 [SMILES: CCOc1cc(F)c(Cn2nc(-c3ncc(OC)c(Nc4ccnc(F)c4)n3)c3ccccc32)c(F)c1] | 2-[1-(4-ethoxy-2,6- difluorobenzyl)-1H- indazol-3-yl]-N-(2- fluoropyridin-4-yl)-5- methoxypyrimidin-4-amine | ¹H-NMR (300 MHz, DMSO-d₆): δ [ppm] = 1.27 (t, 3H), 3.96-4.09 (m, 5H), 5.69 (s, 2H), 6.69-6.81 (m, 2H), 7.28 (t, 1H), 7.49 (t, 1H), 7.85 (d, 1H), 7.97-8.11 (m, 3H), 8.40 (s, 1H), 8.45 (d, 1H), 9.81 (s, 1H). |
| 1-3-10 SM = 1-4-1 | 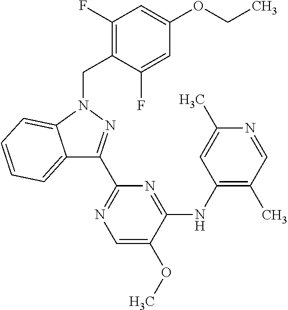 [SMILES: CCOc1cc(F)c(Cn2nc(-c3ncc(OC)c(Nc4cc(C)ncc4C)n3)c3ccccc32)c(F)c1] | N-(2,5-dimethylpyridin-4- yl)-2-[1-(4-ethoxy-2,6- difluorobenzyl)-1H- indazol-3-yl]-5- methoxypyrimidin-4-amine | ¹H-NMR (300 MHz, DMSO-d₆): δ [ppm] = 1.28 (t, 3H), 2.22 (s, 3H), 2.45 (s, 3H), 3.94-4.11 (m, 5H), 5.66 (s, 2H), 6.67-6.81 (m, 2H), 7.11-7.21 (m, 1H), 7.33-7.52 (m, 2H), 7.78 (d, 1H), 8.16 (d, 2H), 8.22-8.35 (m, 2H). |
| 1-3-11 SM = 1-9-1 | 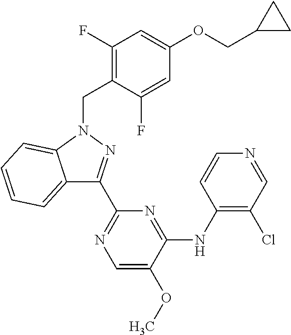 [SMILES: COc1cnc(-c2nn(Cc3c(F)cc(OCC4CC4)cc3F)c3ccccc23)nc1Nc1ccncc1Cl] | N-(3-chloropyridin-4-yl)-2- {1-[4- (cyclopropylmethoxy)-2,6- difluorobenzyl]-1H- indazol-3-yl}-5- methoxypyrimidin-4-amine | ¹H NMR (400 MHz, DMSO-d₆) δ [ppm] = 0.21-0.37 (m, 2 H) 0.52-0.57 (m, 2 H) 1.15-1.22 (m, 1 H) 3.83 (d, 2 H) 4.08 (s, 3 H) 5.68 (s, 2 H) 6.80 (s, 1H) 6.82 (m, 2 H) 7.26 (t, 1H) 7.49 (t, 1 H) 7.85 (d, 1 H) 8.25 (s, 1 H) 8.35-8.57 (m, 2 H) 8.65 (br. s., 1 H) 8.97 (d, 1H). |
| 1-3-12 SM = 1-4-1 | 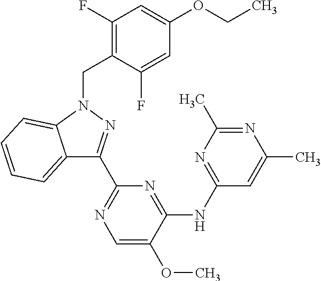 [SMILES: CCOc1cc(F)c(Cn2nc(-c3ncc(OC)c(Nc4cc(C)nc(C)n4)n3)c3ccccc32)c(F)c1] | N-(2,6-Dimethylpyrimidin- 4-yl)-2-[1-(4-ethoxy-2,6- difluorobenzyl)-1H- indazol-3-yl]-5- methoxypyrimidin-4-amine | ¹H-NMR (300 MHz, CHLOROFORM-d): δ [ppm] = 1.40 (t, 3H), 2.60 (s, 3H), 2.64 (s, 3H), 3.98 (q, 2H), 4.06 (s, 3H), 5.74 (s, 2H), 6.41-6.52 (m, 2H), 7.28 (t, 1H), 7.41-7.49 (m, 1H), 7.62 (d, 1H), 8.04 (s, 1H), 8.27 (s, 1H), 8.55-8.67 (m, 2H). |
| 1-3-13 SM = 1-4-1 + 1-14-1 | 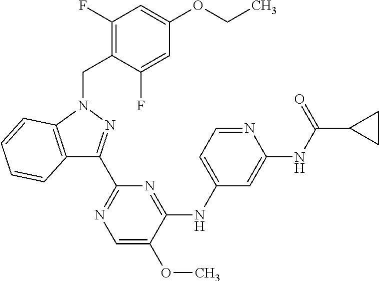 [SMILES: CCOc1cc(F)c(Cn2nc(-c3ncc(OC)c(Nc4ccnc(NC(=O)C5CC5)c4)n3)c3ccccc32)c(F)c1] | N-[4-({2-[1-(4-Ethoxy-2,6- difluorobenzyl)-1H- indazol-3-yl]-5- methoxypyrimidin-4- yl}amino)pyridin-2- yl]cyclopropancarboxamide | ¹H-NMR (300 MHz, DMSO-d₆): δ [ppm] = 0.66-0.86 (m, 4H), 1.28 (t, 3H), 1.98-2.06 (m, 1H), 3.94-4.12 (m, 5H), 5.67 (s, 2H), 6.68-6.84 (m, 2H), 7.15-7.26 (m, 1H), 7.41-7.53 (m, 1H), 7.81 (d, 1H), 8.15 (d, 1H), 8.28-8.37 (m, 3H), 8.43 (d, 1H), 9.43 (s, 1H), 10.61 (s, 1H). |
| 1-3-14 SM = 1-4-1 | 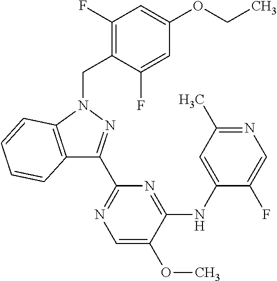 [SMILES: CCOc1cc(F)c(Cn2nc(-c3ncc(OC)c(Nc4cc(C)ncc4F)n3)c3ccccc32)c(F)c1] | 2-[1-(4-Ethoxy-2,6- difluorobenzyl)-1H- indazol-3-yl]-N-(5-fluoro- 2-methylpyridin-4-yl)-5- methoxypyrimidin-4-amine | ¹H-NMR (300 MHz, CHLOROFORM-d): δ [ppm] = 1.40 (t, 3H), 2.63 (s, 3H), 3.98 (q, 2H), 4.09 (s, 3H), 5.75 (s, 2H), 6.42-6.52 (m, 2H), 7.25-7.33 (m, 1H), 7.40-7.49 (m, 1H), 7.61 (d, 1H), 7.70 (d, 1H), 8.28 (s, 1H), 8.34 (d, 1H), 8.61 (d, 1H), 8.95 (d, 1H). |
| 1-3-15 SM = 1-4-1 + 1-13-1 | 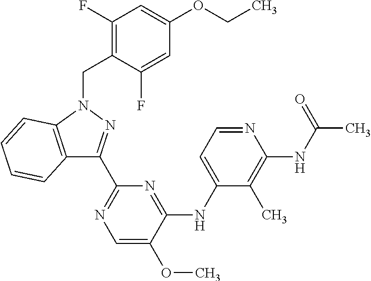 [SMILES: CCOc1cc(F)c(Cn2nc(-c3ncc(OC)c(Nc4ccnc(NC(C)=O)c4C)n3)c3ccccc32)c(F)c1] | N-[4-({2-[1-(4-ethoxy-2,6- difluorobenzyl)-1H- indazol-3-yl]-5- methoxypyrimidin-4- yl}amino)-3- methylpyridin-2- yl]acetamide | ¹H-NMR (300 MHz, DMSO-d₆): δ [ppm] = 1.28 (t, 3H), 2.21 (s, 3H), 2.25 (s, 3H), 3.96-4.13 (m, 5H), 5.69 (s, 2H), 6.70-6.83 (m, 2H), 7.21 (t, 1H), 7.48 (t, 1H), 7.81 (d, 1H), 8.13-8.22 (m, 1H), 8.29-8.35 (m, 2H), 8.43 (s, 1H), 9.15 (br. s, 1H), 10.65 (br. s, 1H). |
| 1-3-17 SM = 1-9-3 | 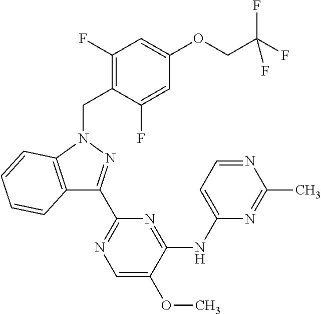 [SMILES: COc1cnc(-c2nn(Cc3c(F)cc(OCC(F)(F)F)cc3F)c3ccccc23)nc1Nc1ccnc(C)n1] | 2-{1-[2,6-difluoro-4-(2,2,2- trifluoroethoxy)benzyl]- 1H-indazol-3-yl}-5- methoxy-N-(2- methylpyrimidin-4- yl)pyrimidin-4-amine | ¹H-NMR (400 MHz, DMSO-d₆): δ [ppm] = 2.55 (s, 3H), 4.02 (s, 3H), 4.83 (q, 2H), 5.72 (s, 2H), 7.03 (m, 2H), 7.29 (dd, 1H), 7.51 (dd, 1H), 7.87 (d, 1H), 8.44 (s, 1H), 8.48 (d, 1H), 8.55 (d, 1H), 8.58 (d, 1H), 8.94 (br. s., 1H). |
| 1-3-18 SM = 1-9-3 | 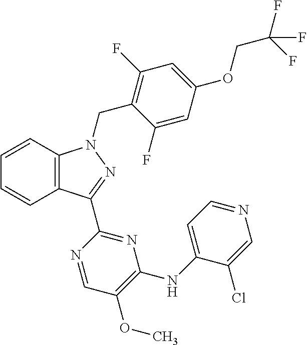 [SMILES: COc1cnc(-c2nn(Cc3c(F)cc(OCC(F)(F)F)cc3F)c3ccccc23)nc1Nc1ccncc1Cl] | N-(3-chloropyridin-4-yl)-2- {1-[2,6-difluoro-4-(2,2,2- trifluoroethoxy)benzyl]- 1H-indazol-3-yl}-5- methoxypyrimidin-4-amine | ¹H-NMR (400 MHz, DMSO-d₆): δ [ppm] = 4.08 (s, 3H), 4.83 (q, 2H), 5.72 (s, 2H), 7.03 (m, 2H), 7.26 (dd, 1H), 7.50 (dd, 1H), 7.87 (d, 1H), 8.28 (br. s., 1H), 8.39 (d, 1H), 8.46 (s, 1H), 8.47 (d, 1H), 8.65 (s, 1H), 8.97 (d, 1H). |
| 1-3-19 SM = 1-9-2 | 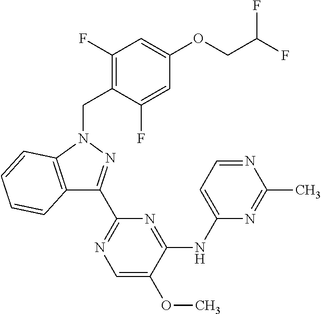 [SMILES: COc1cnc(-c2nn(Cc3c(F)cc(OCC(F)F)cc3F)c3ccccc23)nc1Nc1ccnc(C)n1] | 2-{1-[4-(2,2- difluoroethoxy)-2,6- difluorobenzyl]-1H- indazol-3-yl}-5-methoxy- N-(2-methylpyrimidin-4- yl)pyrimidin-4-amine | ¹H-NMR (400 MHz, DMSO-d₆): δ [ppm] = 2.55 (s, 3H), 4.01 (s, 3H), 4.36 (td, 2H), 5.71 (s, 2H), 6.37 (tt, 1H), 7.03 (m, 2H), 7.29 (dd, 1H), 7.50 (dd, 1H), 7.86 (d, 1H), 8.43 (s, 1H), 8.48 (d, 1H), 8.55 (d, 1H), 8.58 (d, 1H), 8.93 (br. s., 1H). |
| 1-3-20 SM = 1-9-3 | 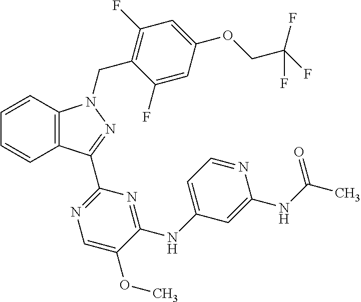 [SMILES: COc1cnc(-c2nn(Cc3c(F)cc(OCC(F)(F)F)cc3F)c3ccccc23)nc1Nc1ccnc(NC(C)=O)c1] | N-{4-[(2-{1-[2,6-difluoro- 4-(2,2,2- trifluoroethoxy)benzyl]- 1H-indazol-3-yl}-5- methoxypyrimidin-4- yl)amino]pyridin-2- yl}acetamide | ¹H-NMR (400 MHz, DMSO-d₆): δ [ppm] = 2.08 (s, 3H), 4.01 (s, 3H), 4.82 (q, 2H), 5.71 (s, 2H), 7.00 (m, 2H), 7.23 (dd, 1H), 7.48 (dd, 1H), 7.83 (d, 1H), 8.16 (d, 1H), 8.31 (br. s., 1H), 8.32 (s, 1H), 8.36 (dd, 1H), 8.45 (d, 1H), 9.51 (br. s., 1H), 10.31 (br. s., 1H). |
| 1-3-21 SM = 1-9-2 | 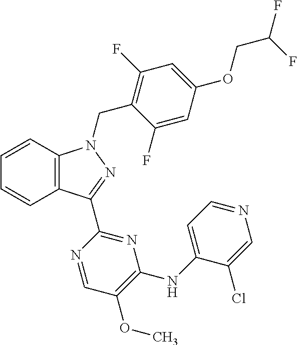 [SMILES: COc1cnc(-c2nn(Cc3c(F)cc(OCC(F)F)cc3F)c3ccccc23)nc1Nc1ccncc1Cl] | N-(3-chloropyridin-4-yl)-2- {1-[4-(2,2-difluoroethoxy)- 2,6-difluorobenzyl]-1H- indazol-3-yl}-5- methoxypyrimidin-4-amine | ¹H-NMR (400 MHz, DMSO-d₆): δ [ppm] = 4.08 (s, 3H), 4.36 (td, 2H), 5.71 (s, 2H), 6.37 (tt, 1H), 6.96 (m, 2H), 7.26 (dd, 1H), 7.50 (dd, 1H), 7.86 (d, 1H), 8.28 (br. s., 1H), 8.39 (d, 1H), 8.46 (s, 1H), 8.48 (d, 1H), 8.65 (s, 1H), 8.97 (d, 1H). |
| 1-3-22 SM = 1-9-4 | 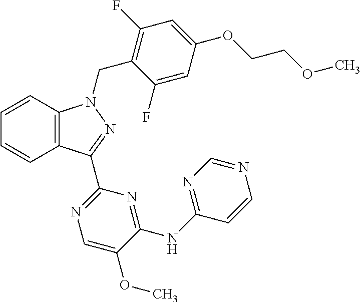 [SMILES: COCCOc1cc(F)c(Cn2nc(-c3ncc(OC)c(Nc4ccncn4)n3)c3ccccc32)c(F)c1] | 2-{1-[2,6-difluoro-4-(2- methoxyethoxy)benzyl]- 1H-indazol-3-yl}-5- methoxy-N-(pyrimidin-4- yl)pyrimidin-4-amine | ¹H-NMR (400 MHz, DMSO-d₆): δ [ppm] = 3.26 (s, 3H), 3.57-3.65 (m, 2H), 4.09-4.16 (m, 2H), 5.69 (s, 2H), 6.81-6.91 (m, 2H), 7.28 (t, 1H), 7.46-7.53 (m, 1H), 7.85 (d, 1H), 8.26 (s, 1H), 8.49 (d, 1H), 8.62 (d, 2H), 8.87 (s, 1H), 9.15 (s, 1H), 11.22 (br. s, 1H). |

### Intermediate 1-4-1

### Preparation of 2-[1-(4-ethoxy-2,6-difluorobenzyl)-1H-indazol-3-yl]-5-methoxypyrimidin-4-amine

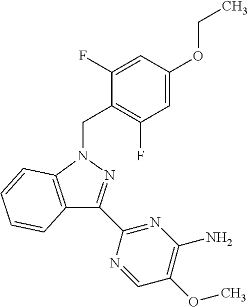 [SMILES: CCOc1cc(F)c(Cn2nc(-c3ncc(OC)c(N)n3)c3ccccc32)c(F)c1]

165 g of 1-(4-ethoxy-2,6-difluorobenzyl)-1H-Indazole-3-carboximidamide hydrochloride 1-5-1 (450 mmol, 1.0 eq.), 185 g of 3,3-bis(dimethylamino)-2-methoxypropanenitrile 1-6-1 (1079 mmol, 2.4 eq.) and 19.1 mL of piperidine (225 mmol, 0.5 eq.) were dissolved in 1470 mL of dry 3-methylbutan-1-ol, put under a nitrogen atmosphere and stirred at 110° C. over night. The mixture was cooled down at 0° C. and stirred for crystallization. The resulting suspension was filtered off. The crystals were washed with 1 L hexane and dried in vacuo at 60° C. to provide 65 g (158 mmol. 35%) of the analytically pure target compound.

1H-NMR (400 MHz, DMSO-d6): δ [ppm]=1.26 (t, 3H), 3.84 (s, 3H), 4.00 (q, 2H), 5.60 (s, 2H), 6.66-6.76 (m, 2H), 6.76-6.91 (m, 2H), 7.17 (t, 1H), 7.40 (t, 1H), 7.69 (d, 1H). 7.93 (s, 1H), 8.52 (d, 1H).

The following intermediate was prepared according to the same procedure from the indicated starting material (SM=starting material):

| | | | |
|---|---|---|---|
| 1-4-2 SM = 1-6-1 + 1-5-2 | 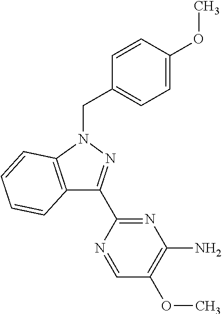 [SMILES: COc1ccc(Cn2nc(-c3ncc(OC)c(N)n3)c3ccccc32)cc1] | 5-methoxy-2-[1-(4- methoxybenzyl)-1H- indazol-3- yl]pyrimidin-4-amine | ¹H-NMR (300 MHz, DMSO-d₆): δ [ppm] = 3.62-3.69 (s, 3H), 3.85 (s, 3H), 5.59 (s, 2H), 6.78-6.90 (m, 4H), 7.11-7.23 (m, 3H), 7.35 (ddd, 1H), 7.68 (d, 1H), 7.95 (s, 1H), 8.53 (d, 1H). |

### Intermediate 1-5-1

### Preparation of 1-(4-ethoxy-2,6-difluorobenzyl)-1H-indazole-3-carboximidamide, salt with hydrochloric acid

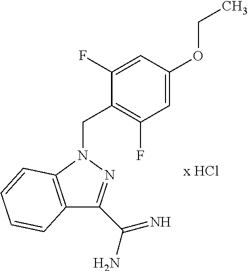 [SMILES: CCOc1cc(F)c(Cn2nc(C(=N)N)c3ccccc32)c(F)c1]

58 g of ammonium chloride (1083 mmol, 5.0 eq.) were suspended in 1 L of dry toluene under nitrogen atmosphere and cooled down to 0° C. bath temperature. 541 mL of 2M trimethylaluminium solution in toluene (1083 mmol, 5.0 eq.) were added drop wise. The mixture was stirred at room temperature until disappearance of gassing. 75 g of methyl 1-(4-ethoxy-2,6-difluorobenzyl)-1H-indazole-3-carboxylate 1-7-1 (217 mmol, 1.0 eq.) were dissolved in 1 L of dry toluene and added drop wise to the reaction mixture and stirred over night at 80° C. bath temperature The mixture was cooled down with an ice bath to 0° C. bath temperature, 1.4 L of methanol we re added and stirred for one hour at rt. The resulting suspension was filtered over celite and washed with methanol. The filtrate was concentrated in vacuo and dried in vacuo at 50° C. and the crude product was used without any further purification: 67.3 g (183 mmol, 84%).

¹H-NMR (300 MHz, DMSO-d6) δ[ppm]=1.26 (t, 3H), 4.01 (q, 2H), 5.75 (s, 2H), 6.68-6.78 (m, 2H), 7.34-7.43 (m, 1H), 7.56-7.61 (m, 1H), 7.93 (dd, 2H), 9.29 (br. s, 3H).

The following intermediate was prepared according to the same procedure from the indicated starting material (SM=starting material):

| | | | |
|---|---|---|---|
| 1-5-2 SM = 1-7-2 | 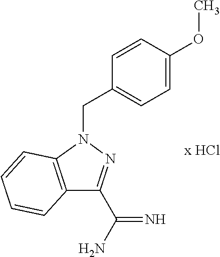 [SMILES: COc1ccc(Cn2nc(C(=N)N)c3ccccc32)cc1] | 1-(4- methoxybenzyl)- 1H-indazole-3- carboximidamide, salt with hydrochloric acid | ¹H-NMR (300 MHz, DMSO-d₆): δ [ppm] = 3.62-3.70 (s, 3 H), 5.57 (s, 2 H), 6.37 (br. s., 3 H), 6.78-6.88 (m, 2 H), 7.10-7.23 (m, 3 H), 7.35 (ddd, 1 H), 7.68 (d, 1H), 8.27 (d, 1 H). |

### Intermediate 1-6-1

### Preparation of 3,3-bis(dimethylamino)-2-methoxypropanenitrile

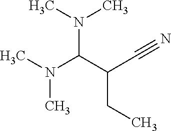

360 g of 1-tert-butoxy-N,N,N′,N′-tetramethylmethanediamine (Bredereck's reagent) (2068 mmol, 1.0 eq.) and 150 g of methoxyacetonitrile (2068 mmol, 1.0 eq.) were stirred for 18 hours at 80° C. The reaction mixture was concentrated in vacuo. The residue was purified by vacuum distillation (8-23 mmbar; bp 80-83° C.) to yield 117 g (687 mmol, 33%) of the analytical pure target compound as a yellowish liquid.

¹H-NMR (400 MHz, DMSO-d6): δ [ppm]=2.23 (s, 6H), 2.29 (s, 6H), 3.23 (d, 1H), 3.36-3.41 (s, 3H), 4.73 (d, 1H).

### Intermediate 1-7-1

### Preparation of methyl 1-(4-ethoxy-2,6-difluorobenzyl)-1H-indazole-3-carboxylate

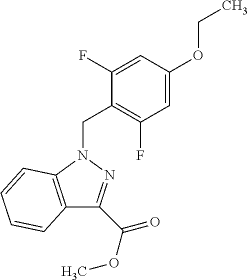 [SMILES: CCOc1cc(F)c(Cn2nc(C(=O)OC)c3ccccc32)c(F)c1]

185 g of methyl 1H-indazole-3-carboxylate (1050 mmol, 1.0 eq.) were dissolved in 3 L of dry THF and cooled to 5° C. 411 g of cesium carbonate (1260 mmol, 1.2 eq.) were added stirred for 15 min. 290 g of 2-(bromomethyl)-5-ethoxy-1,3-difluorobenzene (1155 mmol, 1.1 eq.) dissolved in 250 ml THF were added drop wise at 5° C. The precipitate was filtered off. The filtrate was concentrated in vacuo. The residue was crystallized from ethyl acetate/hexane (1:1) to provide 310 g (895 mmol, 85%) of analytically pure target compound.

¹H-NMR (400 MHz, DMSO-d6) δ[ppm]=1.27 (t, 3H), 3.86 (s, 3H), 4.01 (q, 2H), 5.68 (s, 2H), 6.70-6.76 (m, 2H), 7.32 (t, 1H), 7.50 (t, 1H), 7.84 (d, 1H), 8.00-8.12 (m, 1H).

The following intermediate was prepared according to the same procedure from commercial available starting material:

| | | | |
|---|---|---|---|
| 1-7-2 | 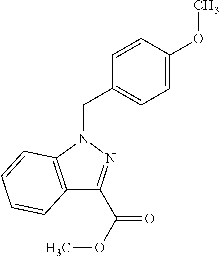 [SMILES: COC(=O)c1nn(Cc2ccc(OC)cc2)c2ccccc12] | methyl 1-(4- methoxybenzyl)- 1H-indazole-3- carboxylate | ¹H-NMR (400 MHz, DMSO-d₆): δ [ppm] = 3.66 (s, 3H), 3.89 (s, 3H), 5.67 (s, 2H), 6.79-6.90 (m, 2H), 7.20-7.26 (m, 2H), 7.29-7.33 (m, 1H), 7.43-7.47 (m, 1H), 7.84 (d, 1H), 8.05 (dt, 1H). |

### Intermediate 1-8-1

### Preparation of N-[4-({2-[1-(4-ethoxy-2,6-difluorobenzyl)-1H-indazol-3-yl]-5-methoxypyrimidin-4-yl}amino)-5-methylpyridin-2-yl]acetamide

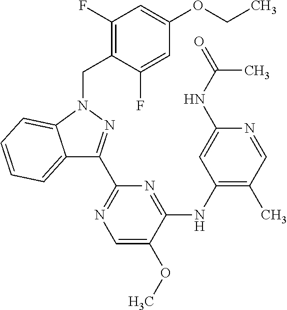 [SMILES: CCOc1cc(F)c(Cn2nc(-c3ncc(OC)c(Nc4cc(NC(C)=O)ncc4C)n3)c3ccccc32)c(F)c1]

1.0 g of 2-[1-(4-ethoxy-2,6-difluorobenzyl)-1H-indazol-3-yl]-5-methoxypyrimidin-4-amine 1-4-1 (2.43 mmol. 1.0 eq.) were dissolved in 122 mL DMF. 494 mg N-(4-chloro-5-methylpyridin-2-yl)acetamide (2.67 mmol, 1.1 eq.), 1.01 g potassium carbonate (7.3 mmol, 3.0 eq.), 55 mg palladium(II)acetate and 348 mg 2-Dicyclohexylphosphino-2′,4′,6′-trisopropylbiphenyl (0.73 mmol, 0.3 eq.) were added and stirred at 105° C. over night under argon atmosphere. The reaction mixture was treated with water and dichloromethane. The aqueous layer was extracted with dichloromethane three times and the combined organic layers were washed with water and brine. The organic layer was and filtered through a silicone coated filter and concentrated under reduced pressure. The crude product was treated with ethyl acetate. The unsolved residue was filtered off. The filtrate was concentrated in vacuo. The residue was purified by flash chromatography to provide the analytically pure target compound: 149 mg, 0.27 mmol, 11%.

¹H-NMR (400 MHz, DMSO-d₆): δ [ppm]=1.27 (t, 3H), 2.03 (s, 3H). 2.12 (s, 3H), 3.96-4.09 (m, 5H), 5.60 (s, 2H), 6.67-6.78 (m, 2H), 6.99 (t, 1H), 7.34-7.41 (m, 1H), 7.70 (d, 1H), 8.05 (d, 1H), 8.17 (s, 1H), 8.22 (s, 1H). 8.28 (s, 1H), 8.80 (s, 1H), 10.39 (s, 1H).

### Intermediate 1-9-1

### Preparation of 2-1-[4-(cyclopropylmethoxy)-2,6-difluorobenzyl]-1H-indazol-3-yl)-5-methoxypyrimidin-4-amine

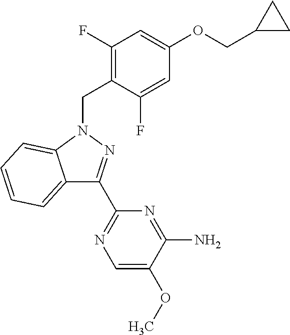 [SMILES: COc1cnc(-c2nn(Cc3c(F)cc(OCC4CC4)cc3F)c3ccccc23)nc1N]

2.37 g (9.84 mmol) of 2-(1H-indazol-3-yl)-5-methoxypyrimidin-4-amine 1-10-1 were dissolved in 19 mL DMF. 433 mg (10.83 mmol) of sodium hydride (60% dispersion in mineral oil) were added portionwise under inert atmosphere and stirred 15 minutes at room temperature. The reaction mixture was cooled to 0° C. and 363 mg (0.98 mmol) tetra-n-butylammonium iodide and 3.00 g (10.83 mmol) 2-(bromomethyl)-5-(cyclopropylmethoxy)-1,3-difluorobenzene 1-11-1 dissolved in 1 mL DMF were added subsequently. The mixture was stirred overnight, then poured into water and extracted with DCM. The organic layer was dried over sodium sulfate and concentrated under reduced pressure. The crude product was purified by column chromatography (silica gel, 9:1 DCM/methanol) to furnish 1.90 g (44% yield) of 2-{1-[4-(cyclopropylmethoxy)-2,6-difluorobenzyl]-1H-indazol-3-yl}-5-methoxypyrimidin-4-amine.

¹H-NMR (400 MHz, DMSO-de): 5 [ppm]=0.22-0.37 (m, 2H) 0.50-0.62 (m, 2H) 1.19 (ddd, 1H) 3.73-4.00 (m, 5H) 5.63 (s, 2H) 6.68-6.83 (m, 3H) 6.87 (br. s., 1H) 7.16-7.30 (m, 1H) 7.44 (ddd, 1H) 7.73 (d, 1H) 7.97 (s, 1H) 8.56 (d, 1H).

The following intermediates were prepared according to the same procedure from the indicated starting material (SM=starting material):

| | | | |
|---|---|---|---|
| 1-9-2 SM = 1-10-1, 1-11-2 | 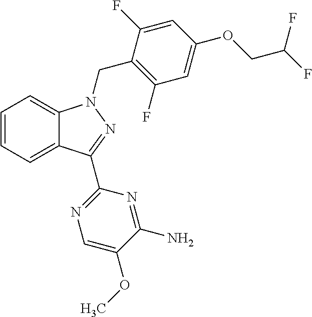 [SMILES: COc1cnc(-c2nn(Cc3c(F)cc(OCC(F)F)cc3F)c3ccccc23)nc1N] | 2-{1-[4-(2,2- difluoroethoxy)-2,6- difluorobenzyl]-1H- indazol-3-yl}-5- methoxypyrimidin-4- amine | ¹H-NMR (400 MHz, DMSO-d₆): δ [ppm] = 3.80-3.92 (s, 3 H) 4.37 (td, 2 H) 5.66 (s, 2 H) 6.38 (t, 1 H) 6.74-7.04 (m, 4 H) 7.22 (t, 1 H) 7.45 (ddd, 1 H) 7.75 (d, 1 H) 7.90-8.08 (m, 1 H) 8.56 (d, 1 H), |
| 1-9-3 SM = 1-10-1, 1-11-3 | 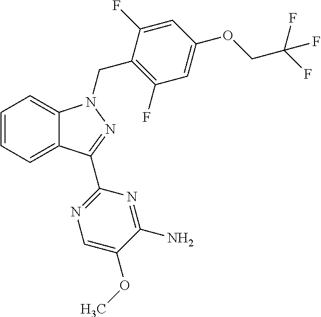 [SMILES: COc1cnc(-c2nn(Cc3c(F)cc(OCC(F)(F)F)cc3F)c3ccccc23)nc1N] | 2-{1-[2,6-difluoro-4- (2,2,2- trifluoroethoxy)benzyl]- 1H-indazol-3-yl}-5- methoxypyrimidin-4- amine | ¹H-NMR (400 MHz, DMSO-d₆): δ [ppm] = 3.87 (s, 3H), 4.83 (q, 2H), 5.66 (s, 2H), 6.88 (br. s., 2H), 6.98 (m, 2H), 7.22 (dd, 1H), 7.44 (dd, 1H), 7.75 (d, 1H), 7.97 (s, 1H), 8.56 (d, 1H). |
| 1-9-4 SM = 1-10-1, 1-19-1 | 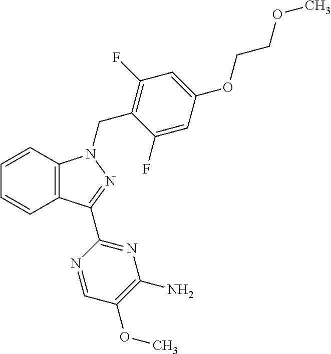 [SMILES: COCCOc1cc(F)c(Cn2nc(-c3ncc(OC)c(N)n3)c3ccccc32)c(F)c1] | 2-{1-[2,6-difluoro-4-(2- methoxyethoxy)benzyl]- 1H-indazol-3-yl}-5- methoxypyrimidin-4- amine | ¹H-NMR (400 MHz, DMSO-d₆): δ [ppm] = 3.26 (s, 3H), 3.54-3.64 (m, 2H), 3.87 (s, 3H), 4.05-4.15 (m, 2H), 5.62 (s, 2H), 6.68-6.95 (m, 4H), 7.15-7.26 (m, 1H), 7.37-7.48 (m, 1H), 7.72 (d, 1H), 7.96 (s, 1H), 8.55 (d, 1H). |

### Intermediate 1-10-1

### Preparation of 2-(1H-indazol-3-yl)-5-methoxypyrimidin-4-amine

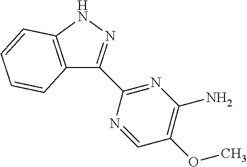 [SMILES: COc1cnc(-c2n[nH]c3ccccc23)nc1N]

7.0 g of 5-methoxy-2-[1-(4-methoxybenzyl)-1H-indazol-3-yl]pyrimidin-4-amine 1-4-2 (19.4 mmol, 1.0 eq.) was dissolved in 76 mL 1,2-dichloroethane and 44.8 mL trifluoroacetic acid (581 mmol, 30 eq.) and 17.1 mL trifluoromethanesulfonic acid (194 mmol, 10 eq.) were added drop wise. The reaction mixture was warmed to 75° C. and stirred for 2 h. The reaction mixture was treated with half-saturated sodium carbonate solution. White material precipitated and was filtered off. To reduce the salt content the filter cake was suspended in water and stirred for 1 h. The water was filtered off and the new filter cake was dried under reduced pressure to provide the analytically pure product: 3.97 g, 16.5 mmol, 85%.

¹H-NMR (400 MHz, DMSO-de): δ [ppm]=3.90 (s, 3H), 6.83 (br. s., 2H). 7.13-7.22 (m, 1H), 7.32-7.39 (m, 1H). 7.56 (d, 1H), 8.00 (s, 1H). 8.56 (d, 1H), 13.20 (br. s, 1H).

### Intermediate 1-11-1

### Preparation of 2-(bromomethyl)-5-(cyclopropylmethoxy)-1,3-difluorobenzene

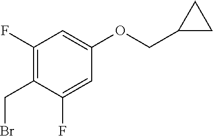 [SMILES: Fc1cc(OCC2CC2)cc(F)c1CBr]

3.00 g (14.00 mmol) [4-(cyclopropylmethoxy)-2,6-difluorophenyl]methanol 1-12-1 were dissolved in 4.50 mL 47% hydrogen bromide in water and stirred overnight. The orange solution was poured into 100 ml diethyl ether and the separated organic layer was added dropwise to a saturated sodium bicarbonate solution (gas evolution!). The water layer was extracted twice with diethyl ether, dried over sodium sulfate and concentrated under reduced pressure. 3.0 g (77%) of 2-(bromomethyl)-5-(cyclopropylmethoxy)-1,3-difluorobenzene were isolated as an oil and used without further purification in the next step.

¹H-NMR (300 MHz, DMSO-de): δ [ppm]=0.21-0.41 (m, 2H) 0.46-0.66 (m, 2H) 1.19 (qdd, 1H) 3.72-3.94 (m, 2H) 4.59 (s, 2H) 6.69-6.90 (m, 2H).

The following intermediates were prepared according to the same procedure from the indicated starting material (SM=starting material):

| | | | |
|---|---|---|---|
| 1-11-2 SM = 1-12-2 | 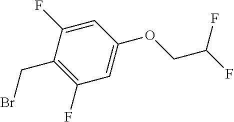 [SMILES: Fc1cc(OCC(F)F)cc(F)c1CBr] | 2-(bromomethyl)- 5-(2,2- difluoroethoxy)- 1,3- difluorobenzene | ¹H-NMR (400 MHz, DMSO-d₆): δ [ppm] = 4.38 (td, 2 H) 4.59 (s, 2 H) 6.38 (t, 1 H) 6.73-7.02 (m, 2 H). |
| 1-11-3 SM = 1-16-1 | 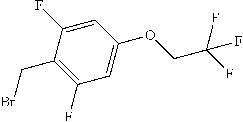 [SMILES: Fc1cc(OCC(F)(F)F)cc(F)c1CBr] | 4-(bromomethyl)- 3,5-difluorophenyl 2,2,2-trifluoroethyl ether | ¹H-NMR (400 MHz, DMSO-d₆): δ [ppm] = 4.62 (s, 2H) 4.86 (q, 2H) 7.01 (m, 2H). |

### Intermediate 1-12-1

### Preparation of [4-(cyclopropyloxy)-2,6-difluorophenyl]methanol

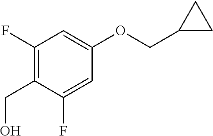 [SMILES: OCc1c(F)cc(OCC2CC2)cc1F]

3.00 g (18.7 mmol) 3,5-difluoro-4-(hydroxymethyl)phenol, 3.52 mL (22.5 mmol) (bromomethyl)cyclopropane and 12.95 g (93.7 mmol) potassium carbonate were suspended under inert atmosphere in 140 mL DMF. The mixture was stirred at 60 V overnight, cooled to room temperature. The suspension was filtered and the filtrate was evaporated in vacuo. The residue was dissolved with ice water and extracted with ethyl acetate. The organic layer was extracted with brine, dried over sodium sulfate and concentrated under reduced pressure. 4.10 g (99% yield) [4-(cycbpropylmethoxy)-2,6-difluorophenyl]methanol was isolated and used without further purification.

¹H-NMR (400 MHz, DMSO-de): δ [ppm]=0.17-0.39 (m, 2H) 0.46-0.62 (m, 2H) 1.08-1.26 (m, 1H) 3.82 (d, 2H) 4.39 (d, 2H) 5.05 (t, 1H) 6.59-6.78 (m, 2H).

The following Intermediates were prepared according to the same procedure from commercial available starting material:

| | | | |
|---|---|---|---|
| 1-12-2 | 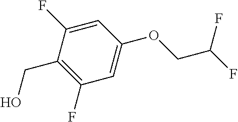 [SMILES: OCc1c(F)cc(OCC(F)F)cc1F] | [4-(2,2-difluoroethoxy)-2,6- difluorophenyl]methanol | ¹H-NMR (300 MHz, DMSO-d₆): δ [ppm] = 4.21-4.55 (m, 4 H) 5.13 (br. s., 1 H) 6.39 (td, 2 H) 6.67-6.96 (m, 2 H). |
| 1-12-2 | 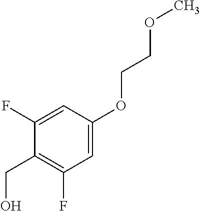 | [2,6-difluoro-4-(2- methoxyethoxy)phenyl]methanol | ¹H-NMR (300 MHz, DMSO-d₆): δ [ppm] = 3.25 (s, 3H), 3.50-3.65 (m, 2H), 3.99-4.14 (m, 2H), 4.37 (br. s., 2H), 5.07 (br. s., 1H), 6.52-6.75 (m, 2H). |

### Intermediate 1-13-1

### Preparation of N-(4-bromo-3-methylpyridin-2-yl)acetamide

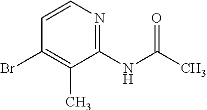

1.22 g 4-bromo-3-methylpyridin-2-amine (6.52 mmol, 1.0 eq.) was dissolved in 23 mL acetic anhydride. Then 0.95 μL 4-dimethylaminopyridine (65 μmol, 0.01 mmol). The reaction mixture was stirred at 140° C. (reflux) over night. The reaction mixture was diluted with icewater. Now about 50 mL of aqueous ammonia-solution (33%) was added under cooling until pH 10. The mixture was diluted with ethyl acetate and extracted three times with ethyl acetate. The combined organic layers were dried in use of a water resistant filter and the filtrate was evaporated under reduced pressure. The crude product was purified by flash chromatography to provide the analytically pure target compound: 1.08 g, 4.7 mmol, 72%.

¹H-NMR (400 MHz, DMSO-de): δ [ppm]=2.05 (s, 3H), 2.18 (s, 3H), 7.54 (d, 1H), 8.10 (d, 1H), 10.20 (s, 1H).

### Intermediate 1-14-1

### Preparation of N-(4-bromopyridin-2-yl)cyclopropanecarboxamide

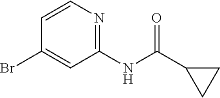 [SMILES: O=C(Nc1cc(Br)ccn1)C1CC1]

250 mg 4-bromopyridin-2-amine (1.44 mmol, 1.0 eq.) and 240 μL triethylamine (1.7 mmol. 1.2 eq.) were dissolved in 1.4 mL dichloromethane and cooled to 0° C. Then 160 μl cyclopropanecarbonyl chloride (1.7 mmol) was added dropwise. The icebath was removed and the mixture was stirred at room temperature over night. The reaction mixture was evaporated under vacuo. The residue was treated with water. A white oily precipitate could not be filtered off. The mixture was extracted with ethyl acetate twice. The organic layer was dried using a water resistant filter and the filtrate was concentrated under reduced pressure. The crude product was purified by flash chromatography and HPLC to provide the the analytically pure target comound: 113 mg, 0.47 mmol, 33%.

¹H-NMR (500 MHz, DMSO-de): δ [ppm]=0.77-0.86 (m, 4H), 1.93-2.04 (m, 1H), 7.33 (dd, 1H), 8.21 (d, 1H), 8.31 (d, 1H), 11.00 (s, 1H).

### Intermediate 1-16-1

### Preparation of [2,6-difluoro-4-(2,2,2-trifluoroethoxy)phenyl]methanol

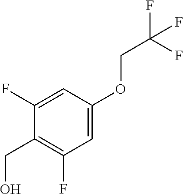 [SMILES: OCc1c(F)cc(OCC(F)(F)F)cc1F]

5 g (31.2 mmol) of 3,5-difluoro-4-(hydroxymethyl)phenol (CAS-RN 438049-36-6) were dissolved in 60 mL DMF. 1.37 g (34.3 mmol) of sodium hydride (60% dispersion in mineral oil) were added portionwise under Inert atmosphere and stirred 30 minutes at room temperature. The reaction mixture was cooled to 0° C. and 1.15 g (3.12 mmol) tetra-n-butylammonium iodide and 4.5 mL (31.2 mmol) 2,2,2-trifluoroethyl trifluoromethanesulfonate were added subsequently. The mixture was stirred overnight at room temperature, then poured into water and extracted with DCM. The organic layer was dried using a water resistant filter and concentrated under reduced pressure. The crude product was used without further purification: 9.63 g (96% yield, 75% purity) of [2,6-difluoro-4-(2,2,2-trifluoroethoxy)phenyl]methanol.

¹H-NMR (400 MHz, DMSO-di): δ [ppm]=4.42 (br s., 2H), 4.83 (q, 2H). 5.17 (br. s., 1H), 6.90 (m, 2H).

### Intermediate 1-17-1

### Preparation of 4-bromo-3-(2,2,2-trifluoroethoxy)pyridine

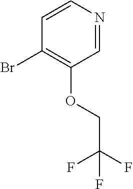 [SMILES: FC(F)(F)COc1cnccc1Br]

300 mg (1.72 mmol) 4-bromopyridin-3-ol (CAS RN 161417-28-3) was dissolved in 7.5 mL N,N-dimethylacetamide. 310 mg (2.24 mmol) Potassium carbonate and 373 μL (2.59 mmol) 2,2,2-trifluoroethyl trifluoromethanesulfonate were added. The reaction mixture was stirred at room temperature for 90 minutes. The reaction mixture was filtered and the filtrate was concentrated under reduced pressure. The crude product was used without further purification: 400 mg (58% yield, 64% purity) of 4-bromo-3-(2,2,2-trifluoroethoxy)pyridine.

¹H-NMR (400 MHz, DMSO-de): δ [ppm]=5.03 (q, 2H), 7.76 (d, 1H), 8.11 (d, 1H), 8.51 (s, 1H).

### Intermediate 1-18-1

### Preparation of 2-[1-(4-ethoxy-2,6-difluorobenzyl)-1H-Indazol-3-yl]-5-methoxy-N-[3-(2,2,2-trifluoroethoxy)pyridin-4-yl]pyrimidin-4-amine

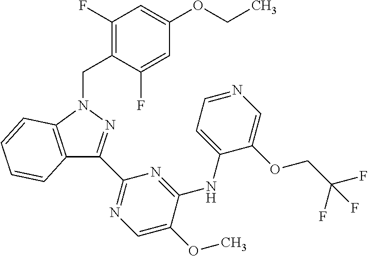 [SMILES: CCOc1cc(F)c(Cn2nc(-c3ncc(OC)c(Nc4ccncc4OCC(F)(F)F)n3)c3ccccc32)c(F)c1]

584 mg of 2-[1-(4-ethoxy-2,6-difluorobenzyl)-1H-indazol-3-yl]-5-methoxypyrimidin-4-amine 1-4-1 (1.42 mmol, 1.0 eq.) were dissolved in 12 mL tetrahydrofuran. 400 mg 4-bromo-3-(2,2,2-trifluoroethoxy)pyridine 1-17-1 (1.56 mmol, 1.1 eq.), 164 mg sodium tert-butylate (1.70 mmol, 1.2 eq.), 39 mg tris(dibenzylideneacetone)dipalladium(0) (42.6 μmol, 0.03 eq.) and 55.9 mg 2′-(dicyclohexylphosphanyl)-N,N-dimethylbiphenyl-2-amine (142 μmol, 0.1 eq.) were added. The reaction mixture was heated for 3 h to 100° C. and subsequently for 7 h to 120° C. In a microwave oven before it was poured onto water. The aqueous layer was extracted three times with ethyl acetate and the combined organic layers were washed with brine. The organic layer was filtered through a silicone coated filter and concentrated under reduced pressure. The residue was purified by flash chromatography to provide the desired title compound: 371 mg, 43%, 97% purity.

¹H-NMR (400 MHz, DMSO-d₆): δ [ppm]=1.27 (t, 3H), 4.02 (q, 2H), 4.03 (s, 3H), 5.04 (q, 2H), 5.66 (s, 2H), 6.79 (m, 2H), 7.26 (dd, 1H), 7.48 (dd, 1H), 7.84 (d, 1H), 8.16 (br. s., 1H), 8.26 (d, 1H), 8.39 (s, 1H), 8.43 (d, 1H), 8.49 (s, 1H), 8.85 (d, 1H).

The following intermediates were prepared according to the same procedure from the indicated starting material (SM=starting material):

| | | | |
|---|---|---|---|
| 1-18-2 SM = 1-9-1 | 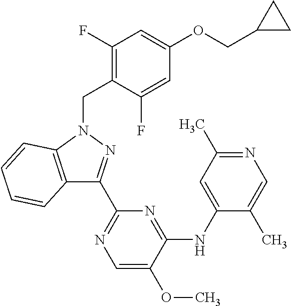 [SMILES: COc1cnc(-c2nn(Cc3c(F)cc(OCC4CC4)cc3F)c3ccccc23)nc1Nc1cc(C)ncc1C] | 2-{1-[4- (cyclopropylmethoxy)- 2,6-difluorobenzyl]- 1H-indazol-3-yl}-N- (2,5-dimethylpyridin-4- yl)-5-methoxypyrimin- 4-yl)pyrimidin-4-amine | ¹H-NMR (400 MHz, DMSO-d₆): δ [ppm] = 0.28 (m, 2H), 0.55 (m, 2H), 1.18 (m, 1H), 2.22 (s, 3H), 2.46 (s, 3H), 3.82 (d, 2H), 4.04 (s, 3H), 5.66 (s, 2H), 6.75 (m, 2H), 7.17 (dd, 1H), 7.45 (dd, 1H), 7.80 (d, 1H), 8.14 (s, 1H), 8.17 (br. s., 1H), 8.26 (s, 1H), 8.31 (s, 1H), 8.31 (d, 1H). |
| 1-18-3 SM = 1-9-1 | 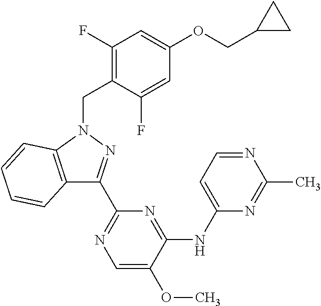 [SMILES: COc1cnc(-c2nn(Cc3c(F)cc(OCC4CC4)cc3F)c3ccccc23)nc1Nc1ccnc(C)n1] | 2-{1-[4- (cyclopropylmethoxy)- 2,6-difluorobenzyl]- 1H-indazol-3-yl}-5- methoxy-N-(2- methylpyrimidin-4- yl)pyrimidin-4-amine | ¹H-NMR (400 MHz, DMSO-d₆): δ [ppm] = 0.29 (m, 2H), 0.54 (m, 2H), 1.18 (m, 1H), 2.55 (s, 3H), 3.82 (d, 2H), 4.01 (s, 3H), 5.68 (s, 2H), 6.81 (m, 2H), 7.28 (dd, 1H), 7.50 (dd, 1H), 7.85 (d, 1H), 8.43 (s, 1H), 8.48 (d, 1H), 8.52 (d, 1H), 8.56 (d, 1H), 8.92 (br. s., 1H). |

### Intermediate 1-19-1

### Preparation of 2-(bromomethyl)-1,3-difluoro-5-(2-methoxyethoxy)benzene

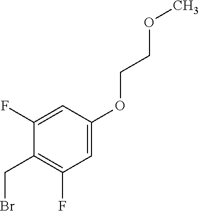

6.0 g [2,6-Difluoro-4-(2-methoxyethoxy)phenyl]methanol 1-12-3 (27.4 mmol, 1.0 eq.) was dissolved in 22 mL dichloromethane. 1.9 mL phosphorous tribromide (20.5 mmol, 0.75 eq.) was dissolved in 22 mL dichloromethane and given to the starting material. The reaction mixture was stirred at room temperature under argon atmosphere for 30 min. The reaction mixture was poured into ice water and the pH was adjusted to 8 by adding saturated sodium hydrogen carbonate solution. It was extracted three times with dichioromethane, washed once with water and brine, filtered through a silicone coated filter and dried under reduced pressure to provide the crude product, which was used without further purification: 8.2 g, 29.2 mmol, 107% yield.

¹H-NMR (300 MHz, DMSO-d6): δ [ppm]=3.28 (s, 3H), 3.58-3.70 (m, 2H), 4.10-4.18 (m, 2H), 4.60 (s, 2H), 6.73-6.90 (m, 2H).

### Example Compounds

### Example 2-1-1

### Preparation of 3-[(4-{bis[2-(difluoromethyl)pyridin-4-yl]amino}-2-[1-(4-ethoxy-2,6-difluorobenzyl)-1H-Indazol-3-yl]pyrimidin-5-yl)oxy]propan-1-ol

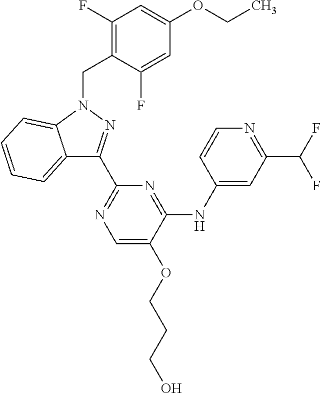 [SMILES: CCOc1cc(F)c(Cn2nc(-c3ncc(OCCCO)c(Nc4ccnc(C(F)F)c4)n3)c3ccccc32)c(F)c1]

1.64 g of 5-(3-{[tert-butyl(dimethyl)silyl]oxy}propoxy)-N-[2-(difluoromethyl)pyridin-4-yl]-2-[1-(4-ethoxy-2,6-difluorobenzyl)-1H-Indazol-3-yl]pyrimidin-4-amine 1-1-1 (98% pure, 2.31 mmol, 1.0 eq.) was dissolved in 9.9 mL dioxane. Then 2.3 mL hydrochloric acid in dioxane (4 M, 9.22 mmol, 4.0 eq.) was added and the reaction mixture was stirred for 1 h at RT. The reaction mixture was diluted with ethyl acetate and then sodium hydrogen carbonate was added until pH8. The layers were separated and the aqueous layer was extracted with ethyl acetate twice. The combined organic layers were dried using a water resistant filter and the filtrate concentrated in vacuo. The crude product recrystallized from ethyl acetate twice to provide the analytically pure target compound: 1.0 g, 1.72 mmol, 75%.

¹H-NMR (400 MHz, DMSO-d₆): δ [ppm]=1.30 (t, 3H), 1.79 (quint, 2H), 3.42 (q, 2H), 4.05 (q, 2H), 4.21 (t, 2H), 4.56 (t, 1H), 5.69 (s, 2H), 6.70-7.17 (m, 4H), 7.20 (t, 1H), 7.47 (t, 1H), 7.72 (d, 1H), 7.77-7.87 (m, 2H), 8.28-8.35 (m, 2H), 8.58-8.67 (m, 3H), 8.76 (s, 1H).

The following examples were prepared according to the same procedure from the indicated starting materials (SM=starting material):

| | | | |
|---|---|---|---|
| 2-1-2 SM = 1-1-2 | 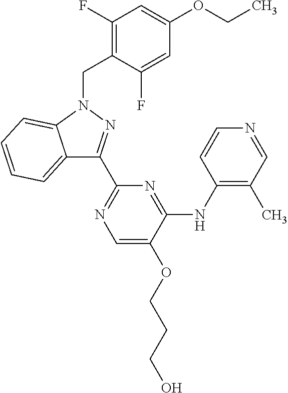 [SMILES: CCOc1cc(F)c(Cn2nc(-c3ncc(OCCCO)c(Nc4ccncc4C)n3)c3ccccc32)c(F)c1] | 3-({2-[1-(4-ethoxy-2,6- difluorobenzyl)-1H- indazol-3-yl]-4-[(3- methylpyridin-4- yl)amino]pyrimidin-5- yl}oxy)propan-1-ol | ¹H-NMR (400 MHz, DMSO-d₆): δ [ppm] = 1.30 (t, 3H), 1.94-2.05 (m, 2H), 2.29 (s, 3H), 3.67 (q, 2H), 4.05 (q, 2H), 4.33 (t, 2H), 4.67 (t, 1H), 5.66 (s, 2H), 6.74-6.84 (m, 2H), 7.20 (t, 1H), 7.42-7.51 (m, 1H), 7.82 (d, 1H), 8.10 (s, 1H), 8.31 (d, 1H), 8.34 (s, 1H), 8.37-8.46 (m, 3H). |
| 2-1-3 SM = 1-1-3 | 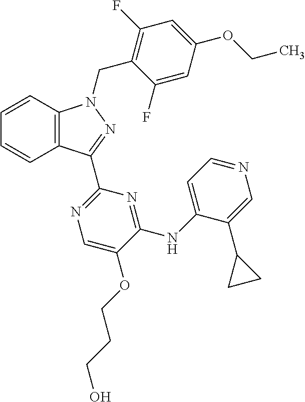 [SMILES: CCOc1cc(F)c(Cn2nc(-c3ncc(OCCCO)c(Nc4ccncc4C4CC4)n3)c3ccccc32)c(F)c1] | 3-({4-[(3- cyclopropylpyridin-4- yl)amino]-2-[1-(4-ethoxy- 2,6-difluorobenzyl)-1H- indazol-3-yl]pyrimidin-5- yl}oxy)propan-1-ol | ¹H-NMR (400 MHz, DMSO-d₆): δ [ppm] = 0.69-0.77 (m, 2H), 1.00-1.07 (m, 2H), 1.30 (t, 3H), 1.86-1.96 (m, 1H), 2.02 (quint, 2H), 3.66 (q, 2H), 4.05 (q, 2H), 4.36 (t, 2H), 4.67 (t, 1H), 5.69 (s, 2H), 6.74-6.85 (m, 2H), 7.29 (t, 1H), 7.47-7.53 (m, 1H), 7.86 (d, 1H), 8.34-8.49 (m, 5H), 8.88 (d, 1H). |
| 2-1-4 SM = 1-1-4 | 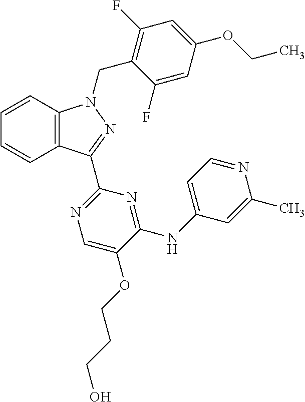 [SMILES: CCOc1cc(F)c(Cn2nc(-c3ncc(OCCCO)c(Nc4ccnc(C)c4)n3)c3ccccc32)c(F)c1] | 3-({2-[1-(4-ethoxy-2,6- difluorobenzyl)-1H- indazol-3-yl]-4-[(2- methylpyridin-4- yl)amino]pyrimidin-5- yl}oxy)propan-1-ol | ¹H-NMR (400 MHz, DMSO-d₆): δ [ppm] = 1.28 (t, 3H), 1.94-2.05 (m, 2H), 2.44 (s, 3H), 3.65 (q, 2H), 4.03 (q, 2H), 4.30 (t, 2H), 4.65 (t, 1H), 5.68 (s, 2H), 6.70-6.82 (m, 2H), 7.25 (t, 1H), 7.42-7.54 (m, 1H), 7.82 (d, 1H), 7.86-7.92 (m, 1H), 8.04 (d, 1H), 8.29 (d, 1H), 8.34 (s, 1H), 8.47 (d, 1H), 9.01 (s, 1H). |
| 2-1-5 SM = 1-1-5 | 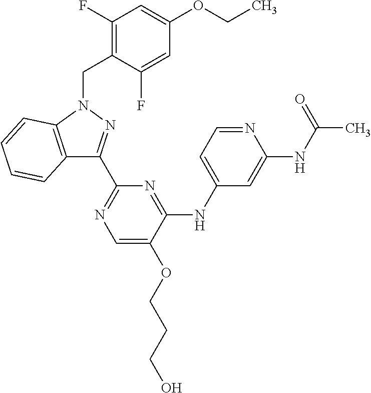 [SMILES: CCOc1cc(F)c(Cn2nc(-c3ncc(OCCCO)c(Nc4ccnc(NC(C)=O)c4)n3)c3ccccc32)c(F)c1] | N-[4-({2-[1-(4-ethoxy-2,6- difluorobenzyl)-1H- indazol-3-yl]-5-(3- hydroxypropoxy)pyrimidin- 4-yl}amino)pyridin-2- yl]acetamide | ¹H-NMR (300 MHz, DMSO-d₆): δ [ppm] = 1.28 (t, 3H), 1.93-2.13 (m, 5H), 3.64 (q, 2H), 4.03 (q, 2H), 4.28 (t, 2H), 4.63 (t, 1H), 5.66 (s, 2H), 6.71-6.83 (m, 2H), 7.22 (t, 1H), 7.46 (t, 1H), 7.81 (d, 1H), 8.15 (d, 1H), 8.22-8.28 (m, 1H), 8.28-8.36 (m, 2H), 8.43 (d, 1H), 9.26 (s, 1H), 10.33 (s, 1H). |
| 2-1-6 SM = 1-1-6 | 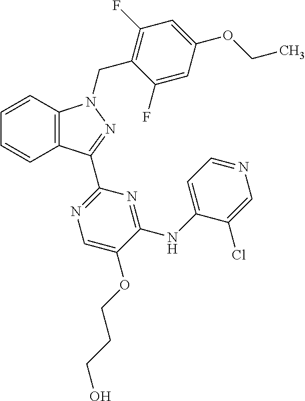 [SMILES: CCOc1cc(F)c(Cn2nc(-c3ncc(OCCCO)c(Nc4ccncc4Cl)n3)c3ccccc32)c(F)c1] | 3-{[4-[(3-chloropyridin-4- yl)amino]-2-[1-(4-ethoxy- 2,6-difluorobenzyl)-1H- indazol-3-yl]pyrimidin-5- yl}oxy)propan-1-ol | ¹H-NMR (300 MHz, DMSO-d₆): δ [ppm] = 1.28 (t, 3H), 1.98 (quin, 2H), 3.65 (q, 2H), 4.03 (q, 2H), 4.36 (t, 2H), 4.65 (t, 1H), 5.68 (s, 2H), 6.74-6.87 (m, 2H), 7.26 (t, 1H), 7.49 (t, 1H), 7.85 (d, 1H), 8.27 (s, 1H), 8.36-8.50 (m, 3H), 8.64 (s, 1H), 9.01 (d, 1H). |
| 2-1-7 SM = 1-1-7 | 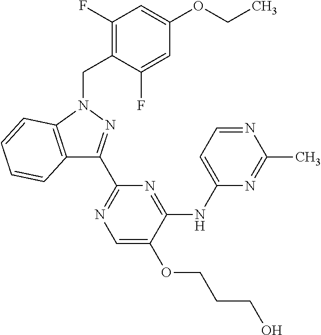 [SMILES: CCOc1cc(F)c(Cn2nc(-c3ncc(OCCCO)c(Nc4ccnc(C)n4)n3)c3ccccc32)c(F)c1] | 3-({2-[1-(4-ethoxy-2,6- difluorobenzyl)-1H- indazol-3-yl]-4-[(2- methylpyrimidin-4- yl)amino]pyrimidin-5- yl}oxy)propan-1-ol | ¹H-NMR (400 MHz, DMSO-d₆): δ [ppm] = 1.28 (t, 3H), 1.98 (quint, 2H), 2.55 (s, 3H), 3.63 (q, 2H), 4.03 (q, 2H), 4.31 (t, 2H), 4.62 (t, 1H), 5.67 (s, 2H), 6.76-6.84 (m, 2H), 7.27 (t, 1H), 7.45-7.53 (m, 1H), 7.84 (d, 1H), 8.40-8.49 (m, 2H), 8.52 (d, 1H), 8.59 (d, 1H), 8.93 (s, 1H). |
| 2-1-8 SM = 1-1-8 | 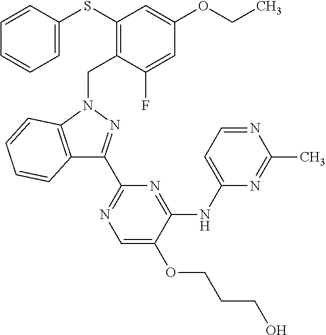 [SMILES: CCOc1cc(F)c(Cn2nc(-c3ncc(OCCCO)c(Nc4ccnc(C)n4)n3)c3ccccc32)c(Sc2ccccc2)c1] | 3-[(2-{1-[4-ethoxy-2- fluoro-6- (phenylsulfanyl)benzyl]- 1H-indazol-3-yl]-4-[(2- methylpyrimidin-4- yl)amino]pyrimidin-5- yl)oxy]propan-1-ol | ¹H-NMR (400 MHz, DMSO-d₆): δ [ppm] = 1.28 (t, 3H), 1.98 (quint, 2H), 2.55 (s, 3H), 3.63 (q, 2H), 4.03 (q, 2H), 4.31 (t, 2H), 4.62 (t, 1H), 5.67 (s, 2H), 6.76-6.84 (m, 2H), 7.27 (t, 1H), 7.45-7.53 (m, 1H), 7.84 (d, 1H), 8.40-8.49 (m, 2H), 8.52 (d, 1H), 8.59 (d, 1H), 8.93 (s, 1H). |
| 2-1-9 SM = 1-1-9 | 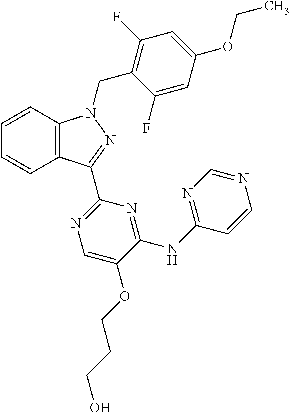 [SMILES: CCOc1cc(F)c(Cn2nc(-c3ncc(OCCCO)c(Nc4ccncn4)n3)c3ccccc32)c(F)c1] | 3-({2-[1-(4-ethoxy-2,6- difluorobenzyl)-1H- indazol-3-yl]-4-(pyrimidin- 4-ylamino)pyrimidin-5- yl}oxy)propan-1-ol | ¹H-NMR (400 MHz, DMSO-d₆): δ [ppm] = 1.30 (t, 3H), 1.99 (quint, 2H), 3.65 (t, 2H), 4.05 (q, 2H), 4.32 (t, 2H), 4.69 (br. s, 1H), 5.69 (s, 2H), 6.79-6.88 (m, 2H), 7.30 (t, 1H), 7.51 (t, 1H), 7.87 (d, 1H), 8.44-8.52 (m, 2H), 8.63 (d, 1H), 8.77 (d, 1H), 8.88 (s, 1H), 9.17 (br. s, 1H). |
| 2-1-10 SM = 1-1-10 | 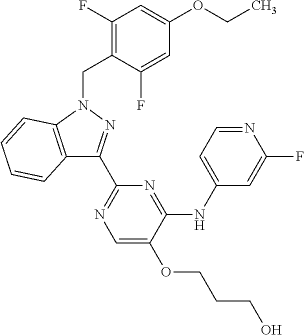 [SMILES: CCOc1cc(F)c(Cn2nc(-c3ncc(OCCCO)c(Nc4ccnc(F)c4)n3)c3ccccc32)c(F)c1] | 3-({2-[1-(4-ethoxy-2,6- difluorobenzyl)-1H- indazol-3-yl]-4-[(2- fluoropyridin-4- yl)amino]pyrimidin-5- yl}oxy)propan-1-ol | ¹H-NMR (400 MHz, DMSO-d₆): δ [ppm] = 1.28 (t, 3H), 2.01 (q, 2H), 3.65 (t, 2H), 4.03 (q, 2H), 4.34 (t, 2H), 4.67 (s, 1H), 5.68 (s, 2H), 6.69-6.85 (m, 2H), 7.27 (t, 1H), 7.50 (t, 1H), 7.85 (d, 1H), 8.00 (d, 1H), 8.07 (d, 2H), 8.43 (s, 1H), 8.47 (d, 1H), 9.47 (s, 1H). |
| 2-1-11 SM = 1-1-11 | 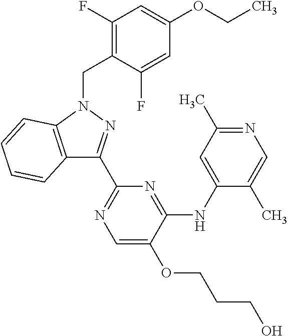 [SMILES: CCOc1cc(F)c(Cn2nc(-c3ncc(OCCCO)c(Nc4cc(C)ncc4C)n3)c3ccccc32)c(F)c1] | 3-{[4-[(2,5- dimethylpyridin-4- yl)amino]-2-[1-(4-ethoxy- 2,6-difluorobenzyl)-1H- indazol-3-yl]pyrimidin-5- yl}oxy)propan-1-ol | ¹H-NMR (400 MHz, DMSO-d₆): δ [ppm] = 1.28 (t, 3H), 1.98 (quin, 2H), 2.23 (s, 3H), 2.46 (s, 3H), 3.65 (q, 2H), 4.03 (q, 2H), 4.32 (t, 2H), 4.64 (t, 1H), 5.66 (s, 2H), 6.67-6.79 (m, 2H), 7.19 (t, 1H), 7.45 (t, 1H), 7.80 (d, 1H), 7.99 (s, 1H), 8.25 (s, 1H), 8.31 (d, 2H), 8.36 (d, 1H), |
| 2-1-12 SM = 1-1-12 | 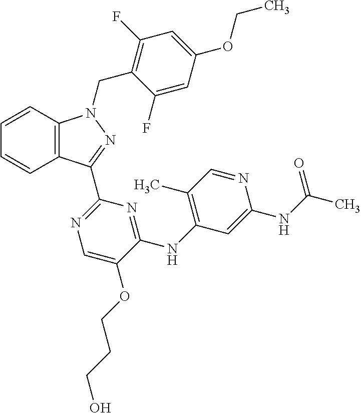 [SMILES: CCOc1cc(F)c(Cn2nc(-c3ncc(OCCCO)c(Nc4cc(NC(C)=O)ncc4C)n3)c3ccccc32)c(F)c1] | N-[4-({2-[1-(4-ethoxy-2,6- difluorobenzyl)-1H- indazol-3-yl]-5-(3- hydroxypropoxy)pyrimidin- 4-yl}amino)-5- methylpyridin-2- yl]acetamide | ¹H-NMR (400 MHz, DMSO-d₆): δ [ppm] = 1.30 (t, 3H), 1.95-2.03 (m, 2H), 2.05 (s, 3H), 2.14 (s, 3H), 3.67 (q, 2H), 4.04 (q, 2H), 4.29 (t, 2H), 4.63 (t, 1H), 5.62 (s, 2H), 6.70-6.81 (m, 2H), 7.02 (t, 1H), 7.41 (t, 1H), 7.72 (d, 1H), 8.09 (d, 1H), 8.19 (s, 1H), 8.24 (s, 1H), 8.35 (s, 1H), 8.67 (s, 1H), 10.44 (s, 1H). |
| 2-1-13 SM = 1-1-13 The reaction mixture was stirred at 60° C. for 6 h. | 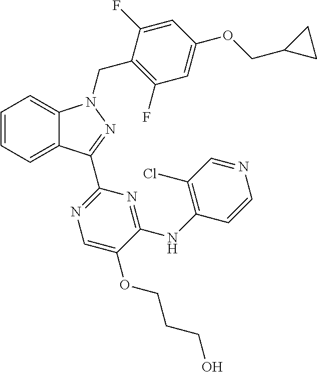 [SMILES: OCCCOc1cnc(-c2nn(Cc3c(F)cc(OCC4CC4)cc3F)c3ccccc23)nc1Nc1ccncc1Cl] | 3-[(4-[(3-chloropyridin-4- yl)amino]-2-{1-[4- (cyclopropylmethoxy)-2,6- difluorobenzyl]-1H- indazol-3-yl}pyrimidin-5- yl)oxy]propan-1-ol | ¹H-NMR (400 MHz, DMSO-d₆): δ [ppm] = 0.17-0.40 (m, 2 H) 0.46-0.64 (m, 2 H) 1.07-1.30 (m, 1 H) 1.98 (quin, 2 H) 3.61-3.73 (m, 2 H) 3.83 (d, 2 H) 4.36 (t, 2 H) 4.68 (t, 1 H) 5.67 (s, 2 H) 6.80 (s, 1 H) 6.82 (s, 1 H) 7.27 (t, 1 H) 7.49 (ddd, 1 H) 7.84 (d, 1 H) 8.25 (s, 1 H) 8.37-8.52 (m, 3 H) 8.64 (s, 1 H) 9.02 (d, 1 H). |
| 2-1-14 SM = 1-1-14 The reaction mixture was stirred at RT over night | 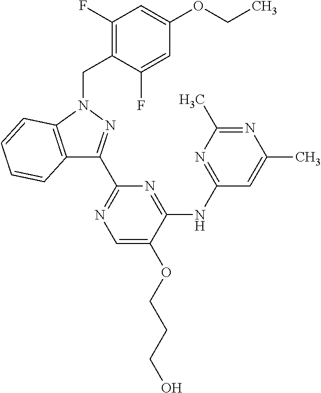 [SMILES: CCOc1cc(F)c(Cn2nc(-c3ncc(OCCCO)c(Nc4cc(C)nc(C)n4)n3)c3ccccc32)c(F)c1] | 3-({4-[(2,6- Dimethylpyrimidin-4- yl)amino]-2-[1-(4-ethoxy- 2,6-difluorobenzyl)-1H- indazol-3-yl]pyrimidin-5- yl}oxy)propan-1-ol | ¹H-NMR (400 MHz, CHLOROFORM-d): δ [ppm] = 1.40 (t, 3H), 1.95 (br. s., 1H), 2.19 (quin, 2H), 2.58 (s, 3H), 2.64 (s, 3H), 3.93-4.03 (m, 4H), 4.38 (t, 2H), 5.74 (s, 2H), 6.42-6.52 (m, 2H), 7.24-7.32 (m, 1H), 7.44 (t, 1H), 7.61 (d, 1H), 8.11 (s, 1H), 8.30 (s, 1H), 8.57-8.64 (m, 2H). |
| 2-1-15 SM = 1-1-15 | 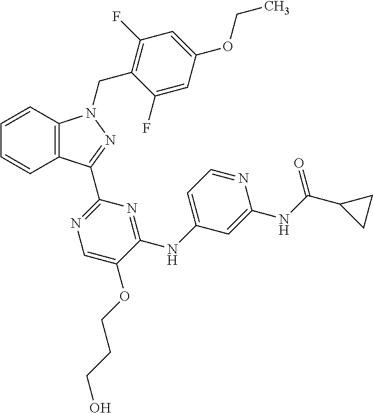 [SMILES: CCOc1cc(F)c(Cn2nc(-c3ncc(OCCCO)c(Nc4ccnc(NC(=O)C5CC5)c4)n3)c3ccccc32)c(F)c1] | N-[4-({2-[1-(4-Ethoxy-2,6- difluorobenzyl)-1H- indazol-3-yl]-5-(3- hydroxypropoxy)pyrimidin- 4-yl}amino)pyridin-2- yl]cyclopropancarboxamide | ¹H-NMR (400 MHz, DMSO-d₆): δ [ppm] = 0.73-0.88 (m, 4H), 1.30 (t, 3H), 1.96-2.06 (m, 3H), 3.65 (t, 2H), 4.04 (q, 2H), 4.30 (t, 2H), 4.65 (br. s, 1H), 5.69 (s, 2H), 6.72-6.85 (m, 2H), 7.19-7.28 (m, 1H), 7.44-7.52 (m, 1H), 7.84 (d, 1H), 8.18 (d, 1H), 8.25 (s, 1H), 8.35 (br. s, 2H), 8.44 (d, 1H), 9.32 (br. s, 1H), 10.77 (br. s, 1H). |
| 2-1-16 SM = 1-1-16 | 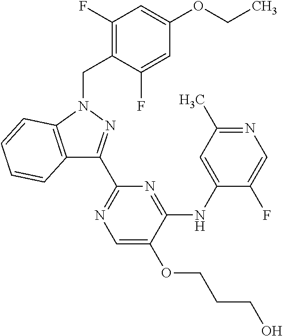 [SMILES: CCOc1cc(F)c(Cn2nc(-c3ncc(OCCCO)c(Nc4cc(C)ncc4F)n3)c3ccccc32)c(F)c1] | 3-({2-[1-(4-Ethoxy-2,6- difluorobenzyl)-1H- indazol-3-yl]-4-[(5-fluor-2- methylpyridin-4- yl)amino]pyrimidin-5- yl}oxy)propan-1-ol | ¹H-NMR (400 MHz, CHLOROFORM-d): δ [ppm] = 1.40 (t, 3H), 2.16-2.25 (m, 2H), 2.63 (s, 3H), 3.94-4.00 (m, 4H), 4.40 (t, 2H), 5.75 (s, 2H), 6.43-6.52 (m, 2H), 7.26-7.32 (m, 1H), 7.42-7.48 (m, 1H), 7.61 (d, 1H), 7.76-7.81 (m, 1H), 8.31 (s, 1H), 8.34 (d, 1H), 8.59-8.63 (m, 1H), 8.92 (d, 1H). - OH not detectable. |
| 2-1-17 SM = 1-1-17 | 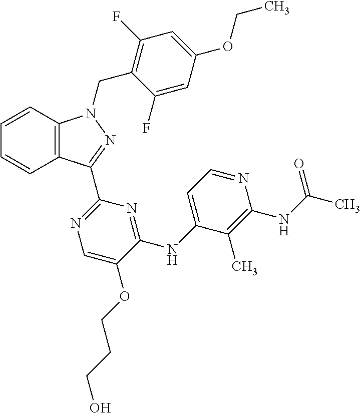 [SMILES: CCOc1cc(F)c(Cn2nc(-c3ncc(OCCCO)c(Nc4ccnc(NC(C)=O)c4C)n3)c3ccccc32)c(F)c1] | N-[4-({2-[1-(4-ethoxy-2,6- difluorobenzyl)-1H- indazol-3-yl]-5-(3- hydroxypropoxy)pyrimidin- 4-yl}amino)-3- methylpyridin-2- yl]acetamide | ¹H-NMR (400 MHz, DMSO-d₆): δ [ppm] = 1.29 (t, 3H), 1.94-2.04 (m, 2H), 2.07 (s, 3H), 2.11 (s, 3H), 3.66 (t, 2H), 4.04 (q, 2H), 4.32 (t, 2H), 4.67 (br. s, 1H), 5.66 (s, 2H), 6.74-6.83 (m, 2H), 7.20 (t, 1H), 7.42-7.50 (m, 1H), 7.81 (d, 1H), 8.15-8.26 (m, 2H), 8.27-8.38 (m, 3H), 10.05 (br. s, 1H). |
| 2-1-19 SM = 1-1-19 | 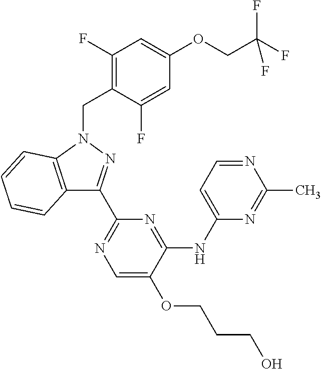 [SMILES: Cc1nccc(Nc2nc(-c3nn(Cc4c(F)cc(OCC(F)(F)F)cc4F)c4ccccc34)ncc2OCCCO)n1] | 3-[(2-{1-[2,6-difluoro-4- (2,2,2- trifluoroethoxy)benzyl]-1H- indazol-3-yl}-4-[(2- methylpyrimidin-4- yl)amino]pyrimidin-5- yl)oxy]propan-1-ol | ¹H-NMR (400 MHz, DMSO-d₆): δ [ppm] = 1.99 (tt, 2H), 2.56 (s, 3H), 3.61 (td, 2H), 4.31 (t, 2H), 4.66 (t, 1H), 4.83 (q, 2H), 5.72 (s, 2H), 7.03 (m, 2H), 7.29 (dd, 1H), 7.51 (dd, 1H), 7.87 (d, 1H), 8.44 (s, 1H), 8.47 (d, 1H), 8.55 (d, 1H), 8.62 (d, 1H), 9.00 (br. s., 1H). |
| 2-1-20 SM = 1-1-20 | 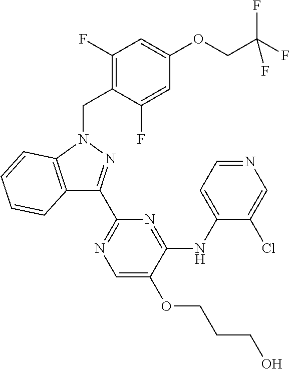 [SMILES: OCCCOc1cnc(-c2nn(Cc3c(F)cc(OCC(F)(F)F)cc3F)c3ccccc23)nc1Nc1ccncc1Cl] | 3-[(4-[(3-chloropyridin-4- yl)amino]-2-{1-[2,6- difluoro-4-(2,2,2- trifluoroethoxy)benzyl]-1H- indazol-3-yl}pyrimidin-5- yl)oxy]propan-1-ol | ¹H-NMR (400 MHz, DMSO-d₆): δ [ppm] = 1.99 (tt, 2H), 3.66 (td, 2H), 4.36 (t, 2H), 4.68 (t, 1H), 4.83 (q, 2H), 5.72 (s, 2H), 7.03 (m, 2H), 7.28 (dd, 1H), 7.50 (dd, 1H), 7.87 (d, 1H), 8.28 (br. s., 1H), 8.41 (d, 1H), 8.47 (s, 1H), 8.48 (d, 1H), 8.65 (s, 1H), 9.03 (d, 1H). |
| 2-1-21 SM = 1-1-21 | 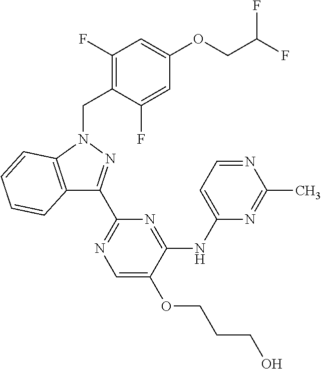 [SMILES: Cc1nccc(Nc2nc(-c3nn(Cc4c(F)cc(OCC(F)F)cc4F)c4ccccc34)ncc2OCCCO)n1] | 3-[(2-{1-[4-(2,2- difluoroethoxy)-2,6- difluorobenzyl]-1H- indazol-3-yl}-4-[(2- methylpyrimidin-4- yl)amino]pyrimidin-5- yl)oxy]propan-1-ol | ¹H-NMR (400 MHz, DMSO-d₆): δ [ppm] = 1.99 (tt, 2H), 2.56 (s, 3H), 3.44 (td, 2H), 4.31 (t, 2H), 4.37 (td, 2H), 4.65 (t, 1H), 5.71 (s, 2H), 6.37 (tt, 1H), 6.96 (m, 2H), 7.29 (dd, 1H), 7.50 (dd, 1H), 7.87 (d, 1H), 8.44 (s 1H), 8.47 (d, 1H), 8.55 (d, 1H), 8.62 (d, 1H), 8.99 (br. s., 1H). |
| 2-1-22 SM = 1-1-22 reaction time: 24 h | 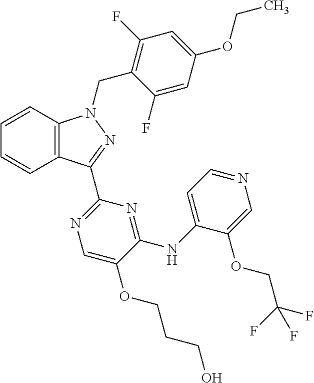 [SMILES: CCOc1cc(F)c(Cn2nc(-c3ncc(OCCCO)c(Nc4ccncc4OCC(F)(F)F)n3)c3ccccc32)c(F)c1] | 3-[(2-[1-(4-ethoxy-2,6- difluorobenzyl)-1H- indazol-3-yl]-4-{[3-(2,2,2- trifluoroethoxy)pyridin-4- yl]amino}pyrimidin-5- yl)oxy]propan-1-ol | ¹H-NMR (400 MHz, DMSO-d₆): δ [ppm] = 1.29 (t, 3H), 1.95 (tt, 2H), 3.63 (td, 2H), 4.04 (q, 2H), 4.33 (t, 2H), 4.65 (t, 1H), 5.07 (q, 2H), 5.69 (s, 2H), 6.82 (m, 2H), 7.29 (dd, 1H), 7.50 (dd, 1H), 7.87 (d, 1H), 8.17 (br. s., 1H), 8.28 (d, 1H), 8.42 (s, 1H), 8.47 (s, 1H), 8.47 (d, 1H), 8.98 (d, 1H). |
| 2-1-23 SM = 1-1-23 reaction time: 19 h | 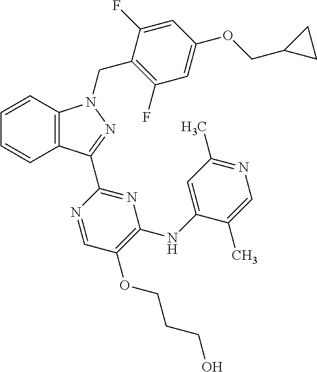 [SMILES: Cc1cc(Nc2nc(-c3nn(Cc4c(F)cc(OCC5CC5)cc4F)c4ccccc34)ncc2OCCCO)c(C)cn1] | 3-[(2-{1-[4- (cyclopropylmethoxy)-2,6- difluorobenzyl]-1H- indazol-3-yl}-4-[(2,5- dimethylpyridin-4- yl)amino]pyrimidin-5- yl)oxy]propan-1-ol | ¹H-NMR (400 MHz, DMSO-d₆): δ [ppm] = 0.28 (m, 2H), 0.54 (m, 2H), 1.18 (m, 1H), 1.99 (tt, 2H), 2.24 (s, 3H), 2.47 (s, 3H), 3.66 (td, 2H), 3.82 (d, 2H), 4.32 (t, 2H), 4.67 (t, 1H), 5.67 (s, 2H), 6.76 (m, 2H), 7.20 (dd, 1H), 7.47 (dd, 1H), 7.81 (d, 1H), 8.01 (br. s., 1H), 8.26 (s, 1H), 8.30 (s, 1H), 8.33 (s, 1H), 8.36 (d, 1H). |
| 2-1-24 SM = 1-1-24 reaction time: 24 h | 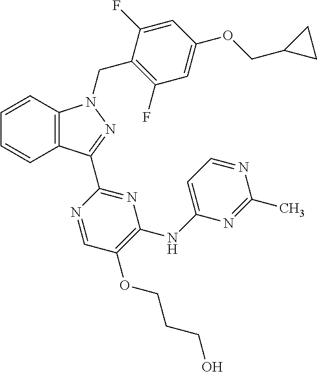 [SMILES: Cc1nccc(Nc2nc(-c3nn(Cc4c(F)cc(OCC5CC5)cc4F)c4ccccc34)ncc2OCCCO)n1] | 3-[(2-{1-[4- (cyclopropylmethoxy)-2,6- difluorobenzyl]-1H- indazol-3-yl}-4-[(2- methylpyrimidin-4- yl)amino]pyrimidin-5- yl)oxy]propan-1-ol | ¹H-NMR (400 MHz, DMSO-d₆): δ [ppm] = 0.29 (m, 2H), 0.54 (m, 2H), 1.18 (m, 1H), 1.99 (tt, 2H), 2.56 (s, 3H), 3.64 (td, 2H), 3.83 (d, 2H), 4.31 (t, 2H), 4.64 (t, 1H), 5.68 (s, 2H), 6.82 (m, 2H), 7.28 (dd, 1H), 7.50 (dd, 1H), 7.86 (d, 1H), 8.44 (s, 1H), 8.47 (d, 1H), 8.53 (d, 1H), 8.60 (d, 1H), 8.97 (br. s., 1H). |
| 2-1-25 SM = 1-1-25 reaction time: 18 h | 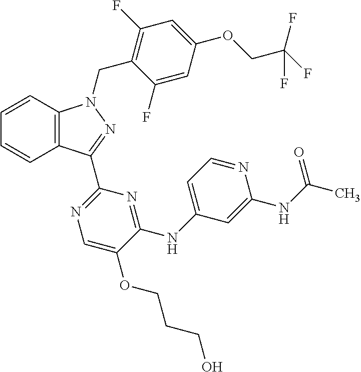 [SMILES: CC(=O)Nc1cc(Nc2nc(-c3nn(Cc4c(F)cc(OCC(F)(F)F)cc4F)c4ccccc34)ncc2OCCCO)ccn1] | N-(4-{[2-{1-[2,6-difluoro- 4-(2,2,2- trifluoroethoxy)benzyl]-1H- indazol-3-yl}-5-(3- hydroxypropoxy)pyrimidin- 4-yl]amino}pyridin-2- yl)acetamide | ¹H-NMR (400 MHz, DMSO-d₆): δ [ppm] = 2.00 (tt, 2H), 2.08 (s, 3H), 3.65 (td, 2H), 4.30 (t, 2H), 4.63 (t, 1H), 4.82 (q, 2H), 5.71 (s, 2H), 6.99 (m, 2H), 7.22 (dd, 1H), 7.48 (dd, 1H), 7.82 (d, 1H), 8.17 (d, 1H), 8.27 (br. s., 1H), 8.31 (dd, 1H), 8.33 (s, 1H), 8.42 (d, 1H), 9.26 (br. s., 1H), 10.32 (br. s., 1H). |
| 2-1-26 SM = 1-1-26 reaction time: over night | 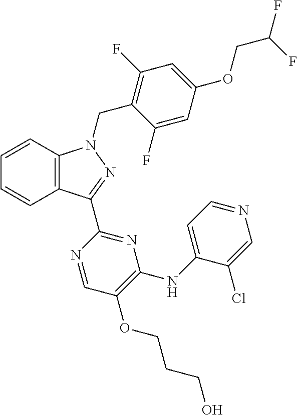 [SMILES: OCCCOc1cnc(-c2nn(Cc3c(F)cc(OCC(F)F)cc3F)c3ccccc23)nc1Nc1ccncc1Cl] | 3-[(4-[(3-chloropyridin-4- yl)amino]-2-{1-[4-(2,2- difluoroethoxy)-2,6- difluorobenzyl]-1H- indazol-3-yl}pyrimidin-5- yl)oxy]propan-1-ol | ¹H-NMR (400 MHz, DMSO-d₆): δ [ppm] = 1.99 (tt, 2H), 3.66 (td, 2H), 4.36 (td, 2H), 4.37 (t, 2H), 4.68 (t, 1H), 5.72 (s, 2H), 6.37 (tt, 1H), 6.97 (m, 2H), 7.28 (dd, 1H), 7.51 (dd, 1H), 7.87 (d, 1H), 8.29 (br. s., 1H), 8.41 (d, 1H), 8.48 (s, 1H), 8.48 (d, 1H), 8.66 (s, 1H), 9.03 (d, 1H). |
| 2-1-27 SM = 1-1-27 reaction time: 1.5 h | 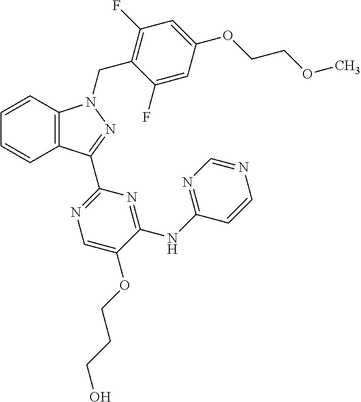 [SMILES: COCCOc1cc(F)c(Cn2nc(-c3ncc(OCCCO)c(Nc4ccncn4)n3)c3ccccc32)c(F)c1] | 3{[2-{1-[2,6-difluoro-4-(2- methoxyethoxy)benzyl]- 1H-indazol-3-yl}-4- (pyrimidin-4- ylamino)pyrimidin-5- yl]oxy}propan-1-ol | ¹H-NMR (400 MHz, DMSO-d₆): δ [ppm] = 1.92-2.04 (m, 2H), 3.26 (s, 3H), 3.58-3.70 (m, 4H), 4.05-4.16 (m, 2H), 4.32 (t, 2H), 4.68 (br. s, 1H), 5.71 (s, 2H), 6.82-6.92 (m, 2H), 7.25-7.33 (m, 1H), 7.47-7.56 (m, 1H), 7.87 (d, 1H), 8.42-8.52 (m, 2H), 8.61-8.68 (m, 1H), 8.74-8.84 (m, 1H), 8.89 (s, 1H), 9.19 (br. s, 1H). |

### Synthetic Intermediates of Prodrugs

### Intermediate 3-1-1

### Preparation of 3-[(4-{[2-(difluoromethyl)pyridin-4-yl]amino}-2-[1-(4-ethoxy-2,6-difluorobenzyl)-1H-Indazol-3-yl]pyrimidin-5-yl)oxy]propyl N²,N⁶-bis(tert-butoxycarbonyl)-L-lysyl-L-alaninate

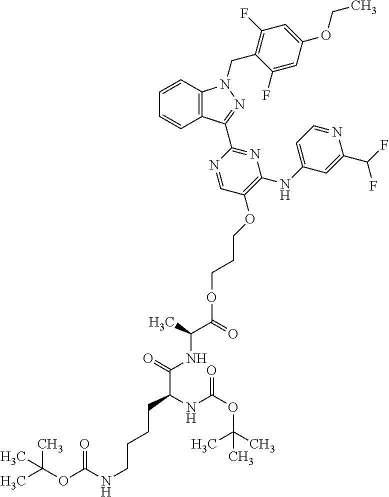 [SMILES: CCOc1cc(F)c(Cn2nc(-c3ncc(OCCCOC(=O)[C@H](C)NC(=O)[C@H](CCCCNC(=O)OC(C)(C)C)NC(=O)OC(C)(C)C)c(Nc4ccnc(C(F)F)c4)n3)c3ccccc32)c(F)c1]

657 mg of N²,N⁶-bis(tert-butoxycarbonyl)-L-lysine (1.89 mmol, 1.5 eq.), 387 mg of 1-Hydroxy-1H-benzotriazol Hydrat (2.53 mmol, 2.0 eq.) and 0.88 mL of N,N-Dilsopropylethylamin (5.1 mmol. 4.0 eq.) were dissolved in 20 mL DMF. Afterwords 436 mg of 1-(3-Dimethylaminopropyl)-3-ethylcarbodiimidhydrochlorid (2.3 mmol, 1.8 eq.) were added. After 30 min. stirring at room temperature 971 mg of 3-[(4-{[2-(difluoromethyl)pyridin-4-yl]amino}-2-[1-(4-ethoxy-2,6-difluorobenzyl)-1-H-Indazol-3-yl]pyrimidin-5-yl)oxy]propyl L-alaninate trifluoroacetate (1:1) 3-2-1 (1.27 mmol, 1.0 eq.) dissolved in 20 mL DMF was given into the reaction mixture. It was stirred at room temperature over night. The DMF was evaporated under vacuo. The residue was dissolved in ethyl acetate and the organic layer was washed with 5%-aqueous citric acid twice. The organic layer was dried using a water resistant filter and the filtrate was concentrated in vacuo. The crude product was purified by flash chromatography and again dissolved in ethyl acetate and washed with saturated sodiumhydrogen carbonate solution twice. The organic layer was dried using a water resistant filter and evaporated under vacuum to provide the 91% pure target compound: 618 mg. 0.57 mmol, 45%.

¹H-NMR (400 MHz, DMSO-de): δ [ppm]=1.18 (t, 3H). 1.27-1.32 (m, 6H), 1.35 (s, 9H), 1.35 (br. s., 9H), 2.13-2.25 (m, 2H), 2.78-2.94 (m, 2H), 3.84-3.96 (m, 1H), 3.97-4.11 (m, 5H), 4.22-4.41 (m, 5H), 5.70 (s, 2H), 6.67-7.06 (m, 5H), 7.20-7.33 (m, 1H), 7.45-7.55 (m, 1H), 7.83 (d, 1H), 8.18-8.27 (m, 1H), 8.26-8.33 (m, 1H), 8.40 (s, 1H), 8.46 (d, 1H), 8.50-8.63 (m, 2H), 9.41 (s, 1H).

The following intermediates were prepared according to the same procedure from the Indicated starting material (SM=starting material):

| | | | |
|---|---|---|---|
| 3-1-2 SM = 3-2-2 | 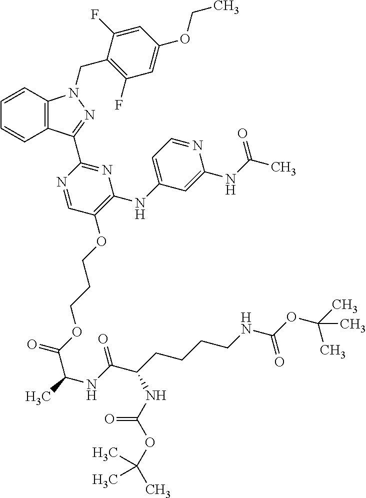 [SMILES: CCOc1cc(F)c(Cn2nc(-c3ncc(OCCCOC(=O)[C@H](C)NC(=O)[C@H](CCCCNC(=O)OC(C)(C)C)NC(=O)OC(C)(C)C)c(Nc4ccnc(NC(C)=O)c4)n3)c3ccccc32)c(F)c1] | 3-({4-[(2- acetamido- pyridin-4- yl)amino]-2- [1-(4-ethoxy- 2,6- difluorobenzyl)- 1H-indazol- 3- yl]pyrimidin- 5- yl}oxy)propyl N²,N⁶- bis(tert- butoxycarbonyl)- L-lysyl-L- alaninate | ¹H-NMR (300 MHz, DMSO-d₆): δ [ppm] = 1.18-1.28 (m, 8H), 1.33 (s, 18H), 2.08 (s, 3H), 2.10-2.23 (m, 2H), 2.76-2.94 (m, 2H), 3.82-3.96 (m, 1H), 4.03 (q, 2H), 4.19- 4.42 (m, 9H), 5.66 (s, 2H), 6.64-6.86 (m, 4H), 7.21 (t, 1H), 7.46 (t, 1H), 7.81 (d, 1H), 8.12-8.49 (m, 6H), 9.26 (s, 1H), 10.33 (s, 1H). |
| 3-1-3 SM = 3-2-2 | 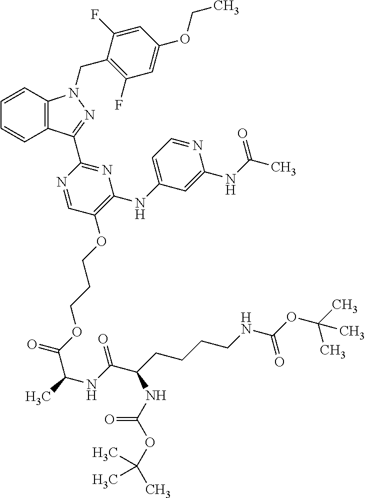 [SMILES: CCOc1cc(F)c(Cn2nc(-c3ncc(OCCCOC(=O)[C@H](C)NC(=O)[C@@H](CCCCNC(=O)OC(C)(C)C)NC(=O)OC(C)(C)C)c(Nc4ccnc(NC(C)=O)c4)n3)c3ccccc32)c(F)c1] | 3-({4-[(2- acetamido- pyridin-4- yl)amino]-2- [1-(4-ethoxy- 2,6- difluorobenzyl)- 1H-indazol- 3- yl]pyrimidin- 5- yl}oxy)propyl N²,N⁶- bis(tert- butoxycarbonyl)- D-lysyl-L- alaninate | ¹H-NMR (400 MHz, DMSO-d₆): δ [ppm] = 1.16-1.61 (m, 30H), 2.09 (s, 3H), 2.13- 2.23 (m, 2H), 2.80- 2.92 (m, 2H), 3.87- 3.98 (m, 1H), 4.04 (q, 2H), 4.23-4.40 (m, 5H), 5.68 (s, 2H), 6.67- 6.83 (m, 4H), 7.22 (t, 1H), 7.43-7.52 (m, 1H), 7.82 (d, 1H), 8.13- 8.24 (m, 2H), 8.27 (d, 1H), 8.31-8.39 (m, 2H), 8.45 (d, 1H), 9.27 (s, 1H), 10.34 (s, 1H). |
| 3-1-4 SM = 3-2-2 | 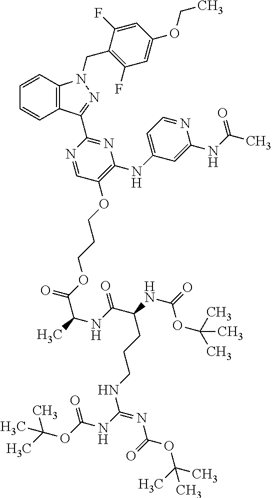 [SMILES: CCOc1cc(F)c(Cn2nc(-c3ncc(OCCCOC(=O)[C@H](C)NC(=O)[C@H](CCCN/C(=N/C(=O)OC(C)(C)C)NC(=O)OC(C)(C)C)NC(=O)OC(C)(C)C)c(Nc4ccnc(NC(C)=O)c4)n3)c3ccccc32)c(F)c1] | 3-({4-[(2- acetamido- pyridin-4- yl)amino]-2- [1-(4-ethoxy- 2,6- difluorobenzyl)- 1H-indazol- 3- yl]pyrimidin- 5- yl}oxy)propyl N⁵-[N,N′- bis(tert- butoxycarbonyl) carbamimdoyl]- N²-(tert- butoxycarbonyl)- L-ornithyl- L-alaninate | ¹H-NMR (400 MHz, DMSO-d₆): δ [ppm] = 1.25-1.31 (m, 6H), 1.33 (s, 9H), 1.36 (s, 9H), 1.42 (s, 9H), 1.62- 1.47 (m, 2H), 2.08 (s, 3H), 2.11-2.20 (m, 2H), 3.39-3.47 (m, 1H), 3.67-3.78 (m, 2H), 3.82-3.96 (m, 1H), 4.03 (q, 2H), 4.23- 4.35 (m, 6H), 5.66 (s, 2H), 6.67-6.82 (m, 2H), 7.15-7.25 (m, 1H), 7.42-7.53 (m, 1H), 7.80 (d, 1H), 8.12- 8.18 (m, 1H), 8.21- 8.28 (m, 2H), 8.29- 8.37 (m, 2H), 8.39- 8.46 (m, 1H), 9.06 (br. s, 2H), 9.24 (s, 1H), 10.30 (s, 1H). -one NH missing. |

### Intermediate 3-2-1

### Preparation of 3-[(4-{[2-(difluoromethyl)pyridin-4-yl]amino}-2-[1-(4-ethoxy-2,6-difluorobenzyl)-1H-indazol-3-yl]pyrimidin-5-yl)oxy]propyl L-alaninate, salt with trifluoroaceticacid

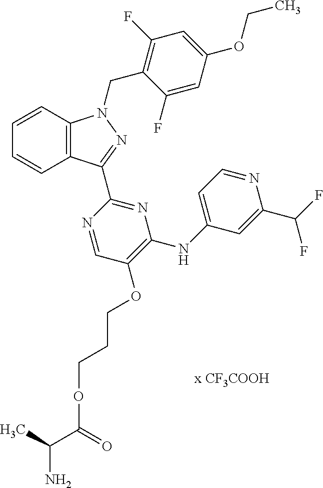 [SMILES: CCOc1cc(F)c(Cn2nc(-c3ncc(OCCCOC(=O)[C@H](C)N)c(Nc4ccnc(C(F)F)c4)n3)c3ccccc32)c(F)c1]

953 mg of 3-[(4-{[2-(difluoromethyl)pyridin-4-yl]amino}-2-[1-(4-ethoxy-2,6-difluorobenzyl)-1H-Indazol-3-yl]pyrimidin-5-yl)oxy]propyl N-(tert-butoxycarbonyl)-L-alaninate 3-3-1 (1.26 mmol, 1.0 eq.) were dissolved in 10 mL dichloromethane. 2.44 mL trifluoracetic acid (31.6 mmol, 25 eq.) was added and the mixture was stirred at room temperature for 1 hour. The reaction mixture was evaporated under vacuo. The crude product was lyophilized from acetonitrile and water to provide analytically pure target compound: 1.43 g, 1.86 mmol, 147%.

¹H-NMR (300 MHz, DMSO-d): δ [ppm]=1.28 (t, 3H), 1.39 (d, 3H), 2.15-2.30 (m, 2H), 4.03 (q, 2H), 4.08-4.21 (m, 1H), 4.29-4.40 (m, 2H), 4.40-4.54 (m, 2H), 5.70 (s, 2H), 6.70-6.84 (m, 2H), 6.92 (t, 1H), 7.20-7.31 (m, 1H), 7.44-7.56 (m, 1H). 7.83 (d, 1H), 8.29 (br. s., 4H), 8.37-8.45 (m, 2H), 8.50-8.61 (m, 2H), 9.59 (s, 1H).

The following intermediates were prepared according to the same procedure from the indicated starting material (SM=starting material):

| | | | |
|---|---|---|---|
| 3-2-2 SM = 3-3-2 | 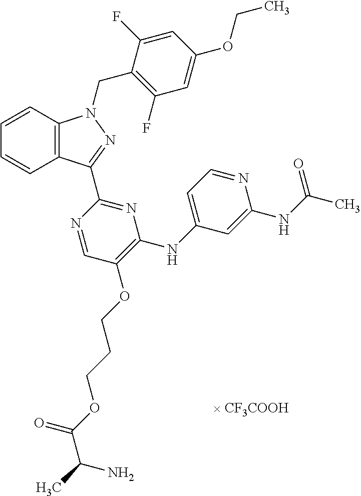 | 3-[(4-{[2- (acetylamino) pyridin-4- yl]amino}-2-[1- (4-ethoxy-2,6- difluorobenzyl)- 1H-indazol-3- yl]pyrimidin-5- yl)oxy]propyl L-alaninate, salt with trifluoroaceticacid | ¹H-NMR (400 MHz, DMSO-d₆): δ [ppm] = 1.28 (t, 3H), 1.37- 1.42 (m, 3H), 2.17 (s, 3H), 3.89-4.07 (m, 4H), 4.08-4.19 (m, 1H), 4.32-4.40 (m, 2H), 4.40-4.51 (m, 2H), 5.70 (s, 2H), 6.71- 6.84 (m, 2H), 7.20- 7.29 (m, 1H), 7.45- 7.54 (m, 1H), 7.81- 7.88 (m, 1H), 7.92- 7.97 (m, 1H), 7.99- 8.05 (m, 1H), 8.21- 8.25 (m, 1H), 8.35- 8.41 (m, 1H), 8.47 (s, 2H), 10.03 (br. s, 1H), 11.27 (br. s, 1H). —NH2 -signal is missing. |
| 3-2-3 SM = 3-3-3 | 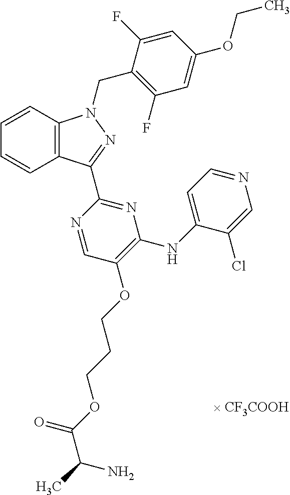 | 3-({4-[(3- chloropyridin- 4-yl)amino]-2- [1-(4-ethoxy- 2,6- difluorobenzyl)- 1H-indazol-3- yl]pyrimidin-5- yl}oxy)propyl L-alaninate, salt with trifluoroaceticacid | LC-MS: retention time: 1.42 min MS ES+: 639.3 [M + H]⁺ Method 5 |
| 3-2-4 SM = 3-3-4 | 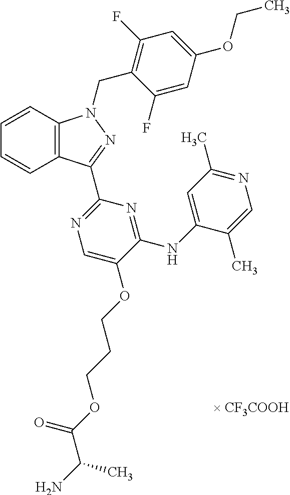 | 3-({4-[(2,5- dimethylpyridin- 4-yl)amino]-2- [1-(4-ethoxy- 2,6- difluorobenzyl)- 1H-indazol-3- yl]pyrimidin-5- yl}oxy)propyl L-alaninate, salt with trifluoroaceticacid | LC-MS: retention time: 1.34 min MS ES+: 632.5 [M + H]⁺ Method 5 |
| 3-2-5 SM = 3-3-5 Workup with sodium hydrogen carbonate solution | 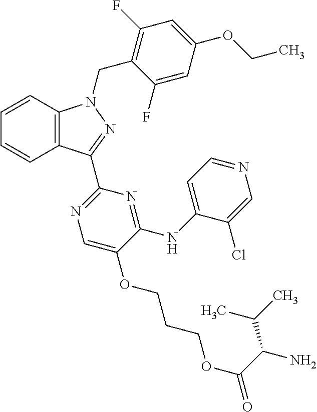 [SMILES: CCOc1cc(F)c(Cn2nc(-c3ncc(OCCCOC(=O)[C@@H](N)C(C)C)c(Nc4ccncc4Cl)n3)c3ccccc32)c(F)c1] | 3-({4-[(3- chloropyridin- 4-yl)amino]-2- [1-(4-ethoxy- 2,6- difluorobenzyl)- 1H-indazol-3- yl]pyrimidin-5- yl}oxy)propyl L-valinate | ¹H-NMR (400 MHz, DMSO-d₆): δ [ppm] = 0.89 (d, 6H), 1.29 (t, 3H), 2.05 (m, 1H), 2.23 (tt, 2H), 3.70 (d, 1H), 4.04 (q, 2H), 4.42 (m, 4H), 5.69 (s, 2H), 6.82 (m, 2H), 7.27 (dd, 1H), 7.50 (dd, 1H), 7.87 (d, 1H), 8.30 (br. s., 1H), 8.40 (d, 1H), 8.47 (d, 1H), 8.48 (s, 1H), 8.66 (s, 1H), 8.97 (d, 1H). |

### Intermediate 3-3-1

### Preparation of 3-[(4-{[2-(difluoromethyl)pyridin-4-yl]amino}-2-[1-(4-ethoxy-2,6-difluorobenzyl)-1H-indazol-3-yl]pyrimidin-5-yl)oxy]propyl N-(tert-butoxycarbonyl)-L-alaninate

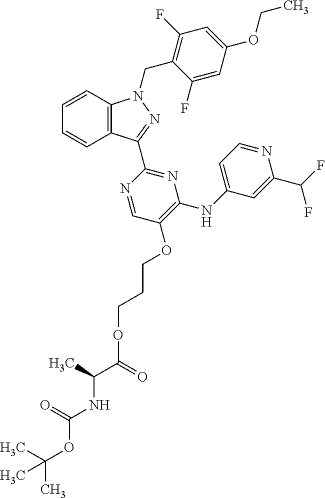 [SMILES: CCOc1cc(F)c(Cn2nc(-c3ncc(OCCCOC(=O)[C@H](C)NC(=O)OC(C)(C)C)c(Nc4ccnc(C(F)F)c4)n3)c3ccccc32)c(F)c1]

3.53 g of 3-[(4-{[2-(difluoromethyl)pyridin-4-yl]amino}-2-[1-(4-ethoxy-2,6-difluorobenzyl)-1H-indazol-3-yl]pyrimidin-5-yl)oxy]propan-1-ol 2-1-1 (6.1 mmol, 1.0 eq.), 3.5 g of N-(tert-butoxycarbonyl)-L-alanine (18 mmol, 3.0 eq.), 0.75 g if 4-Dimethylaminopyridine (6.1 mmol, 1.0 eq.) and 6.3 mL N,N-Diisopropylethylamine (36.4 mmol, 6.0 eq.) were dissolved in 98 mL DMF and 98 mL dichloromethane and 1.5 g of 1-(3-Dimethylaminopropyl)-3-ethylcarbodiimidhydrochloride (7.9 mmol, 1.3 eq.) were added. The reaction mixture was sonicated for 15 minutes under argon atmosphere. Then again 3.5 g of N-(tert-butoxycarbonyl)-L-alanine (18 mmol, 3.0 eq.), 0.75 g of 4-Dimethylaminopyridine (6.1 mmol, 1.0 eq.) and 1.5 g of 1-(3-Dimethylaminopropyl)-3-ethylcarbodiimidhydrochloride (7.9 mmol, 1.3 eq.) were added and the reaction mixture was sonicated for 15 minutes. The reaction mixture was evaporated, the residue was dissolved in methylene chloride and extracted with citric acid 5%. The organic layer was extracted with saturated sodium hydrogen carbonate solution, washed with water and brine, filtered through a silicone coated filter and reduced under reduced pressure. The crude product was dissolved in 10 mL dichloromethane and treated with hexane. A sticky precipiate was formed. The hexane was pipetted off and the precipitate was dried at the rotary evaporator to provide the target compound which was used without further purification: 5.2 g, 6.9 mmol, 113%.

¹H-NMR (400 MHz. DMSO-d): δ [ppm]=1.25 (d, 3H), 1.29 (t. 3H), 1.35 (s, 9H), 2.19 (t. 2H), 3.97-4.10 (m, 3H), 4.25-4.43 (m, 4H), 5.70 (s, 2H), 6.71-7.07 (m, 3H), 7.20-7.36 (m, 2H), 7.49 (td, 1H), 7.83 (d, 1H), 8.29 (d, 1H), 8.40 (s, 1H), 8.45 (d, 1H), 8.50-8.64 (m, 2H), 9.40 (s, 1H).

The following intermediates were prepared according to the same procedure from the Indicated starting material (SM=starting material):

| | | | |
|---|---|---|---|
| 3-3-2 SM = 2-1-5 | 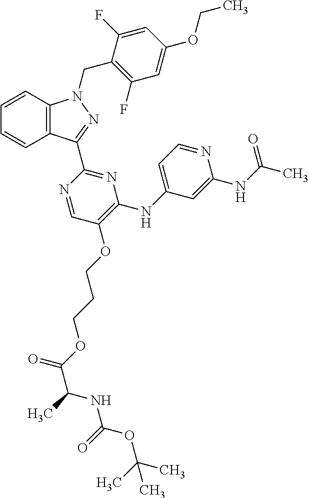 [SMILES: CCOc1cc(F)c(Cn2nc(-c3ncc(OCCCOC(=O)[C@H](C)NC(=O)OC(C)(C)C)c(Nc4ccnc(NC(C)=O)c4)n3)c3ccccc32)c(F)c1] | 3-[(4-{[2- (acetylamino) pyridin-4- yl]amino}-2-[1- (4-ethoxy-2,6- difluorobenzyl)- 1H-indazol-3- yl]pyrimidin-5- yl)oxy]propyl N-(tert- butoxycarbonyl)- L-alaninate | ¹H-NMR (300 MHz, DMSO-d₆): δ [ppm] = 1.32-1.38 (m, 12H), 1.45 (d, 3H), 2.08 (s, 3H), 3.82-3.96 (m, 2H), 3.98-4.09 (m, 3H), 4.22-4.40 (m, 4H), 5.66 (s, 2H), 6.71-6.82 (m, 2H), 7.08 (d, 1H), 7.15-7.34 (m, 1H), 7.43-7.51 (m, 1H), 7.81 (d, 1H), 8.15 (d, 1H), 8.26 (s, 1H), 8.28- 8.37 (m, 2H), 8.42 (d, 1H), 9.25 (s, 1H), 10.33 (s, 1H). |
| 3-3-3 SM = 2-1-6 | 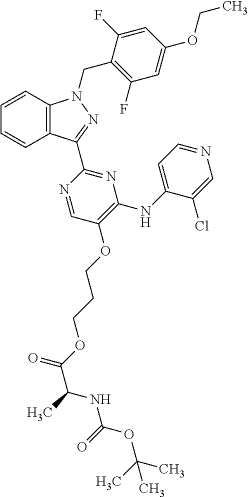 [SMILES: CCOc1cc(F)c(Cn2nc(-c3ncc(OCCCOC(=O)[C@H](C)NC(=O)OC(C)(C)C)c(Nc4ccncc4Cl)n3)c3ccccc32)c(F)c1] | 3-({4-[(3- chloropyridin- 4-yl)amino]-2- [1-(4-ethoxy- 2,6- difluorobenzyl)- 1H-indazol-3- yl]pyrimidin-5- yl}oxy)propyl N-(tert- butoxycarbonyl)- L-alaninate | ¹H NMR (400 MHz, DMSO-d₆) δ [ppm] = 1.14-1.36 (m, 12 H) 1.38 (s, 2 H) 2.17 (t, 2 H) 3.95-4.11 (m, 4 H) 4.23-4.46 (m, 4 H) 5.68 (s, 2 H) 6.79 (s, 1 H) 6.82 (d, 1 H) 7.20- 7.37 (m, 2 H) 7.43- 7.60 (m, 1 H) 7.85 (d, 1 H) 8.27 (s, 1 H) 8.35- 8.53 (m, 3 H) 8.64 (s, 1 H) 8.99 (d, 1 H). |
| 3-3-4 SM = 2-1-11 | 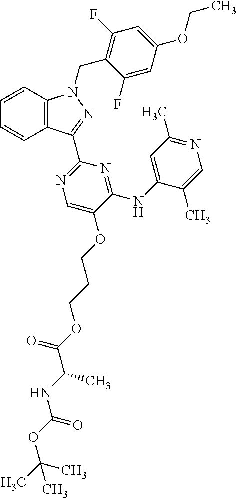 [SMILES: CCOc1cc(F)c(Cn2nc(-c3ncc(OCCCOC(=O)[C@H](C)NC(=O)OC(C)(C)C)c(Nc4cc(C)ncc4C)n3)c3ccccc32)c(F)c1] | 3-({4-[(2,5- dimethylpyridin- 4-yl)amino]-2- [1-(4-ethoxy- 2,6- difluorobenzyl)- 1H-indazol-3- yl]pyrimidin-5- yl}oxy)propyl N-(tert- butoxycarbonyl)- L-alaninate | LC-MS: retention time: 1.24 min MS ES+: 732.6 [M + H]⁺ Method 1 |
| 3-3-5 SM = 2-1-6 | 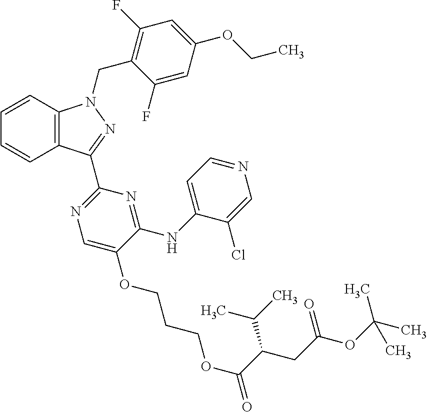 [SMILES: CCOc1cc(F)c(Cn2nc(-c3ncc(OCCCOC(=O)[C@@H](CC(=O)OC(C)(C)C)C(C)C)c(Nc4ccncc4Cl)n3)c3ccccc32)c(F)c1] | 3-({4-[(3- chloropyridin- 4-yl)amino]-2- [1-(4-ethoxy- 2,6- difluorobenzyl)- 1H-indazol-3- yl]pyrimidin-5- yl}oxy)propyl N-(tert- butoxycarbonyl)- L-valinate | LC-MS: retention time: 1.67 min MS ES+: 766.5 [M + H]⁺ Method 1 |

### Intermediate 3-4-1

### Preparation of 3-({4-[(3-chloropyridin-4-yl)amino]-2-[1-(4-ethoxy-2,6-difluorobenzyl)-1H-indazol-3-yl]pyrimidin-5-yl}oxy)propy N²,N⁶-bis(tert-butoxycarbonyl)-L-lysyl-L-alaninate

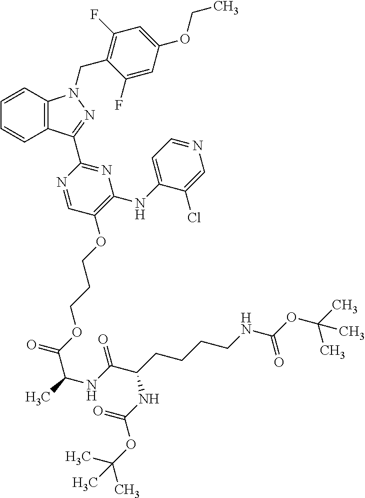 [SMILES: CCOc1cc(F)c(Cn2nc(-c3ncc(OCCCOC(=O)[C@H](C)NC(=O)[C@H](CCCCNC(=O)OC(C)(C)C)NC(=O)OC(C)(C)C)c(Nc4ccncc4Cl)n3)c3ccccc32)c(F)c1]

1.00 g of 3-({4-[(3-chloropyridin-4-yl)amino]-2-[1-(4-ethoxy-2,6-difluorobenzyl)-1H-indazol-3-yl]pyrimidin-5-yl}oxy)propyl-alaninate trifluoroacetate (1:1) 3-2-3 (1.33 mmol, 1.0 eq.), 921 mg of N²,N⁶-bis(tert-butoxycarbonyl)-L-lysine (2.66 mmol, 2.0 eq.) and 1.16 mL N,N-Diisopropylethylamine (6.64 mmol, 5.0 eq.) were dissolved in 80 mL DMF and 1.21 g of N-[(dimethylamino)(3H-[1,2,3]triazolo[4,5-b]pyridin-3-yloxy)methylidene]-N-methylmethanaminium hexafluorophosphate (7.9 mmol. 2.4 eq.) were added and stirred over night at room temperature. The DMF was evaporated under vacuo. The residue was dissolved in ethyl acetate and the organic phase was washed with 5%-aqueous citric acid twice. The organic layer was dried using a water resistant filter and the filtrate was concentrated in vacuo. The crude product was purified by flash chromatography and again dissolved in ethyl acetate and washed with saturated sodiumhydrogen carbonate-solution twice. The organic layer was dried using a water resistant filer and evaporated under vacuum to provide the 85% pure target compound: 1.10 g, 1.14 mmol, 85%.

LC-MS: retention time: 1.61 min, MS ES+: 667.8 [M+H]⁺, Method 5

The following intermediates were prepared according to the same procedure from the indicated starting material (SM=starting material):

| | | | |
|---|---|---|---|
| 3-4-2 SM = 3-2-4 | 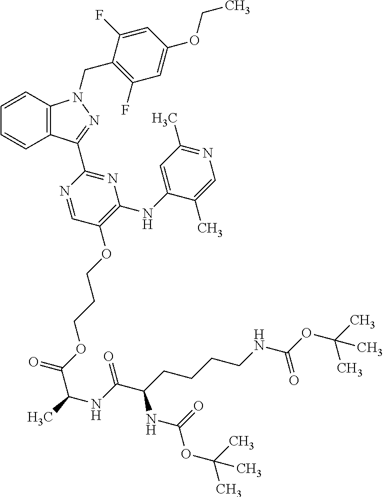 [SMILES: CCOc1cc(F)c(Cn2nc(-c3ncc(OCCCOC(=O)[C@H](C)NC(=O)[C@@H](CCCCNC(=O)OC(C)(C)C)NC(=O)OC(C)(C)C)c(Nc4cc(C)ncc4C)n3)c3ccccc32)c(F)c1] | 3-({4-[(2,5- dimethylpyridin- 4-yl)amino]-2- [1-(4-ethoxy- 2,6- difluorobenzyl)- 1H-indazol-3- yl]pyrimidin-5- yl}oxy)propyl N2,N6-bis(tert- butoxycarbonyl)- D-lysyl-L- alaninate | LC-MS: retention time: 1.54 min MS ES+: 960.9 [M + H]⁺ Method 5 |
| 3-4-3 SM = 3-2-4 | 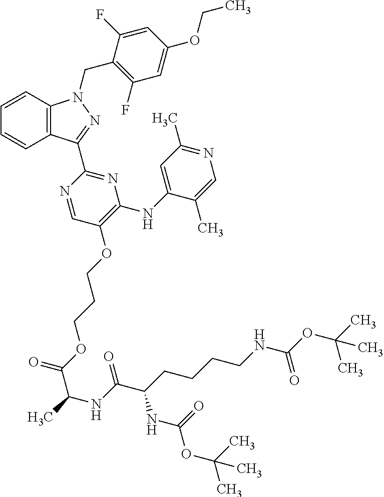 [SMILES: CCOc1cc(F)c(Cn2nc(-c3ncc(OCCCOC(=O)[C@H](C)NC(=O)[C@H](CCCCNC(=O)OC(C)(C)C)NC(=O)OC(C)(C)C)c(Nc4cc(C)ncc4C)n3)c3ccccc32)c(F)c1] | 3-({4-[(2,5- dimethylpyridin- 4-yl)amino]-2- [1-(4-ethoxy- 2,6- difluorobenzyl)- 1H-indazol-3- yl]pyrimidin-5- yl}oxy)propyl N2,N6-bis(tert- butoxycarbonyl)- L-lysyl-L- alaninate | LC-MS: retention time: 1.26 min MS ES+: 960.9 [M + H]⁺ Method 1 |
| 3-4-4 SM = 3-2-3 | 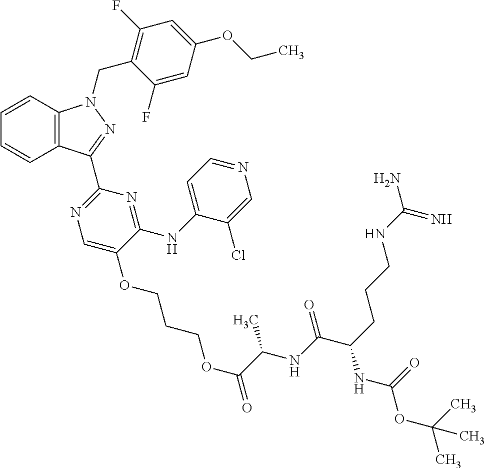 [SMILES: CCOc1cc(F)c(Cn2nc(-c3ncc(OCCCOC(=O)[C@H](C)NC(=O)[C@H](CCCNC(=N)N)NC(=O)OC(C)(C)C)c(Nc4ccncc4Cl)n3)c3ccccc32)c(F)c1] | 3-({4-[(3- chloropyridin- 4-yl)amino]-2- [1-(4-ethoxy- 2,6- difiuorobenzyl)- 1H-indazol-3- yl]pyrimidin-5- yl}oxy)propyl N2-(tert- butoxycarbonyl)- L-arginyl-L- alaninate | LC-MS: retention time: 1.26 min MS ES+: 894.4 [M + H]⁺ Method 1 |
| 3-4-5 SM = 3-2-5 | 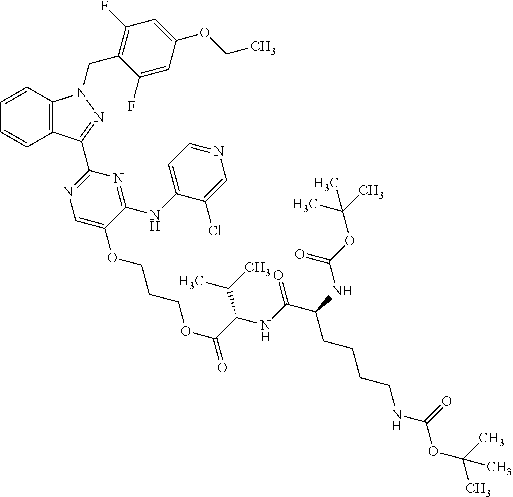 [SMILES: CCOc1cc(F)c(Cn2nc(-c3ncc(OCCCOC(=O)[C@@H](NC(=O)[C@H](CCCCNC(=O)OC(C)(C)C)NC(=O)OC(C)(C)C)C(C)C)c(Nc4ccncc4Cl)n3)c3ccccc32)c(F)c1] | 3-({4-[(3- chloropyridin- 4-yl)amino]-2- [1-(4-ethoxy- 2,6- difluorobenzyl)- 1H-indazol-3- yl]pyrimidin-5- yl}oxy)propyl N2,N6-bis(tert- butoxycarbonyl)- L-lysyl-L- valinate | LC-MS: retention time: 1.65 min MS ES+: 994.8 [M + H]⁺ Method 1 |
| 3-4-6 SM = 3-2-5 | 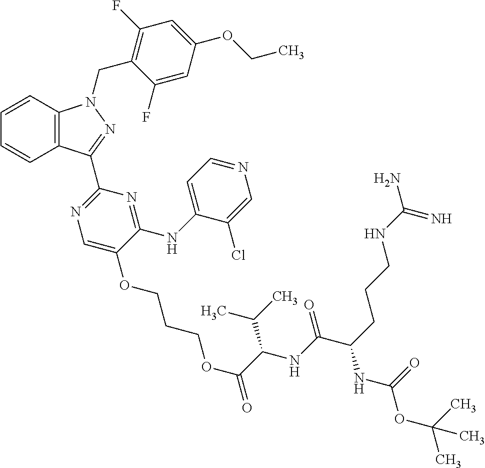 [SMILES: CCOc1cc(F)c(Cn2nc(-c3ncc(OCCCOC(=O)[C@@H](NC(=O)[C@H](CCCNC(=N)N)NC(=O)OC(C)(C)C)C(C)C)c(Nc4ccncc4Cl)n3)c3ccccc32)c(F)c1] | 3-({4-[(3- chloropyridin- 4-yl)amino]-2- [1-(4-ethoxy- 2,6- difluorobenzyl)- 1H-indazol-3- yl]pyrimidin-5- yl}oxy)propyl N2-(tert- butoxycarbonyl)- L-arginyl-L- valinate | LC-MS: retention time: 1.34 min MS ES+: 922.5 [M + H]⁺ Method 1 |

### Example Compounds of Prodrugs

### Example 4-1-1

### Preparation of 3-[(4-{[2-(difluoromethyl)pyridin-4-yl]amino}-2-[1-(4-ethoxy-2,6-difluorobenzyl)-1H-Indazol-3-yl]pyrimidin-5-yl)oxy]propyl L-lysyl-L-alaninate, salt with trifluoroaceticacid

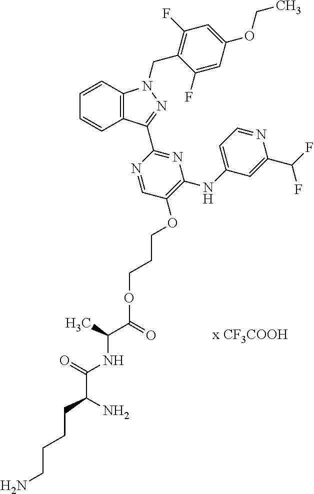 [SMILES: CCOc1cc(F)c(Cn2nc(-c3ncc(OCCCOC(=O)[C@H](C)NC(=O)[C@@H](N)CCCCN)c(Nc4ccnc(C(F)F)c4)n3)c3ccccc32)c(F)c1]

810 mg of 3-[(4-{[2-(difluoromethyl)pyridin-4-yl]amino}-2-[1-(4-ethoxy-2,6-difluorobenzyl)-1H-indazol-3-yl]pyrimidin-5-yl)oxy]propyl N², N⁶-bis(tert-butoxy-carbonyl)-L-lysyl-L-alaninate 3-1-1 (0.83 mmol, 1.0 eq.) were dissolved in 24 mL dichloromethane and treated with 6.4 mL trifluoracetic acid (82 mmol, 100 eq.). The reaction mixture was stirred at room temperature under argon atmosphere for 30 minutes. The reaction mixture was evaporated at room temperature. The crude product was treated with diethyl ether and stirred. The solvent was pipetted off, the residue was treated with acetonitrile/water 7:3, lyophilized to provide the crude product. The crude product was treated with diethyl ether and stirred. The solvent was pipetted off, the residue was treated with dichloromethane, dried under reduced pressure at room temperature and by oilpump for one hour to provide analytically pure target compound: 752 mg, 0.67 mmol, 81%.

¹H-NMR (400 MHz, DMSO-d₆): δ [ppm]=1.28 (t, 3H), 1.34 (d, 3H). 1.36-1.42 (m, 2H). 1.45-1.59 (m, 2H), 1.64-1.77 (m, 2H), 2.13-2.26 (m, 2H), 2.68-2.80 (m, 1H), 3.71-3.80 (m, 2H), 4.03 (q, 2H), 4.27-4.44 (m, 5H), 5.69 (s, 2H), 6.69-7.07 (m, 3H), 7.20-7.30 (m, 1H), 7.44-7.54 (m, 1H), 7.73 (br. s., 3H), 7.82 (d, 1H), 8.05-8.21 (m, 3H), 8.28 (s, 1H), 8.37-8.47 (m, 2H), 8.49-8.58 (m, 2H), 8.85 (d, 1H), 9.43 (s, 1H).

The following examples were prepared according to the same procedure from the Indicated starting materials (SM=starting material):

| | | | |
|---|---|---|---|
| 4-1-2 SM = 3-1-2 | 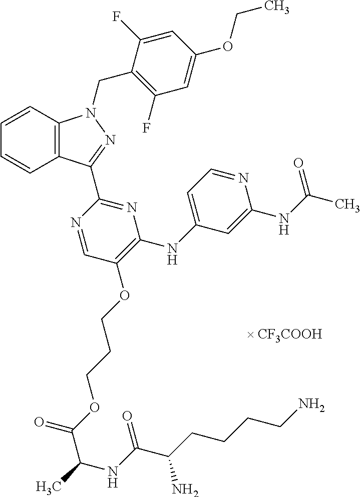 [SMILES: CCOc1cc(F)c(Cn2nc(-c3ncc(OCCCOC(=O)[C@H](C)NC(=O)[C@@H](N)CCCCN)c(Nc4ccnc(NC(C)=O)c4)n3)c3ccccc32)c(F)c1] | 3-[(4-{[2- (acetylamino) pyridin- 4-yl]amino}- 2-[1-(4-ethoxy- 2,6- difluorobenzyl)- 1H-indazol-3- yl]pyrimidin-5- yl)oxy]propyl L- lysyl-L- alaninate, salt with trifiuoroaceticacid | ¹H-NMR (400 MHz, DMSO-d₆): δ [ppm] = 1.23-1.48 (m, 5H), 1.48-1.63 (m, 2H), 1.64-1.80 (m, 2H), 2.08-2.26 (m, 5H), 2.70-2.83 (m, 2H), 3.73-3.86 (m, 2H), 3.96-4.10 (m, 2H), 4.25-4.46 (m, 5H), 5.70 (s, 2H), 6.68- 6.87 (m, 2H), 7.20- 7.30 (m, 1H), 7.45- 7.56 (m, 1H), 7.68- 7.91 (m, 4H), 8.08- 8.29 (m, 5H), 8.32- 8.49 (m, 3H), 8.89 (d, 1H), 9.72 (br. s, 1H), 10.90 (br. s, 1H). |
| 4-1-3 SM = 3-1-3 | 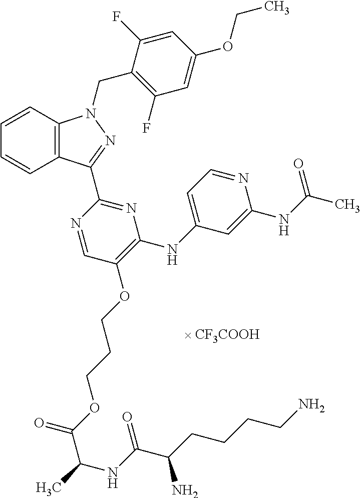 [SMILES: CCOc1cc(F)c(Cn2nc(-c3ncc(OCCCOC(=O)[C@H](C)NC(=O)[C@H](N)CCCCN)c(Nc4ccnc(NC(C)=O)c4)n3)c3ccccc32)c(F)c1] | 3-[(4-{[2- (acetylamino)pyridin- 4-yl]amino}- 2-[1-(4-ethoxy- 2,6- difluorobenzyl)- 1H-indazol-3- yl]pyrimidin-5- yl)oxy]propyl D- lysyl-L- alaninate, salt with trifiuoroaceticacid | ¹H-NMR (400 MHz, DMSO-d₆): δ [ppm] = 1.23-1.38 (m, 8H), 1.43-1.57 (m, 2H), 1.65-1.77 (m, 2H), 2.13 (s, 3H), 2.15- 2.24 (m, 2H), 2.68- 2.79 (m, 2H), 3.73- 3.83 (m, 1H), 4.03 (q, 2H), 4.27-4.42 (m, 5H), 5.69 (s, 2H), 6.73-6.82 (m, 2H), 7.24 (t, 1H), 7.44- 7.53 (m, 1H), 7.69 (br. s, 3H), 7.83 (d, 1H), 8.13 (br. s., 4H), 8.21 (d, 1H), 8.34- 8.44 (m, 3H), 8.89 (d, 1H), 9.70 (br. s, 1H), 10.89 (s, 1H). |
| 4-1-4 SM = 3-1-4 | 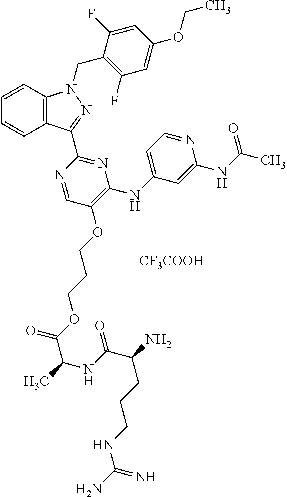 [SMILES: CCOc1cc(F)c(Cn2nc(-c3ncc(OCCCOC(=O)[C@H](C)NC(=O)[C@@H](N)CCCNC(=N)N)c(Nc4ccnc(NC(C)=O)c4)n3)c3ccccc32)c(F)c1] | 3-[(4-{[2- (acetylamino)pyridin- 4-yl]amino}- 2-[1-(4-ethoxy- 2,6- difluorobenzyl)- 1H-indazol-3- yl]pyrimidin-5- yl)oxy]propyl L- arginyl-L- alaninate, salt with trifiuoroaceticacid | ¹H-NMR (400 MHz, DMSO-d₆): δ [ppm] = 1.28 (t, 3H), 1.34 (d, 3H), 1.48-1.61 (m, 2H), 1.64-1.77 (m, 2H), 2.11-2.26 (m, 5H), 2.51-2.53 (m, 1H), 3.05-3.17 (m, 2H), 3.70-3.83 (m, 1H), 3.98-4.09 (m, 2H), 4.25-4.43 (m, 4H), 5.70 (s, 2H), 6.72-6.82 (m, 2H), 7.20-7.29 (m, 1H), 7.48 (t, 1H), 7.57- 7.67 (m, 1H), 7.85 (d, 1H), 8.02-8.10 (m, 1H), 8.10-8.25 (m, 4H), 8.36-8.46 (m, 3H), 8.87 (d, 1H), 9.88 (br. s, 1H), 11.11 (br. s, 1H). |
| 4-1-5 SM = 3-4-1 | 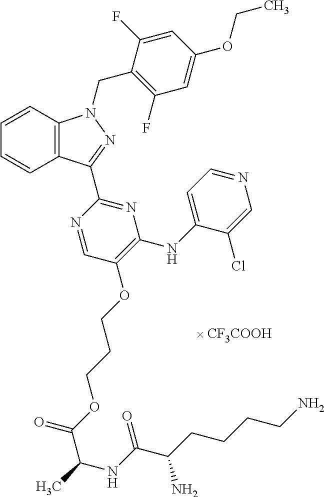 [SMILES: CCOc1cc(F)c(Cn2nc(-c3ncc(OCCCOC(=O)[C@H](C)NC(=O)[C@@H](N)CCCCN)c(Nc4ccncc4Cl)n3)c3ccccc32)c(F)c1] | 3-({4-[(3- chloropyridin-4- yl)amino]-2-[1- (4-ethoxy-2,6- difluorobenzyl)- 1H-indazol-3- yl]pyrimidin-5- yl}oxy)propyl L- lysyl-L- alaninate, salt with trifluoroaceticacid | ¹H NMR (500 MHz, DMSO-d₆) δ ppm 1.46-1.64 (m, 3 H) 1.64-1.77 (m, 3 H) 2.18 (t, 2 H) 2.68 (s, 2 H) 2.70-2.79 (m, 6 H) 3.76 (d, 1 H) 4.03 (q, 2 H) 4.25-4.48 (m, 5 H) 5.69 (s, 2 H) 6.79 (s, 1 H) 6.81 (d, 1 H) 7.26 (t, 1 H) 7.43-7.55 (m, 1 H) 7.74 (br. s., 3 H) 7.85 (d, 1 H) 7.94 (s, 1 H) 8.09-8.20 (m, 3 H) 8.27-8.43 (m, 2 H) 8.69 (s, 1 H) 8.87 (d, 1 H) 8.95 (d, 1 H). |
| 4-1-6 SM = 3-4-2 | 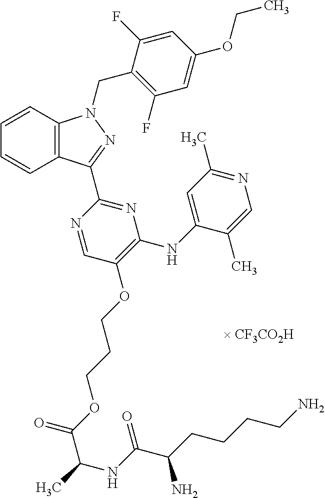 [SMILES: CCOc1cc(F)c(Cn2nc(-c3ncc(OCCCOC(=O)[C@H](C)NC(=O)[C@H](N)CCCCN)c(Nc4cc(C)ncc4C)n3)c3ccccc32)c(F)c1] | 3-({4-[(2,5- dimethylpyridin- 4-yl)amino]-2-[1- (4-ethoxy-2,6- difluorobenzyl)- 1H-indazol-3- yl]pyrimidin-5- yl}oxy)propyl D- lysyl-L- alaninate, salt with trifluoroaceticacid | ¹H-NMR (400 MHz, DMSO-d₆): δ [ppm] = 1.26-1.39 (m, 8H), 1.47-1.58 (m, 2H), 1.64-1.79 (m, 2H), 2.15-2.27 (m, 2H), 2.46 (s, 3H), 2.69 (s, 3H), 2.71-2.81 (m, 2H), 3.77-3.87 (m, 1H), 4.04 (q, 2H), 4.26-4.47 (m, 5H), 5.74 (s, 2H), 6.74- 6.84 (m, 2H), 7.32 (t, 1H), 7.50-7.58 (m, 1H), 7.75-7.92 (m, 3H), 8.23 (br. s., 4H), 8.44-8.50 (m, 1H), 8.55 (s, 1H), 8.59 (s, 1H), 8.66 (s, 1H), 8.97 (d, 1H), 9.05 (s, 1H). |
| 4-1-7 SM = 3-4-3 | 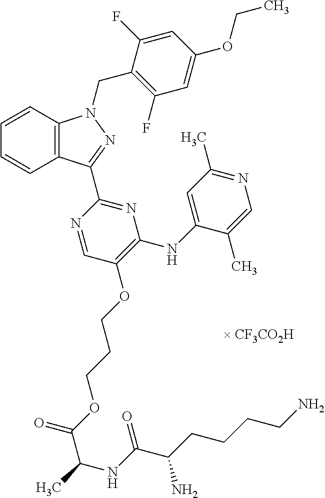 [SMILES: CCOc1cc(F)c(Cn2nc(-c3ncc(OCCCOC(=O)[C@H](C)NC(=O)[C@@H](N)CCCCN)c(Nc4cc(C)ncc4C)n3)c3ccccc32)c(F)c1] | 3-({4-[(2,5- dimethylpyridin- 4-yl)amino]-2-[1- (4-ethoxy-2,6- difluorobenzyl)- 1H-indazol-3- yl]pyrimidin-5- yl}oxy)propyl L- lysyl-L- alaninate, salt with trifluoroaceticacid | ¹H-NMR (400 MHz, DMSO-d₆): δ [ppm] = 1.26-1.45 (m, 8H), 1.49-1.60 (m, 2H), 1.68-1.77 (m, 2H), 2.15-2.24 (m, 2H), 2.46 (s, 3H), 2.67 (s, 3H), 2.71-2.81 (m, 2H), 3.75-3.83 (m, 1H), 4.04 (q, 2H), 4.28-4.46 (m, 5H), 5.74 (s, 2H), 6.75- 6.84 (m, 2H), 7.28- 7.36 (m, 1H), 7.50- 7.57 (m, 1H), 7.76- 7.86 (m, 3H), 7.90 (d, 1H), 8.10-8.25 (m, 3H), 8.47 (d, 1H), 8.54 (s, 1H), 8.58 (s, 1H), 8.66 (s, 1H), 8.92 (d, 1H), 9.06 (s, 1H). |
| 4-1-8 SM = 3-4-4 | 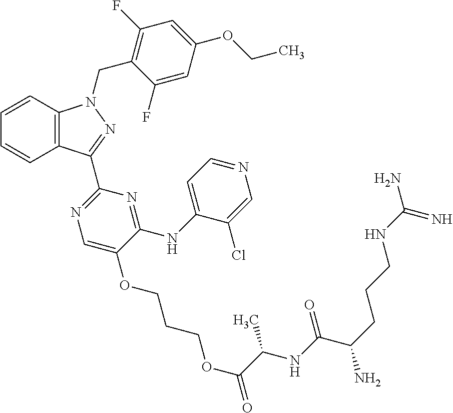 [SMILES: CCOc1cc(F)c(Cn2nc(-c3ncc(OCCCOC(=O)[C@H](C)NC(=O)[C@@H](N)CCCNC(=N)N)c(Nc4ccncc4Cl)n3)c3ccccc32)c(F)c1] | 3-({4-[(3- chloropyridin-4- yl)amino]-2-[1- (4-ethoxy-2,6- difluorobenzyl)- 1H-indazol-3- yl]pyrimidin-5- yl}oxy)propyl L- arginyl-L- alaninate | ¹H-NMR (400 MHz, DMSO-d₆): δ [ppm] = 1.29 (t, 3H), 1.32 (d, 3H), 1.54 (m, 3H), 1.65 (m, 1H), 2.18 (m, 2H), 3.10 (m, 2H), 3.51 (m, 1H), 4.04 (q, 2H), 4.33 (m, 1H), 4.34 (m, 2H), 4.36 (m, 2H), 5.69 (s, 2H), 6.83 (m, 2H), 7.27 (dd, 1H), 7.51 (dd, 1H), 7.87 (d, 1H), 8.31 (br. s., 1H), 8.39 (d, 1H), 8.47 (d, 1H), 8.48 (s, 1H), 8.64 (br. s., 1H), 8.66 (s, 1H), 8.96 (d, 1H). |
| 4-1-9 SM = 3-4-5 Workup with sodium hydrogen- carbonate solution | 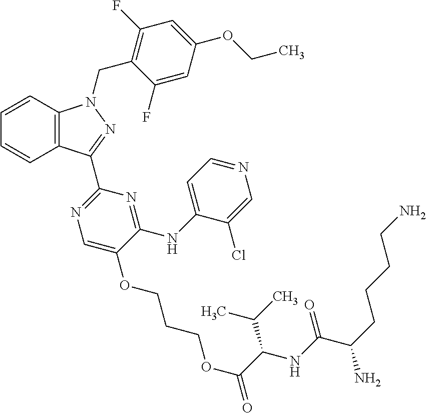 [SMILES: CCOc1cc(F)c(Cn2nc(-c3ncc(OCCCOC(=O)[C@@H](NC(=O)[C@@H](N)CCCCN)C(C)C)c(Nc4ccncc4Cl)n3)c3ccccc32)c(F)c1] | 3-({4-[(3- chloropyridin-4- yl)amino]-2-[1- (4-ethoxy-2,6- difluorobenzyl)- 1H-indazol-3- yl]pyrimidin-5- yl}oxy)propyl L- lysyl-L-valinate | ¹H-NMR (400 MHz, DMSO-d₆): δ [ppm] = 0.84 (d, 6H), 1.29 (t, 3H), 1.21-1.60 (m, 6H), 2.05 (m, 1H), 2.19 (m, 2H), 2.71 (m, 2H), 3.23 (m, 1H), 4.04 (q, 2H), 4.19 (m, 1H), 4.33 (m, 2H), 4.38 (m, 2H), 5.69 (s, 2H), 6.83 (m, 2H), 7.27 (dd, 1H), 7.50 (dd, 1H), 7.87 (d, 1H), 8.19 (m, 1H), 8.37 (br. s., 1H), 8.39 (d, 1H), 8.47 (d, 1H), 8.47 (s, 1H), 8.66 (s, 1H), 8.96 (d, 1H). |
| 4-1-10 SM = 3-4-6 | 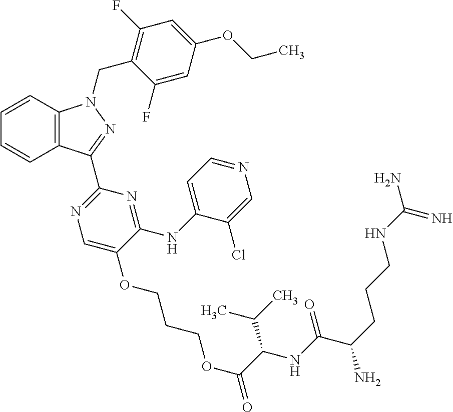 [SMILES: CCOc1cc(F)c(Cn2nc(-c3ncc(OCCCOC(=O)[C@@H](NC(=O)[C@@H](N)CCCNC(=N)N)C(C)C)c(Nc4ccncc4Cl)n3)c3ccccc32)c(F)c1] | 3-({4-[(3- chloropyridin-4- yl)amino]-2-[1- (4-ethoxy-2,6- difluorobenzyl)- 1H-indazol-3- yl]pyrimidin-5- yl}oxy)propyl L- arginyl-L-valinate | ¹H-NMR (400 MHz, DMSO-d₆): δ [ppm] = 0.84 (d, 6H), 1.29 (t, 3H), 1.34 (m, 1H), 1.50 (m, 2H), 1.59 (m, 1H), 2.05 (m, 1H), 2.19 (m, 2H), 3.05 (m, 2H), 3.23 (m, 1H), 4.04 (q, 2H), 4.20 (m, 1H), 4.34 (m, 2H), 4.38 (m, 2H), 5.69 (s, 2H), 6.82 (m, 2H), 7.27 (dd, 1H), 7.51 (dd, 1H), 7.87 (d, 1H), 8.22 (br. s., 1H), 8.31 (br. s., 1H), 8.40 (d, 1H), 8.47 (d, 1H), 8.47 (s, 1H), 8.66 (s, 1H), 8.96 (d, 1H). |

### Reference Compounds

### Example 5-1-1 Preparation of 2-[1-(4-ethoxy-2,6-difluorobenzyl)-1H-indazol-3-yl]-4-(pyridin-4-ylamino)pyrimidin-5-ol

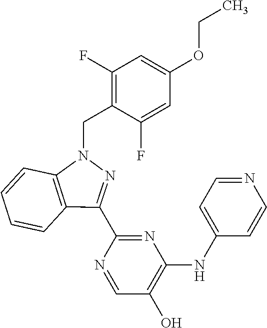 [SMILES: CCOc1cc(F)c(Cn2nc(-c3ncc(O)c(Nc4ccncc4)n3)c3ccccc32)c(F)c1]

Example 5-1-1 was prepared as described in WO 2013050438—Example 3-1

### Example 5-2-1 Preparation of tert-butyl [2-(2-bromoethoxy)ethyl]carbamate

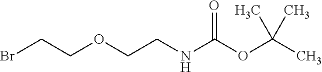

200 mg tert-butyl [2-(2-hydroxyethoxy)ethyl]carbamate (0.97 mmol, 1.0 eq.) and 388 mg tetrabromomethane (1.17 mmol, 1.2 eq.) were dissolved in 1.2 mL THF and cooled to 0° C. 307 mg triphenylphosphane (1.17 mmol, 1.2 eq.) were added. The reaction mixture was stirred at room temperature for 3 d. Hexane was added to form a white precipitate which was filtered off. The filtrate was evaporated under vacuo to provide the crude target compound, which was used without further purification: 381 mg, 1.42 mmol, 145%.

¹H-NMR (400 MHz, DMSO-d6): δ [ppm]=1.34 (a, 9H), 3.04 (q, 2H), 3.38 (t, 2H), 3.54 (t, 2H), 3.66 (t, 2H), 6.76 (t, 1H).

### Example 5-3-1 Preparation of tert-butyl (2-{2-[{5-(2-{2-[(tert-butoxycarbonyl)amino]ethoxy}ethoxy)-2-[1-(4-ethoxy-2,6-difluorobenzyl)-1H-indazol-3-yl]pyrimidin-4-yl}(pyridin-4-yl)amino]ethoxy}ethyl)carbamate

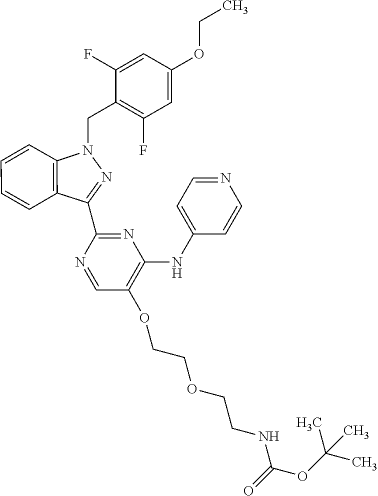 [SMILES: CCOc1cc(F)c(Cn2nc(-c3ncc(OCCOCCNC(=O)OC(C)(C)C)c(Nc4ccncc4)n3)c3ccccc32)c(F)c1]

35 mg 2-[1-(4-ethoxy-2,6-difluorobenzyl)-1H-indazol-3-yl]-4-(pyridin-4-ylamino)-pyrimidin-5-ol 5-1-1 (0.074 mmol, 1.0 eq.) was dissolved in 0.6 mL DMF. 31 mg potassium carbonate (0.22 mmol. 3.0 eq.) and 20 mg tert-butyl [2-(2-bromoethoxy)ethyl]carbamate 5-2-1 (0.074 mmol, 1.0 eq.) were added. The reaction mixture was stirred at room temperature over night. 20 mg tert-butyl [2-(2-bromoethoxy)ethyl]carbamate 5-2-1 (0.074 mmol, 1.0 eq.) was added and the reaction mixture was stirred for 2.5 h at room temperature. Butanone was added and the organic layer was washed with brine. The aqueous layer was extracted with butanone. The combined organic layers were dried with a waterresitent filter and concentrated in vacuo. The crude product was purified by flash chromatography to provide the 85% pure target compound: 20 mg, 0.03 mmol, 34%.

retention time: 1.20 min

MS ES+: 662.3 [M+H]

Method 1

### Example 5-4-1 Preparation of 5-[2-(2-aminoethoxy)ethoxy]-2-[1-(4-ethoxy-2,6-difluorobenzyl)-1H-indazol-3-yl]-N-(pyridin-4-yl)pyrimidin-4-amine, salt with hydrochloric acid

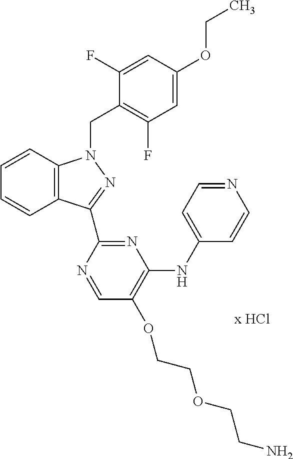 [SMILES: CCOc1cc(F)c(Cn2nc(-c3ncc(OCCOCCN)c(Nc4ccncc4)n3)c3ccccc32)c(F)c1]

20 mg tert-butyl {2-[2-({2-[1-(4-ethoxy-2,6-difluorobenzyl)-1H-indazol-3-yl]-4-(pyridin-4-ylamino)pyrimidin-5-yl}oxy)ethoxy]ethyl}carbamate 5-3-1 (0.03 mmol, 1.0 eq.) was dissolved in 0.1 mL dioxane. 0.03 mL hydrochlorid acid (4 M In dioxane. 0.12 mmol, 4.0 eq.) were added and the reaction mixture was stirred 2 h at room temperature. The solvents were evaporated and the 90% pure crude product was used without further purification: 20 mg, 0.03 mmol, 100%.

¹H-NMR (400 MHz, DMSO-d6): δ [ppm]=1.25 (t, 3H), 2.91-3.03 (m, 2H), 3.67-3.77 (m, 2H), 3.88-3.95 (m, 2H), 4.01 (q, 2H), 4.42-4.46 (m, 2H), 5.68 (s, 2H), 6.75-6.82 (m, 2H), 7.18-7.34 (m, 1H), 7.42-7.53 (m, 1H), 7.83 (d, 1H), 8.15 (br. s, 3H). 8.39 (d, 1H), 8.53-8.65 (m, 3H), 8.81 (d, 2H), 10.62 (s, 1H), 14.98 (br. s, 1H).

### Example 5-5-1 Preparation of 5-[((2-[2-((2-[1-(4-ethoxy-2,6-difluorobenzyl)-1H-indazol-3-yl]-4-(pyridin-4-ylamino)pyrimidin-5-yl)oxy)ethoxy]ethyl)carbamothioyl)amino]-2-(6-hydroxy-3-oxo-3H-xanthen-9-yl)benzoic acid

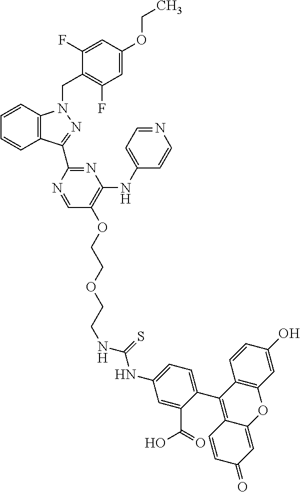 [SMILES: CCOc1cc(F)c(Cn2nc(-c3ncc(OCCOCCNC(=S)Nc4ccc(-c5c6ccc(=O)cc-6oc6cc(O)ccc56)c(C(=O)O)c4)c(Nc4ccncc4)n3)c3ccccc32)c(F)c1]

14 mg 5-[2-(2-aminoethoxy)ethoxy]-2-[1-(4-ethoxy-2,6-difluorobenzyl)-1H-indazol-3-yl]-N-(pyridin-4-yl)pyrimidin-4-amine dihydrochloride 5-4-1 (0.022 mmol, 1.0 eq.), 8.5 mg 3′,6′-dihydroxy-5-isothiocyanato-3H-spiro[2-benzofuran-1,9′-xanthen]-3-one (0.022 mmol, 1.0 eq.) and 11 μL diisopropylethylamine (0.065 mmol, 3.0 eq.) were dissolved in 91 μL dichloromethane. The reaction mixture was stirred at room temperature for 72 h. Ethyl acetate and aqueous acidic acid were added. It was stirred at room temperature over night. The aqueous layer was extracted with ethyl acetate twice. The combined organic layers were washed with brine, dried over a water resistant filter and concentrated in vacuo. The crude product was purified by flash chromatography to provide the 97% pure target compound: 7.5 mg, 0.01 mmol, 35%.

retention time: 1.13 min

MS ES+: 951.3 [M+H]⁺

Method 1

### Example 5-6-1 Preparation of 2-({2-[1-(4-ethoxy-2,6-difluorobenzyl)-1H-indazol-3-yl]-4-(pyridin-4-ylamino)pyrimidin-5-yl}oxy)ethanol

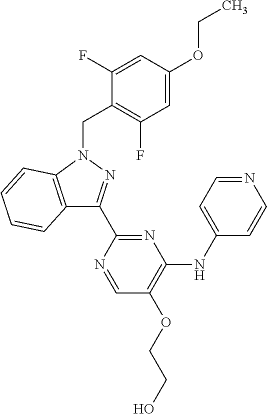 [SMILES: CCOc1cc(F)c(Cn2nc(-c3ncc(OCCO)c(Nc4ccncc4)n3)c3ccccc32)c(F)c1]

Example 5-6-1 was prepared as described in WO 2013050438—Example 13-1

### Biological Investigations

The following assays can be used to illustrate the commercial utility of the compounds according to the present Invention.

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values calculated utilizing data sets obtained from testing of one or more synthetic batch.

The half-life of prodrugs (Table 3) poses a challenge for the verification of their biological activity. Bub1 enzymatic activity assays (as per Biological Assay 1.0 infra) require a minimum reaction time of 60 minutes in order to be able to assess the inhibitory activity of the compounds. During this time typically 20% to 60% of the prodrugs have been already converted to their corresponding drugs at the pH of 7.5 at which the assay is performed. Ideally one would perform the biological assay at a pH at which the prodrug is stable (e.g. pH 4). This is not viable in the case of Bub1, because the kinase is inactive this pH. Alternatively, an assay requiring shorter incubation times would better estimate the binding affinities of the prodrugs. In the present invention we have developed a biological assay (5.0) which allows quantifying the affinity of the prodrugs starting from 15 minutes of compound-enzyme incubation in aqueos buffer. Extrapolating the stability data from Tables 3 and 4, one would expect at this time point most of the compound present in the test solution to be the prodrug.

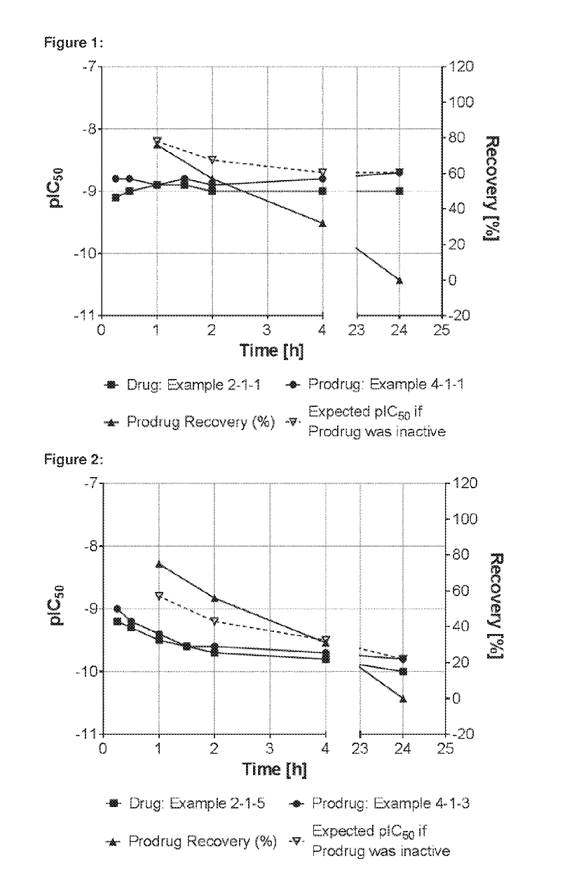

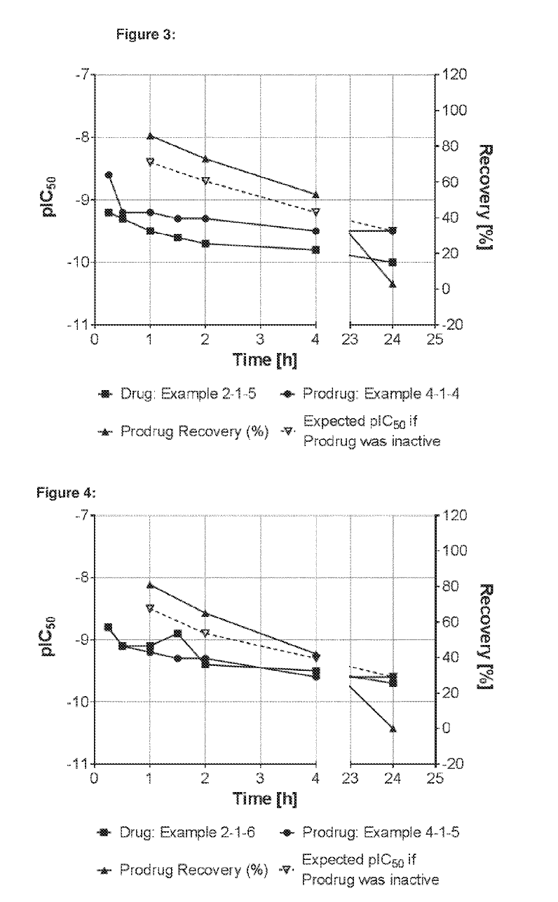

FIGS. 1, 2, 3 and 4 combine the results from Biological Assay 5 with those from Biological Assay 6. The binding affinities over time of drug and prodrug pairs are shown as pIC₅₀ together with the prodrug stability, and the pIC₅₀ to be expected if only the drug present in the sample was responsible for the activity in the assay. Extrapolating the pIC₅₀ from these graphs to time 0 (at which 100% of prodrug is present in the sample), the actual affinity of the drug and prodrug can be inferred. Secondly, looking at the differences between expected and real pIC₅₀ an estimate can be made about the contribution of the prodrug to the observed activity.

These data demonstrate that the prodrugs are Inhibitors of Bub1 kinase activity and therefore they contribute to the biological activities reported by the assays 1.0, 2.0, 3.0 and 4.0.

Biological Assay 1.0:

Bub1 Kinase Assay

Bub1-inhibitory activities of compounds described in the present invention were quantified using a time-resolved fluorescence energy transfer (TR-FRET) kinase assay which measures phosphorylation of the synthetic peptide Biotin-Ahx-VLLPKKSFAEPG (SEQ ID No. 1) (C-terminus in amide form), purchased from e.g. Biosyntan (Berlin, Germany) by the (recombinant) catalytic domain of human Bub1 (amino acids 704-1085), expressed in Hi5 insect cells with an N-terminal His6-tag and purified by affinity-(Ni-NTA) and size exclusion chromatography.

In a typical assay 11 different concentrations of each compound (0.1 nM, 0.33 nM, 1.1 nM, 3.8 nM, 13 nM, 44 nM, ˜0.15 μM, ˜0.51 μM, ˜1.7 μM, ˜5.9 μM and 20 μM) were tested in duplicate within the same microtiter plate. To this end, 100-fold concentrated compound solutions (in DMSO) were previously prepared by serial dilution (1:3.4) of 2 mM stocks in a clear low volume 384-well source microtiter plate (Greiner Bio-One, Frickenhausen, Germany), from which 50 nL of compounds were transferred into a black low volume test microtiter plate from the same supplier. Subsequently, 2 μL of Bub1 (the final concentration of Bub1 was adjusted depending on the activity of the enzyme lot in order to be within the linear dynamic range of the assay: typically ˜200 ng/mL were used) in aqueous assay buffer [50 mM Tris/HCl pH 7.5, 10 mM magnesium chloride (MgCl₂), 200 mM potassium chloride (KCl), 1.0 mM dithiothreitol (DTT), 0.1 mM sodium ortho-vanadate, 1% (v/v) glycerol, 0.01% (w/v) bovine serum albumine (BSA), 0.005% (v/v) Trition X-100 (Sigma), 1× Complete EDTA-free protease inhibitor mixture (Roche)] were added to the compounds in the test plate and the mixture was incubated for 15 min at 22° C. to allow pre-equilibration of the putative enzyme-inhibitor complexes before the start of the kinase reaction, which was initiated by the addition of 3 μL 1.67-fold concentrated solution (in assay buffer) of adenosine-tri-phosphate (ATP, 10 μM final concentration) and peptide substrate (1 μM final concentration). The resulting mixture (5 μL final volume) was incubated at 22° C. during 60 min., and the reaction was stopped by the addition of 5 μL of an aqueous EDTA-solution (50 mM EDTA, in 100 mM HEPES pH 7.5 and 0.2% (w/v) bovine serum albumin) which also contained the TR-FRET detection reagents (0.2 μM streptavdin-XL665 [Cisbio Bioassays, Codolet, France] and 1 nM anti-phosho-Serine antibody [Merck Millipore, cat. #35-001] and 0.4 nM LANCE EU-W1024 labeled anti-mouse IgG antibody [Perkin-Elmer, product no. AD0077, alternatively a Terbium-cryptate-labeled anti-mouse IgG antibody from Cisbio Bioassays can be used]). The stopped reaction mixture was further incubated 1 h at 22° C. in order to allow the formation of complexes between peptides and detection reagents. Subsequently, the amount of product was evaluated by measurement of the resonance energy transfer from the Eu-chelate-antibody complex recognizing the Phosphoserine residue to the streptavidin-XL665 bound to the biotin moiety of the peptide. To this end, the fluorescence emissions at 620 nm and 665 nm alter excitation at 330-350 nm were measured in a TR-FRET plate reader, e.g. a Rubystar or Pherastar (both from BMG Labtechnologies, Offenburg, Germany) or a Viewlux (Perkin-Elmer) and the ratio of the emissions (665 nm/622 nm) was taken as indicator for the amount of phosphorylated substrate. The data were normalised using two sets of (typically 32-) control wells for high-(=enzyme reaction without inhibitor=0%=Minimum inhibition) and low-(=all assay components without enzyme=100%=Maximum inhibition) Bub1 activity. IC₅₀ values were calculated by fitting the normalized inhibition data to a 4-parameter logistic equation (Minimum, Maximum, IC₅₀, Hill; Y=Max+(Min−Max)/(1+(X/IC₅₀)Hill)).

Biological Assay 2.0:

Proliferation Assay:

Human tumour cells were originally obtained from the American Type Culture Collection (ATCC), the Deutsche Sammlung von Mikroorganismen und Zellkulturen (DSMZ, German Collection of Microorganisms and Cell Cultures), or Epo GmbH Berlin. Cultivated HeLa human cervical tumor cells (DSMZ ACC-57) were plated at a density of 3000 cells/well in a 96-well multititer plate in 200 μL of growth medium supplemented 10% fetal calf serum. After 24 hours, the cells of one plate (zero-point plate) were stained with crystal violet (see below), while the medium of the other plates was supplemented with the test substances in various concentrations (0 μM, as well as in the range of 0.001-10 μM; the final concentration of the solvent dimethyl sulfoxide was adjusted to 0.1%) using a Hewlett-Packard HP D300 Digital Dispenser. The cells were incubated for 4 days in the presence of test substances. Cell proliferation was determined by staining the cells with crystal violet: the cells were fixed by adding 20 μL/measuring point of an 11% glutaric aldehyde solution for 15 minutes at room temperature. After three washing cycles of the fixed cells with water, the plates were dried at room temperature. The cells were stained by adding 100 μL/measuring point of a 0.1% crystal violet solution (pH 3.0). After three washing cycles of the stained cells with water, the plates were dried at room temperature. The dye was dissolved by adding 100 μL/measuring point of a 10% acetic acid solution. Absorption was determined by photometry at a wavelength of 595 nm. The change of cell number, in percent, was calculated by normalization of the measured values to the absorption values of the zero-point plate (=0%) and the absorption of the untreated (0 μm) cells (=100%). The IC₅₀ values were determined by means of a 4 parameter fit.

Tab.1. Compounds had been evaluated in the HeLa human cervical cancer cell line to demonstrate antiproliferative activity.

The following table gives the data for the examples of the present invention for the biological assays 1 and 2:

Biological Assay 3.0:

Proliferation Assay (HeLa+Paclitaxel):

Cultivated HeLa human cervical tumor cells (DSMZ ACC-57) were plated at a density of 3000 cells/well in a 96-well multititer plate in 200 μL of growth medium supplemented 10% fetal calf serum. After 24 hours, the cells of one plate (zero-point plate) were tained with crystal violet (see below). The medium of the other plates was supplemented with 3 nM of paclitaxel (Sigma-Aldrich) and the cells were incubated at 37° C. After 4 hours the test substances were added in various concentrations (0 μM, as well as in the range of 0.001-10 μM; the final concentration of the solvent dimethyl sulfoxide was adjusted to 0.1%) using a Hewlett-Packard HP D300 Digital Dispenser. The cells were incubated for another 92 hours at 37° C. in the presence of test substances. Cell proliferation was determined by staining the cells with crystal violet: the cells were fixed by adding 20 μL/measuring point of an 11% glutaric aldehyde solution for 15 minutes at room temperature. After three washing cycles of the fixed cells with water, the plates were dried at room temperature. The cells were stained by adding 100 μL/measuring point of a 0.1% crystal violet solution (pH 3.0). After three washing cycles of the stained cells with water, the plates were dried at room temperature. The dye was dissolved by adding 100 μL/measuring point of a 10% acetic acid solution. Absorption was determined by photometry at a wavelength of 595 nm.

The change of cell number, in percent, was calculated by normalization of the measured values to the absorption values of the zero-point plate (=0%) and the absorption of the untreated (0 μm) cells (=100%). The IC₅₀ values were determined by means of a 4 parameter fit.

Biological Assay 4.1: Formation-Assay

Cell-Based Mechanistic Assay: Changes of Phosphorylation Status of Histone 2A by Inhibition of Kinase Activity of Bub1

This assay determines the suppression of histone 2A phosphorylation by a Bub1 kinase inhibitor during co-treatment with Nocodazole. 25000 cells (cells were ordered from ATCC) were seeded in 96well plate for 5 h at 37° C. Cells were treated with Nocodazole (1 μg/ml) and varying concentrations (between 3 nM and 10 μM) of test compounds for 16 h. Cells were fixed (20 min, Fixing solution R&D), washed three times with PBS and blocked with Odyssey blocking buffer before incubating with the primary antibody against phosphorylated H2A (5 μg/ml ABIN482721) overnight at 2-8° C. After washing, secondary IRDye-labeled antibody mix with cell stains was added for 1 h and washed again with PBS. Plates were scanned with LICor Odyssey Infrared Imager CLx at 800 nm for P-H2A and at 700 nm for cell stains Draq5/Sapphire. The quotient of 800 nm and 700 nm for Nocodazole only treated cells was set as 100% and the quotient of 800 nm and 700 nm of untreated cells was set as 0%. The results given as % reflecting the inhibition of Bub1 kinase activity compared to control and normalized according to cell number. The IC₅₀ values were determined by means of a 4 parameter fit.

Histone H2A is an immediate Intracellular substrate of Bub1 kinase. Determination of the phosphorylation status of Histone H2A provides a direct measure of the intracellular activity of Bub1 kinase. The compounds according to the invention inhibit Bub1 kinase activity in with IC₅₀ values in the nanomolar range in biochemical assays similar as it was described for compounds from WO 2013050438. Surprisingly, it was now found that the compounds according to the invention inhibit intracellular Bub1 kinase activity, in terms of inhibition of Histone H2A phosphorylation, much more potently as compared to compounds from WO 2013050438.

Compounds according to the invention may provide additional surprising benefits, such as,
more potent Inhibition of HeLa human tumor cells, when used in combination with paclitaxel, and/or
reduced drug-drug interaction when used in combination with paclitaxel.

Biological Assay 4.2: Abrogation-Assay

Cell-Based Mechanistic Assay: Changes of Phosphorylation Status of Pre-Induced Phospho-Histone 2A by Inhibition of Kinase Activity of Bub1

This assay measures the inhibition of histone 2A phosphorylation, which was induced by pre-treatment of the cells with Nocodazole, by a Bub1 kinase inhibitor. 25000 cells (cells were ordered from ATCC) were seeded in 96well plate for 5 h at 37° C. Cells were treated with Nocodazole (1 μg/ml). After 16 h varying concentrations (between 3 nM and 10 μM) of test compounds were added and the cells were incubated for another 1 h. Cells were fixed (20 min, Fixing solution R&D), washed three times with PBS and blocked with Odyssey blocking buffer before incubating with the primary antibody against phosphorylated H2A (5 μg/ml ABIN482721) overnight at 2-8° C. After washing, secondary IRDye-labeled antibody mix with cell stains was added for 1 h and washed again with PBS. Plates were scanned with LiCor Odyssey Infrared Imager CLx at 800 nm for P-H2A and at 700 nm for cell stains Draq5/Sapphire. The quotient of 800 nm and 700 nm for Nocodazole only treated cells was set as 100% and the quotient of 800 nm and 700 nm of untreated cells was set as 0%. The results given as % reflecting the inhibition of Bub1 kinase activity compared to control and normalized according to cell number. The IC₅₀ values were determined by means of a 4 parameter fit.

Biological Assay 5.0:

Equilibrium Probe Competition Assay:

Bub1 binding affinity of compounds described in the present invention were quantified using a time-resolved fluorescence energy transfer (TR-FRET) kinase binding assay which measures displacement of the synthetic fluorescent probe 5-[({2-[2-({2-[1-(4-ethoxy-2,6-difluorobenzyl)-1H-indazol-3-yl]-4-(pyridin-4-ylamino)pyrimidin-5-yl}oxy)ethoxy]ethyl}carbamothioyl)amino]-2-(6-hydroxy-3-oxo-3H-xanthen-9-yl)benzoic acid (5-5-1) by the (recombinant) catalytic domain of human Bub1 (amino acids 704-1085). The enzyme was expressed in Hi5 insect cells with N-terminal 6×His/AviTag@ seqences, purified by affinity-(Ni-NTA) and size exclusion chromatography and site-specific biotinylated ex-vivo using a commercially available kit (Avidity). The probe 5-5-1 itself inhibits the kinase activity of Bub1 with an IC₅₀ of 20.8 nM.

In a typical assay 11 different concentrations of each compound (0.1 nM, 0.33 nM, 1.1 nM, 3.8 nM, 13 nM, 44 nM, ˜0.15 μM, ˜0.51 μM, ˜1.7 μM, 5.9 μM and 20 μM) were tested in duplicate within the same microtiter plate. To this end, 100-fold concentrated compound solutions (in DMSO) were made by serial dilution (1:3.4) of freshly prepared 2 mM stocks in a clear low volume 384-well source microtiter plate (Greiner Bio-One, Frickenhausen, Germany), from which 50 nl of compounds were transferred into a black low volume test microtiter plate from the same supplier. Subsequently 2 μL of a solution containing biotinylated Bub1 (the final concentration of Bub1 was adjusted depending on the activity of the enzyme lot in order to be within the linear dynamic range of the assay: typically ˜200 pM were used) and 1 nM Streptavidin Terbium Cryptate (Cisbio, No. 610SATLB) in aqueous assay buffer [50 mM Tris/HCl pH 7.5, 200 mM potassium chloride (KCl), 1.0 mM dithiothreitol (DTT), 0.1 mM sodium ortho-vanadate, 1% (v/v) glycerol, 0.01% (w/v) bovine serum albumine (BSA), 0.005% (v/v) Triton X-100 (Sigma), were added to the compounds in the test plate and the mixture was incubated for 15 min at 22′C to allow pre-equilibration of the putative enzyme-inhibitor complexes before the start of the probe competition reaction, which was initiated by the addition of 3 μL 1.67-fold concentrated solution (in assay buffer) of 5-5-1 (final concentration: 1.5 nM). The resulting mixture (5 μL final volume) was Incubated at 220 during 0, 15, 30, 60, 120, 240 and 1440 min. After each time point the resonance energy transfer from the Streptavidin Tb cryptate to the 3′,6′-dihydroxy-3H-spiro[2-benzofuran-1,9′-xanthen]-3-one moiety of 5-5-1 was measured in order to quantify the amount of fluorescent probe displaced by the test compounds. To this end, the fluorescence emissions at 520 nm and 490 nm after excitation at 330-350 nm were measured in a Pherastar FS TR-FRET plate reader, (BMG Lab technologies, Offenburg, Germany) and the ratio of the emissions (520 nm/490 nm) was taken as Indicator for the amount of Bub1/5-5-1 equilibrium complexes. The data were normalized using two sets of control wells for high-(=enzyme reaction without inhibitor=0%=Minimum inhibition) and low-(=all assay components without enzyme=100%=Maximum inhibition) Bub1 activity. IC50 values were calculated by fitting the normalized inhibition data to a 4-parameter logistic equation (Minimum, Maximum, IC50, Hill; Y=Max+(Min−Max)/(1+(X/IC50)Hill)).

Biological Assay 6.0:

Stability in Buffer at Various pH Values (HPLC Detection)

0.3 mg of the test compound was solved in 0.1 ml dimethylsulfoxide and 0.4 ml acetonitrile. For complete dissolution the HPLC vial with the sample solution was sonified for about 20 seconds. Then 1.0 ml of the respective buffer solution was added and the sample was again treated in the ultrasonic bath.

Buffer Solutions Used:
pH 4.0: Fluka buffer, Order No. 33643 (11.76 g citric acid, 2.57 g sodium chloride and 2.72 g sodium hydroxide)

pH 7.4: 90 g of sodium chloride, 13.61 g of potassium dihydrogen phosphate and 83.35 g of 1 M sodium hydroxide solution were made up to 1 L with Millipore water and then diluted 1:10.

10 μl portions of the sample solution were analysed by HPLC at different times (0 h, 1 h, 2 h, 4 h and 24 h) at 37° C. The peak areas in percentage were used for quantification.

HPLC Method:

Agilent 1100 with DAD (G1315B), binary pump (G1312A), autosampler (G1329A), column oven (G1316A), thermostat (G1330B); column: Kromasil 100 C18, 250 mm×4 mm, 5 μm; column temperature: 37° C.; eluent A: water+5 ml of perchloric acid/litre, eluent B: acetonitrile.

Gradient:

0 min 98% A, 2% B→0-3.0 min 85% A, 15% B→3.0-8.0 min 50% A, 50% B→8.0-16.0 min 50% A, 50% B→16.0-20.0 min 10% A, 90% B→20.0-21.0 10% A, 90% B→21.0-24.0 min 98% A, 2% B→24.0-25.0 min 98% A, 2% B; flow rate: 1.5 ml/min; UV detection: 210 nm.

The ratios of the peak area (F) at different times in relation to the peak area at the starting point are shown in Table 3 for representative examples:

Biological Assay 7.0:

Evaluation of Drug-Drug Interaction Potential with Paclitaxel

To evaluate the drug-drug interaction potential of test compounds with paclitaxel in vivo 8 mg/kg of paclitaxel were injected once intravenously into the tail vein of NMRI nude mice. Immediately thereafter 50 mg/kg of the test compound was administered by gavage to mice. Blood was taken from mice following decapitation 1, 3, 7 and 24 hours after injection of Paclitaxel. Plasma concentrations of test compound and of paclitaxel, respectively, were determined by LC/MSMS. The data from the paclitaxel mono treatment group, the test compound mono treatment group, and the combination

TABLE 1

| | | Biological Assay 2: |
|---|---|---|
| | Biological Assay 1: | Proliferation assay (HeLa |
| Example | Bub1 kinase assay | cell line) |
| No. | median IC₅₀ [mol/L] | median IC₅₀ [mol/L] |
| 2-1-1 | 1.5E−8 | >1.0E−5 |
| 2-1-2 | 1.0E−8 | 3.7E−6 |
| 2-1-3 | 1.1E−8 | 3.7E−6 |
| 2-1-4 | 1.2E−8 | 3.0E−6 |
| 2-1-5 | 3.5E−9 | >1.0E−5 |
| 2-1-6 | 8.3E−9 | 5.9E−6 |
| 2-1-7 | 7.0E−9 | >1.0E−5 |
| 2-1-9 | 3.8E−9 | >1.0E−5 |
| 2-1-10 | 1.5E−8 | 9.0E−6 |
| 2-1-11 | 2.1E−8 | 3.2E−6 |
| 2-1-12 | 3.5E−8 | nd |
| 2-1-13 | 1.1E−8 | 3.9E−6 |
| 2-1-14 | >2.0E−5 | >1.0E−5 |
| 2-1-15 | 8.0E−9 | 3.0E−6 |
| 2-1-16 | 1.6E−8 | nd |
| 2-1-17 | 9.2E−9 | >1.0E−5 |
| 2-1-19 | 2.1E−8 | >1.0E−5 |
| 2-1-20 | 1.3E−8 | 6.20E−6 |
| 2-1-21 | 1.1E−8 | >1.0E−5 |
| 2-1-22 | 5.7E−9 | 1.39E−6 |
| 2-1-23 | 1.5E−8 | 2.32E−6 |
| 2-1-24 | 2.3E−8 | 9.94E−6 |
| 2-1-25 | 1.9E−8 | 1.36E−6 |
| 2-1-26 | 1.4E−8 | 3.79E−6 |
| 2-1-27 | 4.0E−9 | nd |
| Reference | | |
| Compound: | | |
| 5-5-1 | 2.1E−8 | nd |
| 5-6-1 | 1.1E−8 | >1.0E−5 |
| | | 1.5E−6 |

TABLE 2

| | Biological | | |
|---|---|---|---|
| | Assay 4.1: | Biological Assay | Biological Assay 3: |
| | H2A | 4:2: | Proliferation Assay |
| Example | Formation IC₅₀ | H2A Abrogation | (HeLa + Paclitaxel) |
| No. | [mol/L] | IC₅₀ [mol/L] | IC₅₀ [mol/L] |
| 2-1-1 | 4.4E−8 | 1.8E−7 | 2.0E−7 |
| 2-1-2 | 3.1E−8 | 1.7E−8 | <1.0E−7 |
| | | | 5.0E−8 |
| 2-1-3 | 9.7E−10 | 9.3E−10 | 5.4E−8 |
| 2-1-4 | 8.4E−9 | 8.0E−8 | 1.0E−7 |
| 2-1-5 | 2.8E−8 | 4.4E−9 | 1.0E−7 |
| 2-1-6 | 4.5E−8 | 5.8E−8 | 1.2E−7 |
| 2-1-7 | 8.7E−8 | 4.8E−8 | 1.9E−7 |
| 2-1-9 | 17.1E−8 | 3.3E−8 | 3.3E−7 |
| 2-1-10 | 4.4E−8 | 2.3E−8 | 1.4E−6 |
| 2-1-11 | 2.6E−8 | 1.7E−8 | 2.6E−7 |
| 2-1-12 | nd | nd | nd |
| 2-1-13 | 3.6E−7 | 6.4E−8 | 1.8E−7 |
| 2-1-14 | 8.7E−9 | 9.2E−10 | 3.9E−8 |
| 2-1-15 | 3.7E−8 | 4.6E−9 | 1.4E−7 |
| 2-1-16 | nd | nd | nd |
| 2-1-17 | 1.7E−9 | 3.9E−9 | 2.0E−7 |
| 2-1-19 | 3.5E−9 | 2.5E−9 | 1.2E−7 |
| 2-1-20 | 1.1E−7 | >1.0E−6 | 9.8E−8 |
| 2-1-21 | 2.6E−8 | 5.6E−8 | 1.7E−7 |
| 2-1-22 | nd | nd | 1.8E−7 |
| 2-1-23 | nd | nd | 3.0E−7 |
| 2-1-24 | 2.1E−9 | 8.5E−10 | 3.3E−7 |
| 2-1-25 | nd | nd | 2.0E−7 |
| 2-1-26 | 1.4E−8 | 3.8E−8 | 6.4E−8 |
| 2-1-27 | nd | nd | nd |
| Reference | | | |
| Compound: | | | |
| 5-6-1 | 1.1E−6 | nd | 1.4E−6 |

TABLE 3

| | Biological Assay 6: | Biological Assay 6: |
|---|---|---|
| | Stability pH4: % Test | Stability pH7.4: % Test |
| Prodrug No./ | Compound/Parent | Compound/Parent |
| Parent | compound | compound |
| compound | after 4 h | after 4 h |
| No. | [F(t = 4 h) × 100/F(t = 0 h)] | [F(t = 4 h) × 100/F(t = 0 h)] |
| 4-1-1/2-1-1 | 100%/1% | 32%/72% |
| 4-1-2/2-1-5 | 100%/0% | 30%/72% |
| 4-1-3/2-1-5 | 100%/6% | 31%/74% |
| 4-1-4/2-1-5 | 100%/5% | 53%/52% |
| 4-1-5/2-1-6 | 101%/6% | 42%/66% |
| 4-1-6/2-1-11 | 101%/15% | 53%/63% |
| 4-1-7/2-1-11 | 99%/6% | 43%/62% |
| 4-1-8/2-1-6 | 100%/4% | 56%/48% |
| 4-1-9/2-1-6 | 99%/6% | 99%/7% |
| 4-1-10/2-1-6 | nd | nd |

TABLE 4
Assays 5 and 6

| Compound | Type | Time (min.) | IC₅₀ (M) | Recovery (%) |
|---|---|---|---|---|
| 2-1-5 | Drug | 0 | | |
| 2-1-5 | Drug | 15 | 5.9E−10 | |
| 2-1-5 | Drug | 30 | 4.9E−10 | |
| 2-1-5 | Drug | 60 | 3.2E−10 | |
| 2-1-5 | Drug | 90 | 2.6E−10 | |
| 2-1-5 | Drug | 120 | 2.0E -10 | |
| 2-1-5 | Drug | 240 | 1.6E−10 | |
| 2-1-5 | Drug | 1440 | 9.8E−11 | |
| 4-1-4 | Prodrug | 0 | | |
| 4-1-4 | Prodrug | 15 | 2.8E−09 | |
| 4-1-4 | Prodrug | 30 | 5.8E−10 | |
| 4-1-4 | Prodrug | 60 | 6.1E−10 | 86 |
| 4-1-4 | Prodrug | 90 | 5.5E−10 | |
| 4-1-4 | Prodrug | 120 | 5.4E−10 | 73 |
| 4-1-4 | Prodrug | 240 | 3.2E−10 | 53 |
| 4-1-4 | Prodrug | 1440 | 3.3E−10 | 3 |
| 2-1-1 | Drug | 0 | | |
| 2-1-1 | Drug | 15 | 7.6E−10 | |
| 2-1-1 | Drug | 30 | 9.0E−10 | |
| 2-1-1 | Drug | 60 | 1.3E−09 | |
| 2-1-1 | Drug | 90 | 1.2E−09 | |
| 2-1-1 | Drug | 120 | 9.4E−10 | |
| 2-1-1 | Drug | 240 | 9.9E−10 | |
| 2-1-1 | Drug | 1440 | 1.0E−09 | |
| 4-1-1 | Prodrug | 0 | | |
| 4-1-1 | Prodrug | 15 | 1.4E−09 | |
| 4-1-1 | Prodrug | 30 | 1.5E−09 | |
| 4-1-1 | Prodrug | 60 | 1.4E−09 | 76 |
| 4-1-1 | Prodrug | 90 | 1.5E−09 | |
| 4-1-1 | Prodrug | 120 | 1.3E−09 | 57 |
| 4-1-1 | Prodrug | 240 | 1.5E−09 | 32 |
| 4-1-1 | Prodrug | 1440 | 2.0E−09 | 0 |
| 2-1-6 | Drug | 0 | | |
| 2-1-6 | Drug | 15 | 1.7E−09 | |
| 2-1-6 | Drug | 30 | 8.7E−10 | |
| 2-1-6 | Drug | 60 | 8.5E−10 | |
| 2-1-6 | Drug | 90 | 1.2E−09 | |
| 2-1-6 | Drug | 120 | 3.8E−10 | |
| 2-1-6 | Drug | 240 | 3.1E−10 | |
| 2-1-6 | Drug | 1440 | 2.0E−10 | |
| 4-1-5 | Prodrug | 0 | | |
| 4-1-5 | Prodrug | 15 | 1.5E−09 | |
| 4-1-5 | Prodrug | 30 | 8.2E−10 | |
| 4-1-5 | Prodrug | 60 | 6.0E−10 | 81 |
| 4-1-5 | Prodrug | 90 | 5.1E−10 | |
| 4-1-5 | Prodrug | 120 | 4.6E−10 | 65 |
| 4-1-5 | Prodrug | 240 | 2.7E−10 | 42 |
| 4-1-5 | Prodrug | 1440 | 2.5E−10 | 0 |
| 2-1-5 | Drug | 0 | | |
| 2-1-5 | Drug | 15 | 5.9E−10 | |
| 2-1-5 | Drug | 30 | 4.9E−10 | |
| 2-1-5 | Drug | 60 | 3.2E−10 | |
| 2-1-5 | Drug | 90 | 2.6E−10 | |
| 2-1-5 | Drug | 120 | 2.0E−10 | |
| 2-1-5 | Drug | 240 | 1.6E−10 | |
| 2-1-5 | Drug | 1440 | 9.8E−11 | |
| 4-1-3 | Prodrug | 0 | | |
| 4-1-3 | Prodrug | 15 | 1.1E−09 | |
| 4-1-3 | Prodrug | 30 | 6.5E−10 | |
| 4-1-3 | Prodrug | 60 | 4.2E−10 | 75 |
| 4-1-3 | Prodrug | 90 | 2.7E−10 | |
| 4-1-3 | Prodrug | 120 | 2.5E−10 | 56 |
| 4-1-3 | Prodrug | 240 | 2.0E−10 | 31 |
| 4-1-3 | Prodrug | 1440 | 1.7E−10 | 0 |

1: A compound of formula (I) 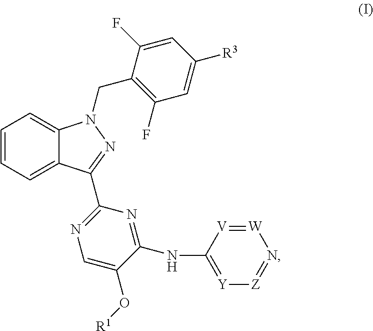
in which
V, W, Y and Z independently of each other represent CH or CR², wherein one of V, W, Y and Z represents CR²,
or,
V represents N, and W, Y and Z independently of each other represent CH or CR²,
or,
V and Y represent N, and W and Z independently of each other represent CH or CR²,
R¹ represents a group selected from:
C₂-C₆-hydroxyalkyl, and R⁴,
wherein said C₂-C₆-hydroxyalkyl group is optionally substituted with one, two or three halogen atoms selected from:
fluorine, and chlorine,
R² represents, independently of each other, halogen or a group selected from:
C₁-C₃-alkyl, C₃-C₄-cycloalkyl, C₁-C₃-haloalkyl, C₁-C₃-alkoxy, C₁-C₃-haloalkoxy, —N(H)C(═O)—(C₁-C₃-alkyl),
—N(H)C(═O)H, —N(H)C(═O)—(C₁-C₃-hydroxyalkyl), —N(H)C(═O)—(C₁-C₃-alkyl)-(C₁-C₃-alkoxy), —N(H)C(═O)-phenyl, —N(H)C(═O)—(C₃-C₄-cycloalkyl), —N(H)C(═O)—(C₁-C₃-alkyl)-(C₃-C₄-cycloalkyl), and —N(H)C(═O)N(H)R⁸,
said —N(H)C(═O)-phenyl being optionally substituted at the phenyl ring, one, two or three times, identically or differently, with a substituent selected from:
halogen, hydroxy, cyano, C₁-C₄-alkyl, C₁-C₄-haloalkyl, C₁-C₄-alkoxy, C₁-C₄-haloalkoxy, C₃-C₄-cycloalkyl, and C₃-C₄-cycloalkyloxy,
said —N(H)C(═O)—(C₃-C₄-cycloalkyl) being optionally substituted at the C₃-C₄-cycloalkyl ring with a substituent selected from:
fluorine, chlorine, trifluoromethyl, and methoxy,
R³ represents a group selected from:
C₁-C₆-alkyl, C₁-C₆-hydroxyalkyl, C₁-C₆-haloalkyl, C₃-C₆-cycloalkyl, (C₁-C₃-alkoxy)-(C₁-C₃-alkyl)-, (C₃-C₆-cycloalkyl)-(C₁-C₃-alkyl)-, C₁-C₆-alkoxy, (C₂-C₆-hydroxyalkyl)-O—, C₁-C₆-haloalkoxy, C₃-C₆-cycloalkyloxy, (C₁-C₃-alkoxy)-(C₂-C₃-alkoxy)-, and (C₃-C₆-cycloalkyl)-(C₁-C₃-alkoxy)-,
wherein said C₂-C₆-hydroxyalkyl group is optionally substituted with one, two or three halogen atoms selected from:
fluorine, and chlorine,
R⁴ represents —(C₂-C₆-alkyl)-OC(═O)—C(H)(R⁵)—N(H)C(═O)—C(H)(R⁷)—NH₂,
in which C₂-C₆-alkyl is optionally substituted with one, two or three halogen atoms selected from:
fluorine, and chlorine,
R⁵ and R⁷ independently of each other represent hydrogen (glycine) or a group selected from:
—CH₃ (alanine), —C(H)(CH₃)₂ (valine), —(CH₂)₂CH₃ (norvaline), —CH₂C(H)(CH₃)₂ (leucine), —C(H)(CH₃)CH₂CH₃ (isoleucine), —(CH₂)₃CH₃ (norleucine), —C(CH₃)₃ (2-tert-butylglycine), benzyl (phenylalanine), 4-hydroxybenzyl (tyrosine), —(CH₂)₃NH₂ (ornithine), —(CH₂)₄NH₂ (lysine), —(CH₂)₂C(H)(OH)CH₂NH₂ (hydroxylysine), —CH₂OH (serine), —(CH₂)₂OH (homoserine), —C(H)(OH)CH₃ (threonine), —(CH₂)₃N(H)C(═NH)NH₂ (arginine), —(CH₂)₃N(H)C(═O)NH₂ (citrulline), —CH₂C(═O)NH₂ (asparagine), —CH₂C(═O)OH (aspartic acid), —(CH₂)₂C(═O)OH (glutamic acid), —(CH₂)₂C(═O)NH₂ (glutamine), —CH₂SH (cysteine), —(CH₂)₂SH (homocysteine), —(CH₂)₂SCH₃ (methionine), —CH₂SCH₃ (S-methylcysteine), (1H-imidazol-4-yl)methyl-(histidine), (1H-indol-3-yl)methyl-(thryptophan), —CH₂NH₂ (2,3-diaminopropanoic acid), and —(CH₂)₂NH₂ (2,4-diaminobutanoic acid), and
R⁸ represents hydrogen or a group selected from:
C₁-C₃-alkyl, C₁-C₃-haloalkyl, C₂-C₃-hydroxyalkyl, C₃-C₄-cycloalkyl, (C₃-C₄-cycloalkyl)-(C₁-C₃-alkyl)-, and (C₁-C₃-alkoxy)-(C₂-C₃-alkyl)-,
or an N-oxide, a salt, a tautomer or a stereoisomer of said compound, or a salt of said N-oxide, tautomer or stereoisomer.

2: The compound of formula (I) according to claim 1, wherein
V, W, Y and Z independently of each other represent CH or CR², wherein one of V, W, Y and Z represents CR²,
or,
V represents N, and W, Y and Z independently of each other represent CH or CR²,
R¹ represents a group selected from:
C₂-C₆-hydroxyalkyl, and R⁴,
wherein said C₂-C₆-hydroxyalkyl group is optionally substituted with one, two or three halogen atoms selected from:
fluorine, and chlorine,
R² represents, independently of each other, halogen or a group selected from:
C₁-C₃-alkyl, C₃-C₄-cycloalkyl, C₁-C₃-haloalkyl, C₁-C₃-alkoxy, C₁-C₃-haloalkoxy, —N(H)C(═O)—(C₁-C₃-alkyl), —N(H)C(═O)H,
—N(H)C(═O)—(C₁-C₃-hydroxyalkyl), —N(H)C(═O)—(C₁-C₃-alkyl)-(C₁-C₃-alkoxy), —N(H)C(═O)-phenyl, —N(H)C(═O)—(C₃-C₄-cycloalkyl), —N(H)C(═O)—(C₁-C₃-alkyl)-(C₃-C₄-cycloalkyl), and —N(H)C(═O)N(H)R⁸,
said —N(H)C(═O)-phenyl being optionally substituted at the phenyl ring, one, two or three times, identically or differently, with a substituent selected from:
halogen, hydroxy, cyano, C₁-C₄-alkyl, C₁-C₄-haloalkyl, C₁-C₄-alkoxy, C₁-C₄-haloalkoxy, C₃-C₄-cycloalkyl, and C₃-C₄-cycloalkyloxy,
said —N(H)C(═O)—(C₃-C₄-cycloalkyl) being optionally substituted at the C₃-C₄-cycloalkyl ring with a substituent selected from:
fluorine, chlorine, trifluoromethyl, and methoxy,
R³ represents a group selected from:
C₁-C₆-hydroxyalkyl, C₁-C₆-alkoxy, (C₂-C₆-hydroxyalkyl)-O—, C₁-C₆-haloalkoxy, and (C₃-C₆-cycloalkyl)-(C₁-C₃-alkoxy)-,
wherein said C₂-C₆-hydroxyalkyl group is optionally substituted with one, two or three halogen atoms selected from:
fluorine, and chlorine,
R⁴ represents —(C₂-C₆-alkyl)-OC(═O)—C(H)(R⁵)—N(H)C(═O)—C(H)(R⁷)—NH₂,
in which C₂-C₆-alkyl is optionally substituted with one, two or three halogen atoms selected from:
fluorine, and chlorine,
R⁵ and R⁷ independently of each other represent a group selected from:
—CH₃ (alanine), —C(H)(CH₃)₂ (valine), —(CH₂)₂CH₃ (norvaline), —(CH₂)₃NH₂ (ornithine), —(CH₂)₄NH₂ (lysine), and —(CH₂)₃N(H)C(═NH)NH₂ (arginine), and
R⁸ represents hydrogen or a group selected from:
C₁-C₃-alkyl, C₁-C₃-haloalkyl, C₂-C₃-hydroxyalkyl, C₃-C₄-cycloalkyl, (C₃-C₄-cycloalkyl)-(C₁-C₃-alkyl)-, and (C₁-C₃-alkoxy)-(C₂-C₃-alkyl)-,
or an N-oxide, a salt, a tautomer or a stereoisomer of said compound, or a salt of said N-oxide, tautomer or stereoisomer.

3: The compound of formula (I) according to claim 1, wherein
V, W, Y and Z independently of each other represent CH or CR², wherein one of V, W, Y and Z represents CR²,
or,
V represents N, and W, Y and Z independently of each other represent CH or CR²,
R¹ represents a group selected from:
C₂-C₆-hydroxyalkyl, and R⁴,
R² represents, independently of each other, halogen or a group selected from:
C₁-C₃-alkyl, C₃-C₄-cycloalkyl, C₁-C₃-haloalkyl, and —N(H)C(═O)—(C₁-C₃-alkyl),
R³ represents a group selected from:
C₁-C₆-alkoxy, and (C₃-C₆-cycloalkyl)-(C₁-C₃-alkoxy)-,
R⁴ represents —(C₂-C₆-alkyl)-OC(═O)—C(H)(R⁵)—N(H)C(═O)—C(H)(R⁷)—NH₂, and
R⁵ and R⁷ independently of each other represent a group selected from:
—CH₃ (alanine), —(CH₂)₄NH₂ (lysine), and —(CH₂)₃N(H)C(═NH)NH₂ (arginine),
or an N-oxide, a salt, a tautomer or a stereoisomer of said compound, or a salt of said N-oxide, tautomer or stereoisomer.

4: The compound of formula (I) according to claim 1,
wherein
V, W, Y and Z independently of each other represent CH or CR², wherein one of V, W, Y and Z represents CR²,
or,
V represents N, and W, Y and Z independently of each other represent CH or CR²,
R¹ represents a group selected from:
—(CH₂)₃OH, and R⁴,
R² represents, independently of each other, fluorine, chlorine or a group selected from:
methyl, cyclopropyl, difluoromethyl, and —N(H)C(═O)—CH₃,
R³ represents a group selected from:
ethoxy, and cyclopropylmethoxy,
R⁴ represents —(CH₂)₃—OC(═O)—C(H)(R⁵)—N(H)C(═O)—C(H)(R⁷)—NH₂,
R⁵ represents —CH₃ (alanine), and
R⁷ represents a group selected from:
—(CH₂)₄NH₂ (lysine), and —(CH₂)₃N(H)C(═NH)NH₂ (arginine),
or an N-oxide, a salt, a tautomer or a stereoisomer of said compound, or a salt of said N-oxide, tautomer or stereoisomer.

5: The compound of formula (I) according to claim 1, wherein
V, W, Y and Z independently of each other represent CH or CR², wherein one of V, W, Y and Z represents CR²,
or,
V represents N, and W, Y and Z independently of each other represent CH or CR²,
R¹ represents a group selected from:
—(CH₂)₃OH, and R⁴,
R² represents, independently of each other, fluorine, chlorine or a group selected from:
methyl, cyclopropyl, difluoromethyl, 2,2,2-trifluoroethoxy, —N(H)C(═O)—CH₃, and —N(H)C(═O)-(cyclopropyl),
R³ represents a group selected from:
ethoxy, 2,2-difluoroethoxy, 2,2,2-trifluoroethoxy, 2-methoxyethoxy, and cyclopropylmethoxy,
R⁴ represents —(CH₂)₃—OC(═O)—C(H)(R⁵)—N(H)C(═O)—C(H)(R⁷)—NH₂,
R⁵ represents a group selected from:
—CH₃ (alanine), and —C(H)(CH₃)₂ (valine), and
R⁷ represents a group selected from:
—(CH₂)₄NH₂ (lysine), and —(CH₂)₃N(H)C(═NH)NH₂ (arginine),
or an N-oxide, a salt, a tautomer or a stereoisomer of said compound, or a salt of said N-oxide, tautomer or stereoisomer.

6: The compound of formula (I) according to claim 1, which is selected from the group consisting of:
3-[(4-{bis[2-(difluoromethyl)pyridin-4-yl]amino}-2-[1-(4-ethoxy-2,6-difluorobenzyl)-1H-indazol-3-yl]pyrimidin-5-yl)oxy]propan-1-ol;
3-({2-[1-(4-ethoxy-2,6-difluorobenzyl)-1H-indazol-3-yl]-4-[(3-methylpyridin-4-yl)amino]pyrimidin-5-yl}oxy)propan-1-ol;
3-({4-[(3-cyclopropylpyridin-4-yl)amino]-2-[1-(4-ethoxy-2,6-difluorobenzyl)-1H-indazol-3-yl]pyrimidin-5-yl}oxy)propan-1-ol;
3-({2-[1-(4-ethoxy-2,6-difluorobenzyl)-1H-indazol-3-yl]-4-[(2-methylpyridin-4-yl)amino]pyrimidin-5-yl}oxy)propan-1-ol;
N-[4-({2-[1-(4-ethoxy-2,6-difluorobenzyl)-1H-indazol-3-yl]-5-(3-hydroxy-propoxy)pyrimidin-4-yl}amino)pyridin-2-yl]acetamide;
3-({4-[(3-chloropyridin-4-yl)amino]-2-[1-(4-ethoxy-2,6-difluorobenzyl)-1H-indazol-3-yl]pyrimidin-5-yl} oxy)propan-1-ol;
3-({2-[1-(4-ethoxy-2,6-difluorobenzyl)-1H-indazol-3-yl]-4-[(2-methylpyrimidin-4-yl)amino]pyrimidin-5-yl}oxy)propan-1-ol;
3-({2-[1-(4-ethoxy-2,6-difluorobenzyl)-1H-indazol-3-yl]-4-(pyrimidin-4-ylamino)-pyrimidin-5-yl} oxy)propan-1-ol;
3-({2-[1-(4-ethoxy-2,6-difluorobenzyl)-1H-indazol-3-yl]-4-[(2-fluoropyridin-4-yl)amino]pyrimidin-5-yl}oxy)propan-1-ol;
3-({4-[(2,5-dimethylpyridin-4-yl)amino]-2-[1-(4-ethoxy-2,6-difluorobenzyl)-1H-indazol-3-yl]pyrimidin-5-yl}oxy)propan-1-ol;
N-[4-({2-[1-(4-ethoxy-2,6-difluorobenzyl)-1H-indazol-3-yl]-5-(3-hydroxy-propoxy)pyrimidin-4-yl}amino)-5-methylpyridin-2-yl]acetamide;
3-[(4-[(3-chloropyridin-4-yl)amino]-2-{1-[4-(cyclopropylmethoxy)-2,6-difluorobenzyl]-1H-indazol-3-yl}pyrimidin-5-yl)oxy]propan-1-ol;
3[(4-{[2-(difluoromethyl)pyridin-4-yl]amino}-2-[1-(4-ethoxy-2,6-difluorobenzyl)-1H-indazol-3-yl]pyrimidin-5-yl)oxy]propyl L-lysyl-L-alaninate, salt with trifluoroacetic acid;
3-[(4-{[2-(difluoromethyl)pyridin-4-yl]amino}-2-[1-(4-ethoxy-2,6-difluorobenzyl)-1H-indazol-3-yl]pyrimidin-5-yl)oxy]propyl L-lysyl-L-alaninate;
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-lysyl-L-alaninate, salt with trifluoroacetic acid;
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-lysyl-L-alaninate;
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl D-lysyl-L-alaninate, salt with trifluoroacetic acid;
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl D-lysyl-L-alaninate;
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-arginyl-L-alaninate, salt with trifluoroacetic acid;
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-arginyl-L-alaninate;
3-({4-[(3-chloropyridin-4-yl)amino]-2-[1-(4-ethoxy-2,6-difluorobenzyl)-1H-indazol-3-yl]pyrimidin-5-yl}oxy)propyl L-lysyl-L-alaninate, salt with trifluoroacetic acid;
3-({4-[(3-chloropyridin-4-yl)amino]-2-[1-(4-ethoxy-2,6-difluorobenzyl)-1H-indazol-3-yl]pyrimidin-5-yl}oxy)propyl L-lysyl-L-alaninate;
3-({4-[(2,6-Dimethylpyrimidin-4-yl)amino]-2-[1-(4-ethoxy-2,6-difluorbenzyl)-1H-indazol-3-yl]pyrimidin-5-yl}oxy)propan-1-ol;
N-[4-({2-[1-(4-Ethoxy-2,6-difluorobenzyl)-1H-indazol-3-yl]-5-(3-hydroxypropoxy)-pyrimidin-4-yl}amino)pyridin-2-yl]cyclopropancarboxamide;
3-({2-[1-(4-Ethoxy-2,6-difluorobenzyl)-1H-indazol-3-yl]-4-[(5-fluor-2-methylpyridin-4-yl)amino]pyrimidin-5-yl}oxy)propan-1-ol;
N-[4-({2-[1-(4-ethoxy-2,6-difluorobenzyl)-1H-indazol-3-yl]-5-(3-hydroxypropoxy)-pyrimidin-4-yl}amino)-3-methylpyridin-2-yl]acetamide;
3-[(2-{1-[2,6-difluoro-4-(2,2,2-trifluoroethoxy)benzyl]-1H-indazol-3-yl}-4-[(2-methylpyrimidin-4-yl)amino]pyrimidin-5-yl)oxy]propan-1-ol;
3-[(4-[(3-chloropyridin-4-yl)amino]-2-{1-[2,6-difluoro-4-(2,2,2-trifluoroethoxy)benzyl]-1H-indazol-3-yl}pyrimidin-5-yl)oxy]propan-1-ol;
3-[(2-{1-[4-(2,2-difluoroethoxy)-2,6-difluorobenzyl]-1H-indazol-3-yl}-4-[(2-methylpyrimidin-4-yl)amino]pyrimidin-5-yl)oxy]propan-1-ol;
3-[(2-[1-(4-ethoxy-2,6-difluorobenzyl)-1H-indazol-3-yl]-4-{[3-(2,2,2-trifluoroethoxy)-pyridin-4-yl]amino}pyrimidin-5-yl)oxy]propan-1-ol;
3-[(2-{1-[4-(cyclopropylmethoxy)-2,6-difluorobenzyl]-1H-indazol-3-yl}-4-[(2,5-dimethylpyridin-4-yl)amino]pyrimidin-5-yl)oxy]propan-1-ol;
3-[(2-{1-[4-(cyclopropylmethoxy)-2,6-difluorobenzyl]-1H-indazol-3-yl}-4-[(2-methylpyrimidin-4-yl)amino]pyrimidin-5-yl)oxy]propan-1-ol;
N-(4-{[2-{1-[2,6-difluoro-4-(2,2,2-trifluoroethoxy)benzyl]-1H-indazol-3-yl}-5-(3-hydroxypropoxy)pyrimidin-4-yl]amino}pyridin-2-yl)acetamide;
3-[(4-[(3-chloropyridin-4-yl)amino]-2-{1-[4-(2,2-difluoroethoxy)-2,6-difluorobenzyl]-1H-indazol-3-yl}pyrimidin-5-yl)oxy]propan-1-ol;
3-{[2-{1-[2,6-difluoro-4-(2-methoxyethoxy)benzyl]-1H-indazol-3-yl}-4-(pyrimidin-4-ylamino)pyrimidin-5-yl]oxy}propan-1-ol;
3-({4-[(2,5-dimethylpyridin-4-yl)amino]-2-[1-(4-ethoxy-2,6-difluorobenzyl)-1H-indazol-3-yl]pyrimidin-5-yl}oxy)propyl D-lysyl-L-alaninate, salt with trifluoroaceticacid trifluoroacetic acid;
3-({4-[(2,5-dimethylpyridin-4-yl)amino]-2-[1-(4-ethoxy-2,6-difluorobenzyl)-1H-indazol-3-yl]pyrimidin-5-yl}oxy)propyl D-lysyl-L-alaninate;
3-({4-[(2,5-dimethylpyridin-4-yl)amino]-2-[1-(4-ethoxy-2,6-difluorobenzyl)-1H-indazol-3-yl]pyrimidin-5-yl}oxy)propyl L-lysyl-L-alaninate, salt with trifluoroaceticacid trifluoroacetic acid;
3-({4-[(2,5-dimethylpyridin-4-yl)amino]-2-[1-(4-ethoxy-2,6-difluorobenzyl)-1H-indazol-3-yl]pyrimidin-5-yl}oxy)propyl L-lysyl-L-alaninate;
3-({4-[(3-chloropyridin-4-yl)amino]-2-[1-(4-ethoxy-2,6-difluorobenzyl)-1H-indazol-3-yl]pyrimidin-5-yl}oxy)propyl L-arginyl-L-alaninate;
3-({4-[(3-chloropyridin-4-yl)amino]-2-[1-(4-ethoxy-2,6-difluorobenzyl)-1H-indazol-3-yl]pyrimidin-5-yl}oxy)propyl L-lysyl-L-valinate; and
3-({4-[(3-chloropyridin-4-yl)amino]-2-[1-(4-ethoxy-2,6-difluorobenzyl)-1H-indazol-3-yl]pyrimidin-5-yl}oxy)propyl L-arginyl-L-valinate;
or an N-oxide, a salt, a tautomer or a stereoisomer of said compound, or a salt of said N-oxide, tautomer or stereoisomer.

7: The compound of formula (I) according to claim 1, which is selected from the group consisting of:
3-[(4-{bis[2-(difluoromethyl)pyridin-4-yl]amino}-2-[1-(4-ethoxy-2,6-difluorobenzyl)-1H-indazol-3-yl]pyrimidin-5-yl)oxy]propan-1-ol;
3-({2-[1-(4-ethoxy-2,6-difluorobenzyl)-1H-indazol-3-yl]-4-[(3-methylpyridin-4-yl)amino]pyrimidin-5-yl}oxy)propan-1-ol;
3-({4-[(3-cyclopropylpyridin-4-yl)amino]-2-[1-(4-ethoxy-2,6-difluorobenzyl)-1H-indazol-3-yl]pyrimidin-5-yl}oxy)propan-1-ol;
3-({2-[1-(4-ethoxy-2,6-difluorobenzyl)-1H-indazol-3-yl]-4-[(2-methylpyridin-4-yl)amino]pyrimidin-5-yl}oxy)propan-1-ol;
N-[4-({2-[1-(4-ethoxy-2,6-difluorobenzyl)-1H-indazol-3-yl]-5-(3-hydroxy-propoxy)pyrimidin-4-yl}amino)pyridin-2-yl]acetamide;
3-({4-[(3-chloropyridin-4-yl)amino]-2-[1-(4-ethoxy-2,6-difluorobenzyl)-1H-indazol-3-yl]pyrimidin-5-yl} oxy)propan-1-ol;
3-({2-[1-(4-ethoxy-2,6-difluorobenzyl)-1H-indazol-3-yl]-4-[(2-methylpyrimidin-4-yl)amino]pyrimidin-5-yl}oxy)propan-1-ol;
3-({2-[1-(4-ethoxy-2,6-difluorobenzyl)-1H-indazol-3-yl]-4-(pyrimidin-4-ylamino)-pyrimidin-5-yl} oxy)propan-1-ol;
3-({2-[1-(4-ethoxy-2,6-difluorobenzyl)-1H-indazol-3-yl]-4-[(2-fluoropyridin-4-yl)amino]pyrimidin-5-yl}oxy)propan-1-ol;
3-({4-[(2,5-dimethylpyridin-4-yl)amino]-2-[1-(4-ethoxy-2,6-difluorobenzyl)-1H-indazol-3-yl]pyrimidin-5-yl}oxy)propan-1-ol;
N-[4-({2-[1-(4-ethoxy-2,6-difluorobenzyl)-1H-indazol-3-yl]-5-(3-hydroxy-propoxy)pyrimidin-4-yl}amino)-5-methylpyridin-2-yl]acetamide;
3-[(4-[(3-chloropyridin-4-yl)amino]-2-{1-[4-(cyclopropylmethoxy)-2,6-difluorobenzyl]-1H-indazol-3-yl}pyrimidin-5-yl)oxy]propan-1-ol;
3-[(4-{[2-(difluoromethyl)pyridin-4-yl]amino}-2-[1-(4-ethoxy-2,6-difluorobenzyl)-1H-indazol-3-yl]pyrimidin-5-yl)oxy]propyl L-lysyl-L-alaninate, salt with trifluoroacetic acid;
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-lysyl-L-alaninate, salt with trifluoroacetic acid;
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl D-lysyl-L-alaninate, salt with trifluoroacetic acid;
3-[(4-{[2-(acetylamino)pyridin-4-yl]amino}-2-[1-(4-ethoxy-2,6-difluorobenzyl)-1H-indazol-3-yl]pyrimidin-5-yl)oxy]propyl L-arginyl-L-alaninate, salt with trifluoroaceticacid, trifluoroacetic acid;
3-({4-[(3-chloropyridin-4-yl)amino]-2-[1-(4-ethoxy-2,6-difluorobenzyl)-1H-indazol-3-yl]pyrimidin-5-yl}oxy)propyl L-lysyl-L-alaninate, salt with trifluoroaceticacid trifluoroacetic acid;
3-({4-[(2,6-Dimethylpyrimidin-4-yl)amino]-2-[1-(4-ethoxy-2,6-difluorbenzyl)-1H-indazol-3-yl]pyrimidin-5-yl}oxy)propan-1-ol;
N-[4-({2-[1-(4-Ethoxy-2,6-difluorobenzyl)-1H-indazol-3-yl]-5-(3-hydroxypropoxy)-pyrimidin-4-yl}amino)pyridin-2-yl]cyclopropancarboxamide;
3-({2-[1-(4-Ethoxy-2,6-difluorobenzyl)-1H-indazol-3-yl]-4-[(5-fluor-2-methylpyridin-4-yl)amino]pyrimidin-5-yl}oxy)propan-1-ol;
N-[4-({2-[1-(4-ethoxy-2,6-difluorobenzyl)-1H-indazol-3-yl]-5-(3-hydroxypropoxy)-pyrimidin-4-yl}amino)-3-methylpyridin-2-yl]acetamide;
3-[(2-{1-[2,6-difluoro-4-(2,2,2-trifluoroethoxy)benzyl]-1H-indazol-3-yl}-4-[(2-methylpyrimidin-4-yl)amino]pyrimidin-5-yl)oxy]propan-1-ol;
3-[(4-[(3-chloropyridin-4-yl)amino]-2-{1-[2,6-difluoro-4-(2,2,2-trifluoroethoxy)benzyl]-1H-indazol-3-yl}pyrimidin-5-yl)oxy]propan-1-ol;
3-[(2-{1-[4-(2,2-difluoroethoxy)-2,6-difluorobenzyl]-1H-indazol-3-yl}-4-[(2-methylpyrimidin-4-yl)amino]pyrimidin-5-yl)oxy]propan-1-ol;
3-[(2-[1-(4-ethoxy-2,6-difluorobenzyl)-1H-indazol-3-yl]-4-{[3-(2,2,2-trifluoroethoxy)-pyridin-4-yl]amino}pyrimidin-5-yl)oxy]propan-1-ol;
3-[(2-{1-[4-(cyclopropylmethoxy)-2,6-difluorobenzyl]-1H-indazol-3-yl}-4-[(2,5-dimethylpyridin-4-yl)amino]pyrimidin-5-yl)oxy]propan-1-ol;
3-[(2-{1-[4-(cyclopropylmethoxy)-2,6-difluorobenzyl]-1H-indazol-3-yl}-4-[(2-methylpyrimidin-4-yl)amino]pyrimidin-5-yl)oxy]propan-1-ol;
N-(4-{[2-{1-[2,6-difluoro-4-(2,2,2-trifluoroethoxy)benzyl]-1H-indazol-3-yl}-5-(3-hydroxypropoxy)pyrimidin-4-yl]amino}pyridin-2-yl)acetamide;
3-[(4-[(3-chloropyridin-4-yl)amino]-2-{1-[4-(2,2-difluoroethoxy)-2,6-difluorobenzyl]-1H-indazol-3-yl}pyrimidin-5-yl)oxy]propan-1-ol;
3-{[2-{1-[2,6-difluoro-4-(2-methoxyethoxy)benzyl]-1H-indazol-3-yl}-4-(pyrimidin-4-ylamino)pyrimidin-5-yl]oxy}propan-1-ol;
3-({4-[(2,5-dimethylpyridin-4-yl)amino]-2-[1-(4-ethoxy-2,6-difluorobenzyl)-1H-indazol-3-yl]pyrimidin-5-yl}oxy)propyl D-lysyl-L-alaninate, salt with trifluoroacetic acid;
3-({4-[(2,5-dimethylpyridin-4-yl)amino]-2-[1-(4-ethoxy-2,6-difluorobenzyl)-1H-indazol-3-yl]pyrimidin-5-yl}oxy)propyl L-lysyl-L-alaninate, salt with trifluoroacetic acid;
3-({4-[(3-chloropyridin-4-yl)amino]-2-[1-(4-ethoxy-2,6-difluorobenzyl)-1H-indazol-3-yl]pyrimidin-5-yl}oxy)propyl L-arginyl-L-alaninate;
3-({4-[(3-chloropyridin-4-yl)amino]-2-[1-(4-ethoxy-2,6-difluorobenzyl)-1H-indazol-3-yl]pyrimidin-5-yl}oxy)propyl L-lysyl-L-valinate; and
3-({4-[(3-chloropyridin-4-yl)amino]-2-[1-(4-ethoxy-2,6-difluorobenzyl)-1H-indazol-3-yl]pyrimidin-5-yl}oxy)propyl L-arginyl-L-valinate;
or an N-oxide, a salt, a tautomer or a stereoisomer of said compound, or a salt of said N-oxide, tautomer or stereoisomer.

8. (canceled)

9: A method for the treatment or prophylaxis of a disease, comprising administering to a patient in need thereof an effective amount of a compound of general formula (I) according to claim 1, or an N-oxide, a salt, a tautomer or a stereoisomer of said compound, or a salt of said N-oxide, tautomer or stereoisomer, wherein the disease is a hyperproliferative disease and/or a disorder responsive to induction of cell death.

10: The method according to claim 9, wherein the hyperproliferative disease and/or disorder responsive to induction of cell death is a haematological tumour, a solid tumour and/or metastases thereof.

11: The method according to according to claim 9, wherein the disease is a hyperproliferative disease, and wherein the hyperproliferative disease is cervical cancer.

12: A pharmaceutical composition comprising at least one compound of general formula (I) according to claim 1, or an N-oxide, a salt, a tautomer or a stereoisomer of said compound, or a salt of said N-oxide, tautomer or stereoisomer, together with at least one pharmaceutically acceptable carrier or auxiliary.

13: A method for the treatment of a haematological tumour, a solid tumour and/or metastases thereof, comprising administering to a patient in need thereof the composition according to claim 12.

14: A combination comprising one or more first active ingredients selected from a compound of general formula (I) according to claim 1, or an N-oxide, a salt, a tautomer or a stereoisomer of said compound, or a salt of said N-oxide, tautomer or stereoisomer, and one or more second active ingredients selected from chemotherapeutic anti-cancer agents and target-specific anti-cancer agents.

15: A method of preparing a compound of general formula (I), said method comprising the step of allowing an intermediate compound of general formula (1-7): 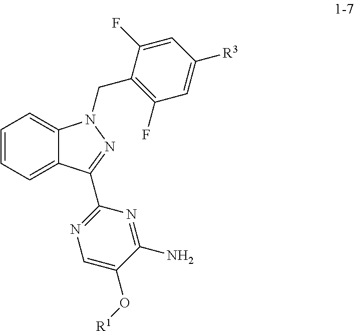
in which R¹, R³, are as defined in claim 1,
to react with a compound of general formula (1-8), 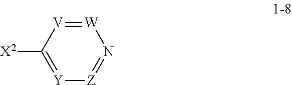
in which V, W, Y, and Z are as defined claim 1, and X² represents F, Cl, Br, I, boronic acid or a boronic acid ester,
thereby giving a compound of general formula (I): 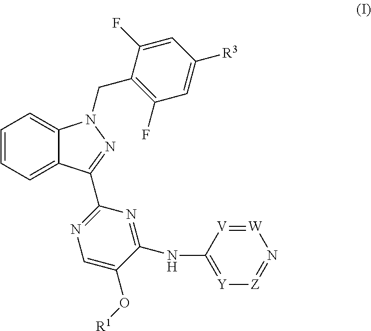
in which R¹, R³, V, W, Y, and Z are as defined in claim 1.

16: A compound of formula (1-7): 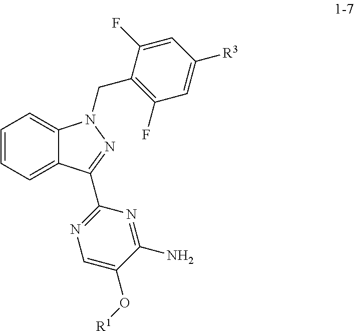
in which R¹, R³ are as defined in claim 1.

17. (canceled)
